(19) United States
(12) Patent Application Publication
(10) Pub. No.: US 2024/0398812 A1
(43) Pub. Date: Dec. 5, 2024

(54) MODULATORS OF TRPML, THEIR COMPOSITIONS AND METHODS OF USE

(71) Applicant: Caraway Therapeutics, Inc., Cambridge, MA (US)

(72) Inventors: Rajesh R. IYENGAR, West Newton, MA (US); Thomas Wai-Ho Lee, Lexington, MA (US); Casey Cameron McComas, Phoenixville, PA (US); Darby R. Schmidt, Arlington, MA (US); John J. Graziotto, Somerville, MA (US)

(73) Assignee: Caraway Therapeutics, Inc., Cambridge, MA (US)

(21) Appl. No.: 18/695,885

(22) PCT Filed: Sep. 29, 2022
(86) PCT No.: PCT/US22/45199

§ 371 Date: Mar. 27, 2024

Related U.S. Application Data
(60) Provisional application No. 63/250,820, filed on Sep. 30, 2021.

(51) Int. Cl. A61K 31/519; C07D 487/04

(52) U.S. Cl. CPC A61K 31/519; C07D 487/04

(57) ABSTRACT

The present disclosure relates to pharmaceutical compounds of the Formulas I, Ia, Ib, or Ic or a pharmaceutically acceptable salt or composition thereof. Also provided are methods of use of TRPML modulators for treating disorders, the modulators including compounds of the Formulas I, Ia, Ib, or Ic. Such methods of use include treatment of ciliopathies.

### CROSS-REFERENCE TO RELATED APPLICATIONS

The application claims the benefit of, and priority to, U.S. Ser. No. 63/250,820, filed Sep. 30, 2021, the contents of which are incorporated herein by reference in their entirety.

### TECHNICAL FIELD

The present disclosure relates to compounds and compositions which are modulators of TRPML and are useful for treatment of a variety of disorders.

### BACKGROUND

The lysosome is a key organelle that serves as the cell's recycling center. In a highly regulated manner, it breaks down various biomaterials (proteins, lipids, and membranes) into smaller molecules or chemical building blocks, that the cell then employs for energy or as CWTstarting materials for new proteins or membranes [see e.g., de Duve, C., The lysosome turns fifty. Nat Cell Biol, 2005. 7(9): p. 847-9. Parkinson-Lawrence, E. J., et al., Lysosomal storage disease: revealing lysosomal function and physiology. Physiology (Bethesda), 2010. 25(2): p. 102-15]. Lysosomal dysfunction due to mutations in the hydrolytic enzyme of lysosomal transport occur in the more than 50 genetically defined Lysosomal Storage Diseases. Interestingly, defects in lysosomal processing can have substantial effects on the function of the organelle beyond the actual enzyme that is mutated—in effect, the system can be gummed up—altering lysosomal degradation and membrane transport/trafficking, creating a positive feedback loop. Because lysosome storage is also seen in common neurodegenerative diseases such as Alzheimer's and Parkinson's, understanding the mechanisms underlying the positive feedback loop may provide therapeutic approaches not only for LSDs, but also for common sporadic neurodegenerative diseases. A lysosome-localized cation channel, TRPML1, has been recently identified as a key regulator of lysosomal function and membrane trafficking processes in the lysosome. Human mutations of TRPML1 cause an inherited lysosomal storage disease, Mucolipidosis IV. This disease is typified by neurodegenerative effects likely driven by the accumulation of lipids and other biomaterials in the cell. The related channels TRPML2 and TRPML3 also regulate lysosomal function.

Many reports suggest that TRPML channel activation is involved in multiple, key lysosomal functions. It can drive the translocation of the Transcription factor (TF)EB to the nucleus. TFEB regulates autophagy and lysosome biogenesis. Overexpression of TFEB has been reported to induce cellular clearance in several lysosome storage diseases, including Pompe Disease, Cystinosis, multiple sulfatase deficiency, as well as common neurodegenerative diseases, including Parkinson's disease and Huntington's disease (Settembre, C., et al., Signals from the lysosome: a control centre for cellular clearance and energy metabolism. Nat Rev Mol Cell Biol, 2013. 14 (5): p. 283-96). Therefore, activation of TRPML channels by TRPML agonists may also lead to cellular clearance in all the aforementioned diseases, providing therapeutic targets for these devastating diseases.

Recently, a potent synthetic agonist for TRPML1 has been reported [Shen, D., et al., Lipid storage disorders block lysosomal trafficking by inhibiting a TRP channel and lysosomal calcium release. Nat Commun, 2012. 3: p. 731]. This SF-51-related compound (Mucolipin Synthetic Agonist 1 or ML-SA1) that could induce significant [Ca2+] increases in HEK293 cells stably or transiently expressing TRPML1 protein that has been forced to the plasma membrane via deletion of its lysosomal targeting sequence. High concentrations of ML-SA1 (˜10 μM) are needed to effectively activate TRPMLs. Since that concentration is usually difficult to achieve in vivo, ML-SA1 cannot be used to treat the above TRPML related diseases. Liang et al. recently reported a new class of compounds as more potent TRPML activators [WO 2018/005713A1]. These compounds were thought to be useful in treating disorders related to TRPML activities such as lysosome storage diseases, muscular dystrophy, age-related common neurodegenerative diseases, ROS or oxidative stress related diseases, and aging. TRPML activators may also be useful in other disorders.

### SUMMARY OF THE DISCLOSURE

The present disclosure provides for a compound of Formula (I) and pharmaceutically acceptable salts, solvates, hydrates, tautomers, and stereoisomers thereof:

In an aspect, provided is a compound of Formula (I):

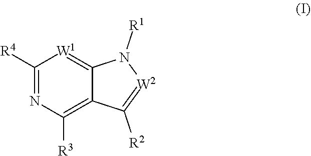

or a pharmaceutically acceptable salt thereof, wherein
W¹ is N or CR⁵;
W² is N or CR⁶;
R¹ is aryl, heteroaryl, cycloalkyl, or heterocycloalkyl, each R¹ optionally substituted by 1-5 independently selected R⁷;
R² is aryl, heteroaryl, cycloalkyl, heterocycloalkyl, C₁₋₆ alkyl, —(CH₂)₁₋₂-cycloalkyl, —(CH₂)₁₋₂-heterocycloalkyl, or NR^{a}R^{b}, each R² optionally substituted by 1-5 independently selected R⁸;
R³ is

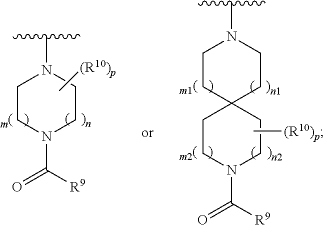

each of R⁴, R⁵, and R⁶ is independently selected from the group consisting of H, hydroxy, halogen, cyano, C₁₋₆ alkyl, C₁₋₆ haloalkyl, C₁₋₆ alkoxy, C₁₋₆ haloalkoxy, C₁₋₆ alkylthio, and NR^{a}R^{b}.
each of R⁷ and R⁸ are independently selected at each occurrence from the group consisting of deuterium, hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇ cycloalkyl is optionally substituted with substituents independently selected from the group consisting of halogen, hydroxyl, C₁₋₆ alkoxy and C₁₋₆ alkyl; or
when R¹ or R² is cycloalkyl or heterocycloalkyl, two R⁷ or two R⁸ can be taken together to form oxo, or any two R⁷ or two R⁸ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
R⁹ is C₁₋₆ alkyl or —(CH₂)₀₋₂—C₃₋₇ cycloalkyl, optionally substituted with substituents independently selected from the group consisting of deuterium, halogen, hydroxyl, C₁₋₃ alkyl and C₁₋₆ alkoxy, wherein C₁₋₃ alkyl and C₁₋₆ alkoxy are optionally substituted with substituents independently selected from the group consisting of halogen and hydroxy;
each R¹⁰ is selected independently from the group consisting of C₁₋₆ alkyl, and C₁₋₆ haloalkyl, optionally substituted with substituents independently selected from the group consisting of deuterium, hydroxyl, C₁₋₃ haloalkyl, C₁₋₃ alkoxy and C₁₋₃ alkyl; or
two R¹⁰ on the same carbon can be taken together to form oxo; or
any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of deuterium, halogen, hydroxyl, C₁₋₆ haloalkyl, and C₁₋₆ alkyl;
each R^{a} and R^{b} is independently selected from H, C₁₋₆ alkyl, —(CH₂)₀₋₂—C₃₋₇ cycloalkyl, and 3-7 membered heterocycloalkyl;
m is 1 or 2;
n is 1, 2, or 3;
m1 is 0, 1 or 2;
n1 is 0, 1, 2, or 3;
m2 is 0, 1 or 2;
n2 is 0, 1, 2, or 3;
p is 0, 1, 2, 3, 4, 5, 6, 7, or 8;
wherein m+n is 2, 3, or 4;
m1+n1 is 0, 1, 2, 3, or 4; and
m2+n2 is 0, 1, 2, 3, or 4; and
R¹ and R² are not both aryl.

In another aspect, the disclosure provides a method of treating a disease or disorder that can be treated by modulation of TRPML, the method comprising administering to a patient in need thereof a compound described herein or a composition described herein.

Still other objects and advantages of the disclosure will become apparent to those of skill in the art from the disclosure herein, which is simply illustrative and not restrictive. Thus, other embodiments will be recognized by the skilled artisan without departing from the spirit and scope of the disclosure.

### DETAILED DESCRIPTION OF THE DISCLOSURE

As generally described herein, the present disclosure provides compounds (e.g., compounds of Formula (I), (Ia), (Ib), or (Ic)), or compounds of Table 1, or pharmaceutically acceptable salts thereof) that are useful for disorders (e.g., polycystic kidney disease) associated with modulation of TRPML. “TRPML”, “TRPML ion channel” and “TRPML channel” are used interchangeably throughout.

### Compounds

In an aspect, provided is a compound of Formula (I):

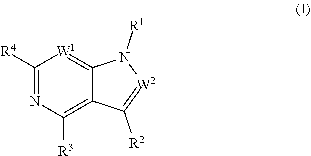

or a pharmaceutically acceptable salt thereof, wherein
W¹ is N or CR⁵;
W² is N or CR⁶;
R¹ is aryl, heteroaryl, cycloalkyl, or heterocycloalkyl, each R¹ optionally substituted by 1-5 independently selected R⁷;
R² is aryl, heteroaryl, cycloalkyl, heterocycloalkyl, C₁₋₆ alkyl, —(CH₂)₁₋₂-cycloalkyl, —(CH₂)₁₋₂-heterocycloalkyl, or NR^{a}R^{b}, each R² optionally substituted by 1-5 independently selected R⁸;
R³ is

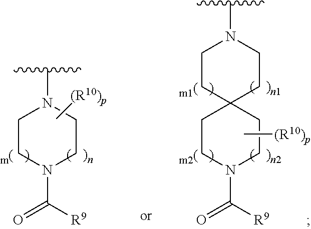

each of R⁴, R⁵, and R⁶ is independently selected from the group consisting of H, hydroxy, halogen, cyano, C₁₋₆ alkyl, C₁₋₆ haloalkyl, C₁₋₆ alkoxy, C₁₋₆ haloalkoxy, C₁₋₆ alkylthio, and NR^{a}R^{b};
each of R⁷ and R⁸ are independently selected at each occurrence from the group consisting of deuterium, hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇ cycloalkyl is optionally substituted with substituents independently selected from the group consisting of halogen, hydroxyl, C₁₋₆ alkoxy and C₁₋₆ alkyl; or
when R¹ or R² is cycloalkyl or heterocycloalkyl, two R⁷ or two R⁸ can be taken together to form oxo, or any two R⁷ or two R⁸ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
R⁹ is C₁₋₆ alkyl or —(CH₂)₀₋₂—C₃₋₇ cycloalkyl, optionally substituted with substituents independently selected from the group consisting of deuterium, halogen, hydroxyl, C₁₋₃ alkyl and C₁₋₆ alkoxy, wherein C₁₋₃ alkyl and C₁₋₆ alkoxy are optionally substituted with substituents independently selected from the group consisting of halogen and hydroxy;
each R¹⁰ is selected independently from the group consisting of C₁₋₆ alkyl, and C₁₋₆ haloalkyl, optionally substituted with substituents independently selected from the group consisting of deuterium, hydroxyl, C₁₋₃ haloalkyl, C₁₋₃ alkoxy and C₁₋₃ alkyl; or
two R¹⁰ on the same carbon can be taken together to form oxo; or
any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of deuterium, halogen, hydroxyl, C₁₋₆ haloalkyl, and C₁₋₆ alkyl;
each R^{a} and R^{b} is independently selected from H, C₁₋₆ alkyl, —(CH₂)₀₋₂—C₃₋₇ cycloalkyl, and 3-7 membered heterocycloalkyl;
m is 1 or 2;
n is 1, 2, or 3;
m1 is 0, 1 or 2;
n1 is 0, 1, 2, or 3;
m2 is 0, 1 or 2;
n2 is 0, 1, 2, or 3;
p is 0, 1, 2, 3, 4, 5, 6, 7, or 8;
wherein m+n is 2, 3, or 4;
m1+n1 is 0, 1, 2, 3, or 4; and
m2+n2 is 0, 1, 2, 3, or 4; and
R¹ and R² are not both aryl.

In some embodiments, W¹ is N. In some embodiments, W² is CR⁶. In some embodiments, W¹ is N and W² is CR⁶. In some embodiments, W¹ is CR⁵ and W² is N. In some embodiments, W¹ is N and W² is N. In some embodiments, W¹ is CR⁵ and W² is CR⁶.

In some embodiments, the compound is of formula (Ia)

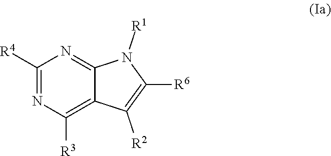

or a pharmaceutically acceptable salt thereof, wherein
R¹ is aryl, heteroaryl, cycloalkyl, or heterocycloalkyl, each R¹ optionally substituted by 1-5 independently selected R⁷;
R² is aryl, heteroaryl, cycloalkyl, heterocycloalkyl, C₁₋₆ alkyl, or NR^{a}R^{b}, each R² optionally substituted by 1-5 independently selected R⁸;
R³ is

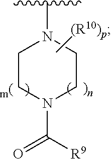

each of R⁴ and R⁶ is independently selected from the group consisting of H, hydroxy, halogen, cyano, C₁₋₆ alkyl, C₁₋₆ haloalkyl, C₁₋₆ alkoxy, C₁₋₆ haloalkoxy, C₁₋₆ alkylthio, and NR^{a}R^{b};
each of R⁷ and R⁸ are independently selected at each occurrence from the group consisting of hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇ cycloalkyl is optionally substituted with substituents independently selected from the group consisting of halogen, hydroxyl, C₁₋₆ alkoxy and C₁₋₆ alkyl; or
when R¹ or R² is cycloalkyl or heterocycloalkyl, two R⁷ or two R⁸ can be taken together to form oxo, or any two R⁷ or two R⁸ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
R⁹ is C₁₋₆ alkyl or C₃₋₇ cycloalkyl, optionally substituted with 1-9 substituents independently selected from the group consisting of deuterium, halogen, C₁₋₃ alkyl, hydroxyl, and C₁₋₆ alkoxy, wherein C₁₋₃ alkyl and C₁₋₆ alkoxy are optionally substituted with 1-3 substituents independently selected from the group consisting of halogen and hydroxy;
each R¹⁰ is selected independently from the group consisting of C₁₋₆ alkyl, and C₁₋₆ haloalkyl; or
two R¹⁰ on the same carbon can be taken together to form oxo; or
any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of deuterium, halogen, hydroxyl, C₁₋₆ haloalkyl, and C₁₋₆ alkyl;
each R^{a} and R^{b} is independently selected from H, C₁₋₆ alkyl, —(CH₂)₀₋₂—C₃₋₇ cycloalkyl, and 3-7 membered heterocycloalkyl;
m is 1 or 2;
n is 1, 2, or 3;
p is 0, 1, 2, 3, 4, 5, 6, 7, or 8;
wherein m+n is 2, 3, or 4; and
R¹ and R² are not both aryl.

In some embodiments, the compound is of formula (Ia)

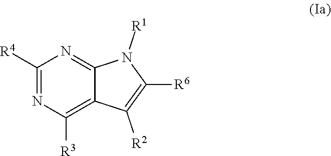

or a pharmaceutically acceptable salt thereof, wherein
R¹ is heteroaryl optionally substituted by 1-5 independently selected R⁷;
R² is aryl, heteroaryl, cycloalkyl, heterocycloalkyl, C₁₋₆ alkyl, or NR^{a}R^{b}, each R² optionally substituted by 1-5 independently selected R⁸;
R³ is

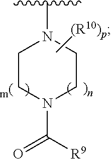

each of R⁴ and R⁶ is independently selected from the group consisting of H, hydroxy, halogen, cyano, C₁₋₆ alkyl, C₁₋₆ haloalkyl, C₁₋₆ alkoxy, C₁₋₆ haloalkoxy, C₁₋₆ alkylthio, and NR^{a}R^{b};
each of R⁷ and R⁸ are independently selected at each occurrence from the group consisting of hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇ cycloalkyl is optionally substituted with substituents independently selected from the group consisting of halogen, hydroxyl, C₁₋₆ alkoxy and C₁₋₆ alkyl; or
when R¹ or R² is cycloalkyl or heterocycloalkyl, two R⁷ or two R⁸ can be taken together to form oxo, or any two R⁷ or two R⁸ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
R⁹ is C₁₋₆ alkyl or C₃₋₇ cycloalkyl, optionally substituted with 1-9 substituents independently selected from the group consisting of deuterium, halogen, C₁₋₃ alkyl, hydroxyl, and C₁₋₆ alkoxy, wherein C₁₋₃ alkyl and C₁₋₆ alkoxy are optionally substituted with 1-3 substituents independently selected from the group consisting of halogen and hydroxy;
each R¹⁰ is selected independently from the group consisting of C₁₋₆ alkyl, and C₁₋₆ haloalkyl, optionally substituted with 1-5 substituents independently selected from the group consisting of deuterium, hydroxyl, C₁₋₃ haloalkyl, C₁₋₃ alkoxy and C₁₋₃ alkyl; or
two R¹⁰ on the same carbon can be taken together to form oxo; or
any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of deuterium, halogen, hydroxyl, C₁₋₆ haloalkyl, and C₁₋₆ alkyl;
each R^{a} and R^{b} is independently selected from H, C₁₋₆ alkyl, —(CH₂)₀₋₂—C₃₋₇ cycloalkyl, and 3-7 membered heterocycloalkyl;
m is 1 or 2;
n is 1, 2, or 3;
p is 0, 1, 2, 3, 4, 5, 6, 7, or 8; and
wherein m+n is 2, 3, or 4.

In some embodiments, the compound of formula (Ia)

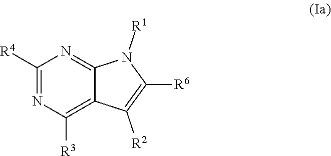

or a pharmaceutically acceptable salt thereof, wherein
R¹ is pyridyl optionally substituted by 1-5 independently selected R⁷;
R² is aryl, heteroaryl, cycloalkyl, heterocycloalkyl, C₁₋₆ alkyl, or NR^{a}R^{b}, each R² optionally substituted by 1-5 independently selected R⁸;
R³ is

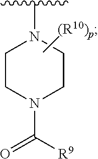

each of R⁴ and R⁶ is H;
each of R⁷ and R⁸ are independently selected at each occurrence from the group consisting of hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇ cycloalkyl is optionally substituted with substituents independently selected from the group consisting of halogen, hydroxyl, C₁₋₆ alkoxy and C₁₋₆ alkyl;
R⁹ is C₁₋₆ alkyl or C₃₋₇ cycloalkyl, optionally substituted with 1-9 substituents independently selected from the group consisting of deuterium, halogen, C₁₋₃ alkyl, hydroxyl, and C₁₋₆ alkoxy, wherein C₁₋₃ alkyl and C₁₋₆ alkoxy are optionally substituted with 1-3 substituents independently selected from the group consisting of halogen and hydroxy;
each R¹⁰ is selected independently from the group consisting of C₁₋₆ alkyl, and C₁₋₆ haloalkyl, optionally substituted with 1-5 substituents independently selected from the group consisting of deuterium, hydroxyl, C₁₋₃ haloalkyl, C₁₋₃ alkoxy and C₁₋₃ alkyl; or
two R¹⁰ on the same carbon can be taken together to form oxo; or
any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of deuterium, halogen, hydroxyl, C₁₋₆ haloalkyl, and C₁₋₆ alkyl;
each R^{a} and R^{b} is independently selected from H, C₁₋₆ alkyl, —(CH₂)₀₋₂—C₃₋₇ cycloalkyl, and 3-7 membered heterocycloalkyl; and
p is 0, 1, 2, 3, 4, 5, 6, 7, or 8.

In some embodiments, the compound is of formula (Ia)

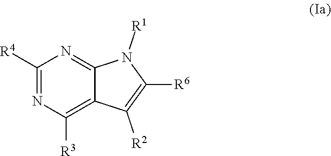

or a pharmaceutically acceptable salt thereof, wherein
R¹ is heteroaryl optionally substituted by 1-5 independently selected R⁷;
R² is aryl optionally substituted by 1-5 independently selected R⁸;
R³ is

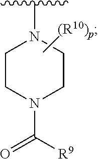

each of R⁴ and R⁶ is H;
each of R⁷ and R⁸ are independently selected at each occurrence from the group consisting of hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇ cycloalkyl is optionally substituted with substituents independently selected from the group consisting of halogen, hydroxyl, C₁₋₆ alkoxy and C₁₋₆ alkyl;
R⁹ is C₁₋₆ alkyl or C₃₋₇ cycloalkyl, optionally substituted with 1-9 substituents independently selected from the group consisting of deuterium, halogen, C₁₋₃ alkyl, hydroxyl, and C₁₋₆ alkoxy, wherein C₁₋₃ alkyl and C₁₋₆ alkoxy are optionally substituted with 1-3 substituents independently selected from the group consisting of halogen and hydroxy;
each R¹⁰ is selected independently from the group consisting of C₁₋₆ alkyl, and C₁₋₆ haloalkyl, optionally substituted with 1-5 substituents independently selected from the group consisting of deuterium, hydroxyl, C₁₋₃ haloalkyl, C₁₋₃ alkoxy and C₁₋₃ alkyl; or
two R¹⁰ on the same carbon can be taken together to form oxo; or
any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of deuterium, halogen, hydroxyl, C₁₋₆ haloalkyl, and C₁₋₆ alkyl;
each R^{a} and R^{b} is independently selected from H, C₁₋₆ alkyl, —(CH₂)₀₋₂—C₃₋₇ cycloalkyl, and 3-7 membered heterocycloalkyl; and
p is 0, 1, 2, 3, 4, 5, 6, 7, or 8.

In some embodiments, the compound is of formula (Ia)

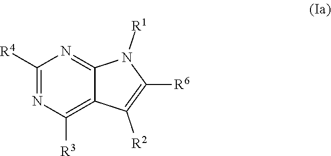

or a pharmaceutically acceptable salt thereof, wherein
R¹ is pyridyl optionally substituted by 1-5 independently selected R⁷;
R² is phenyl optionally substituted by 1-5 independently selected R⁸;
R³ is

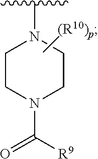

each of R⁴ and R⁶ is H;
each of R⁷ and R⁸ are independently selected at each occurrence from the group consisting of hydroxy, halogen, cyano, C₁₋₆ alkyl, and C₁₋₆ alkoxy;
R⁹ is C₁₋₆ alkyl or C₃₋₇ cycloalkyl, optionally substituted with 1-9 substituents independently selected from the group consisting of deuterium, halogen, C₁₋₃ alkyl, hydroxyl, and C₁₋₆ alkoxy, wherein C₁₋₃ alkyl and C₁₋₆ alkoxy are optionally substituted with 1-3 substituents independently selected from the group consisting of halogen and hydroxy;
each R¹⁰ is selected independently from the group consisting of C₁₋₆ alkyl, and C₁₋₆ haloalkyl, each optionally substituted with 1-5 deuterium; and
p is 0, 1, 2, 3, 4, 5, 6, 7, or 8.

In some embodiments, R¹ is aryl optionally substituted by 1-5 independently selected R⁷. R¹ is phenyl optionally substituted with 1-3 independently selected R⁷.

In some embodiments, each R⁷ is independently selected from H, halogen, cyano, C₁₋₆ alkyl, C₁₋₆ alkoxy, C₁₋₆ haloalkyl, or C₁₋₆ haloalkoxy.

In some embodiments, R¹ is heteroaryl, cycloalkyl, or heterocycloalkyl, each R¹ optionally substituted by 1-5 independently selected R⁷. In some embodiments, R¹ is heteroaryl or heterocycloalkyl, each R¹ optionally substituted by 1-5 independently selected R⁷. In some embodiments, R¹ is heteroaryl optionally substituted by 1-5 independently selected R⁷. In some embodiments, R¹ is monocyclic heteroaryl optionally substituted by 1-5 independently selected R⁷. In some embodiments, R¹ is monocyclic heteroaryl of 5-6 ring atoms with 1, 2 or 3 ring atoms selected independently from N, O, and S, wherein R¹ is optionally substituted by 1-4 independently selected R⁷. In some embodiments, R¹ is monocyclic nitrogen-containing heteroaryl of 5-6 ring atoms with 1, 2 or 3 ring heteroatoms selected from N only, wherein R¹ is optionally substituted by 1-4 independently selected R⁷. In some embodiments, R¹ is pyridine, pyrimidine, pyrazine, pyridazine, thiazole, oxazole, pyrrole, imidazole, or pyrazole, optionally substituted by 1-4 independently selected R⁷. In some embodiments, R¹ is pyridine, thiazole, or pyrazole, optionally substituted by 1-4 independently selected R⁷. In some embodiments, R¹ is pyridine, optionally substituted by 1-4 independently selected R⁷. In some embodiments, R¹ is 2-pyridyl, optionally substituted by 1-4 independently selected R⁷.

In some embodiments, R¹ is

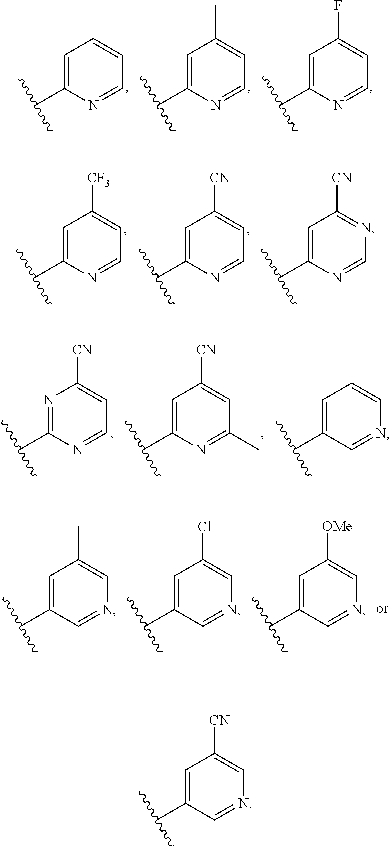

In some embodiments, R¹ is

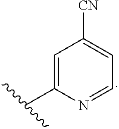 [SMILES: *c1cc(C#N)ccn1]

In some embodiments, R¹ is

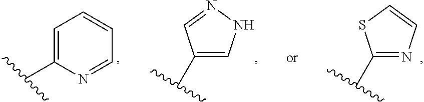 [SMILES: *c1ccccn1 ; *c1cn[nH]c1 ; *c1nccs1]

optionally substituted by 1-4 independently selected R⁷.

In some embodiments, R¹ is heterocycloalkyl of 4-8 ring atoms, wherein 1-3 ring atoms are selected from N, O, and S, and R¹ is optionally substituted by 1-4 independently selected R⁷. In some embodiments, R¹ is tetrahydropyran, azetidine, pyrrolidine, morpholine, or piperidine, and R¹ is optionally substituted by 1-4 independently selected R⁷.

In some embodiments, R¹ is C₃₋₇ cycloalkyl, optionally substituted by 1-4 independently selected R⁷. In some embodiments, R¹ is a cyclohexyl with an optional one or two carbon bridged ring, and R¹ is optionally substituted by 1-4 independently selected R⁷.

In some embodiments, R² is aryl, heteroaryl, cycloalkyl, heterocycloalkyl, C₁₋₅ alkyl, or NR^{a}R^{b}, each R² optionally substituted by 1-5 independently selected R⁸, and wherein R^{a} and R^{b} of the R² group are not both H. In some embodiments, R² is aryl optionally substituted by 1-5 independently selected R⁸. In some embodiments, R² is phenyl optionally substituted with 1-3 independently selected R⁸.

In some embodiments, each R⁸ is independently selected from H, halogen, cyano, C₁₋₆ alkyl, C₁₋₆ alkoxy, C₁₋₆ haloalkyl, or C₁₋₆ haloalkoxy.

In some embodiments, R² is

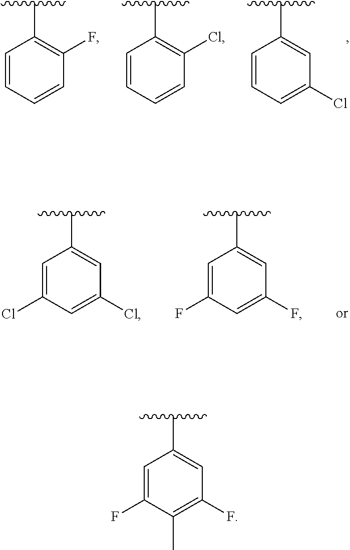

In some embodiments, R² is

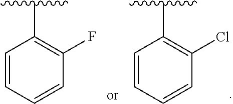

In some embodiments, R² is heteroaryl, cycloalkyl, heterocycloalkyl, or NR^{a}R^{b}, each R² optionally substituted by 1-5 independently selected R⁸, and wherein R^{a} and R^{b} of the R² group are not both H. In some embodiments, R² is heteroaryl or heterocycloalkyl, each R² optionally substituted by 1-5 independently selected R⁸. In some embodiments, R² is heteroaryl optionally substituted by 1-5 independently selected R⁸. In some embodiments, R² is monocyclic heteroaryl optionally substituted by 1-5 independently selected R⁸. In some embodiments, R² is monocyclic heteroaryl of 5-6 ring atoms with 1, 2 or 3 ring atoms selected independently from N, O, and S, wherein R² is optionally substituted by 1-4 independently selected R⁸. In some embodiments, R² is pyridine, pyrimidine, pyrazine, pyridazine, thiazole, oxazole, pyrrole, imidazole, or pyrazole, optionally substituted by 1-4 independently selected R⁸. In some embodiments, R² is pyridine, pyrimidine, pyrazine, or pyrazole, optionally substituted by 1-4 independently selected R⁸. In some embodiments, R² is pyridine, optionally substituted by 1-4 independently selected R⁸.

In some embodiments, R² is

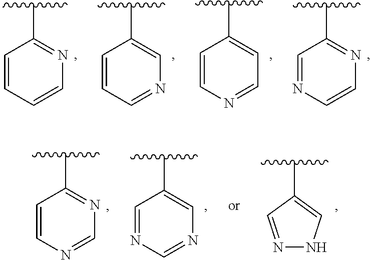

optionally substituted by 1-4 independently selected R⁸.

In some embodiments, R² is

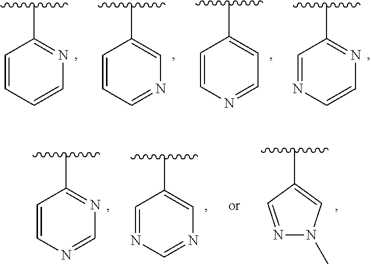

and wherein R² is not further substituted.

In some embodiments, R² is

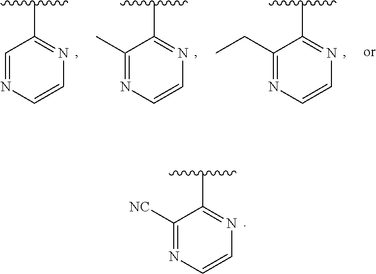

In some embodiments, R² is cycloalkyl optionally substituted by 1-5 independently selected R⁸. In some embodiments, R² is C₃₋₈ cycloalkyl optionally substituted with 1-5 independently selected R⁸. In some embodiments, R² is cyclopropyl, cyclobutyl, cyclopentyl, or cyclohexyl, each optionally substituted with 1-3 independently selected R⁸. In some embodiments, R² is cyclopropyl.

In some embodiments, R² is not substituted.

In some embodiments, R² is heterocycloalkyl optionally substituted by 1-5 independently selected R⁸. In some embodiments, R² is monocyclic heterocycloalkyl optionally substituted by 1-5 independently selected R⁸. In some embodiments, R² is monocyclic heterocycloalkyl of 4-6 ring atoms with 1, 2 or 3 ring atoms selected independently from N, O, and S, wherein R² is optionally substituted by 1-4 independently selected R⁸. In some embodiments, R² is azetidine, oxetane, pyrrolidine, tetrahydrofuran, piperidine, piperazine, tetrahydropyran, or morpholine, optionally substituted by 1-4 independently selected R⁸. In some embodiments, R² is azetidine, pyrrolidine, piperazine, or morpholine, optionally substituted by 1-4 independently selected R⁸. In some embodiments, R² contains a ring nitrogen atom and is bound to formula (I) at the ring nitrogen atom.

In some embodiments, R² is

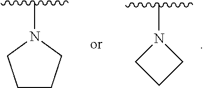 [SMILES: *N1CCCC1 ; *N1CCC1]

In some embodiments, R² is not substituted.

In some embodiments, R² is NR^{a}R^{b}, each R² optionally substituted by 1-5 independently selected R⁸, and wherein R^{a} and R^{b} of the R² group are not both H. In some embodiments, R^{a} is C₁₋₆ alkyl, and R^{b} is C₁₋₆ alkyl, C₃₋₇ cycloalkyl, or 3-7 membered heterocycloalkyl. In some embodiments, R^{a} and R^{b} are each independently selected C₁₋₆ alkyl.

In some embodiments, R² is C₁₋₆ alkyl optionally substituted by 1-5 independently selected R⁸. In some embodiments, R² is C₁₋₆ alkyl optionally substituted by 1-5 independently selected halogens. In some embodiments, R² is Me, Et, or CF₃.

In some embodiments, m is 1 and n is 1.

In some embodiments, p is 0, 1, 2, 3, 4, 5, or 6.

In some embodiments, each R¹⁰ is independently selected from the group consisting of C₁₋₆ alkyl and C₁₋₆ haloalkyl, each optionally substituted with 1-5 deuteriums. In some embodiments, each R¹⁰ is methyl.

In some embodiments, p is 1, 2, 3, 4, 5, or 6.

In some embodiments, R³ is substituted with an edge fused or spiro fused cyclopropane; or R³ includes a one or two carbon bridge; and R³ is optionally additionally substituted by 1-4 R¹⁰.

In some embodiments, R³ is

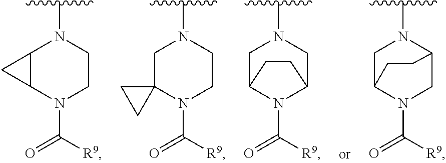

and R³ is optionally additionally substituted by 1-4 R¹⁰.

In some embodiments, R³ is

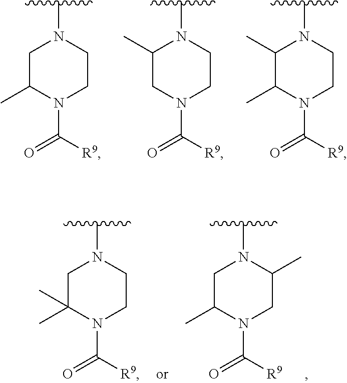

and R³ is optionally additionally substituted by 1-4 R¹⁰.

In some embodiments, R³ is

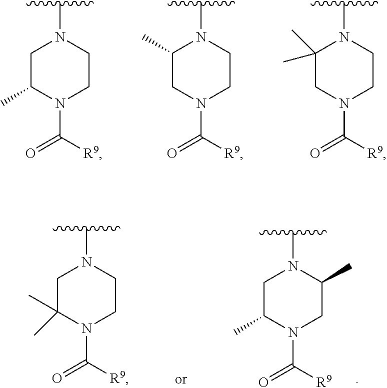

In some embodiments, R³ is not substituted by any additional R¹⁰.

In some embodiments, R⁴ is H. In some embodiments, R⁵ is H. In some embodiments, R⁶ is H. In some embodiments, R⁴ and R⁶ are H. In some embodiments, R⁴, R⁵, and R⁶ are H.

In some embodiments, each of R⁷ and R⁸ are independently selected at each occurrence from the group consisting of hydroxy, halogen, cyano, C₁₋₆ alkyl, C₁₋₆ alkoxy, and C₃₋₇ cycloalkyl, wherein each C₁₋₆ alkyl and C₁₋₆ alkoxy is optionally substituted with 1-3 halogens. In some embodiments, each R⁷ is independently selected at each occurrence from the group consisting of halogen, cyano, C₁₋₆ alkyl, C₁₋₆ alkoxy, and CF₃.

In some embodiments, R⁹ is C₁₋₆ alkyl, optionally substituted with 1-3 substituents independently selected from the group consisting of halogen, hydroxyl, and C₁₋₆ alkoxy, or with 1-9 substituents selected from deuterium and halogen. In some embodiments, R⁹ is t-butyl, ethyl, or isopropyl, optionally substituted with 1-9 substituents selected from deuterium, hydroxy, and halogen or with 1-3 hydroxyl. In some embodiments, R⁹ is t-butyl.

In some embodiments, R⁹ is C₃₋₇ cycloalkyl, optionally substituted with 1-3 substituents independently selected from the group consisting of deuterium, halogen, hydroxyl, C₁₋₃ alkyl, and C₁₋₆ alkoxy, wherein C₁₋₃ alkyl and C₁₋₆ alkoxy are optionally substituted with 1-3 substituents independently selected from the group consisting of halogen and hydroxy. In some embodiments, R⁹ is C₃₋₇ cycloalkyl, optionally substituted with 1-3 substituents independently selected from the group consisting of halogen, hydroxyl, and C₁₋₆ alkoxy. In some embodiments, R⁹ is cyclopropyl.

In some embodiments, R⁹ is Me, Et, t-butyl, isopropyl, cyclopropyl, isobutyl, neopentyl, F₃C—CH₂—, F₃C—CH(CH₃)—, F₃C—C(CH₃)₂—, or FCH₂—C(CH₃)₂—,

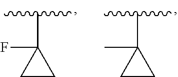

CD₃, —CD(CD₃)₂, —C(CD₃)₃, or

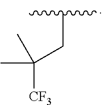 [SMILES: *CC(C)(C)C(F)(F)F]

In some embodiments, R⁹ is Me, Et, t-butyl, isopropyl, cyclopropyl, isobutyl, neopentyl, F₃C—CH₂—, F₃C—CH(CH₃)—, F₃C—C(CH₃)₂—, or FCH₂—C(CH₃)₂—,

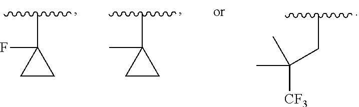

In some embodiments, the compound is of formula (Ib)

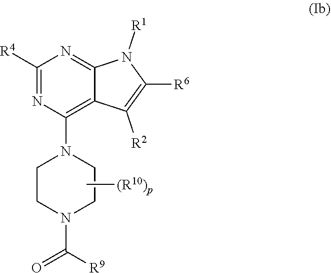

wherein the variable definitions are as described in the specification and claims.

In some embodiments, the compound is of formula (Ic)

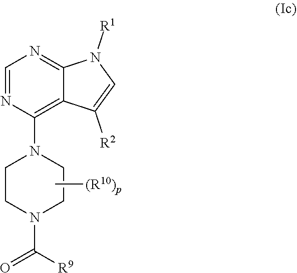

wherein the variable definitions are as described in the specification and claims.

In some embodiments, the compound of Formula (I), or any subformula thereof, is selected from the compounds disclosed in the specification, or a pharmaceutically acceptable salt thereof.

In some embodiments, the compound achieves at least 50% of the maximal current obtained with 30 μM ML-SA1 in a patch clamp assay for a TRPML and has an EC₅₀ less than 1 μM. In some embodiments, the compound achieves at least 50% of the maximal current obtained with 30 μM ML-SA1 in a patch clamp assay for TRPML1 and has an EC₅₀ less than 1 μM.

In some embodiments, the compound achieves a maximal current obtained with 30 μM ML-SA1 in a patch clamp assay for TRPML1 which is at least 10 fold the maximal current achieved for any other TRPML.

Also provided is a pharmaceutical composition comprising a pharmaceutically acceptable excipient and a compound disclosed herein.

In some embodiments, the compound is a compound identified in Table 1 below or a pharmaceutically acceptable salt thereof.

TABLE 1
Exemplary compounds

| Cmpd | |
|---|---|
| No. | Structure |
| 101 | 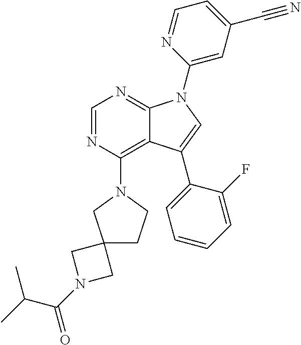 [SMILES: CC(C)C(=O)N1CC2(CCN(c3ncnc4c3c(-c3ccccc3F)cn4-c3cc(C#N)ccn3)C2)C1] |
| 102 | 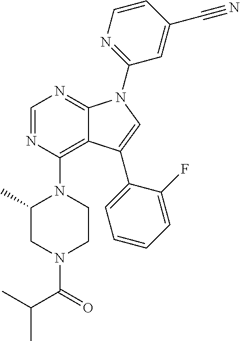 [SMILES: CC(C)C(=O)N1CCN(c2ncnc3c2c(-c2ccccc2F)cn3-c2cc(C#N)ccn2)[C@@H](C)C1] |
| 103 | 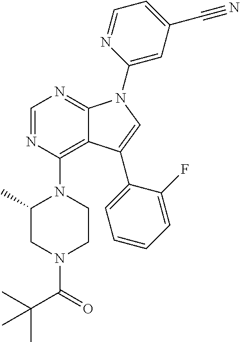 [SMILES: C[C@H]1CN(C(=O)C(C)(C)C)CCN1c1ncnc2c1c(-c1ccccc1F)cn2-c1cc(C#N)ccn1] |
| 104 | 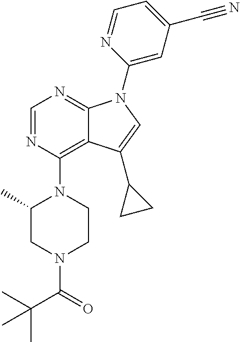 [SMILES: C[C@H]1CN(C(=O)C(C)(C)C)CCN1c1ncnc2c1c(C1CC1)cn2-c1cc(C#N)ccn1] |
| 105 | 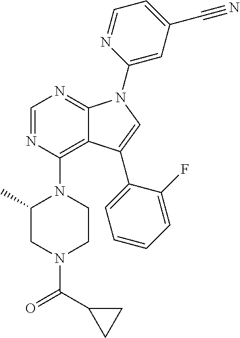 [SMILES: C[C@H]1CN(C(=O)C2CC2)CCN1c1ncnc2c1c(-c1ccccc1F)cn2-c1cc(C#N)ccn1] |
| 106 | 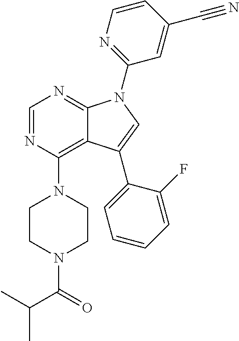 [SMILES: CC(C)C(=O)N1CCN(c2ncnc3c2c(-c2ccccc2F)cn3-c2cc(C#N)ccn2)CC1] |
| 107 | 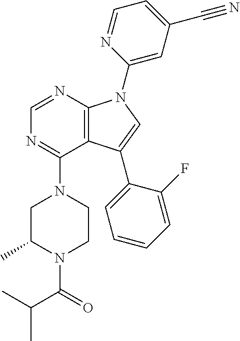 [SMILES: CC(C)C(=O)N1CCN(c2ncnc3c2c(-c2ccccc2F)cn3-c2cc(C#N)ccn2)C[C@H]1C] |
| 108 | 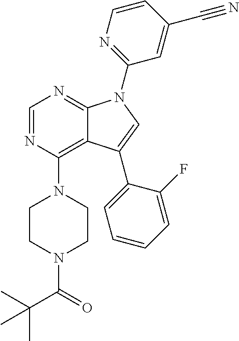 [SMILES: CC(C)(C)C(=O)N1CCN(c2ncnc3c2c(-c2ccccc2F)cn3-c2cc(C#N)ccn2)CC1] |
| 109 | 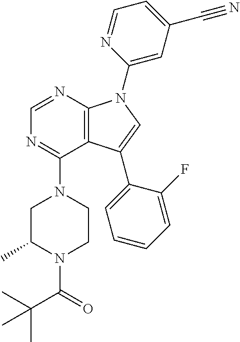 [SMILES: C[C@@H]1CN(c2ncnc3c2c(-c2ccccc2F)cn3-c2cc(C#N)ccn2)CCN1C(=O)C(C)(C)C] |
| 110 | 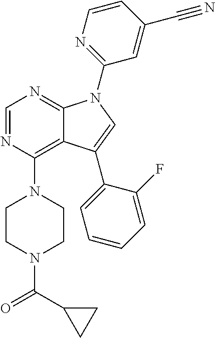 [SMILES: N#Cc1ccnc(-n2cc(-c3ccccc3F)c3c(N4CCN(C(=O)C5CC5)CC4)ncnc32)c1] |
| 111 | 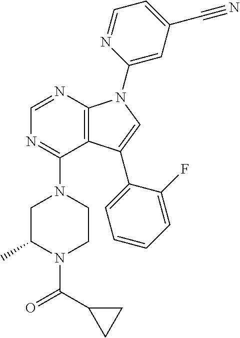 [SMILES: C[C@@H]1CN(c2ncnc3c2c(-c2ccccc2F)cn3-c2cc(C#N)ccn2)CCN1C(=O)C1CC1] |
| 112 | 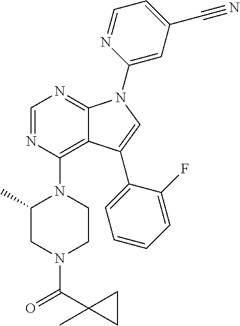 [SMILES: C[C@H]1CN(C(=O)C2(C)CC2)CCN1c1ncnc2c1c(-c1ccccc1F)cn2-c1cc(C#N)ccn1] |
| 113 | 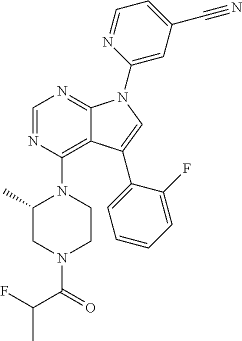 [SMILES: CC(F)C(=O)N1CCN(c2ncnc3c2c(-c2ccccc2F)cn3-c2cc(C#N)ccn2)[C@@H](C)C1] |
| 114 | 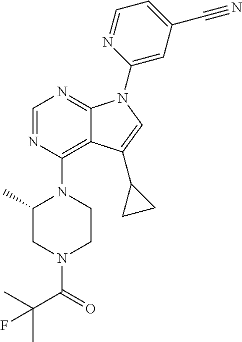 [SMILES: C[C@H]1CN(C(=O)C(C)(C)F)CCN1c1ncnc2c1c(C1CC1)cn2-c1cc(C#N)ccn1] |
| 115 | 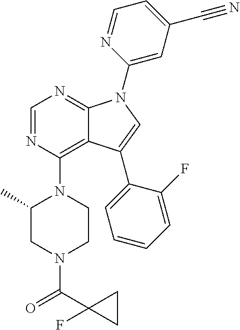 [SMILES: C[C@H]1CN(C(=O)C2(F)CC2)CCN1c1ncnc2c1c(-c1ccccc1F)cn2-c1cc(C#N)ccn1] |
| 116 | 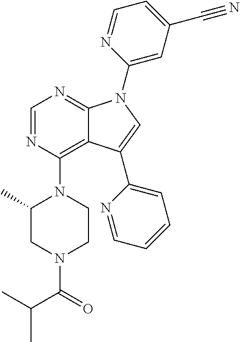 [SMILES: CC(C)C(=O)N1CCN(c2ncnc3c2c(-c2ccccn2)cn3-c2cc(C#N)ccn2)[C@@H](C)C1] |
| 117 | 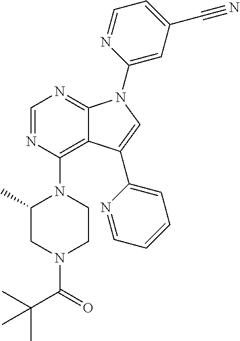 [SMILES: C[C@H]1CN(C(=O)C(C)(C)C)CCN1c1ncnc2c1c(-c1ccccn1)cn2-c1cc(C#N)ccn1] |
| 118 | 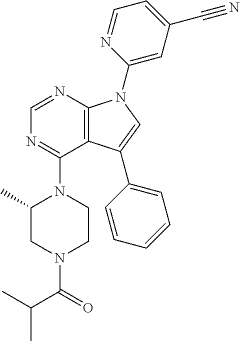 [SMILES: CC(C)C(=O)N1CCN(c2ncnc3c2c(-c2ccccc2)cn3-c2cc(C#N)ccn2)[C@@H](C)C1] |
| 119 | 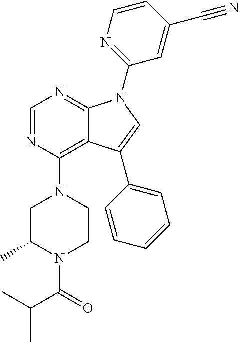 [SMILES: CC(C)C(=O)N1CCN(c2ncnc3c2c(-c2ccccc2)cn3-c2cc(C#N)ccn2)C[C@H]1C] |
| 120 | 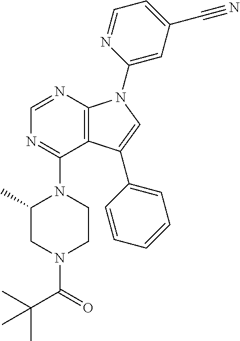 [SMILES: C[C@H]1CN(C(=O)C(C)(C)C)CCN1c1ncnc2c1c(-c1ccccc1)cn2-c1cc(C#N)ccn1] |
| 121 | 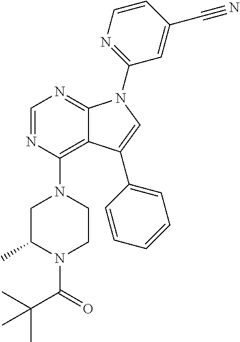 [SMILES: C[C@@H]1CN(c2ncnc3c2c(-c2ccccc2)cn3-c2cc(C#N)ccn2)CCN1C(=O)C(C)(C)C] |
| 122 | 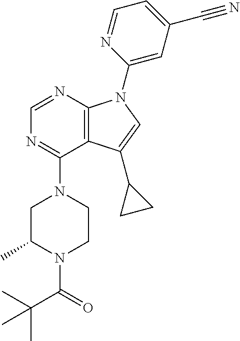 [SMILES: C[C@@H]1CN(c2ncnc3c2c(C2CC2)cn3-c2cc(C#N)ccn2)CCN1C(=O)C(C)(C)C] |
| 123 | 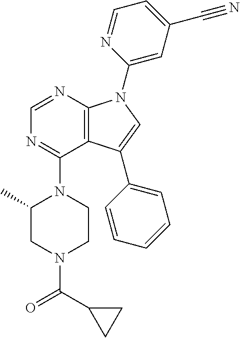 [SMILES: C[C@H]1CN(C(=O)C2CC2)CCN1c1ncnc2c1c(-c1ccccc1)cn2-c1cc(C#N)ccn1] |
| 124 | 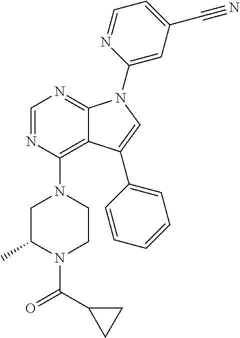 [SMILES: C[C@@H]1CN(c2ncnc3c2c(-c2ccccc2)cn3-c2cc(C#N)ccn2)CCN1C(=O)C1CC1] |
| 125 | 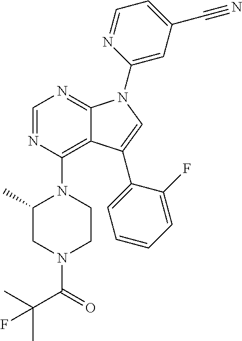 [SMILES: C[C@H]1CN(C(=O)C(C)(C)F)CCN1c1ncnc2c1c(-c1ccccc1F)cn2-c1cc(C#N)ccn1] |
| 126 | 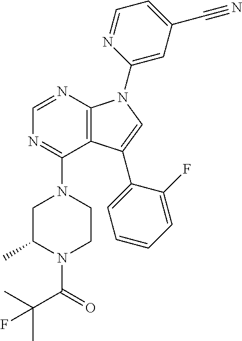 [SMILES: C[C@@H]1CN(c2ncnc3c2c(-c2ccccc2F)cn3-c2cc(C#N)ccn2)CCN1C(=O)C(C)(C)F] |
| 127 | 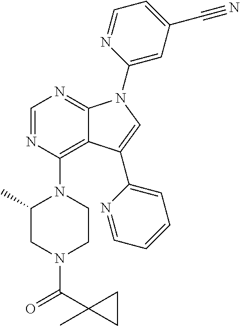 [SMILES: C[C@H]1CN(C(=O)C2(C)CC2)CCN1c1ncnc2c1c(-c1ccccn1)cn2-c1cc(C#N)ccn1] |
| 128 | 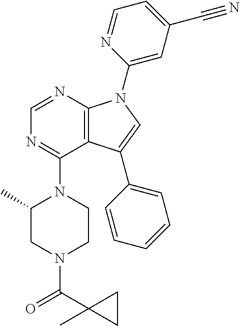 [SMILES: C[C@H]1CN(C(=O)C2(C)CC2)CCN1c1ncnc2c1c(-c1ccccc1)cn2-c1cc(C#N)ccn1] |
| 129 | 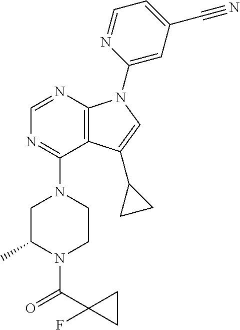 [SMILES: C[C@@H]1CN(c2ncnc3c2c(C2CC2)cn3-c2cc(C#N)ccn2)CCN1C(=O)C1(F)CC1] |
| 130 | 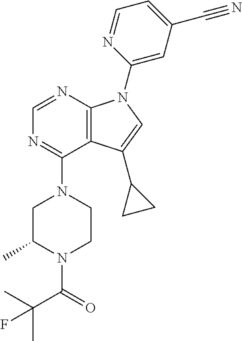 [SMILES: C[C@@H]1CN(c2ncnc3c2c(C2CC2)cn3-c2cc(C#N)ccn2)CCN1C(=O)C(C)(C)F] |
| 131 | 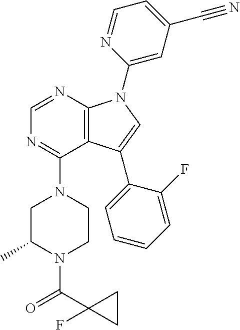 [SMILES: C[C@@H]1CN(c2ncnc3c2c(-c2ccccc2F)cn3-c2cc(C#N)ccn2)CCN1C(=O)C1(F)CC1] |
| 132 | 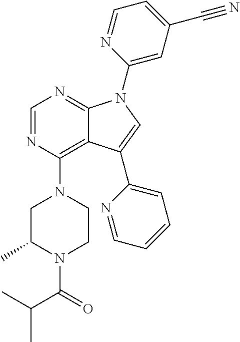 [SMILES: CC(C)C(=O)N1CCN(c2ncnc3c2c(-c2ccccn2)cn3-c2cc(C#N)ccn2)C[C@H]1C] |
| 133 | 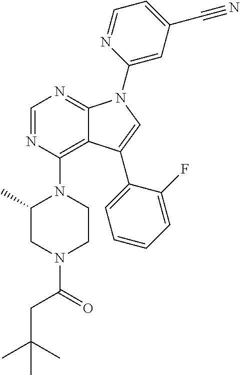 [SMILES: C[C@H]1CN(C(=O)CC(C)(C)C)CCN1c1ncnc2c1c(-c1ccccc1F)cn2-c1cc(C#N)ccn1] |
| 134 | 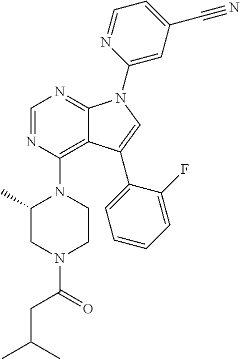 [SMILES: CC(C)CC(=O)N1CCN(c2ncnc3c2c(-c2ccccc2F)cn3-c2cc(C#N)ccn2)[C@@H](C)C1] |
| 135 | 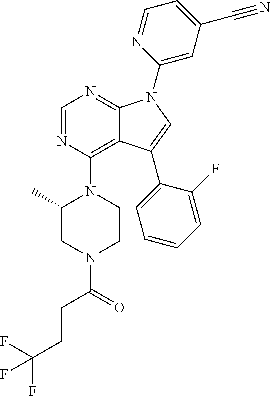 [SMILES: C[C@H]1CN(C(=O)CCC(F)(F)F)CCN1c1ncnc2c1c(-c1ccccc1F)cn2-c1cc(C#N)ccn1] |
| 136 | 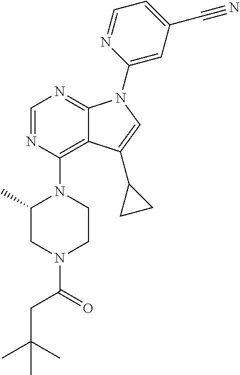 [SMILES: C[C@H]1CN(C(=O)CC(C)(C)C)CCN1c1ncnc2c1c(C1CC1)cn2-c1cc(C#N)ccn1] |
| 137 | 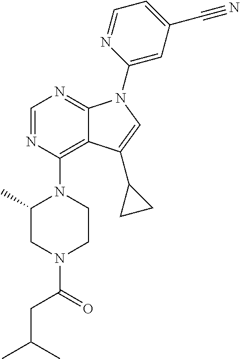 [SMILES: CC(C)CC(=O)N1CCN(c2ncnc3c2c(C2CC2)cn3-c2cc(C#N)ccn2)[C@@H](C)C1] |
| 138 | 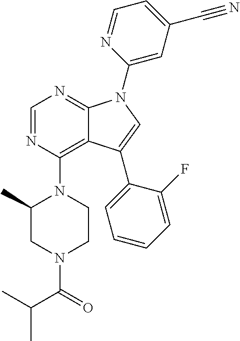 [SMILES: CC(C)C(=O)N1CCN(c2ncnc3c2c(-c2ccccc2F)cn3-c2cc(C#N)ccn2)[C@H](C)C1] |
| 139 | 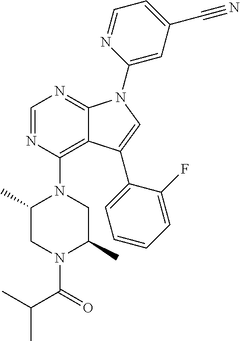 [SMILES: CC(C)C(=O)N1C[C@H](C)N(c2ncnc3c2c(-c2ccccc2F)cn3-c2cc(C#N)ccn2)C[C@H]1C] |
| 140 | 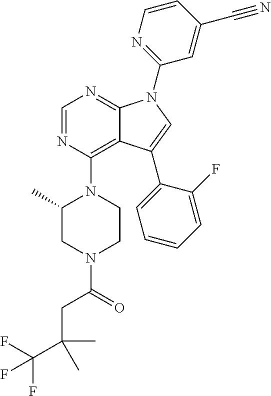 [SMILES: C[C@H]1CN(C(=O)CC(C)(C)C(F)(F)F)CCN1c1ncnc2c1c(-c1ccccc1F)cn2-c1cc(C#N)ccn1] |
| 141 | 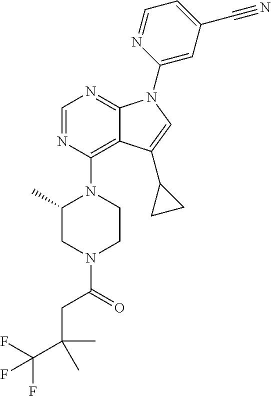 [SMILES: C[C@H]1CN(C(=O)CC(C)(C)C(F)(F)F)CCN1c1ncnc2c1c(C1CC1)cn2-c1cc(C#N)ccn1] |
| 142 | 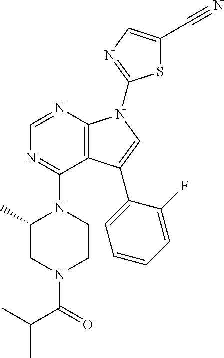 [SMILES: CC(C)C(=O)N1CCN(c2ncnc3c2c(-c2ccccc2F)cn3-c2ncc(C#N)s2)[C@@H](C)C1] |
| 143 | 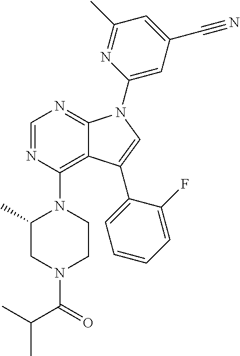 [SMILES: Cc1cc(C#N)cc(-n2cc(-c3ccccc3F)c3c(N4CCN(C(=O)C(C)C)C[C@@H]4C)ncnc32)n1] |
| 144 | 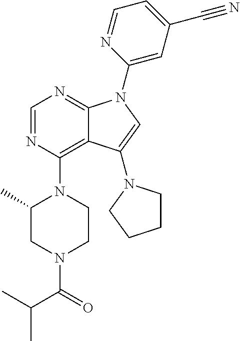 [SMILES: CC(C)C(=O)N1CCN(c2ncnc3c2c(N2CCCC2)cn3-c2cc(C#N)ccn2)[C@@H](C)C1] |
| 145 | 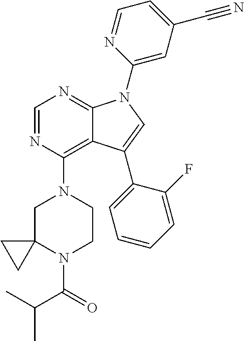 [SMILES: CC(C)C(=O)N1CCN(c2ncnc3c2c(-c2ccccc2F)cn3-c2cc(C#N)ccn2)CC12CC2] |
| 146 | 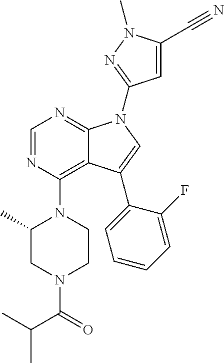 [SMILES: CC(C)C(=O)N1CCN(c2ncnc3c2c(-c2ccccc2F)cn3-c2cc(C#N)n(C)n2)[C@@H](C)C1] |
| 147 | 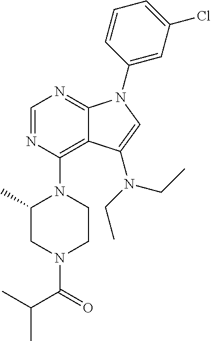 [SMILES: CCN(CC)c1cn(-c2cccc(Cl)c2)c2ncnc(N3CCN(C(=O)C(C)C)C[C@@H]3C)c12] |
| 148 | 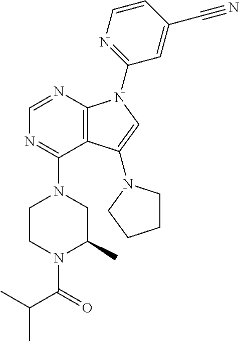 [SMILES: CC(C)C(=O)N1CCN(c2ncnc3c2c(N2CCCC2)cn3-c2cc(C#N)ccn2)C[C@H]1C] |
| 149 | 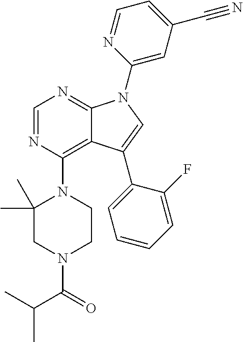 [SMILES: CC(C)C(=O)N1CCN(c2ncnc3c2c(-c2ccccc2F)cn3-c2cc(C#N)ccn2)C(C)(C)C1] |
| 150 | 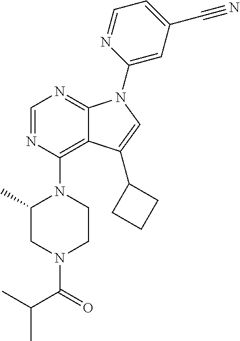 [SMILES: CC(C)C(=O)N1CCN(c2ncnc3c2c(C2CCC2)cn3-c2cc(C#N)ccn2)[C@@H](C)C1] |
| 151 | 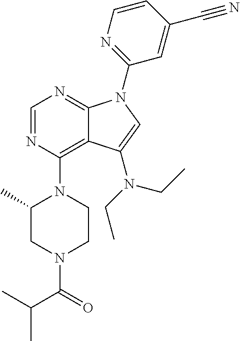 [SMILES: CCN(CC)c1cn(-c2cc(C#N)ccn2)c2ncnc(N3CCN(C(=O)C(C)C)C[C@@H]3C)c12] |
| 152 | 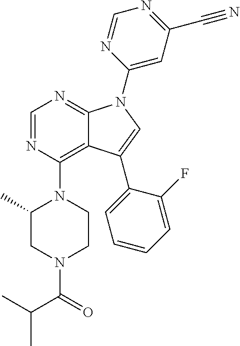 [SMILES: CC(C)C(=O)N1CCN(c2ncnc3c2c(-c2ccccc2F)cn3-c2cc(C#N)ncn2)[C@@H](C)C1] |
| 153 | 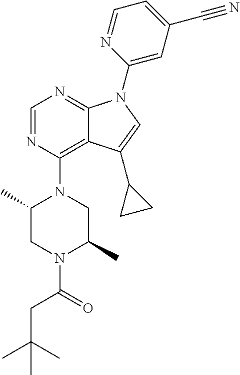 [SMILES: C[C@@H]1CN(c2ncnc3c2c(C2CC2)cn3-c2cc(C#N)ccn2)[C@@H](C)CN1C(=O)CC(C)(C)C] |
| 154 | 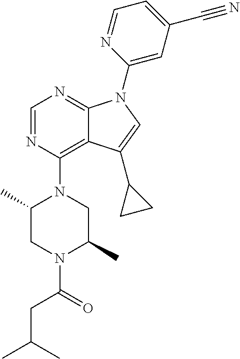 [SMILES: CC(C)CC(=O)N1C[C@H](C)N(c2ncnc3c2c(C2CC2)cn3-c2cc(C#N)ccn2)C[C@H]1C] |
| 155 | 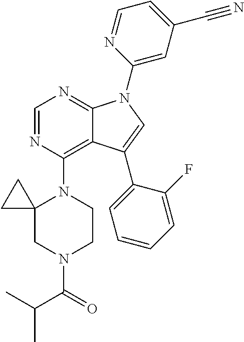 [SMILES: CC(C)C(=O)N1CCN(c2ncnc3c2c(-c2ccccc2F)cn3-c2cc(C#N)ccn2)C2(CC2)C1] |
| 156 | 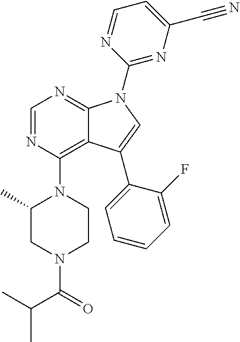 [SMILES: CC(C)C(=O)N1CCN(c2ncnc3c2c(-c2ccccc2F)cn3-c2nccc(C#N)n2)[C@@H](C)C1] |
| 157 | 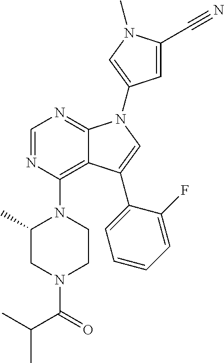 [SMILES: CC(C)C(=O)N1CCN(c2ncnc3c2c(-c2ccccc2F)cn3-c2cc(C#N)n(C)c2)[C@@H](C)C1] |
| 158 | 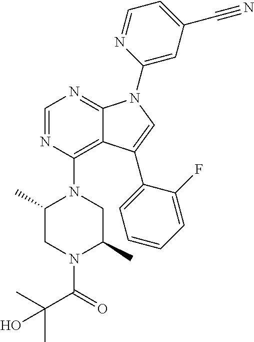 [SMILES: C[C@@H]1CN(c2ncnc3c2c(-c2ccccc2F)cn3-c2cc(C#N)ccn2)[C@@H](C)CN1C(=O)C(C)(C)O] |
| 159 | 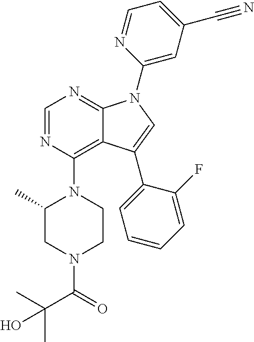 [SMILES: C[C@H]1CN(C(=O)C(C)(C)O)CCN1c1ncnc2c1c(-c1ccccc1F)cn2-c1cc(C#N)ccn1] |
| 160 | 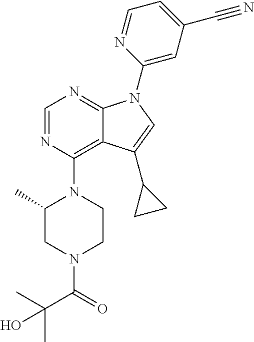 [SMILES: C[C@H]1CN(C(=O)C(C)(C)O)CCN1c1ncnc2c1c(C1CC1)cn2-c1cc(C#N)ccn1] |
| 161 | 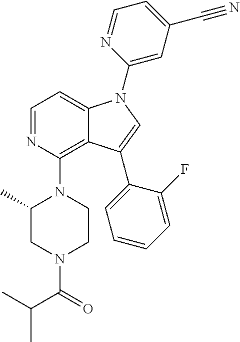 [SMILES: CC(C)C(=O)N1CCN(c2nccc3c2c(-c2ccccc2F)cn3-c2cc(C#N)ccn2)[C@@H](C)C1] |
| 162 | 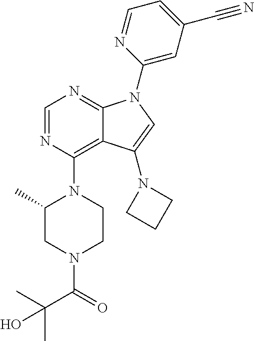 [SMILES: C[C@H]1CN(C(=O)C(C)(C)O)CCN1c1ncnc2c1c(N1CCC1)cn2-c1cc(C#N)ccn1] |
| 163 | 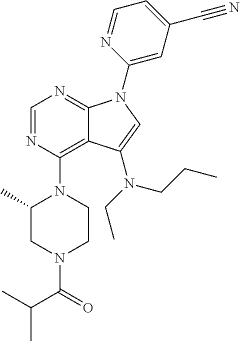 [SMILES: CCCN(CC)c1cn(-c2cc(C#N)ccn2)c2ncnc(N3CCN(C(=O)C(C)C)C[C@@H]3C)c12] |
| 164 | 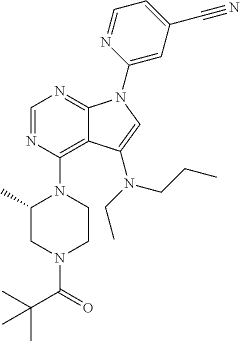 [SMILES: CCCN(CC)c1cn(-c2cc(C#N)ccn2)c2ncnc(N3CCN(C(=O)C(C)(C)C)C[C@@H]3C)c12] |
| 165 | 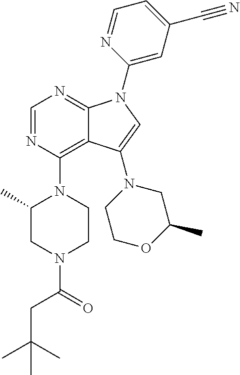 [SMILES: C[C@@H]1CN(c2cn(-c3cc(C#N)ccn3)c3ncnc(N4CCN(C(=O)CC(C)(C)C)C[C@@H]4C)c23)CCO1] |
| 166 | 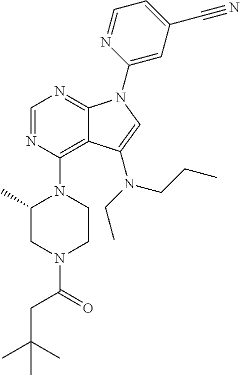 [SMILES: CCCN(CC)c1cn(-c2cc(C#N)ccn2)c2ncnc(N3CCN(C(=O)CC(C)(C)C)C[C@@H]3C)c12] |
| 167 | 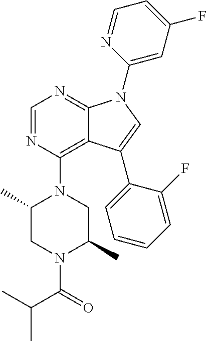 [SMILES: CC(C)C(=O)N1C[C@H](C)N(c2ncnc3c2c(-c2ccccc2F)cn3-c2cc(F)ccn2)C[C@H]1C] |
| 168 | 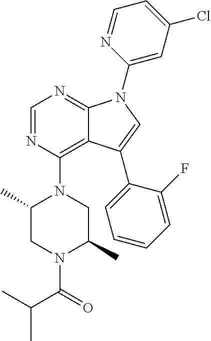 [SMILES: CC(C)C(=O)N1C[C@H](C)N(c2ncnc3c2c(-c2ccccc2F)cn3-c2cc(Cl)ccn2)C[C@H]1C] |
| 169 | 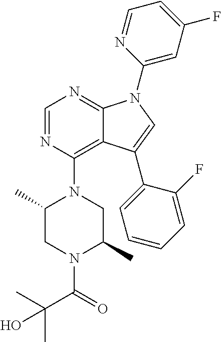 [SMILES: C[C@@H]1CN(c2ncnc3c2c(-c2ccccc2F)cn3-c2cc(F)ccn2)[C@@H](C)CN1C(=O)C(C)(C)O] |
| 170 | 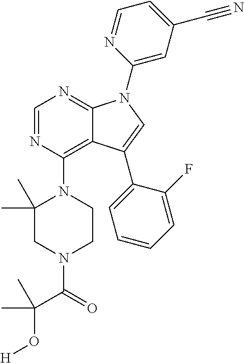 [SMILES: CC(C)(O)C(=O)N1CCN(c2ncnc3c2c(-c2ccccc2F)cn3-c2cc(C#N)ccn2)C(C)(C)C1] |
| 171 | 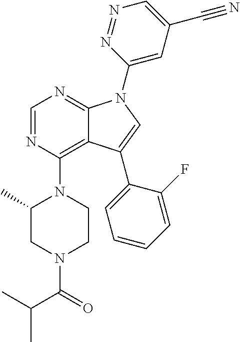 [SMILES: CC(C)C(=O)N1CCN(c2ncnc3c2c(-c2ccccc2F)cn3-c2cc(C#N)cnn2)[C@@H](C)C1] |
| 172 | 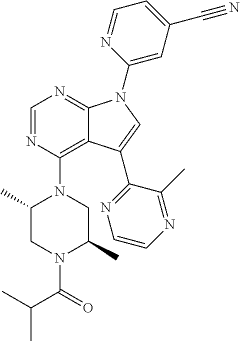 [SMILES: Cc1nccnc1-c1cn(-c2cc(C#N)ccn2)c2ncnc(N3C[C@@H](C)N(C(=O)C(C)C)C[C@@H]3C)c12] |
| 173 | 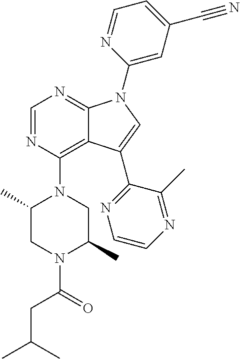 [SMILES: Cc1nccnc1-c1cn(-c2cc(C#N)ccn2)c2ncnc(N3C[C@@H](C)N(C(=O)CC(C)C)C[C@@H]3C)c12] |
| 174 | 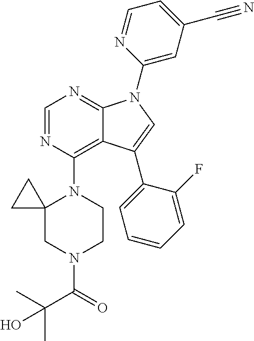 [SMILES: CC(C)(O)C(=O)N1CCN(c2ncnc3c2c(-c2ccccc2F)cn3-c2cc(C#N)ccn2)C2(CC2)C1] |
| 175 | 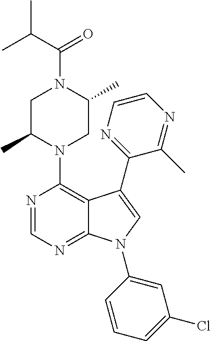 [SMILES: Cc1nccnc1-c1cn(-c2cccc(Cl)c2)c2ncnc(N3C[C@@H](C)N(C(=O)C(C)C)C[C@@H]3C)c12] |
| 176 | 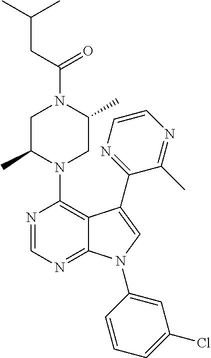 [SMILES: Cc1nccnc1-c1cn(-c2cccc(Cl)c2)c2ncnc(N3C[C@@H](C)N(C(=O)CC(C)C)C[C@@H]3C)c12] |
| 177 | 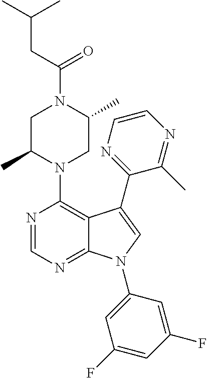 [SMILES: Cc1nccnc1-c1cn(-c2cc(F)cc(F)c2)c2ncnc(N3C[C@@H](C)N(C(=O)CC(C)C)C[C@@H]3C)c12] |
| 201 | 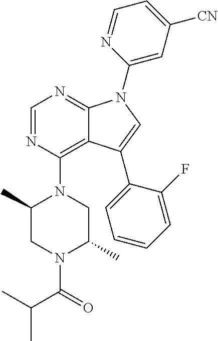 [SMILES: CC(C)C(=O)N1C[C@@H](C)N(c2ncnc3c2c(-c2ccccc2F)cn3-c2cc(C#N)ccn2)C[C@@H]1C] |
| 202 | 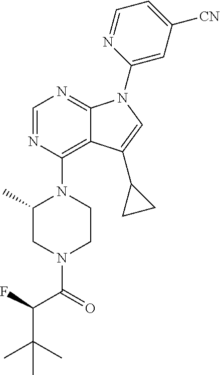 [SMILES: C[C@H]1CN(C(=O)[C@H](F)C(C)(C)C)CCN1c1ncnc2c1c(C1CC1)cn2-c1cc(C#N)ccn1] |
| 203 | 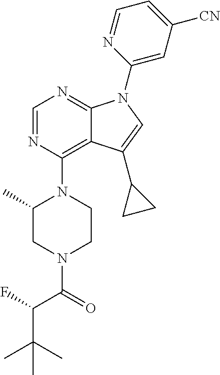 [SMILES: C[C@H]1CN(C(=O)[C@@H](F)C(C)(C)C)CCN1c1ncnc2c1c(C1CC1)cn2-c1cc(C#N)ccn1] |
| 204 | 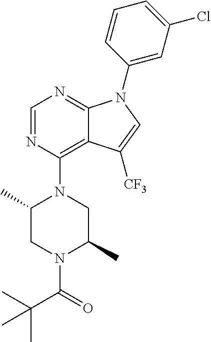 [SMILES: C[C@@H]1CN(c2ncnc3c2c(C(F)(F)F)cn3-c2cccc(Cl)c2)[C@@H](C)CN1C(=O)C(C)(C)C] |
| 205 | 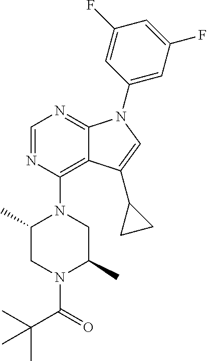 [SMILES: C[C@@H]1CN(c2ncnc3c2c(C2CC2)cn3-c2cc(F)cc(F)c2)[C@@H](C)CN1C(=O)C(C)(C)C] |
| 206 | 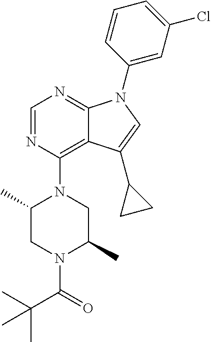 [SMILES: C[C@@H]1CN(c2ncnc3c2c(C2CC2)cn3-c2cccc(Cl)c2)[C@@H](C)CN1C(=O)C(C)(C)C] |
| 207 | 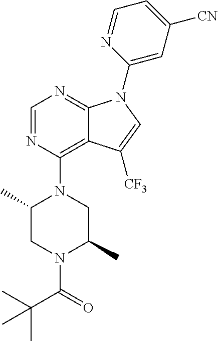 [SMILES: C[C@@H]1CN(c2ncnc3c2c(C(F)(F)F)cn3-c2cc(C#N)ccn2)[C@@H](C)CN1C(=O)C(C)(C)C] |
| 208 | 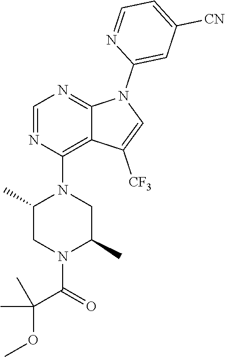 [SMILES: COC(C)(C)C(=O)N1C[C@H](C)N(c2ncnc3c2c(C(F)(F)F)cn3-c2cc(C#N)ccn2)C[C@H]1C] |
| 209 | 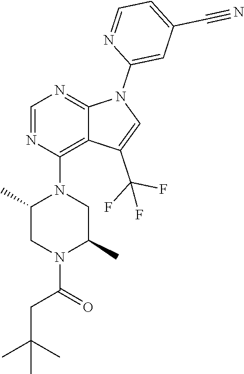 [SMILES: C[C@@H]1CN(c2ncnc3c2c(C(F)(F)F)cn3-c2cc(C#N)ccn2)[C@@H](C)CN1C(=O)CC(C)(C)C] |
| 210 | 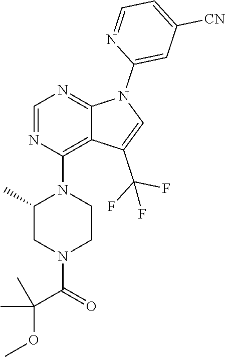 [SMILES: COC(C)(C)C(=O)N1CCN(c2ncnc3c2c(C(F)(F)F)cn3-c2cc(C#N)ccn2)[C@@H](C)C1] |
| 211 | 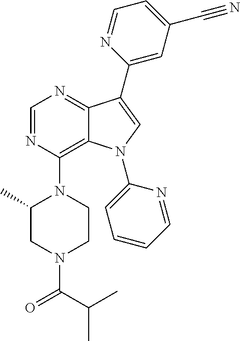 [SMILES: CC(C)C(=O)N1CCN(c2ncnc3c(-c4cc(C#N)ccn4)cn(-c4ccccn4)c23)[C@@H](C)C1] |
| 212 | 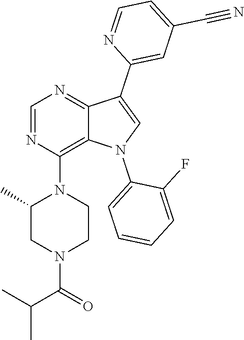 [SMILES: CC(C)C(=O)N1CCN(c2ncnc3c(-c4cc(C#N)ccn4)cn(-c4ccccc4F)c23)[C@@H](C)C1] |
| 213 | 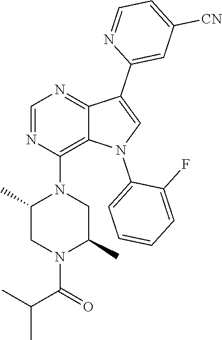 [SMILES: CC(C)C(=O)N1C[C@H](C)N(c2ncnc3c(-c4cc(C#N)ccn4)cn(-c4ccccc4F)c23)C[C@H]1C] |
| 214 | 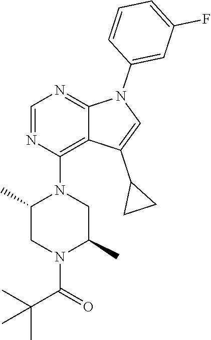 [SMILES: C[C@@H]1CN(c2ncnc3c2c(C2CC2)cn3-c2cccc(F)c2)[C@@H](C)CN1C(=O)C(C)(C)C] |
| 215 | 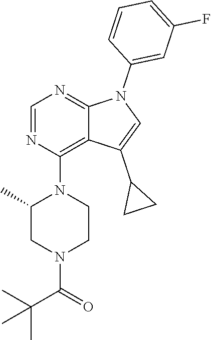 [SMILES: C[C@H]1CN(C(=O)C(C)(C)C)CCN1c1ncnc2c1c(C1CC1)cn2-c1cccc(F)c1] |
| 216 | 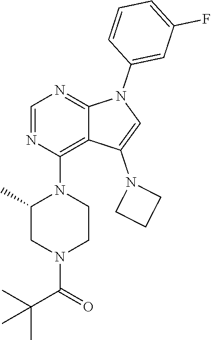 [SMILES: C[C@H]1CN(C(=O)C(C)(C)C)CCN1c1ncnc2c1c(N1CCC1)cn2-c1cccc(F)c1] |
| 217 | 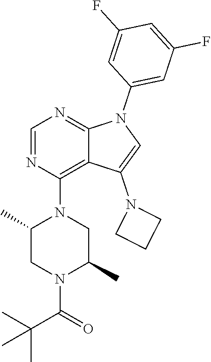 [SMILES: C[C@@H]1CN(c2ncnc3c2c(N2CCC2)cn3-c2cc(F)cc(F)c2)[C@@H](C)CN1C(=O)C(C)(C)C] |
| 218 | 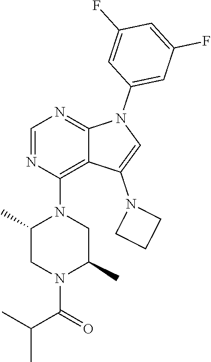 [SMILES: CC(C)C(=O)N1C[C@H](C)N(c2ncnc3c2c(N2CCC2)cn3-c2cc(F)cc(F)c2)C[C@H]1C] |
| 219 | 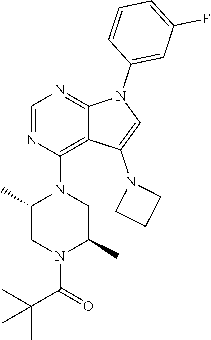 [SMILES: C[C@@H]1CN(c2ncnc3c2c(N2CCC2)cn3-c2cccc(F)c2)[C@@H](C)CN1C(=O)C(C)(C)C] |
| 220 | 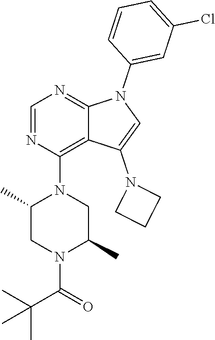 [SMILES: C[C@@H]1CN(c2ncnc3c2c(N2CCC2)cn3-c2cccc(Cl)c2)[C@@H](C)CN1C(=O)C(C)(C)C] |
| 221 | 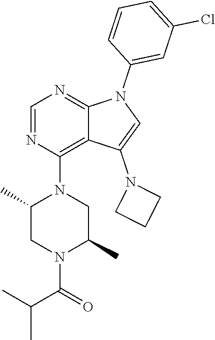 [SMILES: CC(C)C(=O)N1C[C@H](C)N(c2ncnc3c2c(N2CCC2)cn3-c2cccc(Cl)c2)C[C@H]1C] |
| 222 | 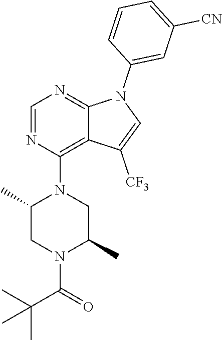 [SMILES: C[C@@H]1CN(c2ncnc3c2c(C(F)(F)F)cn3-c2cccc(C#N)c2)[C@@H](C)CN1C(=O)C(C)(C)C] |
| 223 | 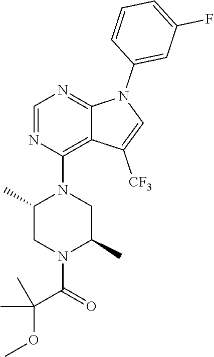 [SMILES: COC(C)(C)C(=O)N1C[C@H](C)N(c2ncnc3c2c(C(F)(F)F)cn3-c2cccc(F)c2)C[C@H]1C] |
| 224 | 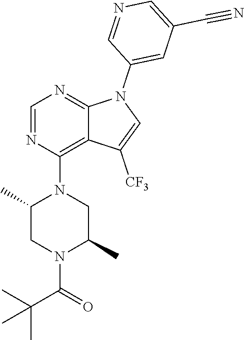 [SMILES: C[C@@H]1CN(c2ncnc3c2c(C(F)(F)F)cn3-c2cncc(C#N)c2)[C@@H](C)CN1C(=O)C(C)(C)C] |
| 225 | 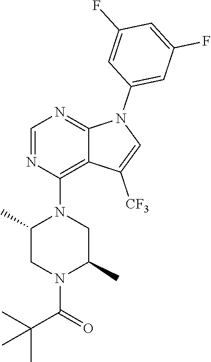 [SMILES: C[C@@H]1CN(c2ncnc3c2c(C(F)(F)F)cn3-c2cc(F)cc(F)c2)[C@@H](C)CN1C(=O)C(C)(C)C] |
| 226 | 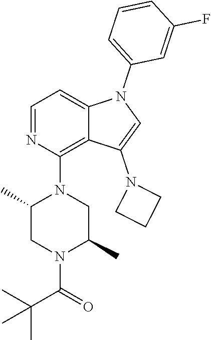 [SMILES: C[C@@H]1CN(c2nccc3c2c(N2CCC2)cn3-c2cccc(F)c2)[C@@H](C)CN1C(=O)C(C)(C)C] |
| 227 | 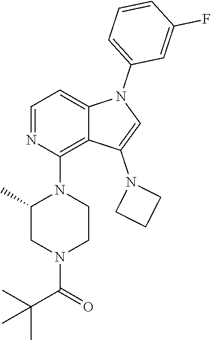 [SMILES: C[C@H]1CN(C(=O)C(C)(C)C)CCN1c1nccc2c1c(N1CCC1)cn2-c1cccc(F)c1] |
| 228 | 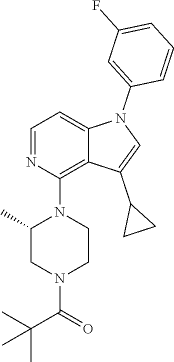 [SMILES: C[C@H]1CN(C(=O)C(C)(C)C)CCN1c1nccc2c1c(C1CC1)cn2-c1cccc(F)c1] |
| 229 | 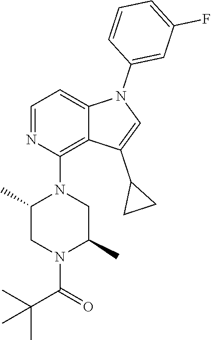 [SMILES: C[C@@H]1CN(c2nccc3c2c(C2CC2)cn3-c2cccc(F)c2)[C@@H](C)CN1C(=O)C(C)(C)C] |
| 230 | 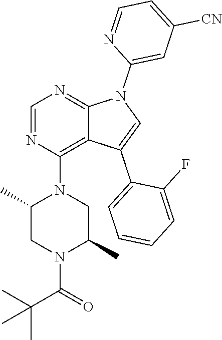 [SMILES: C[C@@H]1CN(c2ncnc3c2c(-c2ccccc2F)cn3-c2cc(C#N)ccn2)[C@@H](C)CN1C(=O)C(C)(C)C] |
| 231 | 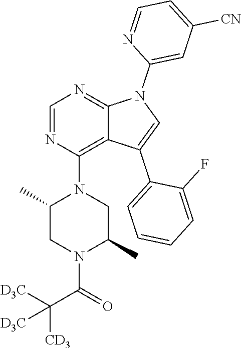 [SMILES: [2H]C([2H])([2H])C(C(=O)N1C[C@H](C)N(c2ncnc3c2c(-c2ccccc2F)cn3-c2cc(C#N)ccn2)C[C@H]1C)(C([2H])([2H])[2H])C([2H])([2H])[2H]] |
| 232 | 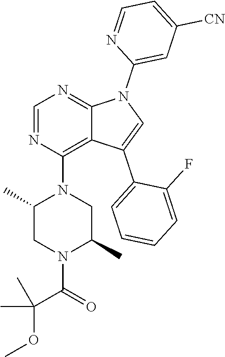 [SMILES: COC(C)(C)C(=O)N1C[C@H](C)N(c2ncnc3c2c(-c2ccccc2F)cn3-c2cc(C#N)ccn2)C[C@H]1C] |
| 233 | 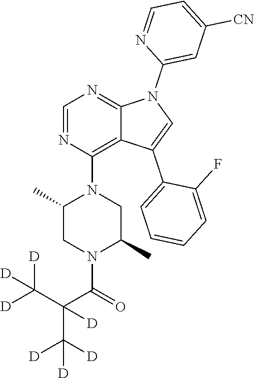 [SMILES: [2H]C([2H])([2H])C([2H])(C(=O)N1C[C@H](C)N(c2ncnc3c2c(-c2ccccc2F)cn3-c2cc(C#N)ccn2)C[C@H]1C)C([2H])([2H])[2H]] |
| 234 | 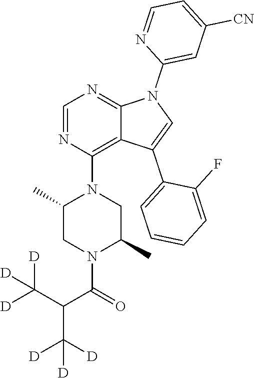 [SMILES: [2H]C([2H])([2H])C(C(=O)N1C[C@H](C)N(c2ncnc3c2c(-c2ccccc2F)cn3-c2cc(C#N)ccn2)C[C@H]1C)C([2H])([2H])[2H]] |
| 235 | 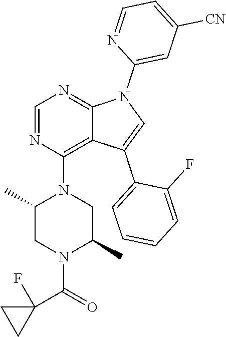 [SMILES: C[C@@H]1CN(c2ncnc3c2c(-c2ccccc2F)cn3-c2cc(C#N)ccn2)[C@@H](C)CN1C(=O)C1(F)CC1] |
| 236 | 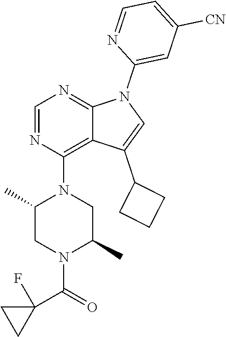 [SMILES: C[C@@H]1CN(c2ncnc3c2c(C2CCC2)cn3-c2cc(C#N)ccn2)[C@@H](C)CN1C(=O)C1(F)CC1] |
| 237 | 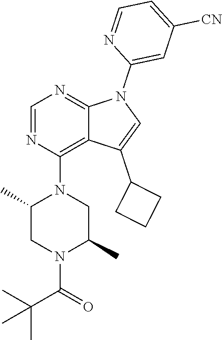 [SMILES: C[C@@H]1CN(c2ncnc3c2c(C2CCC2)cn3-c2cc(C#N)ccn2)[C@@H](C)CN1C(=O)C(C)(C)C] |
| 238 | 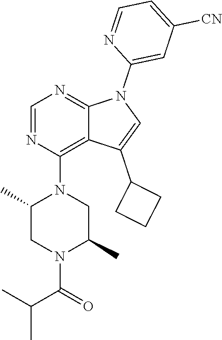 [SMILES: CC(C)C(=O)N1C[C@H](C)N(c2ncnc3c2c(C2CCC2)cn3-c2cc(C#N)ccn2)C[C@H]1C] |
| 239 | 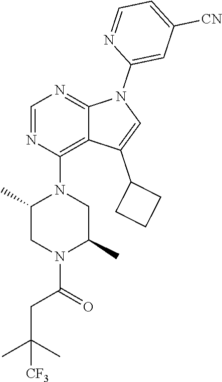 [SMILES: C[C@@H]1CN(c2ncnc3c2c(C2CCC2)cn3-c2cc(C#N)ccn2)[C@@H](C)CN1C(=O)CC(C)(C)C(F)(F)F] |
| 240 | 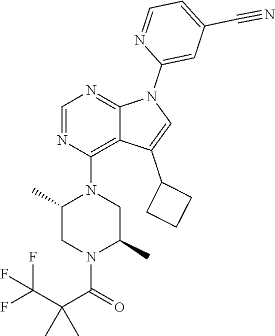 [SMILES: C[C@@H]1CN(c2ncnc3c2c(C2CCC2)cn3-c2cc(C#N)ccn2)[C@@H](C)CN1C(=O)C(C)(C)C(F)(F)F] |
| 241 | 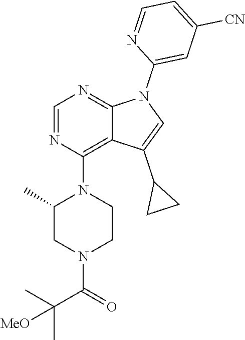 [SMILES: COC(C)(C)C(=O)N1CCN(c2ncnc3c2c(C2CC2)cn3-c2cc(C#N)ccn2)[C@@H](C)C1] |
| 242 | 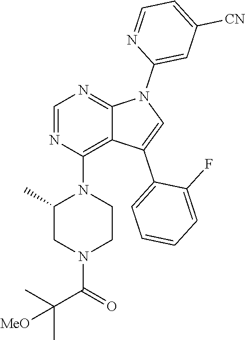 [SMILES: COC(C)(C)C(=O)N1CCN(c2ncnc3c2c(-c2ccccc2F)cn3-c2cc(C#N)ccn2)[C@@H](C)C1] |
| 243 | 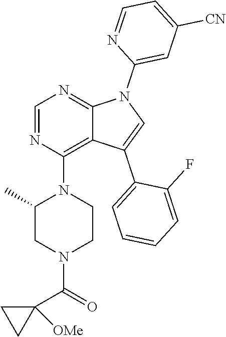 [SMILES: COC1(C(=O)N2CCN(c3ncnc4c3c(-c3ccccc3F)cn4-c3cc(C#N)ccn3)[C@@H](C)C2)CC1] |
| 244 | 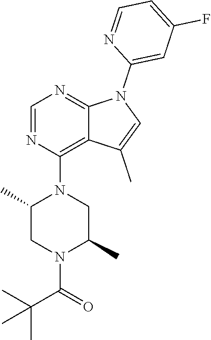 [SMILES: Cc1cn(-c2cc(F)ccn2)c2ncnc(N3C[C@@H](C)N(C(=O)C(C)(C)C)C[C@@H]3C)c12] |
| 245 | 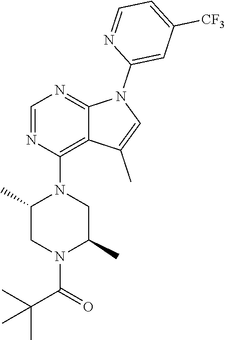 [SMILES: Cc1cn(-c2cc(C(F)(F)F)ccn2)c2ncnc(N3C[C@@H](C)N(C(=O)C(C)(C)C)C[C@@H]3C)c12] |
| 246 | 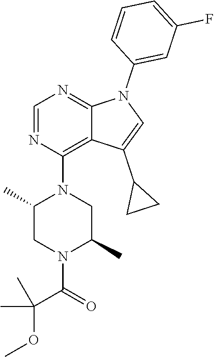 [SMILES: COC(C)(C)C(=O)N1C[C@H](C)N(c2ncnc3c2c(C2CC2)cn3-c2cccc(F)c2)C[C@H]1C] |
| 247 | 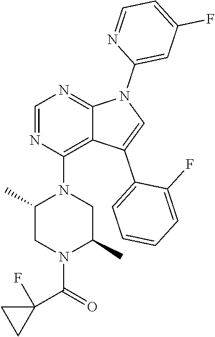 [SMILES: C[C@@H]1CN(c2ncnc3c2c(-c2ccccc2F)cn3-c2cc(F)ccn2)[C@@H](C)CN1C(=O)C1(F)CC1] |
| 248 | 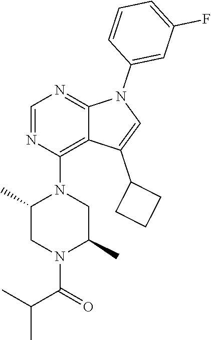 [SMILES: CC(C)C(=O)N1C[C@H](C)N(c2ncnc3c2c(C2CCC2)cn3-c2cccc(F)c2)C[C@H]1C] |
| 249 | 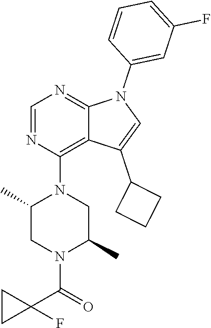 [SMILES: C[C@@H]1CN(c2ncnc3c2c(C2CCC2)cn3-c2cccc(F)c2)[C@@H](C)CN1C(=O)C1(F)CC1] |
| 250 | 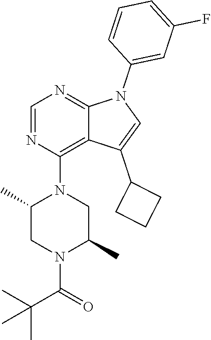 [SMILES: C[C@@H]1CN(c2ncnc3c2c(C2CCC2)cn3-c2cccc(F)c2)[C@@H](C)CN1C(=O)C(C)(C)C] |
| 251 | 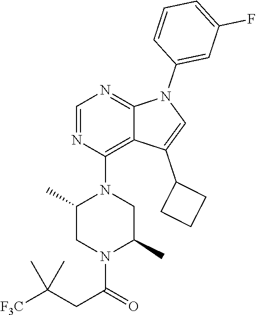 [SMILES: C[C@@H]1CN(c2ncnc3c2c(C2CCC2)cn3-c2cccc(F)c2)[C@@H](C)CN1C(=O)CC(C)(C)C(F)(F)F] |
| 252 | 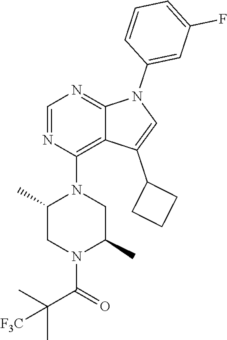 [SMILES: C[C@@H]1CN(c2ncnc3c2c(C2CCC2)cn3-c2cccc(F)c2)[C@@H](C)CN1C(=O)C(C)(C)C(F)(F)F] |
| 253 | 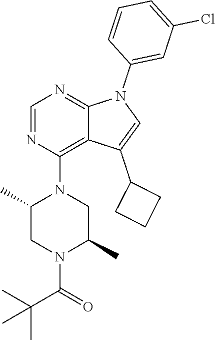 [SMILES: C[C@@H]1CN(c2ncnc3c2c(C2CCC2)cn3-c2cccc(Cl)c2)[C@@H](C)CN1C(=O)C(C)(C)C] |
| 254 | 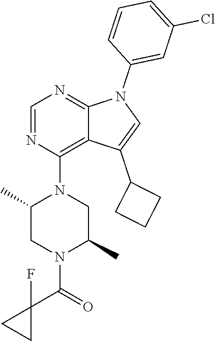 [SMILES: C[C@@H]1CN(c2ncnc3c2c(C2CCC2)cn3-c2cccc(Cl)c2)[C@@H](C)CN1C(=O)C1(F)CC1] |
| 255 | 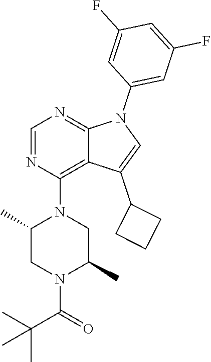 [SMILES: C[C@@H]1CN(c2ncnc3c2c(C2CCC2)cn3-c2cc(F)cc(F)c2)[C@@H](C)CN1C(=O)C(C)(C)C] |
| 256 | 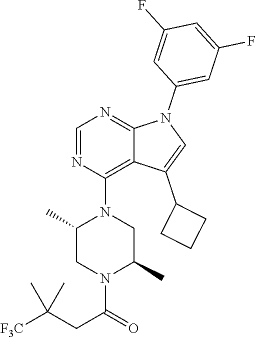 [SMILES: C[C@@H]1CN(c2ncnc3c2c(C2CCC2)cn3-c2cc(F)cc(F)c2)[C@@H](C)CN1C(=O)CC(C)(C)C(F)(F)F] |
| 257 | 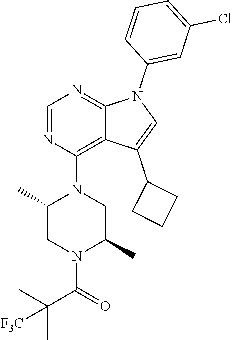 [SMILES: C[C@@H]1CN(c2ncnc3c2c(C2CCC2)cn3-c2cccc(Cl)c2)[C@@H](C)CN1C(=O)C(C)(C)C(F)(F)F] |
| 258 | 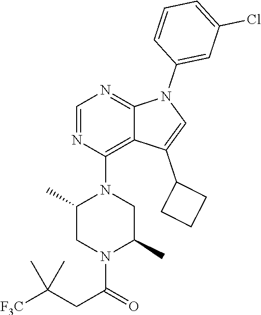 [SMILES: C[C@@H]1CN(c2ncnc3c2c(C2CCC2)cn3-c2cccc(Cl)c2)[C@@H](C)CN1C(=O)CC(C)(C)C(F)(F)F] |
| 259 | 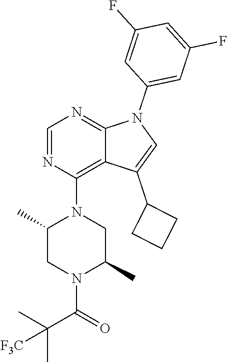 [SMILES: C[C@@H]1CN(c2ncnc3c2c(C2CCC2)cn3-c2cc(F)cc(F)c2)[C@@H](C)CN1C(=O)C(C)(C)C(F)(F)F] |
| 260 | 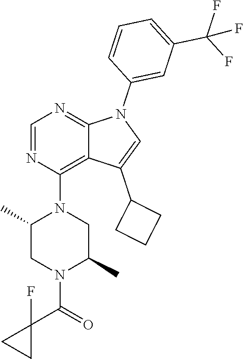 [SMILES: C[C@@H]1CN(c2ncnc3c2c(C2CCC2)cn3-c2cccc(C(F)(F)F)c2)[C@@H](C)CN1C(=O)C1(F)CC1] |
| 261 | 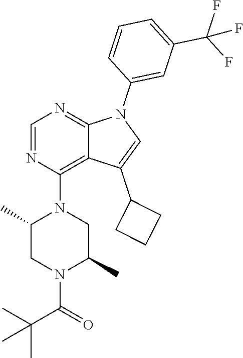 [SMILES: C[C@@H]1CN(c2ncnc3c2c(C2CCC2)cn3-c2cccc(C(F)(F)F)c2)[C@@H](C)CN1C(=O)C(C)(C)C] |
| 262 | 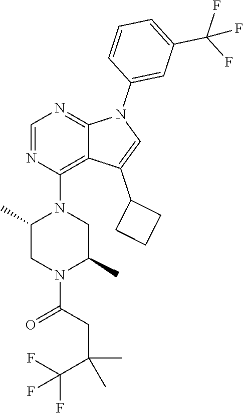 [SMILES: C[C@@H]1CN(c2ncnc3c2c(C2CCC2)cn3-c2cccc(C(F)(F)F)c2)[C@@H](C)CN1C(=O)CC(C)(C)C(F)(F)F] |
| 263 | 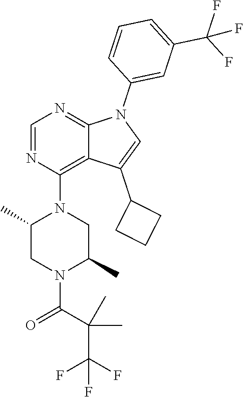 [SMILES: C[C@@H]1CN(c2ncnc3c2c(C2CCC2)cn3-c2cccc(C(F)(F)F)c2)[C@@H](C)CN1C(=O)C(C)(C)C(F)(F)F] |
| 264 | 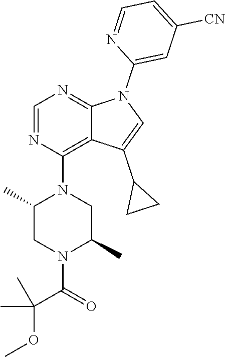 [SMILES: COC(C)(C)C(=O)N1C[C@H](C)N(c2ncnc3c2c(C2CC2)cn3-c2cc(C#N)ccn2)C[C@H]1C] |
| 265 | 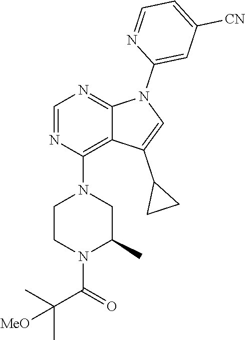 [SMILES: COC(C)(C)C(=O)N1CCN(c2ncnc3c2c(C2CC2)cn3-c2cc(C#N)ccn2)C[C@H]1C] |
| 266 | 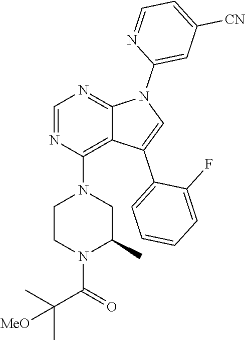 [SMILES: COC(C)(C)C(=O)N1CCN(c2ncnc3c2c(-c2ccccc2F)cn3-c2cc(C#N)ccn2)C[C@H]1C] |
| 267 | 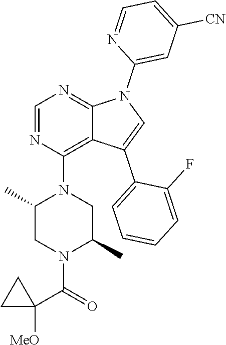 [SMILES: COC1(C(=O)N2C[C@H](C)N(c3ncnc4c3c(-c3ccccc3F)cn4-c3cc(C#N)ccn3)C[C@H]2C)CC1] |
| 268 | 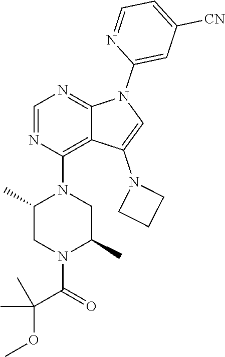 [SMILES: COC(C)(C)C(=O)N1C[C@H](C)N(c2ncnc3c2c(N2CCC2)cn3-c2cc(C#N)ccn2)C[C@H]1C] |
| 269 | 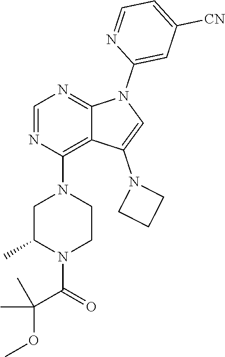 [SMILES: COC(C)(C)C(=O)N1CCN(c2ncnc3c2c(N2CCC2)cn3-c2cc(C#N)ccn2)C[C@H]1C] |
| 270 | 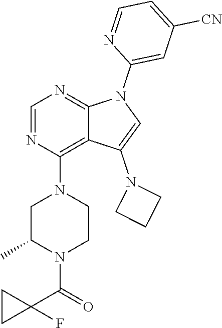 [SMILES: C[C@@H]1CN(c2ncnc3c2c(N2CCC2)cn3-c2cc(C#N)ccn2)CCN1C(=O)C1(F)CC1] |
| 271 | 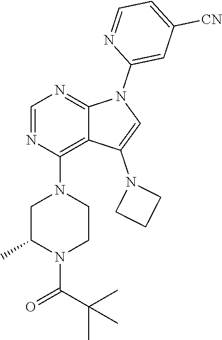 [SMILES: C[C@@H]1CN(c2ncnc3c2c(N2CCC2)cn3-c2cc(C#N)ccn2)CCN1C(=O)C(C)(C)C] |
| 272 | 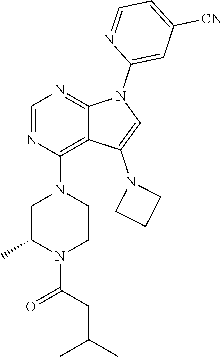 [SMILES: CC(C)CC(=O)N1CCN(c2ncnc3c2c(N2CCC2)cn3-c2cc(C#N)ccn2)C[C@H]1C] |
| 273 | 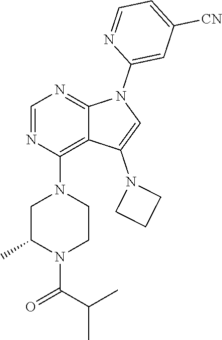 [SMILES: CC(C)C(=O)N1CCN(c2ncnc3c2c(N2CCC2)cn3-c2cc(C#N)ccn2)C[C@H]1C] |
| 274 | 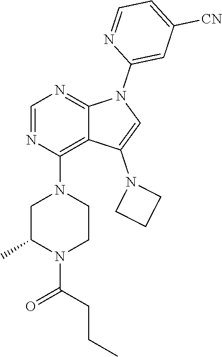 [SMILES: CCCC(=O)N1CCN(c2ncnc3c2c(N2CCC2)cn3-c2cc(C#N)ccn2)C[C@H]1C] |
| 275 | 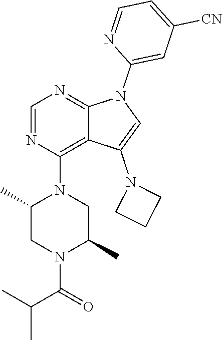 [SMILES: CC(C)C(=O)N1C[C@H](C)N(c2ncnc3c2c(N2CCC2)cn3-c2cc(C#N)ccn2)C[C@H]1C] |
| 276 | 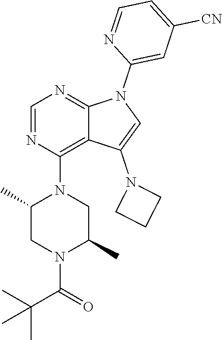 [SMILES: C[C@@H]1CN(c2ncnc3c2c(N2CCC2)cn3-c2cc(C#N)ccn2)[C@@H](C)CN1C(=O)C(C)(C)C] |
| 277 | 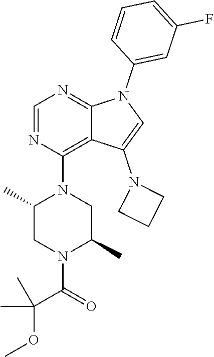 [SMILES: COC(C)(C)C(=O)N1C[C@H](C)N(c2ncnc3c2c(N2CCC2)cn3-c2cccc(F)c2)C[C@H]1C] |
| 278 | 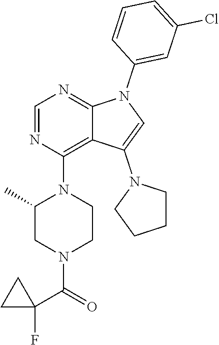 [SMILES: C[C@H]1CN(C(=O)C2(F)CC2)CCN1c1ncnc2c1c(N1CCCC1)cn2-c1cccc(Cl)c1] |
| 279 | 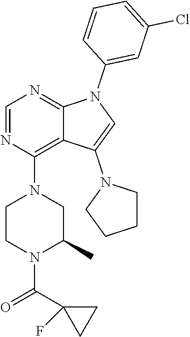 [SMILES: C[C@@H]1CN(c2ncnc3c2c(N2CCCC2)cn3-c2cccc(Cl)c2)CCN1C(=O)C1(F)CC1] |
| 280 | 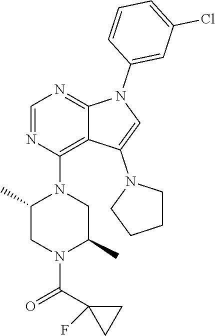 [SMILES: C[C@@H]1CN(c2ncnc3c2c(N2CCCC2)cn3-c2cccc(Cl)c2)[C@@H](C)CN1C(=O)C1(F)CC1] |
| 281 | 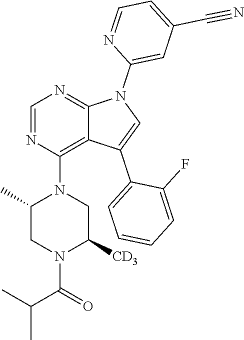 [SMILES: [2H]C([2H])([2H])[C@@H]1CN(c2ncnc3c2c(-c2ccccc2F)cn3-c2cc(C#N)ccn2)[C@@H](C)CN1C(=O)C(C)C] |
| 282 | 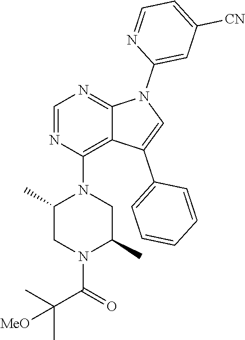 [SMILES: COC(C)(C)C(=O)N1C[C@H](C)N(c2ncnc3c2c(-c2ccccc2)cn3-c2cc(C#N)ccn2)C[C@H]1C] |
| 283 | 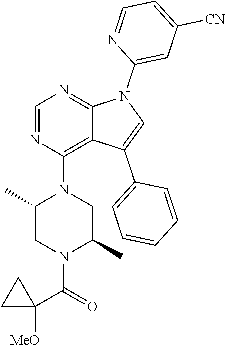 [SMILES: COC1(C(=O)N2C[C@H](C)N(c3ncnc4c3c(-c3ccccc3)cn4-c3cc(C#N)ccn3)C[C@H]2C)CC1] |

In some embodiments, compounds described herein herein (e.g., a compound of Formula I, Ia, Ib, or Ic) are deuterium enriched.

Deuterium (D or ²H) is a stable, non-radioactive isotope of hydrogen and has an atomic weight of 2.0144. Hydrogen naturally occurs as a mixture of the isotopes ¹H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015% should be considered unnatural and, as a result, novel over their non-enriched counterparts.

The effects of deuterium modification on a compound's metabolic properties are not predictable, even when deuterium atoms are incorporated at known sites of metabolism. Only by actually preparing and testing a deuterated compound can one determine if and how the rate of metabolism will differ from that of its non-deuterated counterpart. See, for example, Fukuto et al. (J. Med. Chem. 1991, 34, 2871-76). Many compounds have multiple sites where metabolism is possible. The site(s) where deuterium substitution is required and the extent of deuteration necessary to see an effect on metabolism, if any, will be different for each compound.

Unless otherwise stated, when a position is designated specifically as “H” or “hydrogen,” the position is understood to have hydrogen at its natural abundance isotopic composition. Also, unless otherwise stated, when a position is designated specifically as “D” or “deuterium,” the position is understood to have deuterium at an abundance that is at least 3000 times greater than the natural abundance of deuterium, which is 0.015% (i.e., the term “D” or “deuterium” indicates at least 45% incorporation of deuterium).

The term “isotopic enrichment factor” as used herein means the ratio between the isotopic abundance of D at the specified position in a compound of this invention and the naturally occurring abundance of that isotope.

Increasing the amount of deuterium present in a compound herein (e.g., a compound of Formula I, Ia, Ib, or Ic) is called “deuterium-enrichment,” and such compounds are referred to as “deuterium-enriched” compounds. If not specifically noted, the percentage of enrichment refers to the percentage of deuterium present in the compound.

In other embodiments, a compound of this invention has an isotopic enrichment factor for each deuterium present at a site designated at a potential site of deuteration on the compound of at least 3500 (52.5. % deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6466.7 (97% deuterium incorporation), at least 6633.3 (99.5% deuterium incorporation). It is understood that the isotopic enrichment factor of each deuterium present at a site designated as a site of deuteration is independent of other deuterated sites. For example, if there are two sites of deuteration on a compound one site could be deuterated at 52.5% while the other could be deuterated at 75%. The resulting compound would be considered to be a compound wherein the isotopic enrichment factor is at least 3500 (52.5%).

Because the natural abundance of deuterium is about 0.015%, a small percentage of naturally occurring compounds of herein (e.g., a compound of Formula I, Ia, Ib, or Ic) would be expected to have one naturally occurring compound with one deuterium present.

In some embodiments, the compounds herein (e.g., a compound of Formula I, Ia, Ib, or Ic) comprise an amount of deuterium-enrichment that is more than the amount of deuterium-enrichment present in naturally occurring compounds herein (e.g., a compound of Formula I, Ia, Ib, or Ic)

All percentages given for the amount of deuterium present are mole percentages.

It can be difficult in the laboratory to achieve 100% deuteration at any one site of a lab scale amount of compound (e.g., milligram or greater). When 100% deuteration is recited or a deuterium atom is specifically shown in a structure, it is assumed that a small percentage of hydrogen may still be present. Deuterium-enriched can be achieved by either exchanging protons with deuterium or by synthesizing the molecule with enriched starting materials.

### Methods of Treatment

Provided herein, in certain embodiments, is a method of modulating TRPML ion channels, the method comprising administering to a patient in need thereof a compound described herein (e.g., a compound of Formula I, Ia, Ib, or Ic) or pharmaceutically acceptable salts, solvates, hydrates, tautomers, stereoisomers, isotopically labeled derivatives thereof, or a composition described herein.

Provided herein, in certain embodiments, is a method of treating a disease or disorder that can be treated by modulation of TRPML ion channels, the method comprising administering to a patient in need thereof a compound described herein (e.g., a compound of Formula I, Ia, Ib, or Ic) or pharmaceutically acceptable salts, solvates, hydrates, tautomers, stereoisomers, isotopically labeled derivatives thereof, or a composition described herein.

Provided herein, in certain embodiments, is a method of treating a disease or disorder that can be treated by activation of TRPML ion channels, the method comprising administering to a patient in need thereof a compound described herein (e.g., a compound of Formula I, Ia, Ib, or Ic) or pharmaceutically acceptable salts, solvates, hydrates, tautomers, stereoisomers, isotopically labeled derivatives thereof, or a composition described herein.

Provided herein, in certain embodiments, is a method of treating a disease or disorder that can be treated by activation of TRPML1, the method comprising administering to a patient in need thereof a compound described herein (e.g., a compound of Formula I, Ia, Ib, or Ic) or pharmaceutically acceptable salts, solvates, hydrates, tautomers, stereoisomers, isotopically labeled derivatives thereof, or a composition described herein.

In addition to compounds of Formula I, Ia, Ib, or Ic, modulators of the TRPML channels have been reported in several publications, including WO2018005713 and WO2018208630, which are incorporated herein in their entirety.

In some embodiments, the TRPML ion channel is TRPML1. In some embodiments, the TRPML ion channel is TRPML2. In some embodiments, the TRPML ion channel is TRPML3.

In some embodiments, the compound is a modulator of TRPML1. In some embodiments, the compound is a modulator of TRPML2. In some embodiments, the compound is a modulator of TRPML3.

In some embodiments, modulation of the TRPML ion channel comprises activation of the ion channel.

In some embodiments, the disease or disorder is a ciliopathy (e.g., polycystic kidney disease). Exemplary ciliopathies include, but not limited to, polycystic kidney disease, pancreatic cysts in polycystic kidney disease, Bardet-Biedl syndrome, nephronophthisis, Joubert Syndrome, Mecke-Gruber Syndrome, oral-facial-digital syndrome, Senior Loken Syndrome, Birt-Hogg-Dube syndrome, Leber's congenital amaurosis, Alstrom syndrome, Jeune asphyxiating thoracic dystrophy, Ellis van Creveld syndrome, Sensenbrenner syndrome, and primary ciliary dyskinesia.

In an aspect, provided is a method of treating a disorder which can be treated by modulation of lysosomes, the method comprising administering to a patient in need thereof a therapeutically effective amount of a pharmaceutical composition of the disclosure or a compound of the disclosure.

In an aspect, provided is a method of treating a disorder selected from the group consisting of a ciliopathy, neurodegenerative disorder, lysosomal storage disorder, lysosomal transport disorder, glycogen storage disorder, cholesteryl ester storage disease, a muscular disease (e.g., muscular dystrophy), a disease related to aging (e.g., photo aging of the skin), macular degeneration (e.g., Stargradt's or age-related), and cancer (e.g., cancers of the blood, brain, bone, lung, liver, kidney, bladder, stomach, breast, prostate, ovary, testes, colon, pancreas, or skin), the method comprising administering to a patient in need thereof a therapeutically effective amount of a pharmaceutical composition of the disclosure or a compound of the disclosure.

In some embodiments, the disorder is a ciliopathy.

In some embodiments, the ciliopathy is selected from the group consisting of polycystic kidney disease, pancreatic cysts in polycystic kidney disease, Bardet-Biedl syndrome, nephronophthisis, Joubert Syndrome, Mecke-Gruber Syndrome, oral-facial-digital syndrome, Senior Loken Syndrome, Birt-Hogg-Dube syndrome, Leber's congenital amaurosis, Alstrom syndrome, Jeune asphyxiating thoracic dystrophy, Ellis van Creveld syndrome, Sensenbrenner syndrome, and primary ciliary dyskinesia.

In some embodiments, the disorder is polycystic kidney disease. In some embodiments, the disorder is autosomal dominant polycystic kidney disease, autosomal recessive polycystic kidney disease, or pancreatic cysts associated with autosomal dominant polycystic kidney disease. In some embodiments, the disorder is autosomal dominant polycystic kidney disease. In some embodiments, the disorder is a neurodegenerative disorder.

In some emobidments, the neurodegenerative disorder is selected from the group consisting of Parkinson's disease, GBA-Parkinson's disease, LRRK2 Parkinson's disease, Huntington's disease, amyotrophic lateral sclerosis (ALS), Alzheimer's disease, progressive supranuclear palsy, frontotemporal dementia, FTDP-17, corticobasal degeneration, Lewy body dementia, Pick's disease, and multi system atrophy.

In some embodiments, the disorder is a lysosomal storage disorder.

In some embodiments, the lysosomal storage disorder is selected from the group consisting of Niemann-Pick disease, Gaucher's disease, neuronopathic Gaucher's disease, sphingolipidoses, Farber disease, Krabbe disease, Galactosialidosis, gangliosidoses, Gaucher Disease, Lysosomal acid lipase deficiency, sulfatidoses, mucopolysaccharidoses, mucolipidoses, lipidoses, and oligosaccharidoses.

In some embodiments, the lysosomal storage disorder is selected from the group consisting of sphingolipidoses, Farber disease, Krabbe disease, Galactosialidosis, Fabry disease, Schindler disease, beta-galactosidase disorder, GM1 gangliosidosis, GM2 gangliosidosis AB variant, GM2 gangliosidosis activator deficiency, Sandhoff disease, Tay-Sachs disease, Gaucher disease, lysosomal acid lipase deficiency, Niemann-Pick disease, metachromatic leukodystrophy, Saposin B deficiency, multiple sulfatase deficiency, Hurler syndrome, Scheie sundrome, Hurler-Scheie syndrome, Hunter syndrome, Sanfilippo syndrome, Morquio syndrome, Maroteaux-Lamy syndrome, Sly syndrome, hyaluronidase deficiency, sialidosis, I-cell disease, pseudo-Hurler polydystrophy, phosphotransferease deficiency, mucolipidin 1 deficiency, Santavuori-Haltia disease, Jansky-Bielchowsky disease, Batten-Spielmeyer-Vogt disease, Kufs disease, Finnish variant neuronal ceroid lipfuscinosis, late infantile variant neuronal ceroid lipfuscinosis, type 7 neuronal ceroid lipfuscinosis, northern epilepsy neuronal ceroid lipfuscinosis, Turkish late infantile neuronal ceroid lipfuscinosis, German/Serbian late infantile neuronal ceroid lipfuscinosis, congential cathepsin D deficiency, Wolman disease, alpha-mannosidosis, beta-mannosidosis, aspartylgluosaminuria, and fucosidosis.

In some embodiments, the lysosomal storage disorder is selected from the group consisting of Niemann-Pick disease, Gaucher's disease, and neuronopathic Gaucher's disease.

In some embodiments, the disorder is a lysosomal transport disease selected from the group consisting of cystinosis, pycnodysostosis, Salla disease, sialic acid storage disease, and infantile free sialic acid storage disease.

In some embodiments, the disorder is a glycogen storage disease selected from the group consisting of Pompe disease and Danon disease.

In an aspect, provided is a method of treating a ciliopathy disorder, the method comprising administering to a patient in need thereof a therapeutically effective amount of a compound capable of modulating TRPML, or a therapeutically effective amount of a pharmaceutical composition comprising the compound and a pharmaceutically acceptable excipient.

In some embodiments, the compound is selected from the compounds disclosed in the specification.

In some embodiments, the ciliopathy is selected from the group consisting of polycystic kidney disease, pancreatic cysts in polycystic kidney disease, Bardet-Biedl syndrome, nephronophthisis, Joubert Syndrome, Meckel-Gruber Syndrome, oral-facial-digital syndrome, Senior Loken Syndrome, Birt-Hogg-Dube syndrome, Leber's congenital amaurosis, Alstrom syndrome, Jeune asphyxiating thoracic dystrophy, Ellis van Creveld syndrome, Sensenbrenner syndrome, and primary ciliary dyskinesia.

In some embodiments, the disorder is polycystic kidney disease

In some embodiments, the disorder is autosomal dominant polycystic kidney disease, autosomal recessive polycystic kidney disease, or pancreatic cysts associated with autosomal dominant polycystic kidney disease.

In some embodiments, the disorder is autosomal dominant polycystic kidney disease.

In some embodiments, the method further comprises the use of a second therapeutic agent.

In some embodiments, the method is to treat a ciliopathy.

In some embodiments, the second therapeutic agent is selected from the group consisting of an mTOR inhibitor, V2 receptor antagonist, tyrosine kinase inhibitor, somatostatin analog, glucosylceramide synthase inhibitor, microRNA-17 inhibitor, siRNA against p53, KEAP1-Nrf2 activator, xanthine oxidase inhibitor, PPARy agonist, metformin, and beta hydroxybutyrate.

In some embodiments, the second therapeutic agent is selected from the group consisting of tolvaptan, lixivaptan, mozavaptan, satavaptan, sirolimus, tacrolimus, everolimus, bosutinib, tesavatinib, imatinib, gefitinib, erlotinib, dasatinib, octreotide, pasireotide, venglustat, eliglustat, miglustat, microRNA-17 inhibitor, bardoxolone methyl, allopurinol, oxypurinol, pioglitazone, rosiglitazone, lobeglitazone, metformin, and beta hydroxybutyrate. In some embodiments, the second agent is tolvaptan.

In some embodiments, the second therapeutic agent is selected from the group consisting of an immunomodulator, a calcineurin inhibitor, a renin angiotensin aldosterone system inhibitor, an antiproliferative agent, an alkylating agent, a corticosteroid, an angiotensin converting enzyme inhibitor, an adrenocorticotropic hormone stimulant, an angiotensin receptor blocker, a sodium-glucose transport protein 2 inhibitor, a dual sodium-glucose transport protein ½ inhibitor, a nuclear Factor-1 (erythroid-derived 2)-like 2 agonist, a chemokine receptor 2 inhibitor, a chemokine receptor S inhibitor, an endothelin 1 receptor antagonist, a beta blocker, a mineralocorticoid receptor antagonist, a loop or thiazide diuretic, a calcium channel blocker, a statin, a short-intermediate or long-acting insulin, a dipeptidyl peptidase 4 inhibitor, a glucagon-like peptide 1 receptor agonist, a sulfonylurea, an apoptosis signal-regulating kinase-1, a chymase inhibitor, a selective gly cation inhibitor, a renin inhibitor, an interleukin-33 inhibitor, a farnesoid X receptor agonist, a soluble guanylate cyclase stimulator, a thromboxane receptor antagonist, a xanthine oxidase inhibitor, an erythropoietin receptor agonist, a cannabinoid receptor type I inverse agonist, a NADPH oxidase inhibitor, an anti-vascular endothelial growth factor B, an anti-fibrotic agent, a neprilysin inhibitor, a dual CD80/CD86 inhibitor, a CD40 antagonist, a cellular cholesterol and lipid blocker, a PDGFR antagonist, a Slit guidance ligand 2, an APOL1 inhibitor, an Nrl2 activator/NF-kB inhibitor, a somatostatin receptor agonist, a PPAR gamma agonist, a AMP activated protein kinase stimulator, a tyrosine kinase inhibitor, a glucosylceramide synthase inhibitor, an arginine vasopressin receptor 2 antagonist, a xanthine oxidase inhibitor, a vasopressin receptor 2 antagonist, anti-amyloid beta antibodies, anti-Tau antibodies, anti-synuclein antibodies, dopamine precursors (e.g. L-DOPA), dopamine agonists (e.g. bromocriptine, cabergoline, pergolide, pramipexole and apomorphine), MAO-B inhibitors (e.g. rasagiline and selegiline), anticholinergics (e.g. orphenadrine, procyclidine and trihexyphenidyl), enhancers of b-glucocerebrosidase activity (e.g. ambroxol and afegostat), amantadine, and agents capable of treating Alzheimer's (e.g., acetylcholinesterase inhibitors such as tacrine, rivastigmine, galantamine, donepezil, and NMDA receptor antagonists such as memantine).

In some embodiments, the second therapeutic agent is selected from the group consisting of COX inhibitors including arylcarboxylic acids (salicylic acid, acetylsalicylic acid, diflunisal, choline magnesium trisalicylate, salicylate, benorylate, flufenamic acid, mefenamic acid, meclofenamic acid and triflumic acid), arylalkanoic acids (diclofenac, fenclofenac, alclofenac, fentiazac, ibuprofen, flurbiprofen, ketoprofen, naproxen, fenoprofen, fenbufen, suprofen, indoprofen, tiaprofenic acid, benoxaprofen, pirprofen, tolmetin, zomepirac, clopinac, indomethacin and sulindac) and enolic acids (phenylbutazone, oxyphenbutazone, azapropazone, feprazone, piroxicam, and isoxicam; treatments for pulmonary hypertension including prostanoids (epoprostenol, iloprost, and treprostinil), endothelin receptor antagonists (bosentan, ambrisentan, and macitentan), phosphodiesterase-5 inhibitors (sildenafil and tadalafil), and sGC stimulators (riociguat), rho-kinase inhibitors, such as Y-27632, fasudil, and H-1152P; epoprostenol derivatives, such as prostacyclin, treprostinil, beraprost, and iloprost; serotonin blockers, such as sarpogrelate; endothelin receptor antagonists, such as besentan, sitaxsentan, ambrisentan, and TBC3711; PDE inhibitors, such as sildenafil, tadalafil, udenafil, and vardenafil; soluble gunaylate cyclase inhibtors such as riociguat and vericiguat; calcium channel blockers, such as amlodipine, bepridil, clentiazem, diltiazem, fendiline, gallopamil, mibefradil, prenylamine, semotiadil, terodiline, verapamil, aranidipine, bamidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, and perhexiline; tyrosine kinase inhibitors, such as imatinib; inhaled nitric oxide and nitric oxide-donating agents, such as inhaled nitrite; IκB inhibitors, such as IMD 1041; prostacyclin receptor agonists, such as selexipag; stimulators of hematopoiesis, such as TXA 127 (angiotensin (1-7)), darbepoetin alfa, erythropoetin, and epoetin alfa; anticoagulants and platelet-inhibiting agents; and diuretics; dietary and nutritional supplements such as acetyl-L-carnitine, octacosanol, evening primrose oil, vitamin B6, tyrosine, phenylalanine, vitamin C, L-dopa; immunosuppressants (for transplants and autoimmune-related RKD); anti-hypertensive drugs (for high blood pressure-related RKD, e.g., angiotensin-converting enzyme inhibitors and angiotensin receptor blockers); insulin (for diabetic RKD); lipid/cholesterol-lowering agents (e.g., HMG-CoA reductase inhibitors such as atorvastatin or simvastatin); and treatments for hyperphosphatemia or hyperparathyroidism associated with CKD (e.g., sevelamer acetate, cinacalcet).

The present disclosure further provides pharmaceutical compositions comprising a compound provided herein, or a pharmaceutically acceptable salt thereof, and at least one pharmaceutically acceptable carrier. The present disclosure further provides methods of modulating TRPML in a subject, the method comprising administering to the subject a therapeutically effective amount of a compound provided herein, or a pharmaceutically acceptable salt thereof.

The present disclosure further provides a method of treating a disease or disorder in a subject, the method comprising:
(a) detecting a disease or disorder associated with TRPML; and
(b) administering to the subject a therapeutically effective amount of a compound provided herein, or a pharmaceutically acceptable salt thereof.

In certain embodiments, exemplary compounds of Formula I, Ia, Ib, or Ic include the compounds described in Table 1 and in the Examples, as well as pharmaceutically acceptable salts, solvates, hydrates, tautomers, and stereoisomers thereof.

Accordingly, the present disclosure provides compounds useful for treating ciliopathies and related diseases.

Compounds that modulate TRPML channels may be useful in the prophylaxis and treatment of any of the foregoing injuries, diseases, disorders, or conditions. In addition to in vitro assays of the activity of these compounds, their efficacy can be readily tested in one or more animal models.

This disclosure is not limited in its application to the details of the methods and compositions described herein. Also, the phraseology and terminology used herein is for the purpose of description and should not be regarded as limiting.

### Pharmaceutical Compositions and Routes of Administration

The present disclosure provides pharmaceutical compositions comprising a compound provided herein, or a pharmaceutically acceptable salt thereof, and at least one pharmaceutically acceptable carrier. Also provided herein are methods of modulating TRPML channels in a subject, the method comprising administering to the subject a therapeutically effective amount of a compound provided herein, or a pharmaceutically acceptable salt thereof.

In certain embodiments, pharmaceutical compositions containing compounds described herein such as a compound of Formula I, Ia, Ib, or Ic, or pharmaceutically acceptable salt thereof can be used to treat or ameliorate a disorder described herein, for example, a ciliopathy.

The amount and concentration of compounds of Formula I, Ia, Ib, or Ic in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

While it is possible for a compound disclosed herein to be administered alone, it is preferable to administer the compound as a pharmaceutical formulation, where the compound is combined with one or more pharmaceutically acceptable diluents, excipients or carriers. The compounds according to the disclosure may be formulated for administration in any convenient way for use in human or veterinary medicine. In certain embodiments, the compound included in the pharmaceutical preparation may be active itself, or may be a prodrug, e.g., capable of being converted to an active compound in a physiological setting. Regardless of the route of administration selected, the compounds of the present disclosure, which may be used in a suitable hydrated form, and/or the pharmaceutical compositions of the present disclosure, are formulated into pharmaceutically acceptable dosage forms such as described below or by other conventional methods known to those of skill in the art.

Thus, another aspect of the present disclosure provides pharmaceutically acceptable compositions comprising a therapeutically effective amount of one or more of the compounds described above, formulated together with one or more pharmaceutically acceptable carriers (additives) and/or diluents. As described in detail below, the pharmaceutical compositions of the present disclosure may be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; (9) nasally; or (10) intrathecally. Additionally, compounds can be implanted into a patient or injected using a drug delivery system. See, for example, Urquhart, et al., (1994) Ann Rev Pharmacol Toxicol 24:199-236; Lewis, ed. “Controlled Release of Pesticides and Pharmaceuticals” (Plenum Press, New York, 1981); U.S. Pat. Nos. 3,773,919; and 3,270,960.

The phrase “therapeutically effective amount” as used herein means that amount of a compound, material, or composition comprising a compound of the present disclosure, which is effective for producing some desired therapeutic effect, e.g., by modulating EHMT1 or EHMT2, in at least a sub-population of cells in an animal and thereby blocking the biological consequences of that function in the treated cells, at a reasonable benefit/risk ratio applicable to any medical treatment.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) cyclodextrins such as Captisol®; and (22) other non-toxic compatible substances employed in pharmaceutical formulations.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66:1-19 (1977)). Certain specific compounds of the present disclosure contain both basic and acidic functionalities that allow the compounds to be converted into either base or acid addition salts. These salts may be prepared by methods known to those skilled in the art. Other pharmaceutically acceptable carriers known to those of skill in the art are suitable for the present disclosure.

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present disclosure with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. A compound of the present disclosure may also be administered as a bolus, electuary or paste.

In solid dosage forms of the disclosure for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present disclosure, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the disclosure for rectal, vaginal, or urethral administration may be presented as a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Alternatively or additionally, compositions can be formulated for delivery via a catheter, stent, wire, or other intraluminal device. Delivery via such devices may be especially useful for delivery to the heart, lung, bladder, urethra, ureter, rectum, or intestine. Furthermore, compositions can be formulated for delivery via a dialysis port.

Ophthalmic formulations, eye ointments, powders, solutions and the like, are also contemplated as being within the scope of this disclosure.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the compositions are administered by intravenous infusion or injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions of this disclosure suitable for parenteral administration comprise one or more compounds of the disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

When the compounds of the present disclosure are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the disclosure to animal feed is preferably accomplished by preparing an appropriate feed premix containing the active compound in an effective amount and incorporating the premix into the complete ration. Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” O and B books, Corvallis, Ore., U.S.A., 1977).

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with neurodegenerative disease or disorder, cancer, or viral infections.

In addition, the methods described herein can be used to treat domesticated animals and/or pets. A subject can be male or female. A subject can be one who has been previously diagnosed with or identified as suffering from or having a neurodegenerative disease or disorder, a disease or disorder associated with cancer, a disease or disorder associated with viral infection, or one or more complications related to such diseases or disorders but need not have already undergone treatment.

### Dosages

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present disclosure employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

The compound and the pharmaceutically active agent can be administrated to the subject in the same pharmaceutical composition or in different pharmaceutical compositions (at the same time or at different times). When administrated at different times, the compound and the pharmaceutically active agent can be administered within 5 minutes, 10 minutes, 20 minutes, 60 minutes, 2 hours, 3 hours, 4, hours, 8 hours, 12 hours, 24 hours of administration of the other agent. When the compound and the pharmaceutically active agent are administered in different pharmaceutical compositions, routes of administration can be different.

The amount of compound that can be combined with a carrier material to produce a single dosage form will generally be that amount of the compound that produces a therapeutic effect. Generally, out of one hundred percent, this amount will range from about 0.1% to 99% of compound, preferably from about 5% to about 70%, most preferably from 10% to about 30%.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the EC₅₀ (i.e., the concentration of the therapeutic which achieves a half-maximal effect) as determined in cell culture. Levels in plasma may be measured, for example, by high performance liquid chromatography. The effects of any particular dosage can be monitored by a suitable bioassay.

The dosage may be determined by a physician and adjusted, as necessary, to suit observed effects of the treatment.

With respect to duration and frequency of treatment, it is typical for skilled clinicians to monitor subjects in order to determine when the treatment is providing therapeutic benefit, and to determine whether to increase or decrease dosage, increase, or decrease administration frequency, discontinue treatment, resume treatment or make other alteration to treatment regimen. The dosing schedule can vary from once a week to daily depending on a number of clinical factors, such as the subject's sensitivity to the drugs. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

The present disclosure contemplates formulation of the subject compounds in any of the aforementioned pharmaceutical compositions and preparations. Furthermore, the present disclosure contemplates administration via any of the foregoing routes of administration.

One of skill in the art can select the appropriate formulation and route of administration based on the condition being treated and the overall health, age, and size of the patient being treated.

### Selected Chemical Definitions

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, propyl, butyl, pentyl, and hexyl.

For compounds of the disclosure in which a variable appears more than once, each variable can be a different moiety selected from the Markush group defining the variable. For example, where a structure is described having two R groups that are simultaneously present on the same compound; the two R groups can represent different moieties selected from the Markush group defined for R.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

In case a compound of the present disclosure is depicted in form of a chemical name and as a formula in case of any discrepancy the formula shall prevail.

An asterisk or wavy line may be used in sub-formulas to indicate the bond which is connected to the core molecule as defined.

The term “substituted,” as used herein, means that any one or more hydrogens on the designated atom, usually a carbon, oxygen, or nitrogen atom, is replaced with a selection from the indicated group, provided that the designated atom's normal valency is not exceeded, and that the substitution results in a stable compound. When a substituent is keto or oxo (i.e., ═O), then 2 hydrogens on the atom are replaced. Ring double bonds, as used herein, are double bonds that are formed between two adjacent ring atoms (e.g., C═C, C═N, N═N, etc.).

As used herein, “alkyl” is intended to include both branched and straight-chain saturated aliphatic hydrocarbon groups having the specified number of carbon atoms. For example, C₁₋₄ alkyl is intended to include C₁, C₂, C₃, and C₄. C₁₋₆ alkyl is intended to include C₁ C₂, C₃, C₄, C₅, and C₆ alkyl groups and C₁₋₈ alkyl is intended to include C₁, C₂, C₃, C₄, C₅, C₆, C₇, and C₈. Some examples of alkyl include, but are not limited to, methyl, ethyl, n-propyl, iso-propyl, n-butyl, s-butyl, t-butyl, n-pentyl, s-pentyl, n-hexyl, n-heptyl, and n-octyl.

As used herein, “alkenyl” is intended to include hydrocarbon chains of either straight or branched configuration and one or more unsaturated carbon-carbon bond that can occur in any stable point along the chain, such as ethenyl and propenyl. For example, C₂₋₆ alkenyl is intended to include C₂, C₃, C₄, C₅, and C₆ alkenyl groups and C₂₋₈ alkenyl is intended to include C₂, C₃, C₄, C₅, C₆, C₇, and C₈ alkenyl groups.

As used herein, “alkylene” is intended to include moieties which are diradicals, i.e., having two points of attachment. A non-limiting example of such an alkylene moiety that is a diradical is-CH₂CH₂—, i.e., a C₂ alkyl group that is covalently bonded via each terminal carbon atom to the remainder of the molecule. The alkylene diradicals are also known as “alkylenyl” radicals. Alkylene groups can be saturated or unsaturated (e.g., containing —CH═CH— or —C≡C— subunits), at one or several positions. In some embodiments, alkylene groups include 1 to 9 carbon atoms (for example, 1 to 6 carbon atoms, 1 to 4 carbon atoms, or 1 to 2 carbon atoms). Some examples of alkylene groups include, but are not limited to, methylene, ethylene, n-propylene, iso-propylene, n-butylene, iso-butylene, sec-butylene, tert-butylene, n-pentylene, iso-pentylene, sec-pentylene and neo-pentylene.

As used herein, “cycloalkyl” is intended to include saturated or unsaturated nonaromatic ring groups, such as cyclopropyl, cyclobutyl, cyclopentyl, or cyclohexyl. For example, the term “C₃₋₈ cycloalkyl” is intended to include C₃, C₄, C₅, C₆, C₇, and C₈ cycloalkyl groups. Cycloalkyls may include multiple spiro- or fused or bridged rings. For example, cycloalkyl can include, but is not limited to, spiro butyl, pentyl, hexyl, heptyl, octyl, nonyl, or decyl groups, bicyclo butyl, pentyl, hexyl, heptyl, octyl, nonyl, or decyl groups, adamantyl groups, and norbornyl groups.

As used herein, the term “heterocycloalkyl” refers to a saturated or unsaturated nonaromatic 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, or Se), unless specified otherwise. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, tetrahyrofuranyl, oxiranyl, azetidinyl, oxetanyl, thietanyl, 1,2,3,6-tetrahydropyridinyl, tetrahydropyranyl, dihydropyranyl, pyranyl, morpholinyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl and the like.

As used herein, “amine” or “amino” refers to unsubstituted —H₂ unless otherwise specified. As used herein, “halo” or “halogen” refers to fluoro, chloro, bromo, and iodo substituents.

As used herein, “haloalkyl” is intended to include both branched and straight-chain saturated aliphatic hydrocarbon groups having the specified number of carbon atoms, substituted with one or more halogen (for example —CᵥF_{w}H_{2v·w+1} wherein v=1 to 3 and w=1 to (2v+1)). Examples of haloalkyl include, but are not limited to, trifluoromethyl, trichlorom ethyl, pentafluoroethyl, and pentachloroethyl.

The term “haloalkoxy” as used herein refers to an alkoxy group, as defined herein, which is substituted one or more halogen. Examples of haloalkoxy groups include, but are not limited to, tnfluoromethoxy, difluoromethoxy, pentafluoroethoxy, trichloromethoxy, etc.

As used herein, “alkoxyl” or “alkoxy” refers to an alkyl group as defined above with the indicated number of carbon atoms attached through an oxygen bridge. C₁₋₆alkoxy, is intended to include C₁, C₂, C₃, C₄, C₅, and C₆ alkoxy groups. C₁₋₈ alkoxy, is intended to include C₁, C₂, C₃, C₄, C₅, C₆, C₇, and C₈ alkoxy groups. Examples of alkoxy include, but are not limited to, methoxy, ethoxy, n-propoxy, i-propoxy, n-butoxy, s-butoxy, t-butoxy, n-pentoxy, s-pentoxy, n-heptoxy, and n-octoxy.

As used herein, “aryl” includes groups with aromaticity, including “conjugated,” or multicyclic systems with at least one aromatic ring and do not contain any heteroatom in the ring structure. Aryl may be monocyclic or polycyclic (e.g., having 2, 3 or 4 fused rings). The term “Cn-m aryl” refers to an aryl group having from n to m ring carbon atoms. In some embodiments, aryl groups have from 6 to 10 carbon atoms. In some embodiments, the aryl group is phenyl or naphthyl.

As used herein, the terms “aromatic heterocycle,” “aromatic heterocyclic” or “heteroaryl” ring are intended to mean a stable 5, 6, 7, 8, 9, 10, 11, or 12-membered monocyclic or bicyclic aromatic ring which consists of carbon atoms and one or more heteroatoms, e.g., 1 or 1-2 or 1-3 or 1-4 or 1-5 or 1-6 heteroatoms, independently selected from nitrogen, oxygen, and sulfur. In the case of bicyclic aromatic heterocyclic or heterocycle or heteroaryl rings, only one of the two rings needs to be aromatic (e.g., 2,3-dihydroindole), though both can be (e.g., quinoline). The second ring can also be fused or bridged as defined above for heterocycles. The nitrogen atom can be substituted or unsubstituted (i.e., N or R wherein R is H or another substituent, as defined). The nitrogen and sulfur heteroatoms can optionally be oxidized (i.e., N→O and S(O)_{P}, wherein p=1 or 2). In certain compounds, the total number of S and O atoms in the aromatic heterocycle is not more than 1.

Examples of aromatic heterocycles, aromatic heterocyclics or heteroaryls include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, benzooxadiazoly, carbazolyl, 4aH-carbazolyl, carbolinyl, cinnolinyl, furazanyl, imidazolyl, imidazolonyl, 1H-indazolyl, indolizinyl, indolyl, 3H-indolyl, isobenzofuranyl, isochromanyl, isoindazolyl, isoindolyl, isoquinolinyl, isothiazolyl, isoxazolyl, methylbenztriazolyl, methylfuranyl, methylimidazolyl, methylthiazolyl, naphthyridinyl, oxadiazolyl, 1,2,3-oxadiazolyl, 1,2,4-oxadiazolyl, 1,2,5-oxadiazolyl, 1,3,4-oxadiazolyl, oxazolyl, phenanthridinyl, phenanthrolinyl, phenazinyl, phenothiazinyl, phenoxathinyl, phenoxazinyl, phthalazinyl, pteridinyl, purinyl, pyrazinyl, pyrazolyl, pyridazinyl, pyridooxazolyl, pyridoimidazolyl, pyridothiazolyl, pyridinyl, pyridinonyl, pyridyl, pyrimidinyl, 2H-pyrrolyl, pyrrolyl, quinazolinyl, quinolinyl, 4H-quinolizinyl, quinoxalinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, triazolopyrimidinyl, 1,2,3-triazolyl, 1,2,4-triazolyl, 1,2,5-triazolyl, and 1,3,4-triazolyl.

The term “hydroxyalkyl” means an alkyl group as defined above, where the alkyl group is substituted with one or more OH groups. Examples of hydroxyalkyl groups include HO—CH₂—, HO—CH₂—CH₂— and CH₃—CH(OH)—.

The term “cyano” as used herein means a substituent having a carbon atom joined to a nitrogen atom by a triple bond, i.e., C≡N.

As used herein, “oxo” is means a “═O” group.

As used herein, the phrase “pharmaceutically acceptable” refers to those compounds or tautomers thereof, or salts thereof, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used herein, “pharmaceutically acceptable salts” refer to derivatives of the disclosed compounds or tautomers thereof, wherein the parent compound or a tautomer thereof, is modified by making of the acid or base salts thereof of the parent compound or a tautomer thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound, or a tautomer thereof, formed, for example, from non-toxic inorganic or organic acids. For example, such conventional non-toxic salts include, but are not limited to, those derived from inorganic and organic acids selected from 2-acetoxybenzoic, 2-hydroxy ethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, and toluene sulfonic.

The pharmaceutically acceptable salts of the present disclosure can be synthesized from the parent compound or a tautomer thereof that contains a basic or acidic moiety by conventional chemical methods. Generally, such pharmaceutically acceptable salts can be prepared by reacting the free acid or base forms of these compounds or tautomers thereof with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th ed., Mack Publishing Company, Easton, PA, USA, p. 1445 (1990).

As used herein, “stable compound” and “stable structure” are meant to indicate a compound that is sufficiently robust to survive isolation to a useful degree of purity from a reaction mixture, and formulation into an efficacious therapeutic agent.

As used herein, the term “treating” refers to administering a compound or pharmaceutical composition as provided herein for therapeutic purposes. The term “therapeutic treatment” refers to administering treatment to a patient already suffering from a disease thus causing a therapeutically beneficial effect, such as ameliorating existing symptoms, ameliorating the underlying metabolic causes of symptoms, postponing or preventing the further development of a disorder, and/or reducing the severity of symptoms that will or are expected to develop.

As used herein, “unsaturated” refers to compounds having at least one degree of unsaturation (e.g., at least one multiple bond) and includes partially and fully unsaturated compounds.

As used herein, the term “effective amount” refers to an amount of a compound or a pharmaceutically acceptable salt of the compound or tautomer (including combinations of compounds and/or tautomers thereof, and/or pharmaceutically acceptable salts of said compound or tautomer) of the present disclosure that is effective when administered alone or in combination as an antimicrobial agent. For example, an effective amount refers to an amount of the compound or tautomer thereof, or a pharmaceutically acceptable salt said compound or tautomer that is present in a composition, a formulation given to a recipient patient or subject sufficient to elicit biological activity.

In the specification, the singular forms also include the plural, unless the context clearly dictates otherwise. Unless defined otherwise, all technical and scientific terms used herein have the same meaning as commonly understood by one of ordinary skill in the art to which this disclosure belongs. In the case of conflict, the present specification will control. As used herein, “mammal” refers to human and non-human patients.

As used herein, the term “formulae of the disclosure” or “formulae disclosed herein” includes one or more of the Formula I, its subformulas Ia, Ib, or Ic, and further subformulas thereof.

As used herein, the term “compound of the disclosure” or “compound disclosed herein” includes one or more compounds of the formulae of the disclosure or a compound explicitly disclosed herein.

All percentages and ratios used herein, unless otherwise indicated, are by weight.

Throughout the description, where compositions are described as having, including, or comprising specific components, or where processes are described as having, including, or comprising specific process steps, it is contemplated that compositions of the present disclosure also consist essentially of, or consist of, the recited components, and that the processes of the present disclosure also consist essentially of, or consist of, the recited processing steps. Further, it should be understood that the order of steps or order for performing certain actions are immaterial so long as the disclosure remains operable. Moreover, two or more steps or actions can be conducted simultaneously.

Contemplated equivalents of the compounds described above include compounds which otherwise correspond thereto, and which have the same general properties thereof (e.g., the ability to modulate TRPML), wherein one or more simple variations of substituents are made which do not adversely affect the efficacy of the compound. In general, the compounds of the present disclosure may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The term, “treat” or “treatment,” as used herein, refers to the application or administration of a compound, alone or in combination with, an additional agent to a subject, e.g., a subject who has a disorder (e.g., a disorder as described herein), a symptom of a disorder, or a predisposition toward a disorder, with the purpose to cure, heal, alleviate, relieve, alter, remedy, ameliorate, improve or affect the disorder.

As used herein, the term “subject” is intended to include human and non-human animals. Exemplary human subjects include a human subject having a disorder, e.g., a disorder described herein. The term “non-human animals” of the disclosure includes all vertebrates, e.g., non-mammals (such as chickens, amphibians, reptiles) and mammals, such as non-human primates, domesticated and/or agriculturally useful animals, e.g., sheep, dog, cat, cow, pig, etc.

The terms “antagonist” and “inhibitor” are used interchangeably to refer to an agent that decreases or suppresses a biological activity.

The terms “activator” and “agonist” are used interchangeably to refer to an agent that increases or initiates a biological activity.

The term “hydrate” as used herein, refers to a compound formed by the union of water with the parent compound.

The term “preventing,” when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The term “solvate” as used herein, refers to a compound formed by solvation (e.g., a compound formed by the combination of solvent molecules with molecules or ions of the solute). Another aspect of the disclosure features a pharmaceutical preparation suitable for use in a human patient, or for veterinary use, comprising an effective amount of a compound of the formulae of the disclosure (or a salt thereof, or a solvate, hydrate, oxidative metabolite or prodrug of the compound or its salt), and one or more pharmaceutically acceptable excipients. The disclosure further contemplates the use of compounds of the formulae of the disclosure in the manufacture of a medicament or pharmaceutical preparation to treat or reduce the symptoms of any of the diseases or conditions provided in the specification. The compounds of the formulae of the disclosure for use in treating a particular disease or condition can be formulated for administration via a route appropriate for the particular disease or condition.

Compounds of the formulae of the disclosure can be administered alone or in combination with another therapeutic agent. For instance, the compounds of the formulae of the disclosure can be administered conjointly with one or more of an agent for treating polycystic kidney disease, etc.

Compounds of the formulae of the disclosure can be administered topically, orally, transdermally, rectally, vaginally, parentally, intranasally, intrapulmonary, intraocularly, intravenously, intramuscularly, intraarterially, intrathecally, intracapsularly, intraorbitally, intracardiacly, intradermally, intraperitoneally, transtracheally, subcutaneously, subcuticularly, intraarticularly, subcapsularly, subarachnoidly, intraspinally, intrasternally, sublingually, or by inhalation.

In some embodiments, compounds of Formula I, Ia, Ib, or Ic can be administered topically.

In some embodiments, compounds of Formula I, Ia, Ib, or Ic can be administered orally.

In some embodiments, compounds of Formula I, Ia, Ib, or Ic can be administered parentally.

Compounds of Formula I, Ia, Ib, or Ic include molecules having an aqueous solubility suitable for oral or parenteral (e.g., intravenous) administration leading to or resulting in the treatment of a disorder described herein, for example the treatment of pain. In some embodiments, the compound is formulated into a composition suitable for oral administration.

In some embodiments, a compound of Formula I, Ia, Ib, or Ic can be administered as part of an oral or parenteral (e.g., intravenous) pharmaceutical composition to treat a disorder described herein in a therapeutically effective manner.

Certain compounds disclosed herein may exist in particular geometric or stereoisomeric forms. The present disclosure contemplates all such compounds, including cis- and trans-isomers, R- and S-enantiomers, diastereomers, (d)-isomers, (l)-isomers, the racemic mixtures thereof, and other mixtures thereof, as falling within the scope of the disclosure. For example, if one chiral center is present in a molecule, the disclosure includes racemic mixtures, enantiomerically enriched mixtures, and substantially enantiomerically or diastereomerically pure compounds. The composition can contain, e.g., more than 50%, more than 60%, more than 70%, more than 80%, more than 90%, more than 95%, or more than 99% of a single enantiomer or diastereomer. Additional asymmetric carbon atoms may be present in a substituent such as an alkyl group. All such isomers, as well as mixtures thereof, are intended to be included in this disclosure.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer.

$$\\ ee = ( 90 - 10 ) / 100 = 80 \% .$$

Thus, a composition containing 90% of one enantiomer and 10% of the other enantiomer is said to have an enantiomeric excess of 80%.

The “diastereomeric excess” or “% diastereomeric excess” of a composition can be calculated using the equation shown below. In the example shown below a composition contains 90% of one diastereomer, and 10% of another enantiomer.

$$\\ de = ( 90 - 10 ) / 100 = 80 \% .$$

Thus, a composition containing 90% of one diastereomer and 10% of the other diastereomer is said to have a diastereomeric excess of 80%.

Certain compounds disclosed herein can exist in unsolvated forms as well as solvated forms, including hydrated forms. In general, the solvated forms are equivalent to unsolvated forms and are encompassed within the scope of the present disclosure. Certain compounds disclosed herein may exist in multiple crystalline or amorphous forms. In general, all physical forms are equivalent for the uses contemplated by the present disclosure and are intended to be within the scope of the present disclosure.

### EXAMPLES

Examples are provided below to facilitate a more complete understanding of the invention. The following examples illustrate exemplary modes of making and practicing the invention. However, the scope of the invention is not limited to specific embodiments disclosed in these Examples, which are for purposes of illustration only, since alternative methods can be utilized to obtain similar results.

General. All oxygen and/or moisture sensitive reactions were carried out under N₂ atmosphere in glassware that was flame-dried under vacuum (0.5 mmHg) and purged with N₂ prior to use. All reagents and solvents were purchased from commercial vendors and used as received, or synthesized according to the footnoted references. NMR spectra were recorded on a Bruker 400 (400 MHZ ¹H, 75 MHz ¹³C) or Varian (400 MHz ¹H, 75 MHz ¹³C) spectrometer. Proton and carbon chemical shifts are reported in ppm (δ) referenced to the NMR solvent. Data are reported as follows: chemical shifts, multiplicity (br=broad, s=singlet, t=triplet, q=quartet, m=multiplet; coupling constant(s) in Hz). Unless otherwise indicated NMR data were collected at 25° C. Flash chromatography was performed using 100-200 mesh Silica Gel. Liquid Chromatography/Mass Spectrometry (LCMS) was performed on Agilent 1200HPLC and 6110 MS. Analytical thin layer chromatography (TLC) was performed on 0.2 mm silica gel plates. Visualization was accomplished with UV light and aqueous potassium permanganate (KMnO₄) stain followed by heating.

### LCMS Typical Conditions:

LC-MS conditions Instrument: LCMS2020(E-LCMS 008) Column: Shim-pack GIST C18, 50*4.6 mm 5 um Mobile Phase: A: H2O (0.1% FA) B: CH3CN Temperature: 35iæ Flow rate: 2.5 mL/min Run time: 0.1 min@20% B, 1.7 min gradient (20-95% B), then 0.7 min@95% B, then 0.4 min@20% B Injection volume: 5 uL Detector: UV 220/254 nm Mass range: 100-1000 Scan: Positive/Negative.

### HPLC Typical Conditions:

Instrument: LC-20AD (E-LC 006) Column: YMC Triart C18, 50×4.6 mm, 5 um Mobile phase: Solvent A: H₂O/CH₃CN/TFA=90/10/0.1 Solvent B: H₂O/CH₃CN/TFA=10/90/0.1 Flow rate: 2.5 mL/min Run time: 0.4 min@ 60% B, 3.4 min gradient (60-100% B), then 0.8 min@100% B. Temperature: 35° C. Detector: UV.
Or
Instrument: LC-2010AHT (E-LC 001) Column: Gemini, C18, 50×4.6 mm, 5 um Mobile phase: Solvent A: H₂O/CH₃CN/TFA=90/10/0.1 Solvent B: H₂O/CH₃CN/TFA=10/90/0.1 Flow rate: 2.5 mL/min Run time: 0.4 min@20% B, 3.4 min gradient (20-95% B), then 0.8 min@95% B. Temperature: 40° C. Detector: UV.

TABLE 2
Abbreviations

| | |
|---|---|
| Boc | t-butoxycarbonyl |
| Cu(OAc)₂ | copper(II) acetate |
| DCM | dichloromethane |
| DIEA | N,N-diisopropylethylamine |
| DIPEA | N,N-diisopropylethylamine |
| DMF | N,N-dimethylformamide |
| DPPA | diphenylphosphorylazide |
| dppf | 1,1′-bis(diphenylphosphino)ferrocene |
| dtbpf | 1,1′-bis(di-t-butylphosphino)ferrocene |
| ESI | electrospray ionization |
| EtOAc | ethyl acetate |
| EtOH | ethanol |
| Et₃N | triethylamine |
| HATU | O-(7-azabenzotriazol-1-yl)-N,N,N′,N′- |
| | tetramethyluronium hexafluorophosphate |
| ¹H NMR | proton nuclear magnetic resonance |
| LCMS | liquid chromatography mass spectrometry |
| mCPBA | meta-chloroperbenzoic acid |
| Me | methyl |
| MeOH | methanol |
| MsCl | methanesulfonyl chloride |
| Ms | methanesulfonyl |
| Pd₂(dba)₃ | tris(dibenzylideneacetone)dipalladium |
| Pd(dppf)Cl₂ | 1,1-bis(diphenylphosphino)ferrocene- |
| | palladium(II)dichloride |
| Pd(PPh₃)₄ | tetrakis(triphenylphosphine)palladium(0) |
| Pd-118 or | 1,1-bis(di-t-butylphosphino)ferrocene- |
| Pd(dtbpf)Cl₂ | palladium(II)dichloride |
| SFC | supercritical fluid chromatography |
| TBAF | tetrabutylammonium fluoride |
| TEA | triethylamine |
| TFA | trifluoroacetic acid |
| TFAA | trifluoroacetic anhydride |
| THF | tetrahydrofuran |
| TLC | thin layer chromatography |
| Tol | toluene |
| Ts | p-toluenesulfonyl |
| TsCl | 4-toluenesulfonyl chloride |
| TsOH | tosylic acid |
| x-phos | dicyclohexyl[2′,4′,6′-tris(propan-2- |
| | yl)[1,1′-biphenyl]-2-yl]phosphane |

### Example 1. Synthesis of (S)-2-(5-(2-fluorophenyl)-4-(4-isobutyryl-2-methylpiperazin-1-yl)-7H-pyrrolo[2,3-d]pyrimidin-7-yl)isonicotinonitrile (Compound 102)

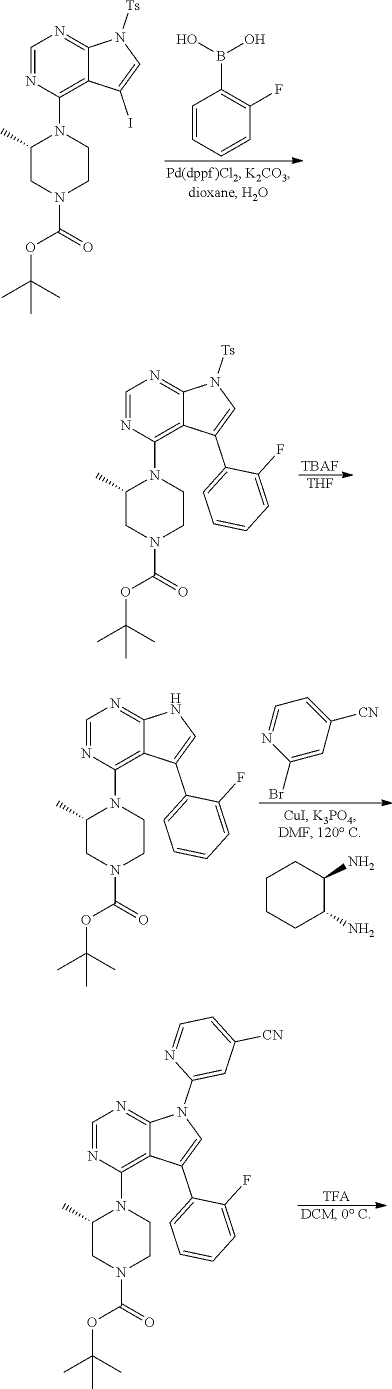

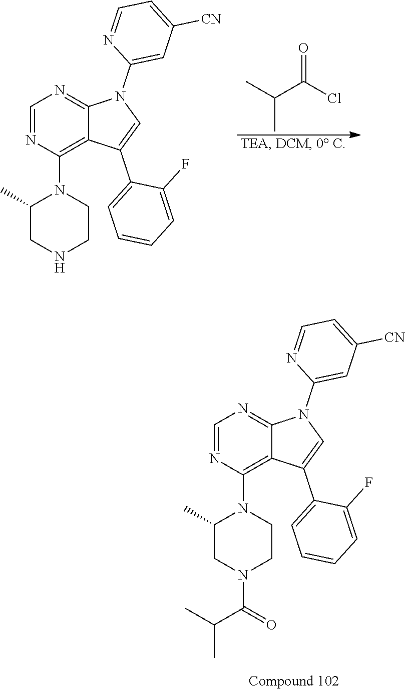

### Step 1. tert-butyl (S)-4-(5-(2-fluorophenyl)-7-tosyl-7H-pyrrolo[2,3-d]pyrimidin-4-yl)-3-methylpiperazine-1-carboxylate

To a solution of tert-butyl (S)-4-(5-iodo-7-tosyl-7H-pyrrolo[2,3-d]pyrimidin-4-yl)-3-methylpiperazine-1-carboxylate (60 g, 100 mmol, prepared following the procedure outlined in the experimental of compound 128) in dioxane/water (500 mL, 5:1) were added (2-fluorophenyl) boronic acid (28 g, 200 mmol), K₂CO₃ (42 g, 301 mmol) and Pd(dppf)Cl₂ (7.4 g, 10 mmol). The resulting mixture was heated to 80° C. overnight. After being cooled to room temperature, the reaction mixture was filtered. The filtrate was partitioned between DCM and water, the organic layer was separated, and the aqueous layer was extracted with DCM twice. The combined organic layers were dried over Na₂SO₄, filtered, and concentrated. The residue was purified by flash chromatography (0˜50% ethyl acetate in petroleum ether) to afford tert-butyl (S)-4-(5-(2-fluorophenyl)-7-tosyl-7H-pyrrolo[2,3-d]pyrimidin-4-yl)-3-methylpiperazine-1-carboxylate (40 g, 70% yield) as a white solid. LC/MS ESI m/z: 566.6 (M+H)⁺.

### Step 2. tert-butyl (S)-4-(5-(2-fluorophenyl)-7H-pyrrolo[2,3-d]pyrimidin-4-yl)-3-methylpiperazine-1-carboxylate

To a solution of tert-butyl (S)-4-(5-(2-fluorophenyl)-7-tosyl-7H-pyrrolo[2,3-d]pyrimidin-4-yl)-3-methylpiperazine-1-carboxylate (37 g, 65 mmol) in THF (300 ml) was added TBAF (220 ml, 1M in THF). The resulting mixture was stirred at room temperature overnight. After removal of ⅔ of the solvent, the remaining mixture was poured into ice water. The crude product was collected by filtration and dried under vacuum to give tert-butyl (S)-4-(5-(2-fluorophenyl)-7H-pyrrolo[2,3-d]pyrimidin-4-yl)-3-methylpiperazine-1-carboxylate (25 g, 93% yield) as a gray solid. LC/MS ESI m/z: 412.5 (M+H)⁺.

### Step 3. tert-butyl (S)-4-(7-(4-cyanopyridin-2-yl)-5-(2-fluorophenyl)-7H-pyrrolo[2,3-d]pyrimidin-4-yl)-3-methylpiperazine-1-carboxylate

To a solution of tert-butyl (S)-4-(5-(2-fluorophenyl)-7H-pyrrolo[2,3-d]pyrimidin-4-yl)-3-methylpiperazine-1-carboxylate (10 g, 24 mmol) in DMF (80 mL) were added 2-bromopyridine-4-carbonitrile (8.9 g, 48 mmol), CuI (1.4 g, 7.3 mmol), trans-cyclohexane-1,2-diamine (0.83 g, 7.3 mmol) and K₃PO₄ (15 g, 73 mmol). The resulting mixture was heated to 120° C. overnight under N₂. After being cooled to room temperature, the reaction was filtered, and the solvent was removed. The residue was purified by flash chromatography (0˜30% EtOAc (with 50% DCM) in petroleum ether) to afford tert-butyl (S)-4-(7-(4-cyanopyridin-2-yl)-5-(2-fluorophenyl)-7H-pyrrolo[2,3-d]pyrimidin-4-yl)-3-methylpiperazine-1-carboxylate (9.3 g, 74.5% yield) as a white solid. LC/MS ESI m/z: 514.6 (M+H)⁺. ¹H NMR (400 MHZ, CDCl₃) δ 9.40 (s, 1H), 8.61 (d, J=4.9 Hz, 1H), 8.56 (s, 1H), 8.27 (s, 1H), 7.46 (t, J=7.2 Hz, 1H), 7.41-7.35 (m, 2H), 7.27-7.18 (m, 2H), 4.24 (s, 1H), 3.77 (s, 1H), 3.56-3.44 (m, 2H), 3.08 (t, J=11.5 Hz, 1H), 2.89-2.62 (m, 2H), 1.43 (s, 9H), 1.02 (d, 3H).

### Step 4. (S)-2-(5-(2-fluorophenyl)-4-(2-methylpiperazin-1-yl)-7H-pyrrolo[2,3-d]pyrimidin-7-yl)isonicotinonitrile

At 0° C., to a solution of tert-butyl (S)-4-(7-(4-cyanopyridin-2-yl)-5-(2-fluorophenyl)-7H-pyrrolo[2,3-d]pyrimidin-4-yl)-3-methylpiperazine-1-carboxylate (2.9 g, 5.6 mmol) in DCM (13 mL) was added TFA (13 mL) and the resulting mixture was stirred at room temperature for 2 h. The reaction mixture was basified with NaHCO₃ (aq.) and extracted with Na₂SO₄, filtered, and concentrated. The product was used in the next step directly. LC/MS ESI m/z: 414.6 (M+H)⁺.

### Step 5. (S)-2-(5-(2-fluorophenyl)-4-(4-isobutyryl-2-methylpiperazin-1-yl)-7H-pyrrolo[2,3-d]pyrimidin-7-yl)isonicotinonitrile

At 0° C., to a solution of (S)-2-(5-(2-fluorophenyl)-4-(2-methylpiperazin-1-yl)-7H-pyrrolo[2,3-d]pyrimidin-7-yl)isonicotinonitrile (2.7 g, 6.5 mmol) in DCM (13 mL) was added TEA (2.7 mL, 19 mmol), followed by di-isobutyryl chloride (1.3 mL, 13 mmol) dropwise. The resulting mixture was stirred at room temperature for 20 minutes. The reaction mixture was quenched with ice water and extracted with DCM twice. The combined organic layers were washed with NaHCO₃ (aq.), dried over Na₂SO₄, filtered, and concentrated. The residue was purified by flash chromatography (0˜50% ethyl acetate in petroleum ether) to afford (S)-2-(5-(2-fluorophenyl)-4-(4-isobutyryl-2-methylpiperazin-1-yl)-7H-pyrrolo[2,3-d]pyrimidin-7-yl)isonicotinonitrile (2.5 g, 81%) as a white solid. LC/MS ESI m/z: 484.6 (M+H)⁺. ¹H NMR (400 MHZ, DMSO-d6) δ 9.18 (s, 1H), 8.79 (d, J=5.0 Hz, 1H), 8.58 (s, 1H), 8.24 (s, 1H), 7.86-7.83 (m, 1H), 7.59-7.54 (m, 1H), 7.52-7.46 (m, 1H), 7.41-7.35 (m, 2H), 4.29-4.13 (m, 1H), 4.00-3.86 (m, 1H), 3.71 (d, J=11.8 Hz, 0.5H, rotamers), 3.62-3.47 (m, 1H), 3.41 (d, J=13.6 Hz, 0.5H, rotamers), 3.10-2.88 (m, 2H), 2.87-2.76 (m, 1H), 2.50-2.32 (m, 1H), 1.01-0.89 (m, 8H), 0.82 (m, 1H).

The following compounds were prepared by procedures analogous to the synthesis of compound 102 from the corresponding acid chlorides.

| Cmpd | | |
|---|---|---|
| No. | Chemical Name | LCMS and ¹H NMR |
| 103 | (S)-2-(5-(2-fluorophenyl)-4-(2- | LC/MS ESI (m/z): 498 (M + H)⁺. ¹H NMR (400 MHz, |
| | methyl-4-pivaloylpiperazin-1- | CDCl₃) δ 9.41 (s, 1H), 8.63 (d, J = 5.0 Hz, 1H), 8.59 (s, |
| | yl)-7H-pyrrolo[2,3-d]pyrimidin- | 1H), 8.29 (s, 1H), 7.51-7.45 (m, 1H), 7.42-7.39 (m, 1H), |
| | 7-yl)isonicotinonitrile | 7.39-7.34 (m, 1H), 7.28-7.17 (m, 2H), 4.21-4.13 (m, |
| | | 1H), 4.02-3.95 (m, 1H), 3.89 (d, J = 13.1 Hz, 1H), 3.57- |
| | | 3.49 (m, 1H), 3.19-3.09 (m, 1H), 3.01-2.91 (m, 1H), |
| | | 2.79 (dd, J = 12.3, 1.8 Hz, 1H), 1.25 (s, 9H), 0.98 (d, J = |
| | | 6.6 Hz, 3H). |
| 105 | (S)-2-(4-(4- | LC/MS ESI (m/z): 482 (M + H)⁺. ¹H NMR (400 MHz, |
| | (cyclopropanecarbonyl)-2- | CDCl₃) δ 9.41 (s, 1H), 8.63 (d, J = 5.0 Hz, 1H), 8.58 (s, |
| | methylpiperazin-1-yl)-5-(2- | 1H), 8.29 (s, 1H), 7.52-7.44 (m, 1H), 7.42-7.40 (m, 1H), |
| | fluorophenyl)-7H-pyrrolo[2,3- | 7.40-7.34 (m, 1H), 7.26-7.17 (m, 2H), 4.50-3.94 (m, |
| | d]pyrimidin-7- | 2H), 3.92-3.42 (m, 2H), 3.31-2.97 (m, 2H), 2.72-2.48 |
| | yl)isonicotinonitrile | (m, 1H), 1.74-1.62 (m, 1H), 1.19-1.03 (m, 2H), 0.99- |
| | | 0.91 (m, 3H), 0.79-0.69 (m, 2H). |
| 133 | (S)-2-(4-(4-(3,3- | LC/MS ESI (m/z): 512 (M + H)⁺. ¹H NMR (400 MHz, |
| | dimethylbutanoyl)-2- | CDCl₃) δ 9.40 (s, 1H), 8.62 (d, J = 5.0 Hz, 1H), 8.58 (d, J = |
| | methylpiperazin-1-yl)-5-(2- | 5.6 Hz, 1H), 8.29 (d, J = 5.8 Hz, 1H), 7.50-7.44 (m, 1H), |
| | fluorophenyl)-7H-pyrrolo[2,3- | 7.40 (d, J = 4.9 Hz, 1H), 7.39-7.34 (m, 1H), 7.26-7.17 |
| | d]pyrimidin-7- | (m, 2H), 4.37 (m, 0.5H, rotamers), 4.20-4.11 (m, 1H), |
| | yl)isonicotinonitrile | 4.05 (d, J = 13.2 Hz, 0.5H, rotamers), 3.68-3.58 (m, 1H), |
| | | 3.44 (d, J = 13.3 Hz, 1H), 3.20-2.97 (m, 2H), 2.59-2.51 |
| | | (m, 1H), 2.30-2.21 (m, 1.5H, rotamers), 2.10-2.04 (m, |
| | | 0.5H, rotamers), 1.10 (d, J = 6.7 Hz, 1.5H, rotamers), 1.03- |
| | | 1.01 (m, 9H), 0.93 (d, J = 6.6 Hz, 1.5H, rotamers). |
| 134 | (S)-2-(5-(2-fluorophenyl)-4-(2- | LC/MS ESI (m/z): 498 (M + H)⁺. ¹H NMR (400 MHz, |
| | methyl-4-(3- | CDCl₃) δ 9.40 (s, 1H), 8.62 (d, J = 5.0 Hz, 1H), 8.58 (d, J = |
| | methylbutanoyl)piperazin-1-yl)- | 6.1 Hz, 1H), 8.29 (d, J = 6.4 Hz, 1H), 7.50-7.44 (m, 1H), |
| | 7H-pyrrolo[2,3-d]pyrimidin-7- | 7.42-7.35 (m, 2H), 7.26-7.16 (m, 2H), 4.41-4.36 (m, |
| | yl)isonicotinonitrile | 0.5H, rotamers), 4.18 (d, J = 13.5 Hz, 0.5H, rotamers), 4.14- |
| | | 4.07 (m, 0.5H, rotamers), 4.01 (d, J = 13.1 Hz, 0.5H, |
| | | rotamers), 3.65-3.57 (m, 1H), 3.47-3.37 (m, 1H), 3.19- |
| | | 2.97 (m, 2H), 2.59-2.49 (m, 1H), 2.26-2.21 (m, 0.5H, |
| | | rotamers), 2.17-2.06 (m, 2.5H, rotamers), 1.09 (d, J = 6.7 |
| | | Hz, 1.5H, rotamers), 0.96-0.90 (m, 7.5H, rotamers). |

### Example 2. Synthesis of of isopropyl(S)-2-(5-cyclopropyl-4-(2-methyl-4-pivaloylpiperazin-1-yl)-7H-pyrrolo[2,3-d]pyrimidin-7-yl)isonicotinonitrile (Compound 104)

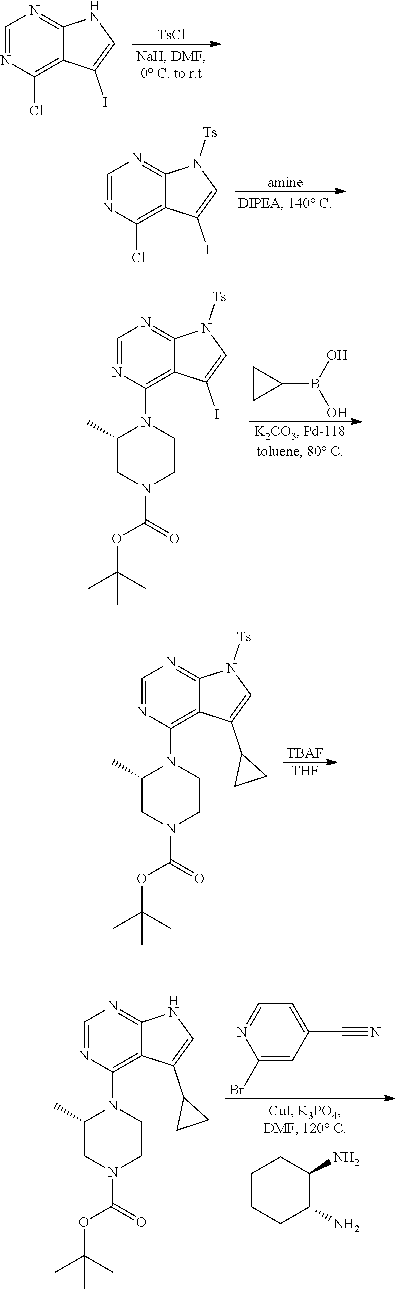

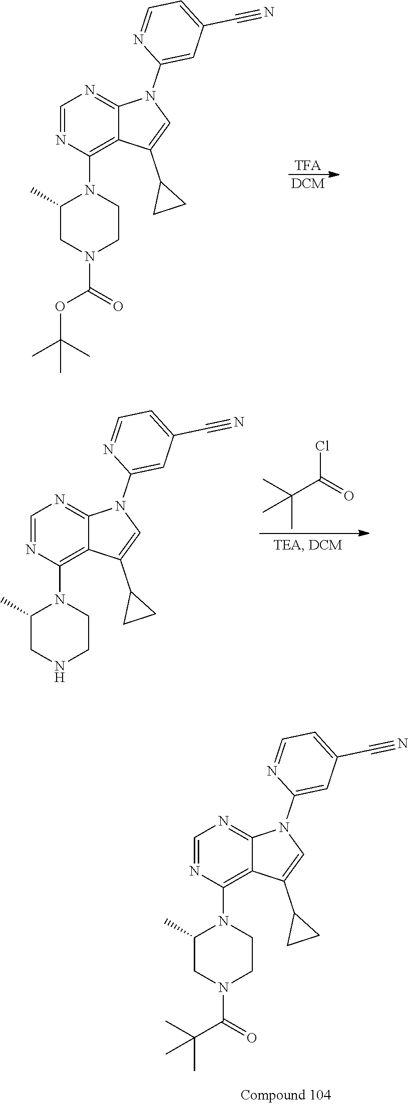

### Step 1. 4-chloro-5-iodo-7-tosyl-7H-pyrrolo[2,3-d]pyrimidine

At 0° C., to a suspension of NaH (11 g, 270 mmol) in DMF (500 mL) was added a solution of 4-chloro-5-iodo-7H-pyrrolo[2,3-d]pyrimidine (50 g, 180 mmol) in DMF (150 ml) dropwise. After being stirred at 0° C. for 10 minutes, a solution of TsCl (50 g, 260 mmol) in DMF (150 ml) was added dropwise into the above reaction mixture. The resulting mixture was stirred at room temperature overnight. The reaction was poured into water (4 L) and filtered to give the product which was further dried under vacuum to give 4-chloro-5-iodo-7-tosyl-7H-pyrrolo[2,3-d]pyrimidine (73 g, 94%) as a light yellow solid. LC/MS ESI m/z: 434.2 (M+H)⁺.

### Step 2. tert-butyl (S)-4-(5-iodo-7-tosyl-7H-pyrrolo[2,3-d]pyrimidin-4-yl)-3-methylpiperazine-1-carboxylate

A mixture of 4-chloro-5-iodo-7-tosyl-7H-pyrrolo[2,3-d]pyrimidine (50 g, 120 mmol) and tert-butyl-(S)-3-methylpiperazine-1-carboxylate (46 g, 230 mmol) in DIPEA (350 ml) was heated to 140° C. for 1.5 h. The mixture was concentrated, and the residue was purified by flash chromatography (silica gel, 0˜30% ethyl acetate in petroleum ether) to afford tert-butyl (S)-4-(5-iodo-7-tosyl-7H-pyrrolo[2,3-d]pyrimidin-4-yl)-3-methylpiperazine-1-carboxylate (52 g, 75%) as a white solid. LC/MS ESI m/z: 598.4 (M+H)⁺.

### Step 3. tert-butyl (S)-4-(5-cyclopropyl-7-tosyl-7H-pyrrolo[2,3-d]pyrimidin-4-yl)-3-methylpiperazine-1-carboxylate

To a solution of tert-butyl (S)-4-(5-iodo-7-tosyl-7H-pyrrolo[2,3-d]pyrimidin-4-yl)-3-methylpiperazine-1-carboxylate (39 g, 65 mmol) in toluene (400 mL) were added cyclopropylboronic acid (8.4 g, 98 mmol), K₂CO₃ (117 g, 849 mmol) and 1,1′-bis (di-tert-butylphosphino) ferrocene palladium dichloride (2.0 g, 3.2 mmol). The resulting mixture was heated to 80° C. overnight. After cooling to room temperature, the reaction mixture was filtered. The reaction was partitioned between DCM and water, the organic layer was separated, and the aqueous layer was extracted with DCM twice. The combined organic layers were dried over Na₂SO₄, filtered, and concentrated. The residue was purified by flash chromatography (0˜50% ethyl acetate in petroleum ether) to afford tert-butyl (S)-4-(5-cyclopropyl-7-tosyl-7H-pyrrolo[2,3-d]pyrimidin-4-yl)-3-methylpiperazine-1-carboxylate (22 g, 67% yield) as a white solid. LC/MS ESI m/z: 512.5 (M+H)⁺.

### Step 4. tert-butyl (S)-4-(5-cyclopropyl-7H-pyrrolo[2,3-d]pyrimidin-4-yl)-3-methylpiperazine-1-carboxylate

To a solution of tert-butyl (S)-4-(5-cyclopropyl-7-tosyl-7H-pyrrolo[2,3-d]pyrimidin-4-yl)-3-methylpiperazine-1-carboxylate (79 g, 160 mmol) in THF (500 ml) was added TBAF (400 ml, 1.0M in THF). The resulting mixture was stirred at room temperature overnight. After removal of solvent, the residue was diluted with H₂O and extracted with DCM twice. The combined organic layers were dried over Na₂SO₄, filtered, and concentrated. The residue was purified by flash chromatography (0˜50% ethyl acetate in petroleum ether) to afford tert-butyl (S)-4-(5-cyclopropyl-7H-pyrrolo[2,3-d]pyrimidin-4-yl)-3-methylpiperazine-1-carboxylate (49 g, 88%) as light yellow solid. LC/MS ESI m/z: 358.5 (M+H)⁺.

### Step 5. tert-butyl (S)-4-(7-(4-cyanopyridin-2-yl)-5-cyclopropyl-7H-pyrrolo[2,3-d]pyrimidin-4-yl)-3-methylpiperazine-1-carboxylate

To a solution of tert-butyl (S)-4-(5-cyclopropyl-7H-pyrrolo[2,3-d]pyrimidin-4-yl)-3-methylpiperazine-1-carboxylate (18 g, 50 mmol) in DMF (180 mL) were added 2-bromoisonicotinonitrile (18 g, 100 mmol), CuI (4.8 g, 25 mmol), (+/−)-trans-1,2-diaminocyclohexane (1.7 g, 15 mmol), and K₃PO₄ (32 g, 150 mmol). The resulting mixture was heated to 120° C. overnight. After cooling to room temperature, the reaction was filtered, and solvent was removed. The residue was purified by flash chromatography (0˜30% EtOAc (with 50% DCM) in petroleum ether) to afford tert-butyl (S)-4-(7-(4-cyanopyridin-2-yl)-5-cyclopropyl-7H-pyrrolo[2,3-d]pyrimidin-4-yl)-3-methylpiperazine-1-carboxylate (12 g, 51% yield) as a light yellow solid. LC/MS ESI m/z: 460.6 (M+H)⁺. ¹H NMR (400 MHz, CDCl₃) δ 9.32 (s, 1H), 8.58 (d, J=5.0 Hz, 1H), 8.50 (s, 1H), 7.77 (s, 1H), 7.33 (dd, J=5.0, 1.2 Hz, 1H), 4.74-4.66 (m, 1H), 4.18-3.83 (m, 3H), 3.55 (t, J=11.6 Hz, 1H), 3.39-3.09 (m, 2H), 2.06-1.99 (m, 1H), 1.50 (s, 9H), 1.24 (d, J=6.5 Hz, 3H), 1.06-1.02 (m, 2H), 0.90-0.84 (m, 1H), 0.80-0.74 (m, 1H).

### Step 6. (S)-2-(5-cyclopropyl-4-(2-methylpiperazin-1-yl)-7H-pyrrolo[2,3-d]pyrimidin-7-yl)isonicotinonitrile

At 0° C., to a solution of tert-butyl (S)-4-(7-(4-cyanopyridin-2-yl)-5-cyclopropyl-7H-pyrrolo[2,3-d]pyrimidin-4-yl)-3-methylpiperazine-1-carboxylate (2.0 g, 4.4 mmol) in DCM (20 mL) was added TFA (0.9 mL, 13 mmol). The resulting mixture was stirred for 2 h then quenched with NaHCO₃ (aq.) and extracted with DCM twice. The combined organic layers were dried over Na₂SO₄, filtered, and concentrated. The carried directly to the next step without further purification. LC/MS ESI m/z: 360 (M+H)⁺.

### Step 7. (S)-2-(5-cyclopropyl-4-(2-methyl-4-pivaloylpiperazin-1-yl)-7H-pyrrolo[2,3-d]pyrimidin-7-yl)isonicotinonitrile

At 0° C., to a solution of (S)-2-(5-cyclopropyl-4-(2-methylpiperazin-1-yl)-7H-pyrrolo[2,3-d]pyrimidin-7-yl)isonicotinonitrile (50 mg, 0.14 mmol) in DCM (5 mL) were added TEA (0.06 mL, 0.42 mmol) followed by pivaloyl chloride (34 mg, 0.28 mmol). The resulting mixture was stirred at that temperature for 20 minutes, then partitioned between DCM and water, and the organic layer was separated. The aqueous layer was extracted with DCM. The combined organic layer was washed with NaHCO₃ (aq.), dried over Na₂SO₄, filtered, and concentrated. The residue was purified by prep-HPLC to give (S)-2-(5-cyclopropyl-4-(2-methyl-4-pivaloylpiperazin-1-yl)-7H-pyrrolo[2,3-d]pyrimidin-7-yl)isonicotinonitrile (30 mg, 49%). LC/MS ESI m/z: 444 (M+H)⁺. ¹HNMR (400 MHZ, CD₃OD) δ 9.18 (s, 1H), 8.64 (d, J=5.0 Hz, 1H), 8.46 (s, 1H), 7.85 (s, 1H), 7.53 (dd, J=5.0, 1.2 Hz, 1H), 4.76 (s, 1H), 4.29 (m, 2H), 3.90 (d, J=13.3 Hz, 1H), 3.66-3.58 (m, 1H), 3.49-3.38 (m, 2H), 2.14-2.07 (m, 1H), 1.34 (s, 9H), 1.19 (d, J=6.6 Hz, 3H), 1.08 (dt, J=8.1, 4.0 Hz, 2H), 0.88 (dd, J=9.2, 4.3 Hz, 1H), 0.78-0.71 (m, 1H).

The following compounds were prepared by an analogous procedure to the synthesis of compound 104 from the corresponding acid chlorides or acids using standard amide coupling reactions with coupling agents like HATU.

| Cmpd | | |
|---|---|---|
| No. | Chemical Name | LCMS and ¹H NMR |
| 136 | (S)-2-(5-cyclopropyl-4-(4-(3,3- | LC/MS ESI m/z: 458 (M + H)+. ¹H NMR (400 MHz, |
| | dimethylbutanoyl)-2- | CD₃OD) δ 9.20 (s, 1H), 8.66 (d, J = 4.9 Hz, 1H), 8.48 (s, |
| | methylpiperazin-1-yl)-7H- | 1H), 7.87 (s, 1H), 7.54 (dd, J = 5.0, 1.3 Hz, 1H), 4.75 (m, |
| | pyrrolo[2,3-d]pyrimidin-7- | 1H), 4.39 (m 1H), 3.98 (m, 2H), 3.69-3.47 (m, 2H), 3.27 |
| | yl)isonicotinonitrile | (m, J = 3.2 Hz, 2H), 3.23-3.10 (m, 1H), 2.16-2.06 (m, |
| | | 1H), 1.25 (m, J = 6.6 Hz, 1H), 1.17 (m, J = 6.6 Hz, 1H), |
| | | 1.08 (d, J = 9.2 Hz, 12H), 0.89 (m, 1H), 0.80-0.71 (m, |
| | | 1H). |
| 137 | (S)-2-(5-cyclopropyl-4-(2- | LC/MS ESI m/z: 444 (M + H)+. ¹H NMR (400 MHz, |
| | methyl-4-(3- | CD₃OD) δ 9.22-9.17 (d, 1H), 8.65 (dd, J = 5.0, 0.7 Hz, |
| | methylbutanoyl)piperazin-1-yl)- | 1H), 8.47 (s, 1H), 7.87 (s, 1H), 7.54 (dd, J = 5.0, 1.3 Hz, |
| | 7H-pyrrolo[2,3-d]pyrimidin-7- | 1H), 4.78 (m, J = 13.6, 5.6 Hz, 1H), 4.38 (dd, J = 52.3, 13.8 |
| | yl)isonicotinonitrile | Hz, 1H), 4.07-3.84 (m, 2H), 3.69-3.47 (m, 2H), 3.27- |
| | | 3.08 (m, 1H), 2.47-2.27 (m, 2H), 2.12 (dt, J = 13.4, 6.7 |
| | | Hz, 2H), 1.21 (m, 3H), 1.12-1.06 (m, 2H), 1.01 (m, 6H), |
| | | 0.88 (m, 1H), 0.76 (m, 1H) |
| 160 | (S)-2-(5-cyclopropyl-4-(4-(2- | LC/MS ESI m/z: 446 (M + H)⁺. ¹H NMR (400 MHz, CDCl₃) |
| | hydroxy-2-methylpropanoyl)-2- | δ 9.32 (s, 1H), 8.59 (d, J = 4.9 Hz, 1H), 8.53 (s, 1H), 7.81 |
| | methylpiperazin-1-yl)-7H- | (s, 1H), 7.35 (d, J = 4.2 Hz, 1H), 4.89-4.66 (m, 1H), 4.58- |
| | pyrrolo[2,3-d]pyrimidin-7- | 4.01 (m, 3H), 3.95 (d, J = 13.1 Hz, 1H), 3.78-3.11 (m, |
| | yl)isonicotinonitrile | 3H), 2.11-1.99 (m, 1H), 1.57 (s, 3H), 1.56 (s, 3H), 1.24 (d, |
| | | J = 5.1 Hz, 3H), 1.10-1.00 (m, 2H), 0.95-0.85 (m, 1H), |
| | | 0.83-0.73 (m, 1H). |

### Example 3. Synthesis of 2-(5-(2-fluorophenyl)-4-(4-isobutyrylpiperazin-1-yl)-7H-pyrrolo[2,3-d]pyrimidin-7-yl)isonicotinonitrile (Compound 106)

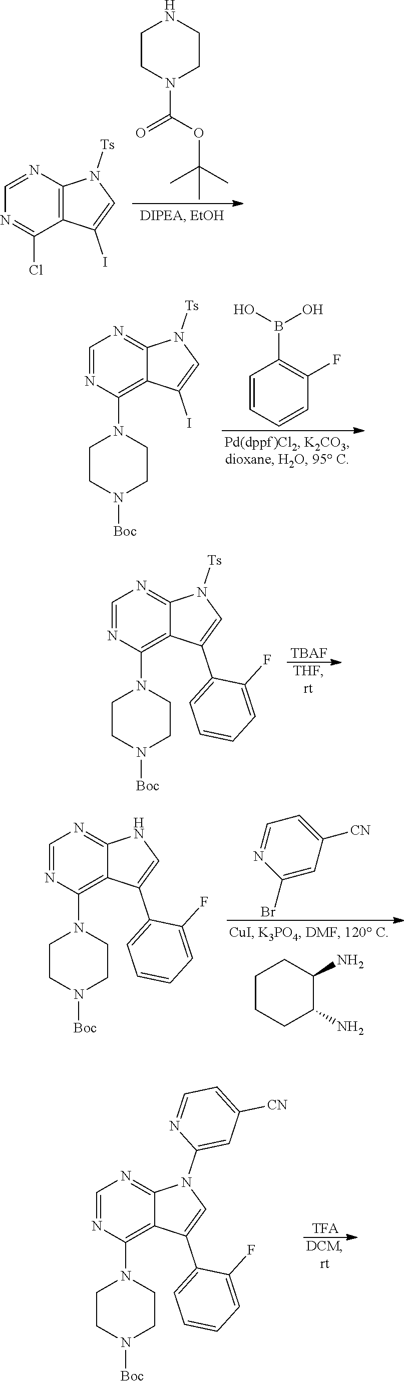

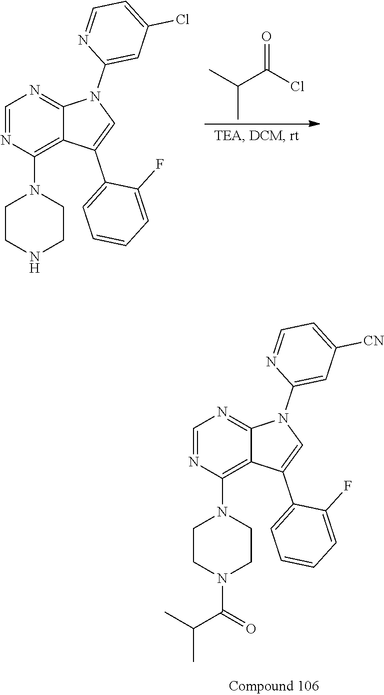

### Step 1. tert-butyl 4-(5-iodo-7-tosyl-7H-pyrrolo[2,3-d]pyrimidin-4-yl)piperazine-1-carboxylate

To a solution of 4-chloro-5-iodo-7-tosyl-7H-pyrrolo[2,3-d]pyrimidine (4.0 g, 9.2 mmol, prepared following the procedure outlined in compound 128) in EtOH (50 mL) were added tert-butyl piperazine-1-carboxylate (1.7 g, 9.2 mmol) and DIPEA (5.0 mL, 28 mmol), and the resulting mixture was heated to 100° C. overnight. The mixture was cooled to room temperature and concentrated. The residue was purified by flash chromatography (silica gel, 0˜30%, ethyl acetate in petroleum ether) to afford tert-butyl 4-(5-iodo-7-tosyl-7H-pyrrolo[2,3-d]pyrimidin-4-yl)piperazine-1-carboxylate (4.5 g, 83%) as a white solid. LC/MS ESI m/z: 584 (M+H)⁺.

### Step 2. tert-butyl 4-(5-(2-fluorophenyl)-7-tosyl-7H-pyrrolo[2,3-d]pyrimidin-4-yl)piperazine-1-carboxylate

A suspension of tert-butyl 4-[5-iodo-7-(4-methylbenzenesulfonyl)-7H-pyrrolo[2,3-d]pyrimidin-4-yl]piperazine-1-carboxylate (2.3 g, 4.0 mmol), (2-fluorophenyl) boronic acid (0.61 g, 4.4 mmol), K₂CO₃ (1.1 g, 8.0 mmol) and Pd(dppf)Cl₂ (0.29 g, 0.40 mmol) in dioxane-water (18 mL, v:v=5:1) was stirred at 95° C. under N₂ for 18 h. After cooling to room temperature, the reaction mixture was partitioned between EtOAc and water. The organic layer was separated, and the aqueous layer was extracted with EtOAc twice. The combined organic phases were washed with brine, dried over anhydrous Na₂SO₄, filtered, and concentrated. The product was used in the next step without further purification. LC/MS ESI m/z: 552 (M+H)⁺

### Step 3. tert-butyl 4-(5-(2-fluorophenyl)-7H-pyrrolo[2,3-d]pyrimidin-4-yl)piperazine-1-carboxylate

To a solution of tert-butyl 4-(5-(2-fluorophenyl)-7-tosyl-7H-pyrrolo[2,3-d]pyrimidin-4-yl)piperazine-1-carboxylate (1.8 g, 3.3 mmol) in THF (15 mL) was added TBAF (6.5 mL, 1.0M in THF). The resulting mixture was stirred at room temperature for 18 h. After removal of solvent, the residue was dissolved in EtOAc, washed with water, and the aqueous layer was extracted with EtOAc twice. The combined organic phases were dried over anhydrous Na₂SO₄, filtered, and concentrated. The residue was purified by column chromatography on silica gel (0˜60% EtOAc in petroleum ether) to give tert-butyl 4-[5-(2-fluorophenyl)-7H-pyrrolo[2,3-d]pyrimidin-4-yl]piperazine-1-carboxylate (1.2 g, 92%) as a white solid. LC/MS ESI m/z: 398 (M+H)⁺

### Step 4. tert-butyl 4-(7-(4-cyanopyridin-2-yl)-5-(2-fluorophenyl)-7H-pyrrolo[2,3-d]pyrimidin-4-yl)piperazine-1-carboxylate

A suspension of tert-butyl 4-[5-(2-fluorophenyl)-7H-pyrrolo[2,3-d]pyrimidin-4-yl]piperazine-1-carboxylate (1.2 g, 3.0 mmol), 2-bromopyridine-4-carbonitrile (0.66 g, 3.6 mmol), K₃PO₄ (1.3 g, 6.0 mmol), CuI (0.17 g, 0.90 mmol) and trans-cyclohexane-1,2-diamine (0.10 g, 0.90 mmol) in DMF (30 mL) was stirred at 120° C. under N₂ for 18 h. After being cooled to room temperature, the solvent was removed. The residue was added to water and EtOAc. The organic layer was separated, and the aqueous layer was extracted with EtOAc twice. The combined organic phases were washed with brine, dried over anhydrous Na₂SO₄, filtered, and concentrated. The residue was purified by column chromatography on silica gel (0˜60% EtOAc in petroleum ether) to give tert-butyl 4-[7-(4-cyanopyridin-2-yl)-5-(2-fluorophenyl)-7H-pyrrolo[2,3-d]pyrimidin-4-yl]piperazine-1-carboxylate (1.0 g, 2.0 mmol, 66%) as a white solid. LC/MS ESI m/z: 500 (M+H)⁺

### Step 5. 2-(5-(2-fluorophenyl)-4-(piperazin-1-yl)-7H-pyrrolo[2,3-d]pyrimidin-7-yl) isonicotinonitrile

To a solution of tert-butyl 4-[7-(4-cyanopyridin-2-yl)-5-(2-fluorophenyl)-7H-pyrrolo[2,3-d]pyrimidin-4-yl]piperazine-1-carboxylate (1.0 g, 2.0 mmol) in DCM (5.0 mL) was added TFA (4.5 mL, 60 mmol) and the reaction was stirred for 18 h at room temperature. The reaction mixture was quenched with NaHCO₃ (aq.) and extracted with DCM twice. The combined organic layers were washed with NaHCO₃ (aq.), dried over Na₂SO₄, filtered, and concentrated. The residue was used directly in next step without further purification. LC/MS ESI m/z: 400 (M+H)⁺.

### Step 6. 2-(5-(2-fluorophenyl)-4-(4-isobutyrylpiperazin-1-yl)-7H-pyrrolo[2,3-d]pyrimidin-7-yl)isonicotinonitrile

To a solution of 2-(5-(2-fluorophenyl)-4-(piperazin-1-yl)-7H-pyrrolo[2,3-d]pyrimidin-7-yl)isonicotinonitrile (80 mg, 0.20 mmol) and TEA (0.14 mL, 1.0 mmol) in DCM (2.0 mL) was added 2-methylpropanoyl chloride (0.03 mL, 0.30 mmol). The resulting mixture was stirred at room temperature for 3 h. The mixture was diluted with water and DCM. The layers were separated, and the aqueous layer was extracted with DCM twice. The combined organic phases were washed with brine, dried over anhydrous Na₂SO₄, filtered, and concentrated. The residue was purified by prep-HPLC to give 2-(5-(2-fluorophenyl)-4-(4-isobutyrylpiperazin-1-yl)-7H-pyrrolo[2,3-d]pyrimidin-7-yl)isonicotinonitrile (12 mg, 12%) as a white solid. LC/MS ESI m/z: 470 (M+H)⁺. ¹H NMR (400 MHZ, CDCl₃) δ 9.39 (s, 1H), 8.62 (d, J=5.0 Hz, 1H), 8.59 (s, 1H), 8.31 (s, 1H), 7.48 (td, J=7.5, 1.7 Hz, 1H), 7.42-7.34 (m, 2H), 7.28-7.18 (m, 2H), 3.45-3.29 (m, 6H), 3.26-3.14 (m, 2H), 2.72 (dt, J=13.5, 6.8 Hz, 1H), 1.08 (d, J=6.8 Hz, 6H).

The following compound was prepared by an analogous procedure to the synthesis of compound 106 from the corresponding acid chlorides.

| Cmpd | | |
|---|---|---|
| No. | Chemical Name | LCMS and ¹H NMR |
| 108 | 2-(5-(2-fluorophenyl)-4-(4- | LC/MS ESI (m/z): 484 (M + H)⁺. ¹H NMR (400 MHz, |
| | pivaloylpiperazin-1-yl)-7H- | CDCl₃) δ 9.40 (s, 1H), 8.63 (d, J = 4.9 Hz, 1H), 8.59 (s, |
| | pyrrolo[2,3-d]pyrimidin-7- | 1H), 8.31 (s, 1H), 7.49 (t, J = 7.4 Hz, 1H), 7.45-7.33 (m, |
| | yl)isonicotinonitrile | 2H), 7.29-7.18 (m, 2H), 3.42 (s, 4H), 3.30 (s, 4H), 1.24 (s, |
| | | 9H). |
| 110 | 2-(4-(4- | LC/MS ESI (m/z): 468 (M + H)⁺. ¹H NMR (400 MHz, |
| | (cyclopropanecarbonyl)piperazin- | CDCl₃) δ 9.40 (s, 1H), 8.63 (d, J = 4.9 Hz, 1H), 8.59 (s, |
| | 1-yl)-5-(2-fluorophenyl)-7H- | 1H), 8.31 (s, 1H), 7.49 (td, J = 7.6, 1.6 Hz, 1H), 7.43-7.34 |
| | pyrrolo[2,3-d]pyrimidin-7- | (m, 2H), 7.28-7.18 (m, 2H), 3.39 (dd, J = 80.1, 36.0 Hz, |
| | yl)isonicotinonitrile | 8H), 1.66 (td, J = 8.0, 4.0 Hz, 1H), 0.99-0.91 (m, 2H), |
| | | 0.78-0.69 (m, 2H). |

### Example 4. Synthesis of (S)-2-(4-(4-(1-fluorocyclopropane-1-carbonyl)-2-methylpiperazin-1-yl)-5-(2-fluorophenyl)-7H-pyrrolo[2,3-d]pyrimidin-7-yl)isonicotinonitrile (Compound 115)

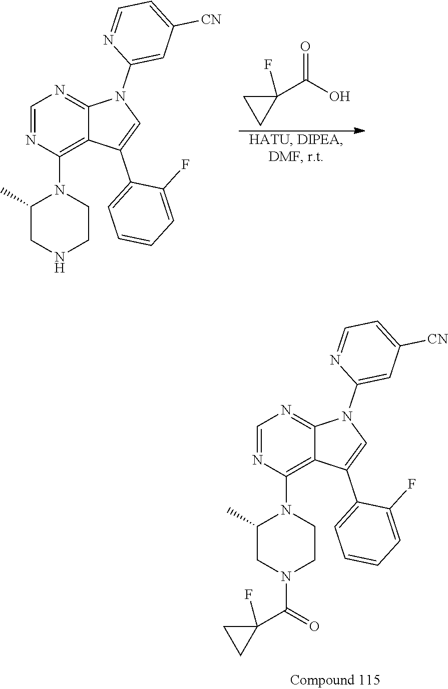

### Step 1. (S)-2-(4-(4-(1-fluorocyclopropane-1-carbonyl)-2-methylpiperazin-1-yl)-5-(2-fluorophenyl)-7H-pyrrolo[2,3-d]pyrimidin-7-yl)isonicotinonitrile

To a solution of (S)-2-(5-(2-fluorophenyl)-4-(2-methylpiperazin-1-yl)-7H-pyrrolo[2,3-d]pyrimidin-7-yl)isonicotinonitrile (50 mg, 0.12 mmol, prepared following the procedure outlined in the experimental of compound 102) in DMF (2 mL) were added DIPEA (0.1 mL, 0.60 mmol), 1-fluorocyclopropane-1-carboxylic acid (35 mg, 0.18 mmol) and HATU (91 mg, 0.24 mmol) respectively. The resulting reaction mixture was stirred at room temperature under N₂ overnight. The reaction mixture was partitioned between EtOAc and NaHCO₃ (aq.). The organic layer was separated, and the aqueous layer was extracted with EtOAc twice. The combined organic layers were washed with brine, dried over Na₂SO₄, filtered, and concentrated. The residue was purified by flash column chromatography (silica gel, 0˜30% EtOAc in petroleum ether), followed by prep-HPLC to provide(S)-2-(4-(4-(1-fluorocyclopropane-1-carbonyl)-2-methylpiperazin-1-yl)-5-(2-fluorophenyl)-7H-pyrrolo[2,3-d]pyrimidin-7-yl)isonicotinonitrile (14 mg, 24%) as a white solid. LC/MS ESI (m/z): 500 (M+H)⁺. ¹H NMR (400 MHZ, CDCl₃) δ 9.41 (s, 1H), 8.64-8.62 (m, 1H), 8.59 (s, 1H), 8.30 (s, 1H), 7.51-7.46 (m, 1H), 7.42-7.35 (m, 2H), 7.26-7.18 (m, 2H), 4.41-4.15 (m, 1H), 4.07 (d, J=12.7 Hz, 1H), 3.88 (d, J=13.2 Hz, 1H), 3.64-3.48 (m, 1H), 3.30-3.06 (m, 2H), 2.82-2.58 (m, 1H), 1.39-1.13 (m, 5H), 1.09-0.98 (m, 2H).

The following compound was prepared by an analogous procedure to the synthesis of compound 115 from the corresponding acids. The acid (4,4,4-trifluoro-3,3-dimethylbutanoic acid) for compound 140 was prepared following the procedure outlined in the experimental of compound 141.

| Cmpd | | |
|---|---|---|
| No. | Chemical Name | LCMS and ¹H NMR |
| 113 | 2-(5-(2-fluorophenyl)-4-((2S)-4- | LC/MS ESI (m/z): 488 (M + H)⁺. ¹H NMR (400 MHz, |
| | (2-fluoropropanoyl)-2- | CDCl₃) δ 9.41 (s, 1H), 8.63 (d, J = 5.0 Hz, 1H), 8.61-8.58 |
| | methylpiperazin-1-yl)-7H- | (m, 1H), 8.31-8.28 (m, 1H), 7.50-7.45 (m, 1H), 7.42- |
| | pyrrolo[2,3-d]pyrimidin-7- | 7.40 (m, 1H), 7.40-7.35 (m, 1H), 7.26-7.17 (m, 2H), |
| | yl)isonicotinonitrile | 5.31-5.09 (m, 1H), 4.38-3.92 (m, 2H), 3.78-3.47 (m, |
| | | 2H), 3.23-3.05 (m, 2H), 2.69-2.60 (m, 1H), 1.60-1.58 |
| | | (m, 1H), 1.55-1.48 (m, 2H), 1.11-0.93 (m, 3H). |
| 112 | (S)-2-(5-(2-fluorophenyl)-4-(2- | LC/MS ESI m/z: 496 (M + H)⁺. ¹H NMR (400 MHz, CDCl₃) |
| | methyl-4-(1- | δ 9.42-9.40 (m, 1H), 8.64-8.62 (m, 1H), 8.59 (s, 1H), |
| | methylcyclopropane-1- | 8.30 (s, 1H), 7.50-7.46 (m, 1H), 7.42-7.40 (m, 1H), 7.39- |
| | carbonyl)piperazin-1-yl)-7H- | 7.34 (m, 1H), 7.25-7.18 (m, 2H), 4.39-4.13 (m, 1H), |
| | pyrrolo[2,3-d]pyrimidin-7- | 4.08-3.99 (m, 1H), 3.84 (d, J = 13.1 Hz, 1H), 3.63-3.48 |
| | yl)isonicotinonitrile | (m, 1H), 3.18-3.08 (m, 1H), 3.06-2.32 (m, 2H), 1.27 (s, |
| | | 3H), 1.03-0.84 (m, 5H), 0.60-0.53 (m, 2H). |
| 125 | (S)-2-(4-(4-(2-fluoro-2- | LC/MS ESI m/z: 502 (M + H)⁺. ¹H NMR (400 MHz, CDCl₃) |
| | methylpropanoyl)-2- | δ 9.42-9.39 (m, 1H), 8.64-8.61 (m, 1H), 8.59 (s, 1H), |
| | methylpiperazin-1-yl)-5-(2- | 8.29 (s, 1H), 7.50-7.45 (m, 1H), 7.41-7.40 (m, 1H), 7.39- |
| | fluorophenyl)-7H-pyrrolo[2,3- | 7.34 (m, 1H), 7.26-7.18 (m, 2H), 4.34-4.05 (m, 2H), |
| | d]pyrimidin-7- | 3.97 (d, J = 13.2 Hz, 1H), 3.61-3.46 (m, 1H), 3.25-3.04 |
| | yl)isonicotinonitrile | (m, 2H), 2.74-2.58 (m, 1H), 1.64-1.60 (m, 2.5H, |
| | | rotamers), 1.58-1.51 (m, 3.5H, rotamers), 1.11-0.90 (m, |
| | | 3H). |
| 135 | (S)-2-(5-(2-fluorophenyl)-4-(2- | LC/MS ESI m/z: 538 (M + H)+. ¹H NMR (400 MHz, CDCl₃) |
| | methyl-4-(4,4,4- | δ 9.40 (s, 1H), 8.63 (d, J = 5.0 Hz, 1H), 8.59 (d, J = 3.7 Hz, |
| | trifluorobutanoyl)piperazin-1- | 1H), 8.30 (d, J = 3.6 Hz, 1H), 7.50-7.45 (m, 1H), 7.42- |
| | yl)-7H-pyrrolo[2,3-d]pyrimidin- | 7.40 (m, 1H), 7.40-7.35 (m, 1H), 7.26-7.18 (m, 2H), |
| | 7-yl)isonicotinonitrile | 4.39-4.34 (m, 0.5H, rotamers), 4.22-4.15 (m, 1H), 3.97 |
| | | (m, 0.5H, rotamers), 3.66-3.57 (m, 0.5H, rotamers), 3.53- |
| | | 3.45 (m, 1H), 3.34-3.29 (m, 0.5H, rotamers), 3.19-3.00 |
| | | (m, 2H), 2.70-2.45 (m, 5H), 1.09 (d, J = 6.7 Hz, 1.7H), |
| | | 0.95 (d, J = 6.7 Hz, 1.3H). |
| 140 | (S)-2-(5-(2-fluorophenyl)-4-(2- | LC/MS ESI m/z: 566 (M + H)⁺. ¹H NMR (400 MHz, CDCl₃) |
| | methyl-4-(4,4,4-trifluoro-3,3- | δ 9.40 (s, 1H), 8.63 (d, J = 5.0 Hz, 1H), 8.59 (d, J = 3.8 Hz, |
| | dimethylbutanoyl)piperazin-1- | 1H), 8.30 (d, J = 3.5 Hz, 1H), 7.50-7.45 (m, 1H), 7.42- |
| | yl)-7H-pyrrolo[2,3-d]pyrimidin- | 7.36 (m, 2H), 7.26-7.18 (m, 2H), 4.39-4.32 (m, 0.5H, |
| | 7-yl)isonicotinonitrile | rotamers), 4.20-4.14 (m, 1H), 4.02 (d, J = 13.1 Hz, 0.5H, |
| | | rotamers), 3.63-3.55 (m, 1H), 3.49-3.35 (m, 1H), 3.20- |
| | | 2.99 (m, 2H), 2.65-2.56 (m, 1H), 2.55-2.48 (m, 1H), |
| | | 2.46-2.41 (m, 0.5H, rotamers), 2.33-2.27 (m, 0.5H, |
| | | rotamers), 1.29-1.24 (m, 6H), 1.08 (d, J = 6.7 Hz, 1.5H, |
| | | rotamers), 0.94 (d, J = 6.6 Hz, 1.5H, rotamers). |
| 159 | (S)-2-(5-(2-fluorophenyl)-4-(4- | LC/MS ESI m/z: 500 (M + H)⁺. ¹H NMR (400 MHz, CDCl₃) |
| | (2-hydroxy-2-methylpropanoyl)- | δ 9.40 (s, 1H), 8.63 (d, J = 5.0 Hz, 1H), 8.59 (s, 1H), 8.30 |
| | 2-methylpiperazin-1-yl)-7H- | (s, 1H), 7.53-7.44 (m, 1H), 7.43-7.35 (m, 2H), 7.29- |
| | pyrrolo[2,3-d]pyrimidin-7- | 7.26 (m, 1H), 7.24-7.18 (m, 1H), 4.15 (s, 2H), 4.00 (d, J = |
| | yl)isonicotinonitrile | 12.6 Hz, 1H), 3.92 (s, 1H), 3.65-3.48 (m, 1H), 3.24-2.58 |
| | | (m, 3H), 1.46 (s, 6H), 0.99 (s, 3H). |
| 158 | 2-(5-(2-fluorophenyl)-4- | LC/MS ESI m/z: 514 (M + H)⁺. ¹H NMR (400 MHz, CDCl₃) |
| | ((2S,5R)-4-(2-hydroxy-2- | δ 9.38 (s, 1H), 8.66-8.59 (m, 2H), 8.32 (s, 1H), 7.50 (t, J = |
| | methylpropanoyl)-2,5- | 7.0 Hz, 1H), 7.45-7.37 (m, 2H), 7.30-7.27 (m, 1H), 7.25- |
| | dimethylpiperazin-1-yl)-7H- | 7.18 (m, 1H), 4.68-4.39 (m, 1.5H, rotamers), 4.05-3.84 |
| | pyrrolo[2,3-d]pyrimidin-7- | (m, 1H), 3.66-3.49 (m, 1H), 3.45-3.18 (m, 2H), 2.64- |
| | yl)isonicotinonitrile | 2.57 (m, 0.5H, rotamers), 1.57-1.23 (m, 9H), 1.07 (m, 3H). |

### Example 5. Synthesis of (S)-2-(4-(4-isobutyryl-2-methylpiperazin-1-yl)-5-(pyridin-2-yl)-7H-pyrrolo[2,3-d]pyrimidin-7-yl)isonicotinonitrile (Compound 116)

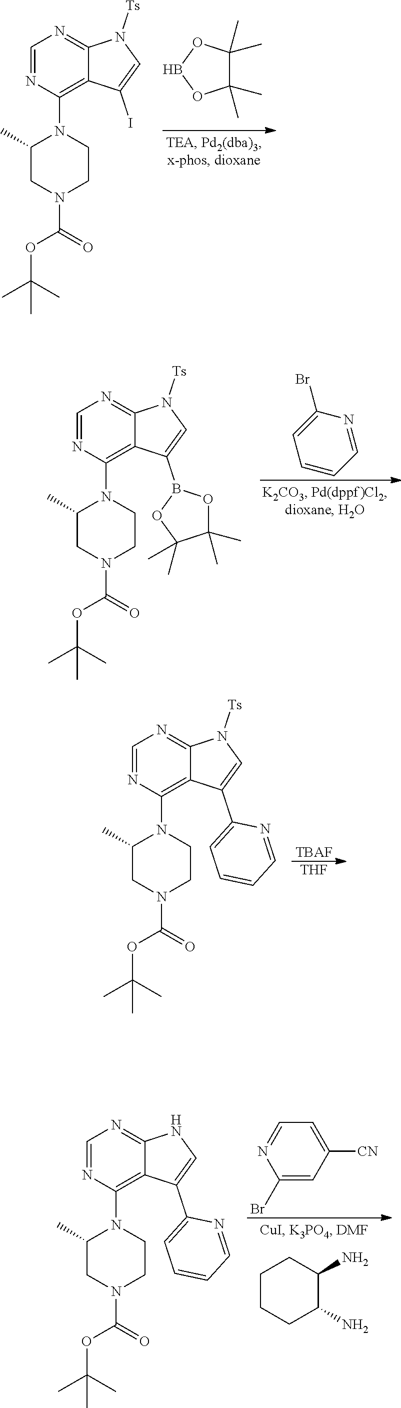

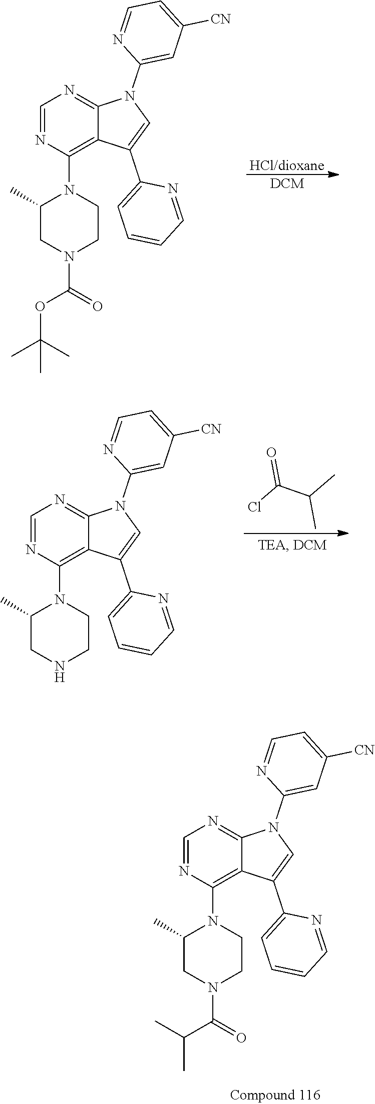

### Step 1. tert-butyl (S)-3-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate

To a solution of tert-butyl (S)-4-(5-iodo-7-tosyl-7H-pyrrolo[2,3-d]pyrimidin-4-yl)-3-methylpiperazine-1-carboxylate (3.0 g, 5.0 mmol, prepared following the procedure outlined in compound 128) in dioxane (40 mL) were added 4,4,5,5-tetramethyl-1,3,2-dioxaborolane (2.9 mL, 20 mmol), X-Phos (0.24 g, 0.50 mmol), TEA (3.5 mL, 25 mmol) and Pd₂(dba)₃ (0.46 g, 0.50 mmol). The resulting mixture was stirred at 95° C. overnight. After cooling to room temperature, the reaction was quenched with water and extracted with DCM twice. The combined organic layers were washed with brine, dried over Na₂SO₄, filtered, and concentrated to afford tert-butyl (S)-3-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate which was used directly in the next step without further purification. LC/MS ESI m/z: 598 (M+H)⁺.

### Step 2. tert-butyl (S)-3-methyl-4-(5-(pyridin-2-yl)-7-tosyl-7H-pyrrolo[2,3-d]pyrimidin-4-yl)piperazine-1-carboxylate

To a solution of tert-butyl (S)-3-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate (3.0 g, 5.0 mmol) in dioxane (40 mL) and H₂O (8 mL) were added 2-bromopyridine (0.96 mL, 10 mmol), K₂CO₃ (3.5 g, 25 mmol) and Pd(dppf)Cl₂ (0.37 g, 0.50 mmol). The resulting mixture was heated to 90° C. overnight. After cooling to room temperature, the solvent was removed and the residue was purified by flash column chromatography (silica gel, 0˜50%, ethyl acetate in petroleum ether) to afford tert-butyl (S)-3-methyl-4-(5-(pyridin-2-yl)-7-tosyl-7H-pyrrolo[2,3-d]pyrimidin-4-yl)piperazine-1-carboxylate (2.0 g, 73%) as a yellow solid. LC/MS ESI m/z: 549 (M+H)⁺.

### Step 3. tert-butyl (S)-3-methyl-4-(5-(pyridin-2-yl)-7H-pyrrolo[2,3-d]pyrimidin-4-yl) piperazine-1-carboxylate

To a solution of tert-butyl (S)-3-methyl-4-(5-(pyridin-2-yl)-7-tosyl-7H-pyrrolo[2,3-d]pyrimidin-4-yl)piperazine-1-carboxylate (2.0 g, 3.6 mmol) in THF (15 mL) was added TBAF (14.5 mL, 1.0M in THF) and the resulting mixture was stirred at room temperature overnight. The reaction was quenched with water and extracted with EtOAc twice. The combined organic layers were washed with brine, dried over Na₂SO₄, filtered, and concentrated. The residue was purified by flash column chromatography (silica gel, 0˜10%, methanol in dichloromethane) to afford tert-butyl (S)-3-methyl-4-(5-(pyridin-2-yl)-7H-pyrrolo[2,3-d]pyrimidin-4-yl)piperazine-1-carboxylate (1.4 g, 97%) as a yellow solid. LC/MS ESI m/z: 395 (M+H)⁺.

### Step 4. tert-butyl (S)-4-(7-(4-cyanopyridin-2-yl)-5-(pyridin-2-yl)-7H-pyrrolo[2,3-d]pyrimidin-4-yl)-3-methylpiperazine-1-carboxylate

To a solution of tert-butyl (S)-3-methyl-4-(5-(pyridin-2-yl)-7H-pyrrolo[2,3-d]pyrimidin-4-yl)piperazine-1-carboxylate (1.4 g, 3.5 mmol) in DMF (20 mL) were added 2-bromoisonicotinonitrile (1.3 g, 7.1 mmol), CuI (0.68 g, 3.5 mmol), trans-cyclohexane-1,2-diamine (0.41 g, 3.5 mmol) and K₃PO₄ (2.26 g, 10.6 mmol). The resulting mixture was heated to 120° C. overnight. After cooling to room temperature. The reaction was partitioned between EtOAc, and water and the organic layer was separated. The aqueous layer was extracted with EtOAc twice, and the combined organic layers were washed with brine, dried over Na₂SO₄, filtered, and concentrated. The residue was purified by flash column chromatography (silica gel, 0˜50%, ethyl acetate in petroleum ether) to afford tert-butyl (S)-4-(7-(4-cyanopyridin-2-yl)-5-(pyridin-2-yl)-7H-pyrrolo[2,3-d]pyrimidin-4-yl)-3-methylpiperazine-1-carboxylate (1.7 g, 96%) as a yellow solid. LC/MS ESI m/z: 497 (M+H)⁺.

### Step 5. (S)-2-(4-(2-methylpiperazin-1-yl)-5-(pyridin-2-yl)-7H-pyrrolo[2,3-d]pyrimidin-7-yl)isonicotinonitrile

To a solution of tert-butyl (S)-4-(7-(4-cyanopyridin-2-yl)-5-(pyridin-2-yl)-7H-pyrrolo[2,3-d]pyrimidin-4-yl)-3-methylpiperazine-1-carboxylate (900 mg, 1.8 mmol) in DCM (5 mL) was added HCl (5 mL, 4M in dioxane). The resulting mixture was stirred at room temperature for 1 h. After removal of solvent, the residue was dissolved in DCM and washed with NaHCO₃ (aq.). The aqueous layer was extracted with DCM twice and the combined organic layers were washed with brine, dried over Na₂SO₄, filtered, and concentrated. The residue was used directly in the next step without further purification. LC/MS ESI m/z: 397 (M+H)⁺.

### Step 6. (S)-2-(4-(4-isobutyryl-2-methylpiperazin-1-yl)-5-(pyridin-2-yl)-7H-pyrrolo[2,3-d]pyrimidin-7-yl)isonicotinonitrile

At 0° C., to a solution of (S)-2-(4-(2-methylpiperazin-1-yl)-5-(pyridin-2-yl)-7H-pyrrolo[2,3-d]pyrimidin-7-yl)isonicotinonitrile (100 mg, 0.25 mmol) and TEA (0.17 mL, 1.2 mmol) in DCM (3 mL) was added isobutyryl chloride (0.05 mL, 0.5 mmol) and the resulting mixture was stirred at room temperature for 1 h. The reaction was quenched with water and extracted with DCM twice. The combined organic layers were washed with NaHCO₃ (aq.), brine, dried over Na₂SO₄, filtered, and concentrated. The residue was purified by prep-HPLC to afford (S)-2-(4-(4-isobutyryl-2-methylpiperazin-1-yl)-5-(pyridin-2-yl)-7H-pyrrolo[2,3-d]pyrimidin-7-yl)isonicotinonitrile (24 mg, 19%) as a light yellow solid. LC/MS ESI m/z: 467 (M+H)⁺. ¹H NMR (400 MHz, CDCl₃) δ 9.40 (s, 1H), 8.82-8.68 (m, 1H), 8.64 (d, J=4.9 Hz, 1H), 8.61-8.50 (m, 2H), 7.87-7.76 (m, 1H), 7.65 (t, J=8.7 Hz, 1H), 7.42 (dd, J=5.0, 1.0 Hz, 1H), 7.35-7.28 (m, 1H), 4.62-4.25 (m, 1H), 4.21-4.06 (m, 1H), 3.77 (d, J=10.1 Hz, 1H), 3.52 (t, J=15.0 Hz, 1H), 3.39-2.99 (m, 2H), 2.83-2.46 (m, 2H), 1.18-1.04 (m, 8H), 0.92 (d, J=6.5 Hz, 1H).

The following compounds were prepared by an analogous procedure to the synthesis of compound 116 from the corresponding acid chlorides and aryl halides.

| Cmpd | | |
|---|---|---|
| No. | Chemical Name | LCMS and ¹H NMR |
| 117 | (S)-2-(4-(2-methyl-4- | LC/MS ESI (m/z): 481 (M + H)⁺. ¹H NMR (400 MHz, |
| | pivaloylpiperazin-1-yl)-5- | CDCl₃) δ 9.40 (s, 1H), 8.73 (dd, J = 4.9, 0.8 Hz, 1H), 8.63 |
| | (pyridin-2-yl)-7H-pyrrolo[2,3- | (dd, J = 5.0, 0.7 Hz, 1H), 8.59 (s, 1H), 8.56 (s, 1H), 7.81 |
| | d]pyrimidin-7- | (td, J = 7.7, 1.6 Hz, 1H), 7.65 (d, J = 7.8 Hz, 1H), 7.42 (dd, |
| | yl)isonicotinonitrile | J = 5.0, 1.3 Hz, 1H), 7.31-7.27 (m, 1H), 4.25 (d, J = 3.5 |
| | | Hz, 1H), 4.14 (dd, J = 13.1, 1.8 Hz, 1H), 4.00 (d, J = 13.1 |
| | | Hz, 1H), 3.65 (d, J = 13.3 Hz, 1H), 3.20 (td, J = 12.9, 3.0 |
| | | Hz, 1H), 3.02 (t, J = 11.7 Hz, 1H), 2.92 (d, J = 11.6 Hz, |
| | | 1H), 1.26 (d, J = 1.3 Hz, 9H), 1.00 (d, J = 6.7 Hz, 3H). |
| 132 | (R)-2-(4-(4-isobutyryl-3- | LC/MS ESI (m/z): 467 (M + H)+. ¹H NMR (400 MHz, |
| | methylpiperazin-1-yl)-5- | CDCl₃) δ 9.41 (s, 1H), 8.74-8.71 (m, 1H), 8.63 (dd, J = |
| | (pyridin-2-yl)-7H-pyrrolo[2,3- | 5.0, 0.7 Hz, 1H), 8.57 (s, 1H), 8.51 (s, 1H), 7.80 (td, J = |
| | d]pyrimidin-7- | 7.7, 1.8 Hz, 1H), 7.63 (d, J = 7.8 Hz, 1H), 7.41 (dd, J = 5.0, |
| | yl)isonicotinonitrile | 1.3 Hz, 1H), 7.31-7.27 (m, 1H), 4.80-4.15 (m, 1H), 4.08 |
| | | (br s, 1H), 3.71 (d, J = 26.3 Hz, 1H), 3.51-2.46 (m, 5H), |
| | | 1.38-1.20 (m, 2H), 1.13-0.89 (m, 7H). |
| 172 | 2-(4-((2S,5R)-4-isobutyryl-2,5- | LC/MS ESI (m/z): 496 (M + H)+. ¹H NMR (400 MHz, |
| | dimethylpiperazin-1-yl)-5-(3- | CDCl₃) δ 9.42 (s, 1H), 8.64-8.59 (m, 2H), 8.56-8.52 (m, |
| | methylpyrazin-2-yl)-7H- | 2H), 8.39 (d, J = 5.0 Hz, 1H), 7.44 (d, J = 4.8 Hz, 1H), 4.59- |
| | pyrrolo[2,3-d]pyrimidin-7- | 4.46 (m, 1H), 4.09-3.95 (m, 1H), 3.88 (d, J = 12.7 Hz, |
| | yl)isonicotinonitrile | 0.5H, rotamers), 3.50 (d, J = 13.1 Hz, 0.5H, rotamers), 3.27- |
| | | 3.19 (m, 2H), 2.97-2.91 (m, 0.5H, rotamers), 2.77-2.71 |
| | | (m, 0.5H, rotamers), 2.64-2.60 (m, 0.5H, rotamers), 2.56 |
| | | (s, 3H), 2.41-2.36 (m, 0.5H, rotamers), 1.20 (d, J = 6.5 |
| | | Hz, 1.5H, rotamers), 1.14 (d, J = 6.9 Hz, 1.5H, rotamers), |
| | | 1.12-1.08 (m, 4.5H, rotamers), 1.03 (d, J = 6.6 Hz, 1.5H, |
| | | rotamers), 0.92 (d, J = 6.7 Hz, 1.5H, rotamers), 0.85 (d, J = |
| | | 6.3 Hz, 1.5H, rotamers). |
| 173 | 2-(4-((2S,5R)-2,5-dimethyl-4-(3- | LC/MS ESI (m/z): 510 (M + H)⁺. ¹H NMR (400 MHz, |
| | methylbutanoyl)piperazin-1-yl)- | CDCl₃) δ 9.42 (s, 1H), 8.65-8.58 (m, 2H), 8.54 (s, 2H), |
| | 5-(3-methylpyrazin-2-yl)-7H- | 8.39 (d, J = 5.4 Hz, 1H), 7.44 (s, 1H), 4.53 (m, 1H), 3.99 (s, |
| | pyrrolo[2,3-d]pyrimidin-7- | 1H), 3.88 (d, J = 13.6 Hz, 0.5H, rotamers), 3.49 (d, J = 13.0 |
| | yl)isonicotinonitrile | Hz, 0.5H, rotamers), 3.26-3.18 (m, 2H), 2.94 (d, J = 13.6 |
| | | Hz, 0.5H, rotamers), 2.56 (s, 3H), 2.42-2.34 (m, 0.5H, |
| | | rotamers), 2.30-2.23 (m, 0.5H, rotamers), 2.13-2.04 (m, |
| | | 2.5H, rotamers), 1.17 (d, J = 6.4 Hz, 1.5H, rotamers), 1.08 |
| | | (d, J = 6.5 Hz, 1.5H, rotamers), 0.97-0.91 (m, 7.5H, |
| | | rotamers), 0.86 (d, J = 6.2 Hz, 1.5H, rotamers). |

### Example 6. Synthesis of 2-(5-(2-fluorophenyl)-4-((2S,5R)-4-isobutyryl-2,5-dimethylpiperazin-1-yl)-7H-pyrrolo[2,3-d]pyrimidin-7-yl)isonicotinonitrile (Compound 118)

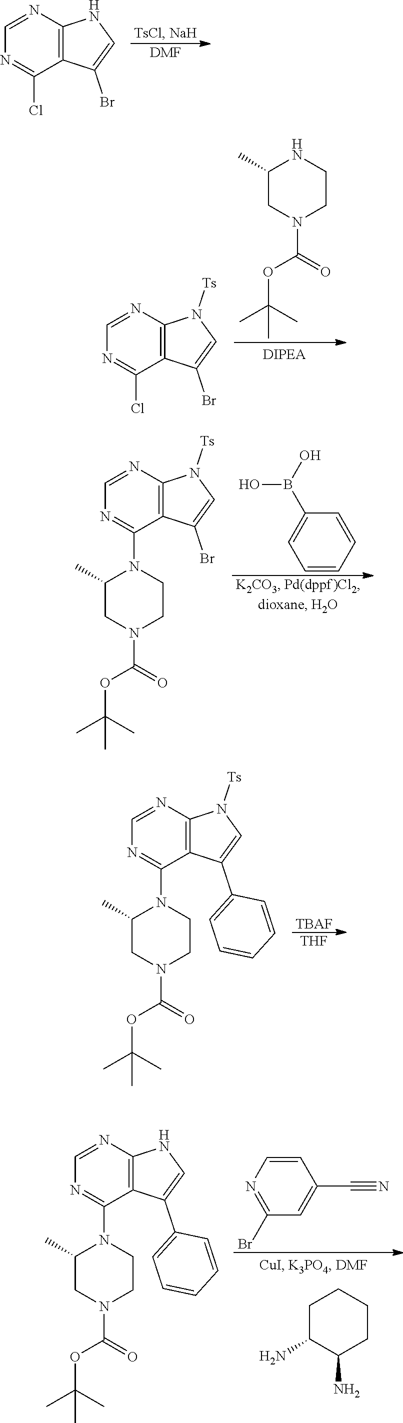

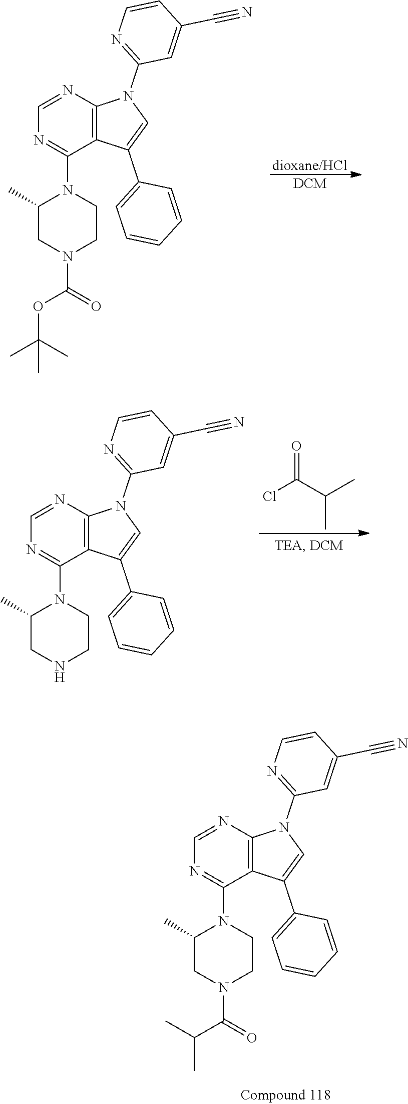

### Step 1. 5-bromo-4-chloro-7-tosyl-7H-pyrrolo[2,3-d]pyrimidine

At 0° C., to a suspension of NaH (1 g, 26 mmol, 60% wt %) in anhydrous DMF (25 mL) was added 5-bromo-4-chloro-7H-pyrrolo[2,3-d]pyrimidine (5.0 g, 22 mmol) in portions. The resulting mixture was stirred at the same temperature for 20 minutes before 4-methylbenzenesulfonyl chloride (4.9 g, 26 mmol) was added in portions. After addition, the reaction was stirred at room temperature overnight. The reaction was poured into ice water and the precipitate was collected by filtration. The solid was dried under vacuum to provide 5-bromo-4-chloro-7-tosyl-7H-pyrrolo[2,3-d]pyrimidine (5.4 g, 65%) as a white solid. LC/MS ESI (m/z): 386, 388 (M+H)⁺.

### Step 2. tert-butyl (S)-4-(5-bromo-7-tosyl-7H-pyrrolo[2,3-d]pyrimidin-4-yl)-3-methylpiperazine-1-carboxylate

To a solution of 5-bromo-4-chloro-7-(4-methylbenzenesulfonyl)-7H-pyrrolo[2,3-d]pyrimidine (3.0 g, 7.8 mmol) in DIPEA (9.5 mL, 55 mmol) was added tert-butyl (S)-3-methylpiperazine-1-carboxylate (6.2 mg, 31 mmol). The resulting mixture was heated to 150° C. for 3 h under N₂. After cooling to room temperature, the solvent was removed and the residue was purified by flash column chromatography to afford tert-butyl (S)-4-(5-bromo-7-tosyl-7H-pyrrolo[2,3-d]pyrimidin-4-yl)-3-methyl piperazine-1-carboxylate (3.5 g, 83%) as a solid. LC/MS ESI m/z: 550, 552 (M+H)⁺.

### Step 3. tert-butyl (S)-3-methyl-4-(5-phenyl-7-tosyl-7H-pyrrolo[2,3-d]pyrimidin-4-yl) piperazine-1-carboxylate

To a solution of tert-butyl (S)-4-(5-bromo-7-tosyl-7H-pyrrolo[2,3-d]pyrimidin-4-yl)-3-methylpiperazine-1-carboxylate (1.5 g, 2.7 mmol) in dioxane (20 mL) and H₂O (2 mL) were added phenylboronic acid (667 mg, 5.46 mmol), K₂CO₃ (1.13 g, 8.19 mmol) and Pd(dppf)Cl₂ (200 mg, 0.27 mmol). The resulting mixture was stirred at 90° C. overnight under N₂. After cooling to room temperature, the solvent was removed and the residue was purified by flash column chromatography (silica gel, 0˜20%, ethyl acetate in petroleum ether) to afford tert-butyl (S)-3-methyl-4-(5-phenyl-7-tosyl-7H-pyrrolo[2,3-d]pyrimidin-4-yl) piperazine-1-carboxylate (1.4 g, 94%) as a yellow solid. LC/MS ESI m/z: 548 (M+H)⁺.

### Step 4. tert-butyl (S)-3-methyl-4-(5-phenyl-7H-pyrrolo[2,3-d]pyrimidin-4-yl) piperazine-1-carboxylate

To a solution of tert-butyl (S)-3-methyl-4-(5-phenyl-7-tosyl-7H-pyrrolo[2,3-d]pyrimidin-4-yl)piperazine-1-carboxylate (1.40 g, 2.56 mmol) in THF (2 mL) was added TBAF (5.0 mL, 1.0 M in THF) and the resulting mixture was stirred at room temperature overnight. The reaction was quenched with water and extracted with EtOAc twice. The combined organic layers were washed with brine, dried over Na₂SO₄, filtered, and concentrated. The residue was purified by flash column chromatography (silica gel, 0˜60%, ethyl acetate in petroleum ether) to afford tert-butyl (S)-3-methyl-4-(5-phenyl-7H-pyrrolo[2,3-d]pyrimidin-4-yl)piperazine-1-carboxylate (900 mg, 90%) as a yellow solid. LC/MS ESI m/z: 394 (M+H)⁺.

### Step 5. tert-butyl (S)-4-(7-(4-cyanopyridin-2-yl)-5-phenyl-7H-pyrrolo[2,3-d]pyrimidin-4-yl)-3-methylpiperazine-1-carboxylate

To a solution of tert-butyl (S)-3-methyl-4-(5-phenyl-7H-pyrrolo[2,3-d]pyrimidin-4-yl)piperazine-1-carboxylate (900 mg, 2.30 mmol) in DMF (20 mL) were added 2-bromoisonicotinonitrile (1.2 g, 6.6 mmol), trans-cyclohexane-1,2-diamine (377 mg, 3.31 mmol), CuI (628 mg, 3.31 mmol) and K₃PO₄ (2.10 g, 9.93 mmol). The resulting mixture was heated to 120° C. overnight. After cooling to room temperature, the reaction mixture was partitioned between EtOAc, and water and the organic layer was separated. The aqueous layer was extracted with EtOAc twice. The combined organic layers were washed with brine, dried over Na₂SO₄, filtered, and concentrated. The residue was purified by flash column chromatography (silica gel, 0˜30%, ethyl acetate in petroleum ether) to afford tert-butyl (S)-4-(7-(4-cyanopyridin-2-yl)-5-phenyl-7H-pyrrolo[2,3-d]pyrimidin-4-yl)-3-methylpiperazine-1-carboxylate (800 mg, 71%) as a yellow solid. LC/MS ESI m/z: 496 (M+H)⁺.

### Step 6. (S)-2-(4-(2-methylpiperazin-1-yl)-5-phenyl-7H-pyrrolo[2,3-d]pyrimidin-7-yl) isonicotinonitrile

At 0° C., to a solution of tert-butyl (S)-4-(7-(4-cyanopyridin-2-yl)-5-phenyl-7H-pyrrolo[2,3-d]pyrimidin-4-yl)-3-methylpiperazine-1-carboxylate (800 mg, 1.62 mmol) in DCM (3 mL) was added HCl (10 mL, 4.0 M in dioxane). The resulting mixture was stirred at room temperature for 0.5 h. After removal of solvent, the residue was diluted with DCM, washed with NaHCO₃ (aq.), and the organic layer was extracted with DCM twice. The combined organic layers were washed with brine, dried over Na₂SO₄, filtered, and concentrated to give (S)-2-(4-(2-methylpiperazin-1-yl)-5-phenyl-7H-pyrrolo[2,3-d]pyrimidin-7-yl)isonicotinonitrile (500 mg, 78%). LC/MS ESI m/z: 396 (M+H)⁺. The product was used directly in the next step without further purification.

### Step 7. (S)-2-(4-(4-isobutyryl-2-methylpiperazin-1-yl)-5-phenyl-7H-pyrrolo[2,3-d]pyrimidin-7-yl)isonicotinonitrile

At 0° C., to a solution of (S)-2-(4-(2-methylpiperazin-1-yl)-5-phenyl-7H-pyrrolo[2,3-d]pyrimidin-7-yl)isonicotinonitrile (40 mg, 0.10 mmol) in DCM (3 mL) was added TEA (0.30 mL, 0.30 mmol), followed by isobutyryl chloride (0.02 mL, 0.2 mmol) dropwise. The resulting mixture was stirred at room temperature overnight. The reaction was quenched with water and extracted with DCM twice. The combined organic layers were washed with NaHCO₃ (aq.), dried over Na₂SO₄, filtered, and concentrated. The residue was purified by flash column chromatography (silica gel, 0˜30%, ethyl acetate in petroleum ether) to give the product which was further purified by prep-HPLC to afford (S)-2-(4-(4-isobutyryl-2-methylpiperazin-1-yl)-5-phenyl-7H-pyrrolo[2,3-d]pyrimidin-7-yl)isonicotinonitrile (22 mg, 46%) as a white solid. LC/MS ESI m/z: 466 (M+H)⁺. ¹H NMR (400 MHZ, CD₃OD) δ 9.27 (s, 1H), 8.69 (d, J=5.0 Hz, 1H), 8.54 (s, 1H), 8.23 (s, 1H), 7.59-7.55 (m, 3H), 7.50 (t, J=7.6 Hz, 2H), 7.43-7.38 (m, 1H), 4.30-4.12 (m, 1.5H, rotamers), 3.88 (m, 1H), 3.67-3.50 (m, 1.5H, rotamers), 3.22-3.03 (m, 2H), 2.96-2.78 (m, 1H), 2.70-2.60 (m, 1H), 1.10-0.99 (m, 7.5H, rotamers), 0.87 (m, 1.5H, rotamers).

The following compounds were prepared by an analogous procedure to the synthesis of compound 118 from the corresponding acid chlorides.

| Cmpd | | |
|---|---|---|
| No. | Chemical Name | LCMS and ¹H NMR |
| 120 | (S)-2-(4-(2-methyl-4- | LC/MS ESI m/z: 480 (M + H)⁺. ¹H NMR (400 MHz, |
| | pivaloylpiperazin-1-yl)-5- | CD₃OD) δ 9.32-9.26 (m, 1H), 8.70 (dd, J = 5.0, 0.8 Hz, |
| | phenyl-7H-pyrrolo[2,3- | 1H), 8.55 (s, 1H), 8.25 (s, 1H), 7.60-7.57 (m, 3H), 7.53- |
| | d]pyrimidin-7- | 7.49 (m, 2H), 7.43-7.38 (m, 1H), 4.19-4.06 (m, 2H), |
| | yl)isonicotinonitrile | 3.91-3.85 (m, 1H), 3.62-3.56 (m, 1H), 3.21-3.13 (m, |
| | | 1H), 3.09-3.00 (m, 1H), 2.86-2.74 (m, 1H), 1.23 (s, 9H), |
| | | 0.92 (d, J = 6.7 Hz, 3H). |
| 123 | (S)-2-(4-(4- | LC/MS ESI m/z: 464 (M + H)⁺. ¹H NMR (400 MHz, |
| | (cyclopropanecarbonyl)-2- | CD₃OD) δ 9.27 (s, 1H), 8.68 (d, J = 5.0 Hz, 1H), 8.53 (s, |
| | methylpiperazin-1-yl)-5-phenyl- | 1H), 8.22 (s, 1H), 7.60-7.56 (m, 3H), 7.51 (dd, J = 10.4, |
| | 7H-pyrrolo[2,3-d]pyrimidin-7- | 4.9 Hz, 2H), 7.43-7.38 (m, 1H), 4.32-4.05 (m, 2H), 3.85 |
| | yl)isonicotinonitrile | (m, 1H), 3.59 (m, 1H), 3.27-3.02 (m, 2H), 2.70 m, 1H), |
| | | 1.94-1.78 (m, 1H), 1.02-0.75 (m, 7H). |

### Example 7. Synthesis of (R)-2-(4-(4-isobutyryl-3-methylpiperazin-1-yl)-5-phenyl-7H-pyrrolo[2,3-d]pyrimidin-7-yl)isonicotinonitrile (Compound 119)

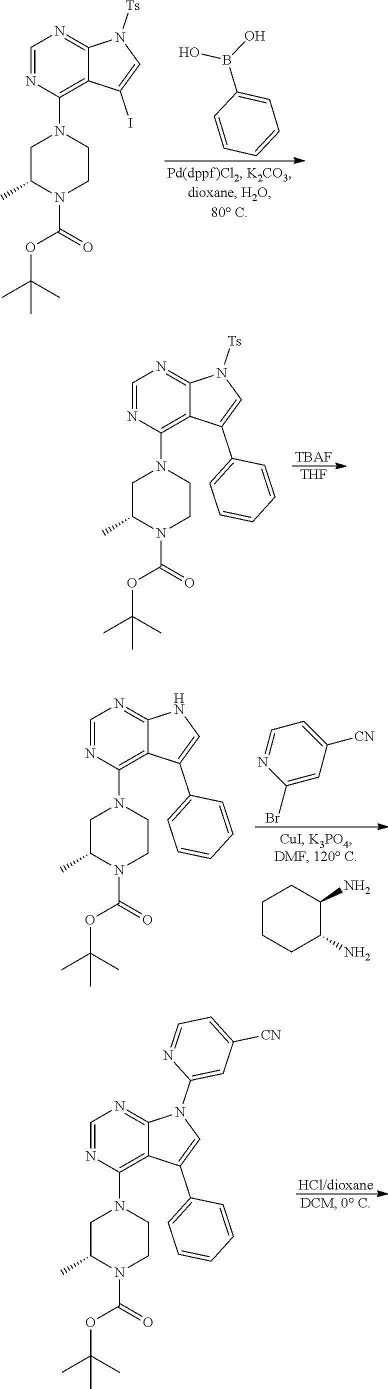

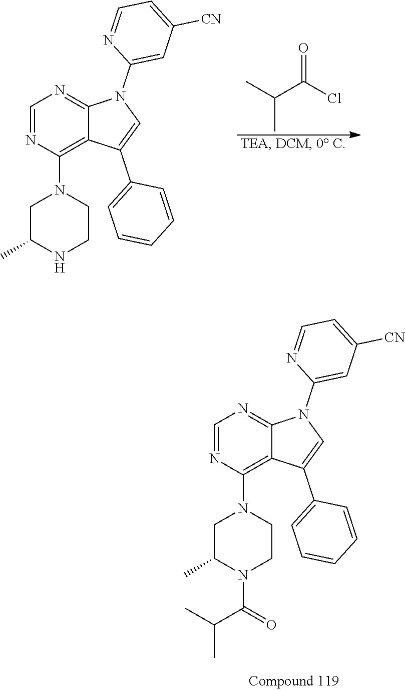

### Step 1. tert-butyl (R)-2-methyl-4-(5-phenyl-7-tosyl-7H-pyrrolo[2,3-d]pyrimidin-4-yl)piperazine-1-carboxylate

To a solution of tert-butyl (R)-4-(5-iodo-7-tosyl-7H-pyrrolo[2,3-d]pyrimidin-4-yl)-2-methylpiperazine-1-carboxylate (1.5 g, 2.1 mmol) in dioxane (10 mL) and water (180 mg, 10 mmol) were added phenylboronic acid (490 mg, 4.0 mmol), K₂CO₃ (1.4 g, 10 mmol) and Pd(dppf)Cl₂ (150 mg, 0.20 mmol). Then the resulting mixture was stirred at 80° C. under N₂ atmosphere overnight. After being cooled down to room temperature, the reaction mixture was filtered. The filtrate was partitioned between EtOAc and water, organic layer was separated, the aqueous layer was extracted with EtOAc twice, the combined organic layers were dried over Na₂SO₄, filtered and concentrated to afford crude product tert-butyl (R)-2-methyl-4-(5-phenyl-7-tosyl-7H-pyrrolo[2,3-d]pyrimidin-4-yl)piperazine-1-carboxylate (1.6 g) as a yellow solid. LC/MS ESI m/z: 548 (M+H)⁺.

### Step 2. tert-butyl (R)-2-methyl-4-(5-phenyl-7H-pyrrolo[2,3-d]pyrimidin-4-yl)piperazine-1-carboxylate

To a solution of tert-butyl (R)-2-methyl-4-(5-phenyl-7-tosyl-7H-pyrrolo[2,3-d]pyrimidin-4-yl)piperazine-1-carboxylate (1.6 g) in THF (5 ml) was added TBAF (4.0 ml, 1.0 M in THF). The resulting mixture was stirred at room temperature overnight. After removal of solvent under reduced pressure, the residue was partitioned between EtOAc and water, organic layer was separated, the aqueous layer was extracted with EtOAc twice, the combined organic layers were dried over Na₂SO₄, filtered and concentrated to afford crude product which was further purified by flash column chromatography (30˜80% EtOAc in petroleum ether) to afford tert-butyl (R)-2-methyl-4-(5-phenyl-7H-pyrrolo[2,3-d]pyrimidin-4-yl)piperazine-1-carboxylate (790 mg, 97% yield in two steps) as a gray solid. LC/MS ESI m/z: 394 (M+H)⁺.

### Step 3. tert-butyl (R)-4-(7-(4-cyanopyridin-2-yl)-5-phenyl-7H-pyrrolo[2,3-d]pyrimidin-4-yl)-2-methylpiperazine-1-carboxylate

To a solution of tert-butyl (R)-2-methyl-4-(5-phenyl-7H-pyrrolo[2,3-d]pyrimidin-4-yl)piperazine-1-carboxylate (790 mg, 2.0 mmol) in DMF (5 mL) was added 2-bromopyridine-4-carbonitrile (730 mg, 4.0 mmol), CuI (110 mg, 0.60 mmol), trans-1,2-diaminocyclohexane (68 mg, 0.60 mmol) and K₃PO₄ (1.3 g, 6.0 mmol). The resulting mixture was heated to 120° C. overnight under N₂ atmosphere. After being cooled down to room temperature, the reaction was partitioned between EtOAc and water, organic layer was separated, aqueous layer was extracted with EtOAc twice, the combined organic layers were washed with brine, dried over Na₂SO₄, filter and concentrated. The residue was purified by flash column chromatography (0˜30% EtOAc in petroleum ether) to afford tert-butyl (R)-4-(7-(4-cyanopyridin-2-yl)-5-phenyl-7H-pyrrolo[2,3-d]pyrimidin-4-yl)-2-methylpiperazine-1-carboxylate (680 mg, 69% yield) as a white solid. LC/MS ESI m/z: 496 (M+H)⁺.

### Step 4. (R)-2-(4-(3-methylpiperazin-1-yl)-5-phenyl-7H-pyrrolo[2,3-d]pyrimidin-7-yl)isonicotinonitrile

At 0° C., to a solution of tert-butyl (R)-4-(7-(4-cyanopyridin-2-yl)-5-phenyl-7H-pyrrolo[2,3-d]pyrimidin-4-yl)-2-methylpiperazine-1-carboxylate (490 mg, 1.0 mmol) in DCM (10 mL) was added HCl (15 mL, 4.0 M in dioxane), the resulting mixture was stirred at room temperature for 4 h. After removal of solvent, the residue was used to the next step directly. (R)-2-(4-(3-methylpiperazin-1-yl)-5-phenyl-7H-pyrrolo[2,3-d]pyrimidin-7-yl)isonicotinonitrile. (500 mg, contain solvent) LC/MS ESI m/z: 396 (M+H)⁺.

### Step 5. (R)-2-(4-(4-isobutyryl-3-methylpiperazin-1-yl)-5-phenyl-7H-pyrrolo[2,3-d]pyrimidin-7-yl)isonicotinonitrile

At 0° C., to a solution of (R)-2-(4-(3-methylpiperazin-1-yl)-5-phenyl-7H-pyrrolo[2,3-d]pyrimidin-7-yl)isonicotinonitrile. (94 mg, 0.20 mmol) in DCM (2 mL) was added TEA (0.11 mL, 80 mg, 0.80 mmol), followed by di-isobutyryl chloride (43 mg, 0.40 mmol) dropwise. The resulting mixture was stirred at room temperature for 1 h. The solvent was removed under reduced pressure. The residue was purified by prep-HPLC to afford (R)-2-(4-(4-isobutyryl-3-methylpiperazin-1-yl)-5-phenyl-7H-pyrrolo[2,3-d]pyrimidin-7-yl)isonicotinonitrile (39 mg, 42%) as a white solid. LC/MS ESI m/z: 466 (M+H)⁺. ¹H NMR (400 MHZ, CDCl₃) δ 9.41 (s, 1H), 8.63 (dd, J=5.0, 0.6 Hz, 1H), 8.59 (s, 1H), 8.23 (s, 1H), 7.63-7.53 (m, 2H), 7.52-7.44 (m, 2H), 7.44-7.34 (m, 2H), 4.86-4.14 (m, 1H), 4.06-3.72 (m, 2H), 3.66-3.21 (m, 1H), 3.16-2.34 (m, 4H), 1.39-0.97 (m, 9H).

The following compound was prepared by the procedures similar to the synthesis of example above from the corresponding acyl chloride.

| Cmpd | | |
|---|---|---|
| No. | Chemical Name | LCMS and ¹H NMR |
| 124 | (R)-2-(4-(4- | LC/MS ESI (m/z): 464 (M + H)⁺. ¹H NMR (400 MHz, |
| | (cyclopropanecarbonyl)-3- | CDCl₃) δ 9.42 (s, 1H), 8.63 (d, J = 5.0 Hz, 1H), 8.59 (s, |
| | methylpiperazin-1-yl)-5-phenyl- | 1H), 8.22 (s, 1H), 7.63-7.52 (m, 2H), 7.52-7.43 (m, 2H), |
| | 7H-pyrrolo[2,3-d]pyrimidin-7- | 7.43-7.33 (m, 2H), 4.96-4.35 (m, 1H), 4.16-3.37 (m, |
| | yl)isonicotinonitrile | 3H), 3.34-2.35 (m, 3H), 1.82-1.60 (m, 1H), 1.40-1.06 |
| | | (m, 3H), 1.04-0.87 (m, 2H), 0.87-0.64 (m, 2H). |
| 121 | (R)-2-(4-(3-methyl-4- | LC/MS ESI (m/z): 480 (M + H)⁺. ¹H NMR (400 MHz, |
| | pivaloylpiperazin-1-yl)-5- | CDCl₃) δ 9.47-9.38 (m, 1H), 8.62 (dd, J = 5.0, 0.8 Hz, |
| | phenyl-7H-pyrrolo[2,3- | 1H), 8.58 (s, 1H), 8.23 (s, 1H), 7.60-7.54 (m, 2H), 7.51- |
| | d]pyrimidin-7- | 7.44 (m, 2H), 7.43-7.35 (m, 2H), 4.74-4.51 (m, 1H), |
| | yl)isonicotinonitrile | 4.00-3.82 (m, 1H), 3.82-3.56 (m, 2H), 3.01 (dd, J = |
| | | 13.1, 3.6 Hz, 1H), 2.94-2.43 (m, 2H), 1.31-1.14 (m, |
| | | 12H). |

### Example 8. Synthesis of (R)-2-(5-cyclopropyl-4-(3-methyl-4-pivaloylpiperazin-1-yl)-7H-pyrrolo[2,3-d]pyrimidin-7-yl)isonicotinonitrile (Compound 122)

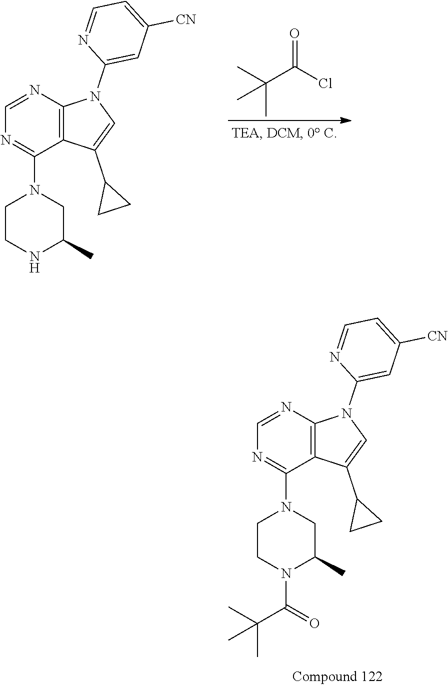

At 0° C., to a solution of (R)-2-(5-cyclopropyl-4-(3-methylpiperazin-1-yl)-7H-pyrrolo[2,3-d]pyrimidin-7-yl)isonicotinonitrile (100 mg, 0.28 mmol) in DCM (3 mL) was added TEA (85 mg, 0.84 mmol), followed by pivaloyl chloride (67 mg, 0.56 mmol) dropwise. The resulting mixture was stirred at room temperature for 30 minutes. The reaction was quenched with ice water and extracted with DCM twice, The combined organic layers were washed with NaHCO₃ (aq.), dried over Na₂SO₄, filtered, concentrated, the residue was purified by flash chromatography (0˜35% ethyl acetate in petroleum ether) to give solid product which was further purified by prep-HPLC to afford (R)-2-(5-cyclopropyl-4-(3-methyl-4-pivaloylpiperazin-1-yl)-7H-pyrrolo[2,3-d]pyrimidin-7-yl)isonicotinonitrile (42 mg, 34%) as a white solid. LC/MS ESI m/z: 444 (M+H)⁺. ¹H NMR (400 MHZ, CD₃OD) δ 9.19-9.15 (m, 1H), 8.65-8.62 (m, 1H), 8.44 (s, 1H), 7.86 (d, 1H), 7.52 (dd, J=5.0, 1.3 Hz, 1H), 4.82-4.76 (m, 1H), 4.53-4.48 (m, 1H), 4.40-4.26 (m, 1H), 4.15-4.10 (m, 1H), 3.72-3.46 (m, 1H), 3.44-3.38 (m, 1H), 3.16-3.06 (m, 1H), 2.11-2.05 (m, 1H), 1.32 (s, 9H), 1.30-1.25 (m, 3H), 1.12-1.06 (m, 2H), 0.95-0.89 (m, 1H), 0.75-0.69 (m, 1H).

### Example 9. Synthesis of (R)-2-(4-(4-(2-fluoro-2-methylpropanoyl)-3-methylpiperazin-1-yl)-5-(2-fluorophenyl)-7H-pyrrolo[2,3-d]pyrimidin-7-yl)isonicotinonitrile (Compound 126)

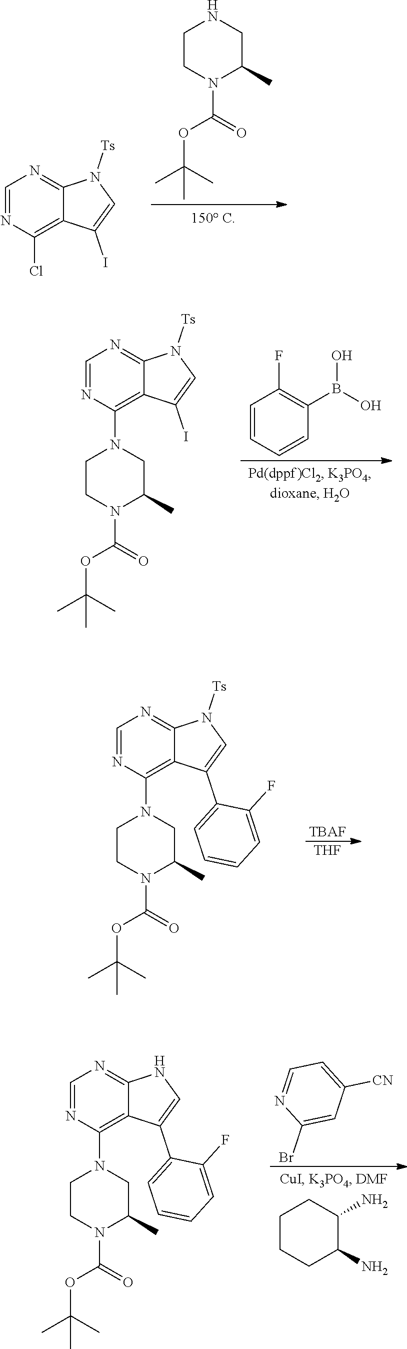

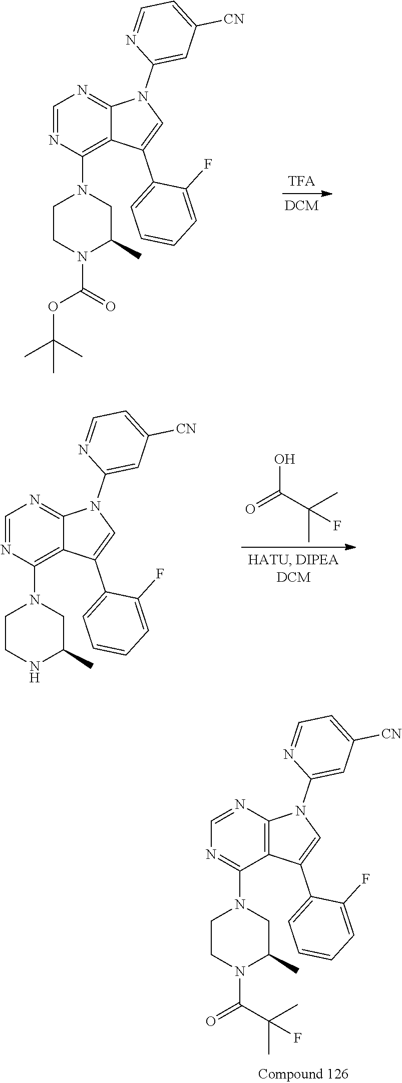

### Step 1. tert-butyl (R)-4-(5-iodo-7-tosyl-7H-pyrrolo[2,3-d]pyrimidin-4-yl)-2-methylpiperazine-1-carboxylate

To a solution of 4-chloro-5-iodo-7-tosyl-7H-pyrrolo[2,3-d]pyrimidine (50 g, 120 mmol) in EtOH (500 mL) was added tert-butyl (R)-2-methylpiperazine-1-carboxylate (27.7 g, 138 mmol). The resulting mixture was stirred at 90° C. under N₂ atmosphere for 16 h. After cooled down to room temperature, solvent was removed and the residue was quenched with H₂O and EtOAc, organic layer was separated, the aqueous phase was extracted with EtOAc twice, the combined organic layers were dried over Na₂SO₄, filtered and concentrated, the residue was triturated with petroleum ether/EtOAc (10:1) and filtered to afford tert-butyl (R)-4-(5-iodo-7-tosyl-7H-pyrrolo[2,3-d]pyrimidin-4-yl)-2-methylpiperazine-1-carboxylate (48 g, 70%) as a light yellow solid. LC/MS ESI (m/z): 598 (M+H)⁺.

### Step 2. tert-butyl (R)-4-(5-(2-fluorophenyl)-7-tosyl-7H-pyrrolo[2,3-d]pyrimidin-4-yl)-2-methylpiperazine-1-carboxylate

To a solution of tert-butyl (R)-4-(5-iodo-7-tosyl-7H-pyrrolo[2,3-d]pyrimidin-4-yl)-2-methylpiperazine-1-carboxylate (1.5 g, 2.5 mmol) in dioxane (50 mL) and water (5 mL) were added (2-fluorophenyl) boronic acid (385 mg, 2.75 mmol), K₃PO₄ (1.0 g, 5.0 mmol), and Pd(dppf)Cl₂ (190 mg, 0.25 mmol), The resulting mixture was heated to 90° C. overnight. After being cooled down to room temperature, solvent was removed and the residue was purified by flash chromatography (silica gel, 0˜60% EtOAc in petroleum ether) to afford tert-butyl (R)-4-(5-(2-fluorophenyl)-7-tosyl-7H-pyrrolo[2,3-d]pyrimidin-4-yl)-2-methylpiperazine-1-carboxylate (1.4 g, 97%) as a white solid. LC/MS ESI (m/z): 566 (M+H)⁺.

### Step 3. tert-butyl (R)-4-(5-(2-fluorophenyl)-7H-pyrrolo[2,3-d]pyrimidin-4-yl)-2-methylpiperazine-1-carboxylate

To a solution of tert-butyl (R)-4-(5-(2-fluorophenyl)-7-tosyl-7H-pyrrolo[2,3-d]pyrimidin-4-yl)-2-methylpiperazine-1-carboxylate (1.38 g, 2.44 mmol) in THF (30 mL) were added TBAF (15 mL, 1.0M in THF). The reaction mixture was stirred at room temperature overnight. The solvent was removed, and the residue was purified by flash column chromatography (silica gel, 0˜64% EtOAc in petroleum ether) to afford tert-butyl (R)-4-(5-(2-fluorophenyl)-7H-pyrrolo[2,3-d]pyrimidin-4-yl)-2-methylpiperazine-1-carboxylate (870 mg, 86%) as a colorless oil. LC/MS ESI (m/z): 412 (M+H)⁺.

### Step 4. tert-butyl (R)-4-(7-(4-cyanopyridin-2-yl)-5-(2-fluorophenyl)-7H-pyrrolo[2,3-d]pyrimidin-4-yl)-2-methylpiperazine-1-carboxylate

To the solution of tert-butyl (R)-4-(5-(2-fluorophenyl)-7H-pyrrolo[2,3-d]pyrimidin-4-yl)-2-methylpiperazine-1-carboxylate (870 mg, 2.1 mmol) in DMF (10 mL) were added CuI (460 mg, 2.1 mmol), K₃PO₄ (900 mg, 4.2 mmol), trans-cyclohexane-1,2-diamine (480 mg, 4.2 mmol) and 2-bromoisonicotinonitrile (770 mg, 4.2 mmol) respectively. The resulting reaction mixture was stirred at 120° C. under N₂ overnight. After being cooled down to room temperature, the reaction mixture was partitioned between EtOAc and water, organic layer was separated, the aqueous layer was extracted with EtOAc twice, the combined organic layers were washed with brine, dried over Na₂SO₄, filtered and concentrated. The residue was purified by flash column chromatography to afford tert-butyl (R)-4-(7-(4-cyanopyridin-2-yl)-5-(2-fluorophenyl)-7H-pyrrolo[2,3-d]pyrimidin-4-yl)-2-methylpiperazine-1-carboxylate (1.0 g, 92%) as white solid. LC/MS ESI m/z: 514 (M+H)⁺.

### Step 5. (R)-2-(5-(2-fluorophenyl)-4-(3-methylpiperazin-1-yl)-7H-pyrrolo[2,3-d]pyrimidin-7-yl)isonicotinonitrile

At 0° C., to a solution of tert-butyl (R)-4-(7-(4-cyanopyridin-2-yl)-5-(2-fluorophenyl)-7H-pyrrolo[2,3-d]pyrimidin-4-yl)-2-methylpiperazine-1-carboxylate (900 mg, 1.7 mmol) in DCM (10 mL) was added TFA (3 mL). The resulting mixture was stirred at the same temperature for 2 h. The reaction was quenched with NaHCO₃ (aq.), extracted with DCM twice, the combined organic layers were washed with brine, dried over Na₂SO₄, filtered and concentrated. The residue was used in the next step directly. LC/MS ESI m/z: 414.5 (M+H)⁺.

### Step 6. (R)-2-(4-(4-(2-fluoro-2-methylpropanoyl)-3-methylpiperazin-1-yl)-5-(2-fluorophenyl)-7H-pyrrolo[2,3-d]pyrimidin-7-yl)isonicotinonitrile

To the solution of (R)-2-(5-(2-fluorophenyl)-4-(3-methylpiperazin-1-yl)-7H-pyrrolo[2,3-d]pyrimidin-7-yl)isonicotinonitrile (100 mg, 0.24 mmol) in DMF (4 mL) were added DIPEA (25 mg, 0.3 mmol), 2-fluoro-2-methylpropanoic acid (0.02 mL, 0.24 mmol) and HATU (180 mg, 0.48 mmol) respectively. The resulting reaction mixture was stirred at room temperature under N₂ overnight. The reaction mixture was partitioned between EtOAc and water, organic layer was separated, aqueous layer was extracted with EtOAc twice, the combined organic payers were washed with brine, dried over Na₂SO₄, filtered, and concentrated under reduced pressure, the residue was purified by flash column chromatography to afford (R)-2-(4-(4-(2-fluoro-2-methylpropanoyl)-3-methylpiperazin-1-yl)-5-(2-fluorophenyl)-7H-pyrrolo[2,3-d]pyrimidin-7-yl)isonicotinonitrile (12 mg, 10%) as a white solid. LC/MS ESI (m/z): 502.6 (M+H)⁺. ¹H NMR (400 MHZ, CDCl₃) δ 9.41 (s, 1H), 8.65-8.61 (m, 1H), 8.58 (s, 1H), 8.30 (s, 1H), 7.53-7.47 (m, 1H), 7.42-7.36 (m, 2H), 7.33-7.27 (m, 1H), 7.25-7.19 (m, 1H), 4.76-4.62 (m, 1H), 3.97-3.55 (m, 3H), 3.10-2.40 (m, 3H), 1.63-1.58 (m, 3H), 1.56-1.52 (m, 3H), 1.33-1.11 (m, 3H).

The following compound was prepared by the procedure similar to the synthesis of example above from the corresponding acid.

| Cmpd | | |
|---|---|---|
| No.. | Chemical Name | LCMS and ¹H NMR |
| 131 | (R)-2-(4-(4-(1- | LC/MS ESI (m/z): 500.6 (M + H)⁺. ¹H NMR (400 MHz, |
| | fluorocyclopropane-1-carbonyl)- | CDCl₃) δ 9.41 (s, 1H), 8.66-8.57 (m, 2H), 8.30 (s, 1H), |
| | 3-methylpiperazin-1-yl)-5-(2- | 7.53-7.47 (m, 1H), 7.43-7.36 (m, 2H), 7.32-7.26 (m, |
| | fluorophenyl)-7H-pyrrolo[2,3- | 1H), 7.25-7.20 (m, 1H), 4.62 (s, 1H), 4.01-3.58 (m, 3H), |
| | d]pyrimidin-7- | 3.14-3.02 (m, 1H), 2.98-2.27 (m, 2H), 1.33-1.13 (m, |
| | yl)isonicotinonitrile | 7H). |

### Example 10. Synthesis of (S)-2-(4-(2-methyl-4-(1-methylcyclopropane-1-carbonyl)piperazin-1-yl)-5-(pyridin-2-yl)-7H-pyrrolo[2,3-d]pyrimidin-7-yl)isonicotinonitrile (Compound 127)

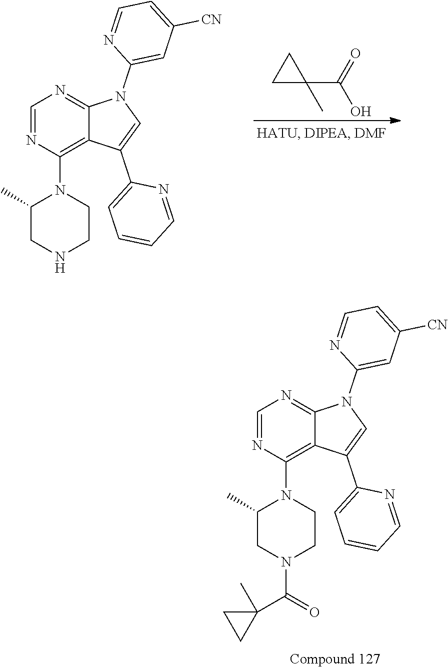

### Step 1. (S)-2-(4-(2-methyl-4-(1-methylcyclopropane-1-carbonyl)piperazin-1-yl)-5-(pyridin-2-yl)-7H-pyrrolo[2,3-d]pyrimidin-7-yl)isonicotinonitrile

To a solution of (S)-2-(4-(2-methylpiperazin-1-yl)-5-(pyridin-2-yl)-7H-pyrrolo[2,3-d]pyrimidin-7-yl)isonicotinonitrile (100 mg, 0.25 mmol) in DMF (3 mL) were added 1-methylcyclopropane-1-carboxylic acid (38 mg, 0.37 mmol), DIPEA (0.13 mL, 0.75 mmol) and HATU (190 mg, 0.50 mmol). The resulting mixture was stirred at room temperature overnight. The reaction was quenched with water, extracted with EtOAc twice, the combined organic layers were washed with NaHCO₃ (aq.) and brine, dried over Na₂SO₄, filtered and concentrated. The residue was purified by prep-HPLC to afford (S)-2-(4-(2-methyl-4-(1-methylcyclopropane-1-carbonyl)piperazin-1-yl)-5-(pyridin-2-yl)-7H-pyrrolo[2,3-d]pyrimidin-7-yl)isonicotinonitrile (27 mg, 21%) as a white solid. LC/MS ESI m/z: 479 (M+H)⁺. ¹H NMR (400 MHZ, CDCl₃) δ 9.40 (s, 1H), 8.74 (d, J=4.3 Hz, 1H), 8.64 (dd, J=5.0, 0.6 Hz, 1H), 8.60 (s, 1H), 8.59 (s, 1H), 7.84 (t, J=7.3 Hz, 1H), 7.66 (d, J=7.8 Hz, 1H), 7.42 (dd, J=5.0, 1.3 Hz, 1H), 7.34-7.29 (m, 1H), 4.38-4.15 (m, 2H), 3.97-3.91 (m, 1H), 3.66 (d, J=8.1 Hz, 1H), 3.21-2.77 (m, 3H), 1.29 (s, 3H), 1.02 (s, 3H), 0.94-0.86 (m, 2H), 0.62-0.50 (m, 2H).

### Example 11. Synthesis of (S)-2-(4-(2-methyl-4-(1-methylcyclopropane-1-carbonyl)piperazin-1-yl)-5-phenyl-7H-pyrrolo[2,3-d]pyrimidin-7-yl)isonicotinonitrile (Compound 128)

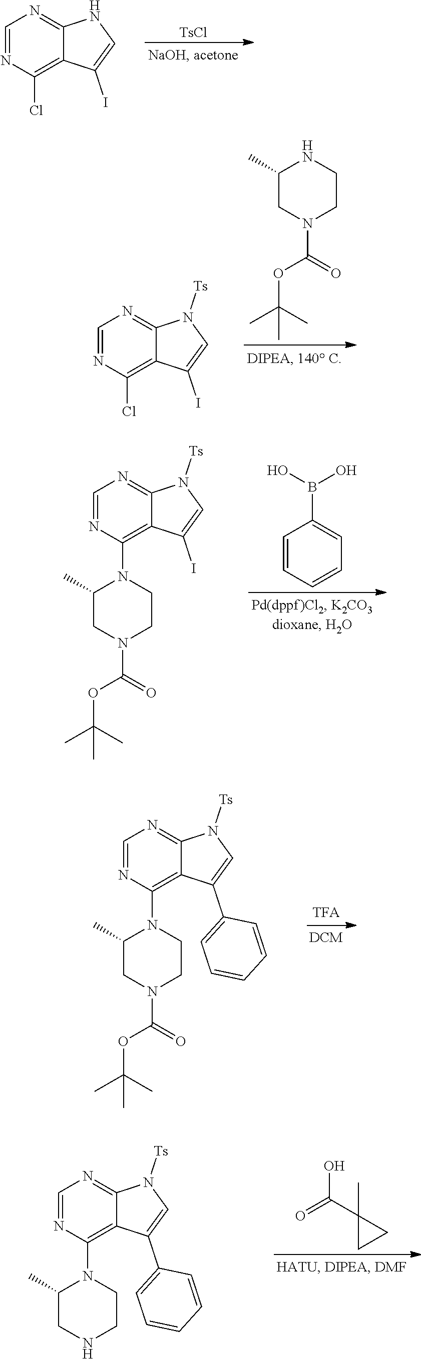

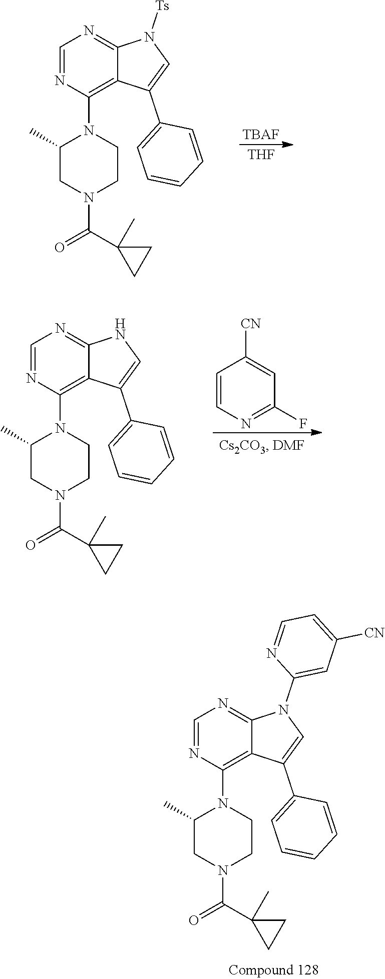

### Step 1. 4-chloro-5-iodo-7-tosyl-7H-pyrrolo[2,3-d]pyrimidine

At 0° C., to a solution of 4-chloro-5-iodo-7H-pyrrolo[2,3-d]pyrimidine (200 g, 0.71 mol) and 4-methylbenzene-1-sulfonyl chloride (180 g, 0.93 mol) in acetone (2 L) was added dropwise 2M NaOH (0.53 L). After addition, the reaction was allowed to warm up to room temperature and stirred for another 3 hours. The precipitation was collected by filtration and washed with water twice and dried over oil pump. 4-chloro-5-iodo-7-tosyl-7H-pyrrolo[2,3-d]pyrimidine (300 g, 95%) as an off white solid. LC/MS ESI (m/z): 434 (M+H)⁺.

### Step 2. tert-butyl (S)-4-(5-iodo-7-tosyl-7H-pyrrolo[2,3-d]pyrimidin-4-yl)-3-methylpiperazine-1-carboxylate

To a solution of 4-chloro-5-iodo-7-tosyl-7H-pyrrolo[2,3-d]pyrimidine (90 g, 0.21 mol) in DIPEA (600 mL) was added tert-butyl (S)-3-methylpiperazine-1-carboxylate (81 g, 0.42 mol). The resulting mixture was heated to 140° C. for 3 h. After being cooled down to room temperature, solvent was removed and the residue was purified by flash column chromatography (silica gel, 0˜30%, ethyl acetate in petroleum ether) to afford tert-butyl (S)-4-(5-iodo-7-tosyl-7H-pyrrolo[2,3-d]pyrimidin-4-yl)-3-methylpiperazine-1-carboxylate (100 g, 73%) as a white solid. LC/MS ESI m/z: 598 (M+H)⁺.

### Step 3. tert-butyl (S)-3-methyl-4-(5-phenyl-7-tosyl-7H-pyrrolo[2,3-d]pyrimidin-4-yl)piperazine-1-carboxylate

To a solution of tert-butyl (S)-4-(5-iodo-7-tosyl-7H-pyrrolo[2,3-d]pyrimidin-4-yl)-3-methylpiperazine-1-carboxylate (30 g, 50 mmol) in dioxane-H₂O (300 mL, V:V=5:1) were added phenylboronic acid (12.3 g, 101 mmol), K₂CO₃ (20.8 g, 151 mmol) and Pd(dppf)Cl₂ (3.7 g, 5.0 mmol). The resulting mixture was heated to 90° C. overnight. After being cooled down to room temperature, solvent was filtered. The filtrate was concentrated and purified by flash column chromatography (silica gel, 0˜30%, ethyl acetate in petroleum ether) to afford tert-butyl (S)-3-methyl-4-(5-phenyl-7-tosyl-7H-pyrrolo[2,3-d]pyrimidin-4-yl)piperazine-1-carboxylate (23 g, 84%) as a white solid. LC/MS ESI m/z: 548 (M+H)⁺.

### Step 4. (S)-4-(2-methylpiperazin-1-yl)-5-phenyl-7-tosyl-7H-pyrrolo[2,3-d]pyrimidine

To a solution of tert-butyl (S)-3-methyl-4-(5-phenyl-7-tosyl-7H-pyrrolo[2,3-d]pyrimidin-4-yl)piperazine-1-carboxylate (23 g, 42 mmol) in DCM (200 mL) was added TFA (30 mL). The resulting mixture was stirred at room temperature for 2 h. After removal of solvent, the residue was diluted with DCM, washed with NaHCO₃ (aq.), the organic layer was extracted with DCM twice, the combined organic layers were washed with brine, dried over Na₂SO₄, filtered and concentrated. The residue was used in the next step directly (S)-4-(2-methylpiperazin-1-yl)-5-phenyl-7-tosyl-7H-pyrrolo[2,3-d]pyrimidine. LC/MS ESI m/z: 448 (M+H)⁺.

### Step 5. (S)-(3-methyl-4-(5-phenyl-7-tosyl-7H-pyrrolo[2,3-d]pyrimidin-4-yl)piperazin-1-yl)(1-methylcyclopropyl)methanone

To the solution of (S)-4-(2-methylpiperazin-1-yl)-5-phenyl-7-tosyl-7H-pyrrolo[2,3-d]pyrimidine (10.5 g, 23.4 mmol) in DMF (200 mL) were added DIPEA (12.2 mL, 70.5 mmol), 1-methylcyclopropane-1-carboxylic acid (3.5 g, 35 mmol) and HATU (1.1 g, 28 mmol) respectively. The resulting reaction mixture was stirred at room temperature under N₂ overnight. The reaction mixture was partitioned between EtOAc and water, organic layer was separated, aqueous layer was extracted with EtOAc twice, the combined organic payers were washed with brine, dried over Na₂SO₄, filtered, and concentrated under reduced pressure. The residue was purified by flash column chromatography (silica gel, 0˜30% EtOAc in petroleum) to afford (S)-(3-methyl-4-(5-phenyl-7-tosyl-7H-pyrrolo[2,3-d]pyrimidin-4-yl)piperazin-1-yl)(1-methylcyclopropyl)methanone (9.6 g, 79%) as a solid. LC/MS ESI m/z: 530 (M+H)⁺.

### Step 6. (S)-(3-methyl-4-(5-phenyl-7H-pyrrolo[2,3-d]pyrimidin-4-yl)piperazin-1-yl)(1-methylcyclopropyl)methanone

To a solution of (S)-(3-methyl-4-(5-phenyl-7-tosyl-7H-pyrrolo[2,3-d]pyrimidin-4-yl)piperazin-1-yl)(1-methylcyclopropyl)methanone (9.8 g, 19 mmol) in THF (100 mL) was added TBAF (93 mL, 1.0 M in THF). The resulting mixture was stirred at room temperature overnight. The reaction was quenched with water, extracted with EtOAc twice, the combined organic layers were washed with brine, dried over Na₂SO₄, filtered and concentrated. The residue was purified by flash column chromatography (silica gel, 0˜60%, ethyl acetate in petroleum ether) to afford (S)-(3-methyl-4-(5-phenyl-7H-pyrrolo[2,3-d]pyrimidin-4-yl)piperazin-1-yl)(1-methylcyclopropyl)methanone (6.0 g, 87%) as a white solid. LC/MS ESI m/z: 376 (M+H)⁺.

### Step 7. (S)-2-(4-(2-methyl-4-(1-methylcyclopropane-1-carbonyl)piperazin-1-yl)-5-phenyl-7H-pyrrolo[2,3-d]pyrimidin-7-yl)isonicotinonitrile

To a solution of (S)-(3-methyl-4-(5-phenyl-7H-pyrrolo[2,3-d]pyrimidin-4-yl)piperazin-1-yl)(1-methylcyclopropyl)methanone (6.2 g, 26 mmol) in DMF (60 mL) were added 2-fluoroisonicotinonitrile (5.6 g, 46 mmol) and Cs₂CO₃ (42.4 g, 131 mmol). The resulting mixture was heated to 40° C. for 3 h. After being cooled down to room temperature. The reaction was quenched with water, extracted with EtOAc twice, the combined organic layers were washed with brine, dried over Na₂SO₄, filtered and concentrated. The residue was purified by flash column chromatography (silica gel, 0˜40%, ethyl acetate in petroleum ether) to give (S)-2-(4-(2-methyl-4-(1-methylcyclopropane-1-carbonyl)piperazin-1-yl)-5-phenyl-7H-pyrrolo[2,3-d]pyrimidin-7-yl)isonicotinonitrile (6.2 g, 82%) as a white solid. LC/MS ESI m/z: 478 (M+H)⁺. ¹H NMR (400 MHZ, CDCl₃) δ 9.41 (s, 1H), 8.65-8.57 (m, 2H), 8.22 (s, 1H), 7.55 (d, J=7.3 Hz, 2H), 7.46 (t, J=7.5 Hz, 2H), 7.38 (dd, J=13.2, 5.8 Hz, 2H), 4.29-4.05 (m, 2H), 3.83 (d, J=12.9 Hz, 1H), 3.59 (d, J=11.5 Hz, 1H), 3.14 (m, 3H), 1.27 (s, 3H), 0.92 (m, 5H), 0.56 (d, J=3.3 Hz, 2H).

### Example 12. Synthesis of (R)-2-(5-cyclopropyl-4-(4-(1-fluorocyclopropane-1-carbonyl)-3-methylpiperazin-1-yl)-7H-pyrrolo[2,3-d]pyrimidin-7-yl)isonicotinonitrile (Compound 129)

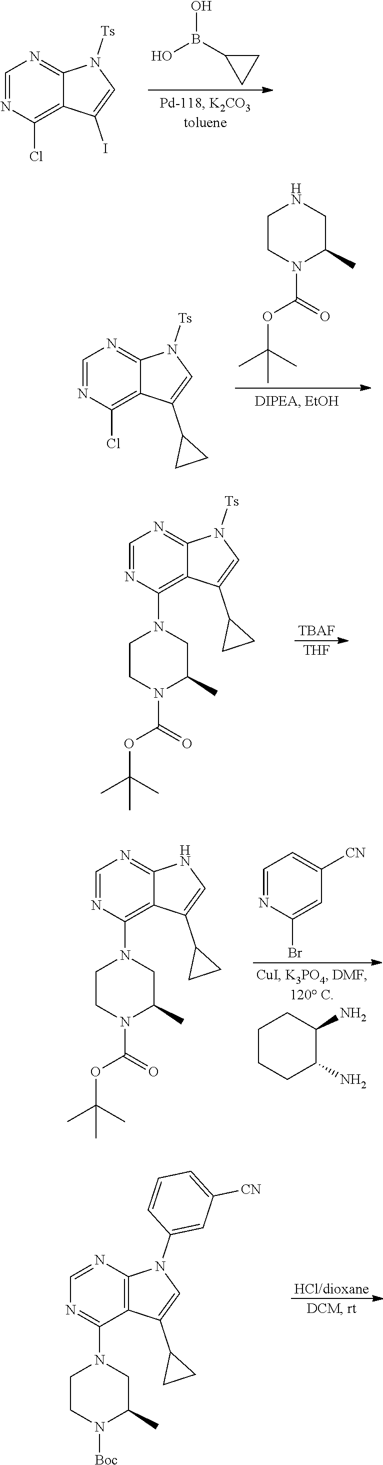

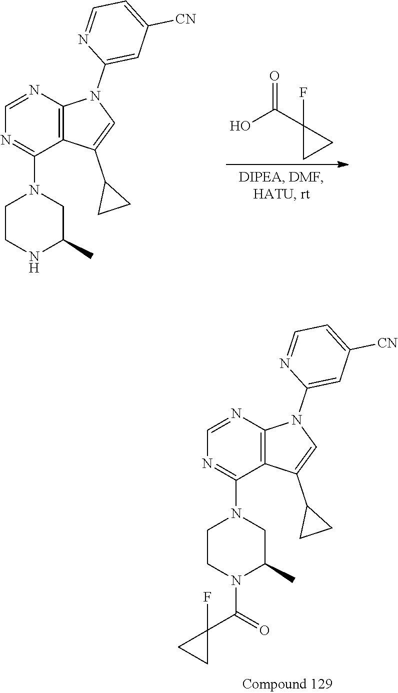 [SMILES: C[C@@H]1CN(c2ncnc3c2c(C2CC2)cn3-c2cc(C#N)ccn2)CCN1 ; O=C(O)C1(F)CC1 ; C[C@@H]1CN(c2ncnc3c2c(C2CC2)cn3-c2cc(C#N)ccn2)CCN1C(=O)C1(F)CC1]

### Step 1. 4-chloro-5-cyclopropyl-7-tosyl-7H-pyrrolo[2,3-d]pyrimidine

To a solution of 4-chloro-5-iodo-7-tosyl-7H-pyrrolo[2,3-d]pyrimidine (5.8 g, 13 mmol) in toluene (50 mL) were added cyclopropylboronic acid (1.09 g, 12.7 mmol), K₂CO₃ (24.0 g, 174 mmol) and Pd-118 (875 mg, 1.34 mmol). The resulting mixture was heated to 80° C. overnight. After being cooled down to room temperature, solvent was filtered. The filtrate was concentrated and purified by flash column chromatography (silica gel, 0˜30%, ethyl acetate in petroleum ether) to give 3.6 g of 4-chloro-5-cyclopropyl-7-tosyl-7H-pyrrolo[2,3-d]pyrimidine (3.7 g, 77% yield) as a solid. LC/MS ESI m/z: 348 (M+H)⁺.

### Step 2. tert-butyl (R)-4-(5-cyclopropyl-7-tosyl-7H-pyrrolo[2,3-d]pyrimidin-4-yl)-2-methylpiperazine-1-carboxylate

To a solution of 4-chloro-5-cyclopropyl-7-tosyl-7H-pyrrolo[2,3-d]pyrimidine (3.0 g, 8.7 mmol) in EtOH (60 mL) were added tert-butyl (R)-2-methylpiperazine-1-carboxylate (3.46 g, 17.3 mmol) and DIPEA (6.74 g, 52.2 mmol). The resulting mixture was heated to 100° C. overnight. After being cooled down to room temperature, solvent was removed and the residue was purified by flash column chromatography (silica gel, 0˜30%, ethyl acetate in petroleum ether) to afford tert-butyl (R)-4-(5-cyclopropyl-7-tosyl-7H-pyrrolo[2,3-d]pyrimidin-4-yl)-2-methylpiperazine-1-carboxylate (3.0 g, 73%) as a white solid. LC/MS ESI m/z: 512 (M+H)⁺.

### Step 3. tert-butyl (R)-4-(5-cyclopropyl-7H-pyrrolo[2,3-d]pyrimidin-4-yl)-2-methylpiperazine-1-carboxylate

To a solution of tert-butyl (R)-4-(5-cyclopropyl-7-tosyl-7H-pyrrolo[2,3-d]pyrimidin-4-yl)-2-methylpiperazine-1-carboxylate (3.0 g, 6.0 mmol) in THF (50 mL) was added TBAF (36 mL, 1.0M in THF). The resulting mixture was stirred at room temperature overnight. The reaction was quenched with water, extracted with EtOAc twice, the combined organic layers were washed with brine, dried over Na₂SO₄, filtered and concentrated. The residue was purified by flash column chromatography (silica gel, 0˜60%, ethyl acetate in petroleum ether) to afford tert-butyl (R)-4-(5-cyclopropyl-7H-pyrrolo[2,3-d]pyrimidin-4-yl)-2-methylpiperazine-1-carboxylate (1.9 g, 88%) as a white solid. LC/MS ESI m/z: 358 (M+H)⁺.

### Step 4. tert-butyl (R)-4-(7-(3-cyanophenyl)-5-cyclopropyl-7H-pyrrolo[2,3-d]pyrimidin-4-yl)-2-methylpiperazine-1-carboxylate

To a solution of tert-butyl (R)-4-(5-cyclopropyl-7H-pyrrolo[2,3-d]pyrimidin-4-yl)-2-methylpiperazine-1-carboxylate (1.2 g, 3.4 mmol) in DMF (15 mL) were added 2-bromoisonicotinonitrile (1.23 mg, 6.72 mmol), trans-cyclohexane-1,2-diamine (383 mg, 3.36 mmol), CuI (638 mg, 3.36 mmol) and K₃PO₄ (2.14 g, 10.1 mmol). The resulting mixture was heated to 120° C. overnight. After being cooled down to room temperature. The reaction was partitioned between EtOAc and water, organic layer was separated, aqueous layer was extracted with EtOAc twice, the combined organic layers were washed with brine, dried over Na₂SO₄, filtered and concentrated. The residue was purified by flash column chromatography (silica gel, 0˜30%, ethyl acetate in petroleum ether) to afford tert-butyl (R)-4-(7-(3-cyanophenyl)-5-cyclopropyl-7H-pyrrolo[2,3-d]pyrimidin-4-yl)-2-methylpiperazine-1-carboxylate (1.4 g, 91%) as a solid. LC/MS ESI m/z: 459 (M+H)⁺.

### Step 5. (R)-2-(5-cyclopropyl-4-(3-methylpiperazin-1-yl)-7H-pyrrolo[2,3-d]pyrimidin-7-yl)isonicotinonitrile

At 0° C., to a solution of tert-butyl (R)-4-(7-(3-cyanophenyl)-5-cyclopropyl-7H-pyrrolo[2,3-d]pyrimidin-4-yl)-2-methylpiperazine-1-carboxylate (1.1 g, 2.5 mmol) in DCM (10 mL) was added HCl (8.0 mL, 4.0M in dioxane). The resulting mixture was stirred at room temperature for 4 h. The reaction mixture was concentrated to afford (R)-2-(5-cyclopropyl-4-(3-methylpiperazin-1-yl)-7H-pyrrolo[2,3-d]pyrimidin-7-yl)isonicotinonitrile (890 mg, 100%) as a solid which was used in the next step directly. LC/MS ESI (m/z): 359 (M+H)⁺.

### Step 6. (R)-2-(5-cyclopropyl-4-(4-(1-fluorocyclopropane-1-carbonyl)-3-methylpiperazin-1-yl)-7H-pyrrolo[2,3-d]pyrimidin-7-yl)isonicotinonitrile

To a solution of (R)-2-(5-cyclopropyl-4-(3-methylpiperazin-1-yl)-7H-pyrrolo[2,3-d]pyrimidin-7-yl)isonicotinonitrile (60 mg, 0.17 mmol) in DMF (3 mL) were added 1-fluorocyclopropane-1-carboxylic acid (32 mg, 0.31 mmol), DIPEA (44 mg, 0.34 mmol) and HATU (129 mg, 0.34 mmol). The resulting mixture was stirred at room temperature overnight. The reaction was quenched with water, extracted with EtOAc twice, the combined organic layers were washed with NaHCO₃ (aq.) and brine, dried over Na₂SO₄, filtered and concentrated. The residue was purified by flash column chromatography (silica gel, 0˜35%, ethyl acetate in petroleum ether) to give crude product which was further purified by prep-HPLC to afford (R)-2-(5-cyclopropyl-4-(4-(1-fluorocyclopropane-1-carbonyl)-3-methylpiperazin-1-yl)-7H-pyrrolo[2,3-d]pyrimidin-7-yl)isonicotinonitrile (28 mg, 38%) as a white solid. LC/MS ESI m/z: 446 (M+H)⁺. ¹H NMR (400 MHZ, CDCl₃) δ 9.35-9.29 (m, 1H), 8.59 (dd, J=5.0, 0.8 Hz, 1H), 8.51 (s, 1H), 7.82 (d, J=0.9 Hz, 1H), 7.35 (dd, J=5.0, 1.3 Hz, 1H), 4.85-4.75 (m, 1H), 4.55-4.50 (m, 1H), 4.45-4.24 (m, 1H), 4.20-4.13 (m, 1H), 3.82-3.36 (m, 2H), 3.21-3.11 (m, 1H), 2.07-2.01 (m, 1H), 1.40-1.24 (m, 7H), 1.09-1.03 (m, 2H), 1.00-0.94 (m, 1H), 0.76-0.70 (m, 1H).

The following compounds were prepared by the procedure similar to the synthesis of compound 129 from the corresponding acids.

| Cmpd | | |
|---|---|---|
| No. | Chemical Name | LCMS and ¹H NMR |
| 130 | (R)-2-(5-cyclopropyl-4-(4-(2- | LC/MS ESI m/z: 448 (M + H)⁺. ¹H NMR (400 MHz, CDCl₃) |
| | fluoro-2-methylpropanoyl)-3- | 8 9.34-9.30 (m, 1H), 8.59 (dd, J = 5.0, 0.7 Hz, 1H), 8.51 |
| | methylpiperazin-1-yl)-7H- | (s, 1H), 7.83-7.80 (m, 1H), 7.35 (dd, J = 5.0, 1.3 Hz, 1H), |
| | pyrrolo[2,3-d]pyrimidin-7- | 4.91-4.83 (m, 1H), 4.56-4.36 (m, 2H), 4.20-4.07 (m, |
| | yl)isonicotinonitrile | 1H), 3.75-3.29 (m, 2H), 3.21-3.06 (m, 1H), 2.05-2.00 |
| | | (m, 1H), 1.68-1.60 (m, 6H), 1.41-1.24 (m, 3H), 1.08- |
| | | 1.03 (m, 2H), 0.99-0.94 (m, 1H), 0.76-0.70 (m, 1H). |

### Example 13. Synthesis of (R)-2-(5-(2-fluorophenyl)-4-(4-isobutyryl-2-methylpiperazin-1-yl)-7H-pyrrolo[2,3-d]pyrimidin-7-yl)isonicotinonitrile (Compound 138)

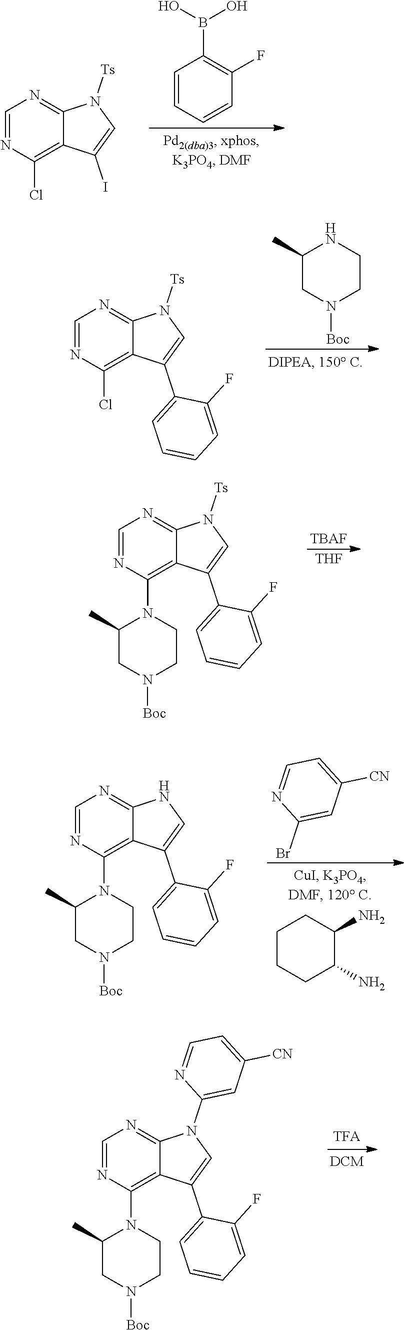

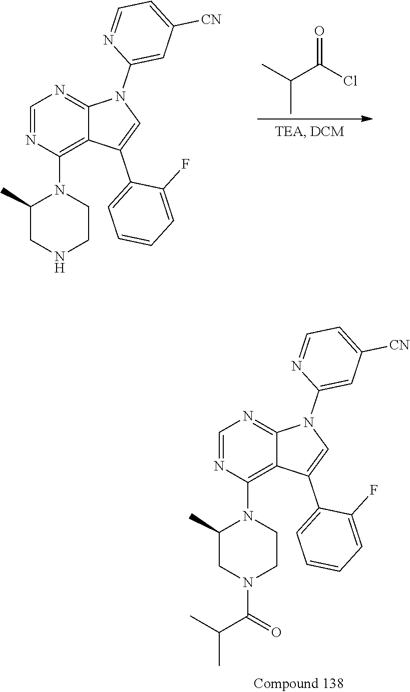

### Step 1. 4-chloro-5-(2-fluorophenyl)-7-tosyl-7H-pyrrolo[2,3-d]pyrimidine

To a solution of 4-chloro-5-iodo-7-tosyl-7H-pyrrolo[2,3-d]pyrimidine (1.1 g, 2.5 mmol) in DMF (15 mL) and water (0.5 mL) were added (2-fluorophenyl) boronic acid (420 mg, 0.60 mmol), X-Phos (180 mg, 0.37 mmol), K₃PO₄ (1.6 g, 7.5 mmol) and Pd₂(dba)₃ (230 mg, 0.25 mmol), the resulting mixture was heated to 60° C. overnight. After being cooled down to room temperature, the reaction mixture was filtered, filtrate was partitioned between EtOAc and water, organic layer was separated, aqueous layer was extracted with EtOAc twice, the combined organic layers were washed with brine, dried over Na₂SO₄, filtered and concentrated. The residue was purified by flash chromatography (silica gel, 0˜50% EtOAc in petroleum ether) to afford 4-chloro-5-(2-fluorophenyl)-7-tosyl-7H-pyrrolo[2,3-d]pyrimidine (600 mg, 60%). LC/MS ESI (m/z): 402 (M+H)⁺.

### Step 2. tert-butyl (R)-4-(5-(2-fluorophenyl)-7-tosyl-7H-pyrrolo[2,3-d]pyrimidin-4-yl)-3-methylpiperazine-1-carboxylate

A mixture of 4-chloro-5-(2-fluorophenyl)-7-tosyl-7H-pyrrolo[2,3-d]pyrimidine (600 mg, 1.49 mmol) and tert-butyl (R)-3-methylpiperazine-1-carboxylate (598 mg, 3.00 mmol) in DIEA (20 mL) was heated at 150° C. for 3 hours. The reaction mixture was concentrated, and the residue was purified by column chromatography on silica gel (0˜30% EtOAc in petroleum ether, V/V) to give the tert-butyl (R)-4-(5-(2-fluorophenyl)-7-tosyl-7H-pyrrolo[2,3-d]pyrimidin-4-yl)-3-methylpiperazine-1-carboxylate (520 mg, 61%) as a yellow solid. LC/MS ESI (m/z): 566 (M+H)⁺.

### Step 3. tert-butyl (R)-4-(5-(2-fluorophenyl)-7H-pyrrolo[2,3-d]pyrimidin-4-yl)-3-methylpiperazine-1-carboxylate

To a solution of tert-butyl (R)-4-(5-(2-fluorophenyl)-7-tosyl-7H-pyrrolo[2,3-d]pyrimidin-4-yl)-3-methylpiperazine-1-carboxylate (500 mg, 0.88 mmol) in THF (10 mL) was added TBAF (5.0 mL, 1.0M in THF). The resulting mixture was stirred at room temperature for 12 h. The reaction mixture was partitioned between EtOAc and water, organic layer was separated, aqueous layer was extracted with EtOAc twice. The combined layers were washed with brine, dried over Na₂SO₄ and concentrated. The residue was purified by column chromatography on silica gel (0˜80% EtOAc in petroleum ether, V/V) to give the tert-butyl (R)-4-(5-(2-fluorophenyl)-7H-pyrrolo[2,3-d]pyrimidin-4-yl)-3-methylpiperazine-1-carboxylate (100 mg, 27%) as a yellow solid. LC/MS ESI (m/z): 412 (M+H)⁺.

### Step 4. tert-butyl (R)-4-(7-(4-cyanopyridin-2-yl)-5-(2-fluorophenyl)-7H-pyrrolo[2,3-d]pyrimidin-4-yl)-3-methylpiperazine-1-carboxylate

A mixture of tert-butyl (R)-4-(5-(2-fluorophenyl)-7H-pyrrolo[2,3-d]pyrimidin-4-yl)-3-methylpiperazine-1-carboxylate (100 mg, 0.24 mmol), 2-bromopyridine-4-carbonitrile (89 mg, 0.49 mmol), CuI (23 mg, 0.12 mmol), trans-1,2-diaminocyclohexane (8.3 mg, 0.07 mmol) and K₃PO₄ (150 mg, 0.73 mmol) in DMF (10 mL) was heated at 120° C. for 12 hours. After being cooled down to room temperature, solvent was concentrated. The residue was purified by column chromatography on silica gel (0˜40% EtOAc in petroleum ether, V/V) to give the tert-butyl (R)-4-(7-(4-cyanopyridin-2-yl)-5-(2-fluorophenyl)-7H-pyrrolo[2,3-d]pyrimidin-4-yl)-3-methylpiperazine-1-carboxylate (80 mg, 64%) as a white solid. LC/MS ESI (m/z): 514 (M+H)⁺. ¹H NMR (400 MHZ, CD₃OD) δ 9.30 (s, 1H), 8.70 (d, J=5.0 Hz, 1H), 8.55 (s, 1H), 8.29 (s, 1H), 7.61 (dd, J=5.0, 1.3 Hz, 1H), 7.54 (td, J=7.5, 1.7 Hz, 1H), 7.50-7.43 (m, 1H), 7.34 (dd, J=7.5, 6.5 Hz, 1H), 7.31-7.24 (m, 1H), 4.19-4.11 (m, 1H), 3.72 (d, J=13.6 Hz, 1H), 3.49 (d, J=11.7 Hz, 2H), 3.13-3.06 (m, 1H), 2.73 (br, 2H), 1.42 (s, 9H), 0.97 (d, J=6.5 Hz, 3H).

### Step 5. (R)-2-(5-(2-fluorophenyl)-4-(2-methylpiperazin-1-yl)-7H-pyrrolo[2,3-d]pyrimidin-7-yl)isonicotinonitrile

At 0° C., to a solution of tert-butyl (R)-4-(7-(4-cyanopyridin-2-yl)-5-(2-fluorophenyl)-7H-pyrrolo[2,3-d]pyrimidin-4-yl)-3-methylpiperazine-1-carboxylate (60 mg, 0.12 mmol) in DCM (10 mL) was added TFA (4 mL). The resulting mixture was stirred at the same temperature for 1 h. The reaction mixture was basified with NaHCO₃ (aq.) and extracted with Na₂SO₄ and concentrated to afford the (R)-2-(5-(2-fluorophenyl)-4-(2-methylpiperazin-1-yl)-7H-pyrrolo[2,3-d]pyrimidin-7-yl)isonicotinonitrile (40 mg, 83%) as a yellow solid which was used in the next step without further purification. LC/MS ESI m/z: 414 (M+H)⁺.

### Step 6. (R)-2-(5-(2-fluorophenyl)-4-(4-isobutyryl-2-methylpiperazin-1-yl)-7H-pyrrolo[2,3-d]pyrimidin-7-yl)isonicotinonitrile

At 0° C., to a solution of (R)-2-(5-(2-fluorophenyl)-4-(2-methylpiperazin-1-yl)-7H-pyrrolo[2,3-d]pyrimidin-7-yl)isonicotinonitrile (40 mg, 0.10 mmol) and TEA (30 mg, 0.30 mmol) in DCM (5 mL) was added di-isobutyryl chloride (12 mg, 0.12 mmol). The resulting mixture was stirred at room temperature for 20 minutes. The reaction was quenched with ice water and extracted with DCM twice. The combined layers were washed with NaHCO₃ (aq.) and brine, dried over Na₂SO₄ and concentrated. The residue was purified by column chromatography on silica gel (0˜40% EtOAc in petroleum ether, V/V) to give the crude product, which was purified by prep-HPLC to give the (R)-2-(5-(2-fluorophenyl)-4-(4-isobutyryl-2-methylpiperazin-1-yl)-7H-pyrrolo[2,3-d]pyrimidin-7-yl)isonicotinonitrile (30 mg, 64%) as a white solid. LC/MS ESI (m/z): 484.1 (M+H)⁺. ¹H NMR (400 MHZ, CDCl₃) δ 9.41 (s, 1H), 8.63 (d, J=5.0 Hz, 1H), 8.58 (d, J=4.6 Hz, 1H), 8.29 (d, J=5.9 Hz, 1H), 7.53-7.43 (m, 1H), 7.43-7.34 (m, 2H), 7.26-7.16 (m, 2H), 4.46-3.98 (m, 2H), 3.65 (t, J=11.7 Hz, 1H), 3.44 (t, J=10.8 Hz, 1H), 3.25-2.97 (m, 2H), 2.81-2.65 (m, 1H), 2.56 (br, 1H), 1.14-1.08 (m, 6H), 1.07-0.87 (m, 3H).

The following compounds were prepared by the procedure analogous to the synthesis of compound 138 from the corresponding amines.

| Cmpd | | |
|---|---|---|
| No. | Chemical Name | LCMS and ¹H NMR |
| 139 | 2-(5-(2-fluorophenyl)-4- | LC/MS ESI (m/z): 498.2 (M + H)⁺. ¹H NMR (400 MHz, |
| | ((2S,5R)-4-isobutyryl-2,5- | CDCl3) δ 9.41 (s, 1H), 8.63 (d, J = 5.0 Hz, 1H), 8.58 (d, J = |
| | dimethylpiperazin-1-yl)-7H- | 6.0 Hz, 1H), 8.30 (d, J = 4.8 Hz, 1H), 7.54-7.46 (m, 1H), |
| | pyrrolo[2,3-d]pyrimidin-7- | 7.42-7.35 (m, 2H), 7.26-7.17 (m, 2H), 4.54 (m, 1H), |
| | yl)isonicotinonitrile | 4.07 (d, 1H), 3.70 (m, 1H), 3.34 (dd, J = 13.8, 5.0 Hz, 1H), |
| | | 3.22 (broad s, 1H), 3.17-2.75 (m, 1H), 2.66-2.51 (m, |
| | | 1H), 1.29-1.09 (m, 6H), 1.06-0.82 (m, 6H). |
| 111 | (R)-2-(4-(4- | LC/MS ESI m/z: 482.6 (M + H)⁺. ¹H NMR (400 MHz, |
| | (cyclopropanecarbonyl)-3- | CDCl₃) δ 9.41 (s, 1H), 8.65-8.62 (m, 1H), 8.59 (s, 1H), |
| | methylpiperazin-1-yl)-5-(2- | 8.29 (s, 1H), 7.52-7.47 (m, 1H), 7.42-7.37 (m, 2H), 7.28- |
| | fluorophenyl)-7H-pyrrolo[2,3- | 7.26 (m, 1H), 7.24-7.18 (m, 1H), 4.73-4.40 (m, 1H), |
| | d]pyrimidin-7- | 4.01-3.55 (m, 3H), 3.17-2.43 (m, 3H), 1.70-1.55 (m, |
| | yl)isonicotinonitrile | 4H), 0.98-0.91 (m, 2H), 0.78-0.70 (m, 2H). |
| 109 | (R)-2-(5-(2-fluorophenyl)-4-(3- | LC/MS ESI m/z: 498.6 (M + H)⁺. ¹H NMR (400 MHz, |
| | methyl-4-pivaloylpiperazin-1- | CDCl₃) δ 9.40 (s, 1H), 8.65-8.60 (m, 1H), 8.58 (s, 1H), |
| | yl)-7H-pyrrolo[2,3-d]pyrimidin- | 8.29 (s, 1H), 7.53-7.47 (m, 1H), 7.42-7.36 (m, 2H), 7.29- |
| | 7-yl)isonicotinonitrile | 7.26 (m, 1H), 7.25-7.19 (m, 1H), 4.68-4.52 (m, 1H), |
| | | 3.92-3.62 (m, 3H), 3.07-2.95 (m, 1H), 2.86-2.44 (m, |
| | | 2H), 1.23 (s, 12H). |
| 107 | (R)-2-(5-(2-fluorophenyl)-4-(4- | LC/MS ESI m/z: 484.5 (M + H)⁺. ¹H NMR (400 MHz, |
| | isobutyryl-3-methylpiperazin-1- | CDCl₃) δ 9.40 (s, 1H), 8.65-8.61 (m, 1H), 8.58 (s, 1H), |
| | yl)-7H-pyrrolo[2,3-d]pyrimidin- | 8.30 (s, 1H), 7.52-7.47 (m, 1H), 7.42-7.37 (m, 2H), 7.29- |
| | 7-yl)isonicotinonitrile | 7.26 (m, 1H), 7.25-7.19 (m, 1H), 4.82-4.16 (m, 1H), |
| | | 4.05-3.73 (m, 2H), 3.59-3.28 (m, 1H), 3.12-2.67 (m, |
| | | 3H), 2.62-2.26 (m, 1H), 1.34-1.23 (m, 2H), 1.13-1.02 |
| | | (m, 7H). |
| 101 | 2-(5-(2-fluorophenyl)-4-(2- | LC/MS ESI m/z: 496 (M + H)⁺. ¹H NMR (400 MHz, CDCl₃) |
| | isobutyryl-2,6- | δ 9.35 (s, 1H), 8.54 (d, J = 5.0 Hz, 1H), 8.40 (s, 1H), 8.06 |
| | diazaspiro[3.4]octan-6-yl)-7H- | (s, 1H), 7.39-7.35 (m, 1H), 7.34-7.29 (m, 2H), 7.17- |
| | pyrrolo[2,3-d]pyrimidin-7- | 7.07 (m, 2H), 3.85-3.81 (m, 2H), 3.73-3.65 (m, 2H), |
| | yl)isonicotinonitrile | 3.42-3.37 (m, 1H), 3.31-3.25 (m, 3H), 2.35-2.27 (m, |
| | | 1H), 1.89-1.85 (m, 2H), 1.00 (d, J = 6.8 Hz, 6H). |

### Example 14. Synthesis of (S)-2-(5-cyclopropyl-4-(2-methyl-4-(4,4,4-trifluoro-3,3-dimethylbutanoyl)piperazin-1-yl)-7H-pyrrolo[2,3-d]pyrimidin-7-yl)isonicotinonitrile (Compound 141)

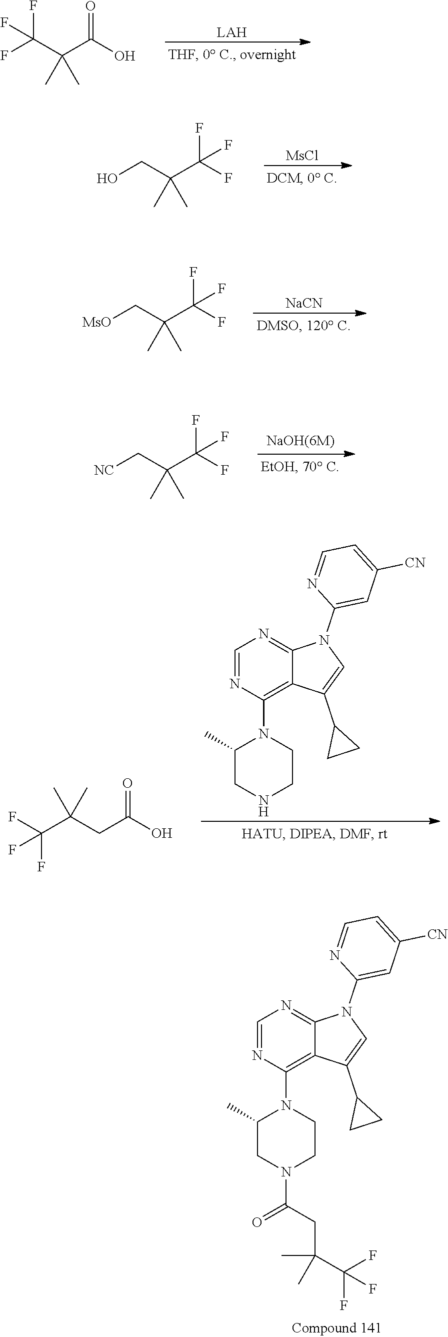

### Step 1. 3,3,3-trifluoro-2,2-dimethylpropan-1-ol

At 0° C., to a solution of 3,3,3-trifluoro-2,2-dimethylpropanoic acid (4.0 g, 26 mmol) in THF (20 mL) was added LAH (21 ml, 2.5M in THF) dropwise. The resulting mixture was stirred at room temperature overnight. The reaction was quenched with water, extracted with DCM twice, the combined organic layers were washed with brine, dried over Na₂SO₄, filtered and concentrated to afford 3,3,3-trifluoro-2,2-dimethylpropan-1-ol (2.5 g, 69%) as a colorless oil. ¹HNMR (400 MHZ, CD₃OD) δ 3.51 (s, 2H), 1.10 (s, 6H).

### Step 2. 3,3,3-trifluoro-2,2-dimethylpropyl methanesulfonate

At 0° C., to a solution of 3,3,3-trifluoro-2,2-dimethylpropan-1-ol (2.5 g, 18 mmol) in DCM (15 ml) was added MsCl (4.0 g, 35 mmol) dropwise. The resulting mixture was stirred at the room temperature for 3 h. The reaction was quenched with ice water, extracted with DCM twice, the combined organic layers were washed with brine, dried over Na₂SO₄, filtered and concentrated. The residue was used in the next step directly. 3,3,3-trifluoro-2,2-dimethylpropyl methanesulfonate. as a colorless oil. ¹HNMR (400 MHZ, CDCl₃) δ 4.15 (s, 2H), 3.05 (s, 3H), 1.23 (d, J=0.5 Hz, 6H).

### Step 3. 4,4,4-trifluoro-3,3-dimethylbutanenitrile

To a solution of 3,3,3-trifluoro-2,2-dimethylpropyl methanesulfonate (4.0 g, crude) in DMSO (10 mL) was added NaCN (1.3 g, 27 mmol). The resulting mixture was stirred at 120° C. for 3 days. After being cooled down to room temperature, the reaction was quenched with NaHCO₃ (aq.), extracted with MTBE twice, the combined organic layers were washed with brine, dried over Na₂SO₄, filtered and concentrated. The residue was used in the next step directly. 4,4,4-trifluoro-3,3-dimethylbutanenitrile (1.5 g, 40%) as a colorless oil. ¹H NMR (400 MHZ, CDCl₃) δ 2.56 (s, 2H), 1.32 (d, J=6.3 Hz, 6H).

### Step 4. 4,4,4-trifluoro-3,3-dimethylbutanoic acid

To a solution of 4,4,4-trifluoro-3,3-dimethylbutanenitrile (1.5 g, 6.8 mmol) in EtOH (10 mL) was added NaOH (5.0 ml of 6.0M). The resulting mixture was stirred at 70° C. overnight. The reaction was quenched with water and adjusted to pH=1 with concentrated HCl, extracted with EtOAc twice. The combined organic layers were washed with brine, dried over Na₂SO₄, filtered and concentrated to afford 4,4,4-trifluoro-3,3-dimethylbutanoic acid (450 mg, 45%) as a colorless oil. LC/MS ESI m/z: 169 (M−H)⁻

### Step 5. (S)-2-(5-cyclopropyl-4-(2-methyl-4-(4,4,4-trifluoro-3,3-dimethylbutanoyl) piperazin-1-yl)-7H-pyrrolo[2,3-d]pyrimidin-7-yl)isonicotinonitrile

To a solution of (S)-2-(5-cyclopropyl-4-(2-methylpiperazin-1-yl)-7H-pyrrolo[2,3-d]pyrimidin-7-yl)isonicotinonitrile (100 mg, 0.28 mmol) in DMF (5 mL) were added 4,4,4-trifluoro-3,3-dimethylbutanoic acid (94 mg, 0.56 mmol), DIEA (110 mg, 0.84 mmol) and HATU (210 mg, 0.56 mmol). The resulting mixture was stirred at room temperature overnight. The reaction was quenched with NaHCO₃ (aq.), extracted with EtOAc twice, the combined organic layers were washed with brine, dried over Na₂SO₄, filtered and concentrated. The residue was purified by flash column chromatography (silica gel, 0˜50%, ethyl acetate in petroleum ether) to give crude product (80 mg) which was further purified by prep-HPLC to afford (S)-2-(5-cyclopropyl-4-(2-methyl-4-(4,4,4-trifluoro-3,3-dimethylbutanoyl)piperazin-1-yl)-7H-pyrrolo[2,3-d]pyrimidin-7-yl)isonicotinonitrile (35 mg, 40%) as a white solid. LC/MS ESI m/z: 512 (M+H)⁺. ¹HNMR (400 MHZ, CDCl₃) δ 9.32 (s, 1H), 8.59 (d, J=5.0 Hz, 1H), 8.52 (s, 1H), 7.80 (s, 1H), 7.35 (d, J=4.2 Hz, 1H), 4.84-4.70 (m, 1H), 4.52-4.37 (m, 1H), 4.02-3.89 (m, 1.5H, rotamers), 3.68-3.45 (m, 2.5H, rotamers), 3.21-3.06 (m, 1H), 2.68-2.43 (m, 2H), 2.07-1.99 (m, 1H), 1.36 (s, 3H), 1.32 (d, J=2.4 Hz, 3H), 1.23 (m, 3H), 1.07-1.01 (m, 2H), 0.93-0.85 (m, 1H), 0.82-0.75 (m, 1H).

The following compounds were prepared by the procedures analogous to the synthesis of example above from the corresponding commercially available carboxylic acid.

| Cmpd | | |
|---|---|---|
| No. | Chemical Name | LCMS and ¹H NMR |
| 114 | (S)-2-(5-cyclopropyl-4-(4-(2- | LC/MS ESI m/z: 448 (M + H)⁺. ¹H NMR(400 MHz, MeOD) |
| | fluoro-2-methylpropanoyl)-2- | δ 9.21-9.15 (s, 1H), 8.64 (dd, J = 5.0, 0.8 Hz, 1H), 8.46 (s, |
| | methylpiperazin-1-yl)-7H- | 1H), 7.85 (d, J = 0.9 Hz, 1H), 7.53 (m, J = 5.0, 1.3 Hz, 1H), |
| | pyrrolo[2,3-d]pyrimidin-7- | 4.77 (m, 1H), 4.43 (m, 1H), 4.28 (d, J = 12.7 Hz, 1H), 3.95 |
| | yl)isonicotinonitrile | (d, J = 13.2 Hz, 1H), 3.79-3.50 (m, 2H), 3.36-3.12 (m, |
| | | 1H), 2.14-2.06 (m, 1H), 1.66 (m, J = 17.3 Hz, 6H), 1.27- |
| | | 1.13 (m, 3H), 1.08 (m, J = 8.1, 4.0 Hz, 2H), 0.88 (dd, J = |
| | | 9.2, 4.1 Hz, 1H), 0.80-0.71 (m, 1H) |

### Example 15. Synthesis of ethyl(S)-4-(7-(4-cyanopyridin-2-yl)-5-(2-fluorophenyl)-7H-pyrrolo[2,3-d]pyrimidin-4-yl)-3-methylpiperazine-1-carboxylate (Compound 142)

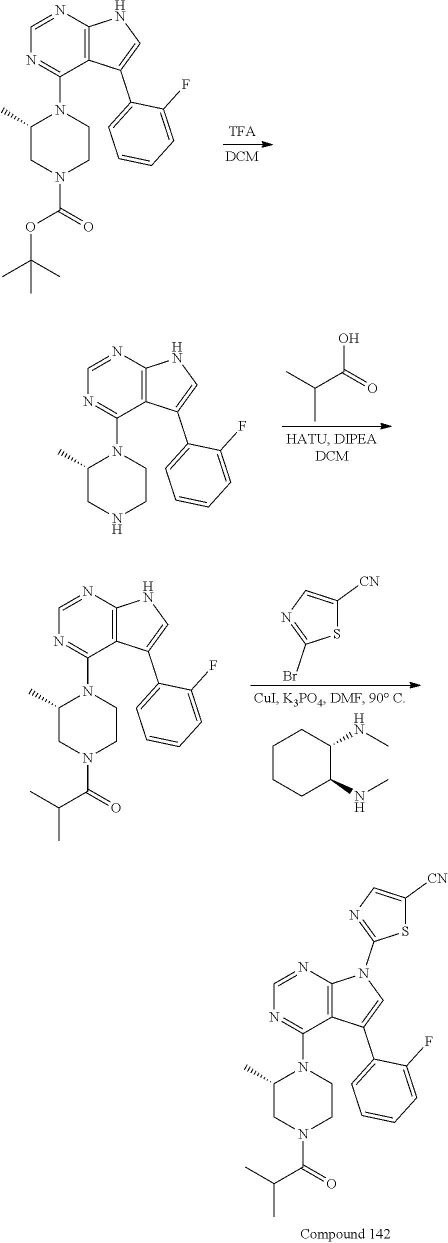

### Step 1. (S)-5-(2-fluorophenyl)-4-(2-methylpiperazin-1-yl)-7H-pyrrolo[2,3-d]pyrimidine

To a solution of tert-butyl (S)-4-(5-(2-fluorophenyl)-7H-pyrrolo[2,3-d]pyrimidin-4-yl)-3-methylpiperazine-1-carboxylate (2.0 g, 5.1 mmol, prepared following the procedure described for compound 143) in DCM (10 mL) at 0° C. was added TFA (3.0 mL). The resulting mixture was stirred at 0° C. for 2 h. The reaction was quenched with NaHCO₃ (aq.) and extracted twice with DCM/IPA (85/15). The combined organic layers were washed with brine, dried over Na₂SO₄, filtered and concentrated. The residue was used in the next step directly. LC/MS ESI m/z: 312 (M+H)⁺.

### Step 2. (S)-1-(4-(5-(2-fluorophenyl)-7H-pyrrolo[2,3-d]pyrimidin-4-yl)-3-methylpiperazin-1-yl)-2-methylpropan-1-one

To the solution of (S)-5-(2-fluorophenyl)-4-(2-methylpiperazin-1-yl)-7H-pyrrolo[2,3-d]pyrimidine (2.0 g, 6.4 mmol)) in DMF (9 mL) were added DIPEA (2.5 g, 19 mmol), 2-methylpropanoic acid (0.59 mL, 6.4 mmol) and HATU (2.4 g, 6.4 mmol) respectively. The resulting reaction mixture was stirred at room temperature for 30 minutes. The reaction was quenched with NaHCO₃ (aq.) and extracted twice with DCM/IPA (85/15). The combined organic layers were dried over Na₂SO₄, filtered and concentrated. The residue was purified by flash column chromatography to afford crude product(S)-1-(4-(5-(2-fluorophenyl)-7H-pyrrolo[2,3-d]pyrimidin-4-yl)-3-methylpiperazin-1-yl)-2-methylpropan-1-one (2.0 g, 82%) as a solid. LC/MS ESI m/z: 382.6 (M+H)⁺.

### Step 3. (S)-2-(5-(2-fluorophenyl)-4-(4-isobutyryl-2-methylpiperazin-1-yl)-7H-pyrrolo[2,3-d]pyrimidin-7-yl) thiazole-5-carbonitrile

To a solution of (S)-1-(4-(5-(2-fluorophenyl)-7H-pyrrolo[2,3-d]pyrimidin-4-yl)-3-methylpiperazin-1-yl)-2-methylpropan-1-one (50 mg, 0.13 mmol) in DMF (3 mL) were added CuI (25 mg, 0.13 mmol), K₃PO₄ (56 mg, 0.26 mmol), trans-N,N′-dimethylcyclohexane-1,2-diamine (37 mg, 0.26 mmol) and 2-bromo-1,3-thiazole-5-carbonitrile (50 mg, 0.26 mmol) respectively. The resulting reaction mixture was stirred at 90° C. under N₂ overnight. After cooling to room temperature, the reaction mixture was partitioned between EtOAc and water. The aqueous layer was extracted twice with EtOAc. The combined organic layers were washed with brine, dried over Na₂SO₄, filtered and concentrated. The residue was purified by flash column chromatography and Prep-HPLC to afford (S)-2-(5-(2-fluorophenyl)-4-(4-isobutyryl-2-methylpiperazin-1-yl)-7H-pyrrolo[2,3-d]pyrimidin-7-yl) thiazole-5-carbonitrile (10 mg, 16%) as a white solid. LC/MS ESI m/z: 490 (M+H)⁺. ¹H NMR (400 MHZ, CDCl₃) δ 8.60 (d, J=5.2 Hz, 1H), 8.14-8.04 (m, 2H), 7.49-7.38 (m, 2H), 7.31-7.27 (m, 1H), 7.25-7.19 (m, 1H), 4.61-4.52 (m, 0.5H, rotamers), 4.26-4.18 (m, 1H), 4.12-4.05 (m, 0.5H, rotamers), 3.76-3.61 (m, 1H), 3.59-3.42 (m, 1H), 3.27-2.97 (m, 2H), 2.78-2.46 (m, 2H), 1.16-1.04 (m, 8H), 0.94-0.90 (m, 1H).

The following compound was prepared by the procedure analogous to the synthesis of compound 142 from the corresponding aryl halide.

| Cmpd | | |
|---|---|---|
| No. | Chemical Name | LCMS and ¹H NMR |
| 146 | (S)-3-(5-(2-fluorophenyl)-4-(4- | LC/MS ESI m/z: 487 (M + H)⁺. ¹H NMR (400 MHz, CDCl₃) |
| | isobutyryl-2-methylpiperazin-1- | δ 8.53 (d, J = 3.5 Hz, 1H), 7.80 (d, J = 5.4 Hz, 1H), 7.71 (s, |
| | yl)-7H-pyrrolo[2,3-d]pyrimidin- | 1H), 7.49-7.43 (m, 1H), 7.39-7.33 (m, 1H), 7.25-7.15 |
| | 7-yl)-1-methyl-1H-pyrazole-5- | (m, 2H), 4.46-4.37 (m, 0.5H, rotamers), 4.19-4.13 (m, |
| | carbonitrile | 0.5H, rotamers), 4.12-4.06 (m, 3.5H, rotamers), 4.01 (d, |
| | | J = 12.9 Hz, 0.5H, rotamers), 3.70-3.61 (m, 1H), 3.49- |
| | | 3.40 (m, 1H), 3.24-2.97 (m, 2H), 2.79-2.66 (m, 1H), |
| | | 2.61-2.51 (m, 1H), 1.14-1.04 (m, 7.5H, rotamers), 0.89 |
| | | (d, J = 6.5 Hz, 1.5H, rotamers). |

### Example 16. Synthesis of (S)-2-(5-(2-fluorophenyl)-4-(4-isobutyryl-2-methylpiperazin-1-yl)-7H-pyrrolo[2,3-d]pyrimidin-7-yl)-6-methylisonicotinonitrile (Compound 143)

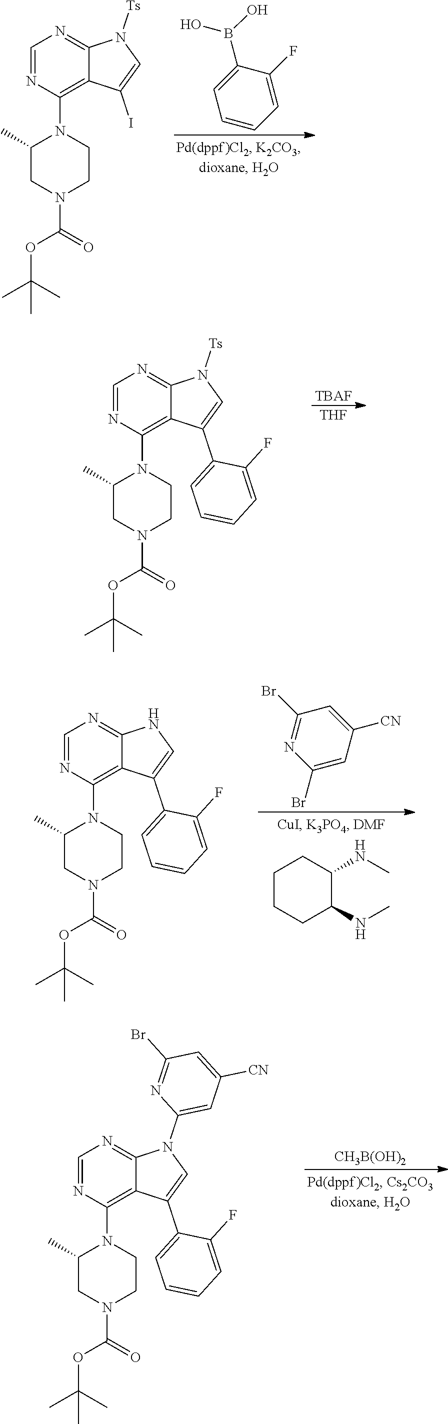

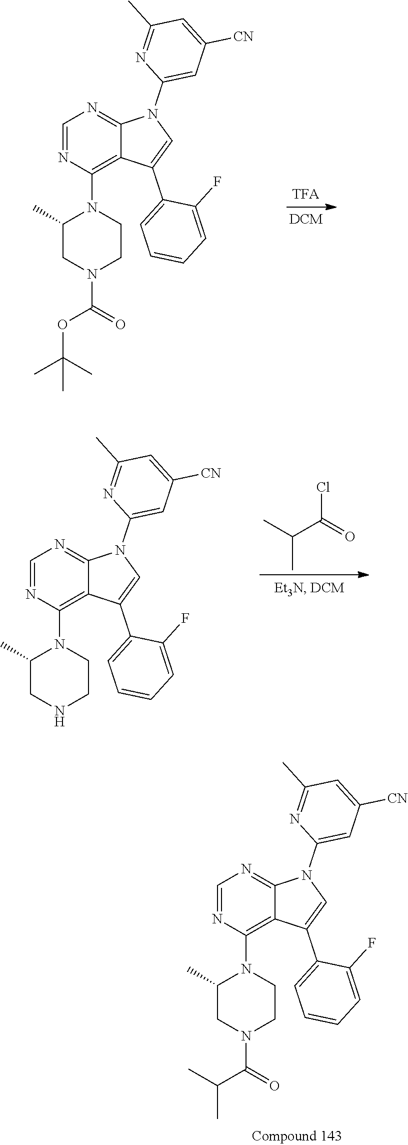

### Step 1. tert-butyl (S)-4-(7-(6-bromo-4-cyanopyridin-2-yl)-5-(2-fluorophenyl)-7H-pyrrolo[2,3-d]pyrimidin-4-yl)-3-methylpiperazine-1-carboxylate

To the solution of tert-butyl (S)-4-(5-iodo-7-tosyl-7H-pyrrolo[2,3-d]pyrimidin-4-yl)-3-methylpiperazine-1-carboxylate (1.0 g, 1.7 mmol, prepared following the procedure of compound 128) in dioxane (20 mL) and water (0.5 mL) were added Pd(dppf)Cl₂ (140 mg, 0.17 mmol), K₂CO₃ (930 mg, 6.7 mmol) and (2-fluorophenyl) boronic acid (280 mg, 2.0 mmol). The resulting reaction mixture was stirred at 90° C. overnight. After cooling to room temperature, solvent was removed and the residue was purified by flash column chromatography (silica gel, 0˜40%, ethyl acetate in petroleum ether) to obtain tert-butyl (S)-4-(5-(2-fluorophenyl)-7-tosyl-7H-pyrrolo[2,3-d]pyrimidin-4-yl)-3-methylpiperazine-1-carboxylate (810 mg, 85%) as a white solid. LC/MS ESI m/z: 566 (M+H)⁺.

### Step 2. tert-butyl (S)-4-(5-(2-fluorophenyl)-7H-pyrrolo[2,3-d]pyrimidin-4-yl)-3-methylpiperazine-1-carboxylate

To the solution of tert-butyl (S)-4-(5-(2-fluorophenyl)-7-tosyl-7H-pyrrolo[2,3-d]pyrimidin-4-yl)-3-methylpiperazine-1-carboxylate (570 mg, 1.0 mmol) in THF (4 mL) was added TBAF (4.0 mL, 1.0M in THF). The resulting reaction mixture was stirred at room temperature under N₂ atmosphere overnight. The reaction mixture was quenched with ice water and extracted twice with EtOAc. The combined organic layers were washed with water and brine, dried over Na₂SO₄, filtered, and concentrated under reduced pressure. The residue was purified by flash column chromatography (silica gel, 0˜60%, ethyl acetate in petroleum ether) to obtain tert-butyl (S)-4-(5-(2-fluorophenyl)-7H-pyrrolo[2,3-d]pyrimidin-4-yl)-3-methylpiperazine-1-carboxylate (330 mg, 80%) as a white solid. LC/MS ESI m/z: 412 (M+H)⁺.

### Step 3. tert-butyl (S)-4-(7-(6-bromo-4-cyanopyridin-2-yl)-5-(2-fluorophenyl)-7H-pyrrolo[2,3-d]pyrimidin-4-yl)-3-methylpiperazine-1-carboxylate

In a sealed tube, tert-butyl (S)-4-(5-(2-fluorophenyl)-7H-pyrrolo[2,3-d]pyrimidin-4-yl)-3-methylpiperazine-1-carboxylate (150 mg, 0.37 mmol), 2,6-dibromoisonicotinonitrile (190 mg, 0.73 mmol), K₃PO₄ (160 mg, 0.73 mmol) and CuI (69 mg, 0.36 mmol) were mixed in dry DMF. Then (±)-trans-1,2-cyclohexanediamine (100 mg, 0.73 mmol) was added. The reaction was stirred at 90° C. under N₂ atmosphere for 3.5 h. After cooling to room temperature, the reaction mixture was diluted with EtOAc, washed with 5% LiCl (aq.) and brine, dried over Na₂SO₄ and concentrated. The residue was purified by flash column chromatography (silica gel, 0˜11% EtOAc in petroleum ether) to provide tert-butyl (S)-4-(7-(6-bromo-4-cyanopyridin-2-yl)-5-(2-fluorophenyl)-7H-pyrrolo[2,3-d]pyrimidin-4-yl)-3-methylpiperazine-1-carboxylate (120 mg, 55%) as a yellow foam. LC/MS ESI (m/z): 592 (M+H)⁺.

### Step 4. tert-butyl (S)-4-(7-(4-cyano-6-methylpyridin-2-yl)-5-(2-fluorophenyl)-7H-pyrrolo[2,3-d]pyrimidin-4-yl)-3-methylpiperazine-1-carboxylate

A mixture of tert-butyl (S)-4-(7-(6-bromo-4-cyanopyridin-2-yl)-5-(2-fluorophenyl)-7H-pyrrolo[2,3-d]pyrimidin-4-yl)-3-methylpiperazine-1-carboxylate (120 mg, 0.20 mmol), methylboronic acid (98 mg, 1.6 mmol), Cs₂CO₃ (200 mg, 0.61 mmol) and Pd(dppf)Cl₂ (20 mg, 0.027 mmol) in dioxane (3 mL) and H₂O (0.6 mL) was stirred at 100° C. under N₂ atmosphere overnight. After cooling to room temperature, the reaction mixture was diluted with EtOAc and DCM and filtered. The filtrate was purified by flash column chromatography (silica gel, 0˜20% EtOAc in petroleum ether followed by Prep-HPLC to provide tert-butyl (S)-4-(7-(4-cyano-6-methylpyridin-2-yl)-5-(2-fluorophenyl)-7H-pyrrolo[2,3-d]pyrimidin-4-yl)-3-methylpiperazine-1-carboxylate (23 mg, 21%) as a light yellow oil. LC/MS ESI (m/z): 528 (M+H)⁺. ¹H NMR (400 MHZ, CDCl₃) δ 9.16 (s, 1H), 8.56 (s, 1H), 8.29 (s, 1H), 7.46 (td, J=7.6, 1.5 Hz, 1H), 7.37 (tdd, J=7.2, 5.1, 1.8 Hz, 1H), 7.26-7.17 (m, 3H), 4.32-4.06 (m, 1H), 3.86-3.61 (m, 1H), 3.56-3.42 (m, 2H), 3.08 (td, J=12.3, 2.5 Hz, 1H), 2.87-2.59 (m, 5H), 1.43 (s, 9H), 1.01 (d, J=5.7 Hz, 3H).

### Step 5. (S)-2-(5-(2-fluorophenyl)-4-(2-methylpiperazin-1-yl)-7H-pyrrolo[2,3-d]pyrimidin-7-yl)-6-methylisonicotinonitrile

To tert-butyl (S)-4-(7-(4-cyano-6-methylpyridin-2-yl)-5-(2-fluorophenyl)-7H-pyrrolo[2,3-d]pyrimidin-4-yl)-3-methylpiperazine-1-carboxylate (28 mg, 0.053 mmol) in DCM (3 mL) at room temperature was added TFA (0.50 mL) dropwise and stirred at room temperature for 2 h. The reaction mixture was then diluted with DCM and poured into saturated NaHCO₃ (aq.). The organic layer was dried over Na₂SO₄, filtered, and concentrated to dryness to give the desired crude product, which was used in the next step directly. LC/MS ESI (m/z): 428 (M+H)⁺.

### Step 6. (S)-2-(5-(2-fluorophenyl)-4-(4-isobutyryl-2-methylpiperazin-1-yl)-7H-pyrrolo[2,3-d]pyrimidin-7-yl)-6-methylisonicotinonitrile

A mixture of (S)-2-(5-(2-fluorophenyl)-4-(2-methylpiperazin-1-yl)-7H-pyrrolo[2,3-d]pyrimidin-7-yl)-6-methylisonicotinonitrile (24 mg, 0.056 mmol) and Et₃N (17 mg, 0.17 mmol) in dry DCM (2.5 mL) under N₂ atmosphere was cooled to 0° C. for 10 min. Isobutyryl chloride (10 mg, 0.094 mmol) was added. The reaction was stirred at 0° C. for 15 min. The mixture was diluted with DCM, washed with NaHCO₃ (aq.), dried over Na₂SO₄ and concentrated. The residue was purified by Prep-HPLC to give (S)-2-(5-(2-fluorophenyl)-4-(4-isobutyryl-2-methylpiperazin-1-yl)-7H-pyrrolo[2,3-d]pyrimidin-7-yl)-6-methylisonicotinonitrile (18 mg, 63%) as a white solid. LC/MS ESI (m/z): 498 (M+H)⁺. ¹H NMR (400 MHZ, CDCl₃) δ 9.16 (s, 1H), 8.58 (d, J=5.0 Hz, 1H), 8.31 (d, J=6.1 Hz, 1H), 7.48 (dt, J=12.8, 6.4 Hz, 1H), 7.41-7.33 (m, 1H), 7.26-7.16 (m, 3H), 4.27 (m, 1H), 4.11-3.94 (m, 1H), 3.70-3.61 (m, 1H), 3.44 (m, 1H), 3.09 (m, 2H), 2.74 (m, 1H), 2.63 (s, 3H), 2.55 (td, J=12.6, 2.6 Hz, 1H), 1.13-1.04 (m, 7H), 0.92 (m, 2H).

### Example 17. Synthesis of (S)-2-(4-(4-isobutyryl-2-methylpiperazin-1-yl)-5-(pyrrolidin-1-yl)-7H-pyrrolo[2,3-d]pyrimidin-7-yl)isonicotinonitrile (Compound 144)

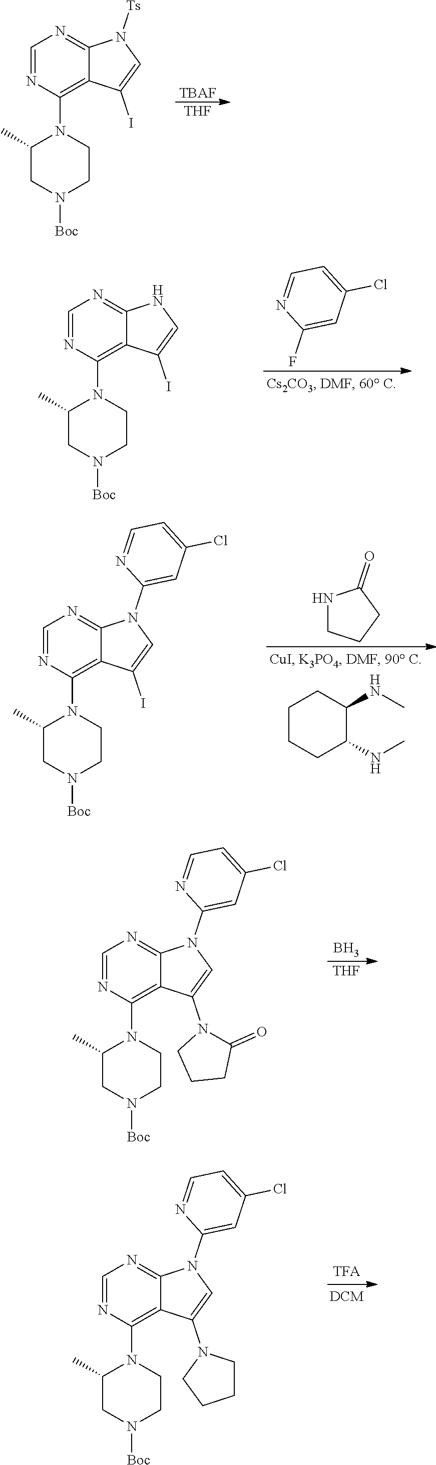

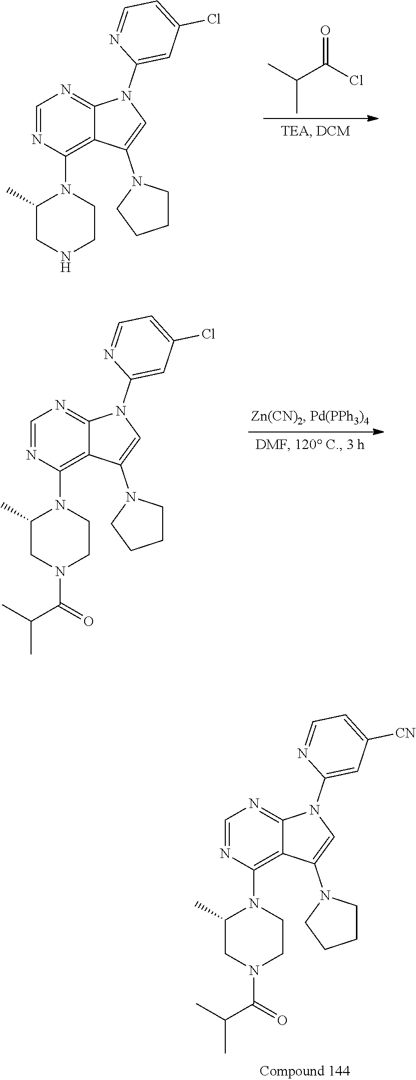

### Step 1. tert-butyl (S)-4-(5-iodo-7H-pyrrolo[2,3-d]pyrimidin-4-yl)-3-methylpiperazine-1-carboxylate

To a solution of tert-butyl (S)-4-(5-iodo-7-tosyl-7H-pyrrolo[2,3-d]pyrimidin-4-yl)-3-methylpiperazine-1-carboxylate (55 g, 110 mmol, prepared following the procedure described for compound 128) in THF (400 mL) at 0° C. was added TBAF (440 mL, 1.0M in THF). The resulting mixture was stirred at 0° C. for 1.5 h. The reaction was quenched with ice water and extracted twice with EtOAc. The combined organic layers were washed with brine, dried over Na₂SO₄, filtered and concentrated. The residue was purified by flash column chromatography (silica gel, 0˜10%, methanol in dichloromethane) to afford tert-butyl (S)-4-(5-iodo-7H-pyrrolo[2,3-d]pyrimidin-4-yl)-3-methylpiperazine-1-carboxylate (37 g, 91%) as a white solid. LC/MS ESI m/z: 444 (M+H)⁺

### Step 2. tert-butyl (S)-4-(7-(4-chloropyridin-2-yl)-5-iodo-7H-pyrrolo[2,3-d]pyrimidin-4-yl)-3-methylpiperazine-1-carboxylate

To a solution of tert-butyl (S)-4-(5-iodo-7H-pyrrolo[2,3-d]pyrimidin-4-yl)-3-methylpiperazine-1-carboxylate (2.0 g, 4.5 mmol) and 4-chloro-2-fluoropyridine (0.71 g, 5.4 mmol) in DMF (20 mL) was added Cs₂CO₃ (2.9 g, 9.0 mmol). The resulting mixture was stirred at 60° C. for 18 h. After cooling to room temperature, the reaction was partitioned between EtOAc and water. The aqueous layer was extracted twice with EtOAc. The combined organic layers were washed with brine, dried over anhydrous Na₂SO₄, filtered and concentrated. The residue was purified by column chromatography on silica gel (0˜35% EtOAc in petroleum ether) to give tert-butyl (S)-4-(7-(4-chloropyridin-2-yl)-5-iodo-7H-pyrrolo[2,3-d]pyrimidin-4-yl)-3-methylpiperazine-1-carboxylate (1.5 g, 60%) as a white solid. LC/MS ESI m/z: 555 (M+H)⁺

### Step 3 tert-butyl (S)-4-(7-(4-chloropyridin-2-yl)-5-(2-oxopyrrolidin-1-yl)-7H-pyrrolo[2,3-d]pyrimidin-4-yl)-3-methylpiperazine-1-carboxylate

A mixture of tert-butyl (S)-4-(7-(4-chloropyridin-2-yl)-5-iodo-7H-pyrrolo[2,3-d]pyrimidin-4-yl)-3-methylpiperazine-1-carboxylate (1.5 g, 2.7 mmol), pyrrolidin-2-one (0.62 mL, 8.1 mmol), K₃PO₄ (1.5 g, 8.1 mmol), CuI (0.29 g, 1.4 mmol) and trans-N,N′-dimethyl-1,2-cyclohexanediamine (0.19 g, 1.4 mmol) in DMF (15 mL) was stirred at 90° C. under N₂ atmosphere overnight. After cooling to room temperature, the reaction was partitioned between EtOAc and water. The aqueous layer was extracted twice with EtOAc. The combined organic layers were washed with brine, dried over anhydrous Na₂SO₄, filtered and concentrated. The residue was purified by column chromatography (0˜50% EtOAc in petroleum ether) to give tert-butyl (S)-4-(7-(4-chloropyridin-2-yl)-5-(2-oxopyrrolidin-1-yl)-7H-pyrrolo[2,3-d]pyrimidin-4-yl)-3-methylpiperazine-1-carboxylate (0.60 g, 43%) as a white solid. LC/MS ESI m/z: 512 (M+H)⁺

### Step 4. tert-butyl (S)-4-(7-(4-chloropyridin-2-yl)-5-(pyrrolidin-1-yl)-7H-pyrrolo[2,3-d]pyrimidin-4-yl)-3-methylpiperazine-1-carboxylate

To a solution of tert-butyl (S)-4-(7-(4-chloropyridin-2-yl)-5-(2-oxopyrrolidin-1-yl)-7H-pyrrolo[2,3-d]pyrimidin-4-yl)-3-methylpiperazine-1-carboxylate (600 mg, 1.2 mmol) in dry THF (5 mL) at 0° C. was added BH₃/THF (12 mL, 1.0M in THF) dropwise. The resulting mixture was stirred for 3 h. The reaction was quenched with saturated NaHCO₃ (aq.) and extracted twice with EtOAc. The combined organic phases were washed with brine, dried over anhydrous Na₂SO₄, filtered and concentrated. The residue was purified by column chromatography on silica gel (0˜50% EtOAc in petroleum ether) to give tert-butyl (S)-4-(7-(4-chloropyridin-2-yl)-5-(pyrrolidin-1-yl)-7H-pyrrolo[2,3-d]pyrimidin-4-yl)-3-methylpiperazine-1-carboxylate (300 mg, 51%) as a yellow solid. LC/MS ESI m/z: 498 (M+H)⁺

### Step 5. (S)-7-(4-chloropyridin-2-yl)-4-(2-methylpiperazin-1-yl)-5-(pyrrolidin-1-yl)-7H-pyrrolo[2,3-d]pyrimidine

To a solution of tert-butyl (S)-4-(7-(4-chloropyridin-2-yl)-5-(pyrrolidin-1-yl)-7H-pyrrolo[2,3-d]pyrimidin-4-yl)-3-methylpiperazine-1-carboxylate (300 mg, 0.60 mmol) in DCM (3 mL) was added TFA (0.89 mL, 12 mmol). The reaction was stirred at room temperature for 3 h. After removal of solvent, the residue was used directly in next step without further purification. LC/MS ESI m/z: 398 (M+H)⁺.

### Step 6. (S)-1-(4-(7-(4-chloropyridin-2-yl)-5-(pyrrolidin-1-yl)-7H-pyrrolo[2,3-d]pyrimidin-4-yl)-3-methylpiperazin-1-yl)-2-methylpropan-1-one

To a solution of (S)-7-(4-chloropyridin-2-yl)-4-(2-methylpiperazin-1-yl)-5-(pyrrolidin-1-yl)-7H-pyrrolo[2,3-d]pyrimidine (230 mg, 0.58 mmol) and TEA (0.48 mL, 3.5 mmol) in DCM (5 mL) at 0° C. was added a solution of 2-methylpropanoyl chloride (0.090 mL, 0.87 mmol) in DCM (0.5 mL). The resulting mixture was stirred at 0° C. for 1 h. The mixture was quenched with NaHCO₃ (aq.) and extracted twice with DCM. The combined organic phases were washed with brine, dried over anhydrous Na₂SO₄, filtered and concentrated. The residue was purified by column chromatography on silica gel (0˜50% EtOAc in petroleum ether) to give (S)-1-(4-(7-(4-chloropyridin-2-yl)-5-(pyrrolidin-1-yl)-7H-pyrrolo[2,3-d]pyrimidin-4-yl)-3-methylpiperazin-1-yl)-2-methylpropan-1-one (90 mg, 33%) as a yellow solid. LC/MS ESI m/z: 468 (M+H)⁺.

### Step 7. (S)-2-(4-(4-isobutyryl-2-methylpiperazin-1-yl)-5-(pyrrolidin-1-yl)-7H-pyrrolo[2,3-d]pyrimidin-7-yl)isonicotinonitrile

A mixture of (S)-1-(4-(7-(4-chloropyridin-2-yl)-5-(pyrrolidin-1-yl)-7H-pyrrolo[2,3-d]pyrimidin-4-yl)-3-methylpiperazin-1-yl)-2-methylpropan-1-one (90 mg, 0.19 mmol), Zn(CN)₂ (68 mg, 0.57 mmol) and Pd(PPh₃)⁺ (67 mg, 0.060 mmol) in DMF (2 mL) was stirred at 120° C. under N₂ atmosphere overnight. After cooling to room temperature, the reaction was partitioned between EtOAc and water. The aqueous layer was extracted twice with EtOAc. The combined organic phases were washed with brine, dried over anhydrous Na₂SO₄, filtered and concentrated. The residue was purified by column chromatography on silica gel (0˜50% EtOAc in petroleum ether) and Prep-HPLC to give (S)-2-(4-(4-isobutyryl-2-methylpiperazin-1-yl)-5-(pyrrolidin-1-yl)-7H-pyrrolo[2,3-d]pyrimidin-7-yl)isonicotinonitrile (30 mg, 34%) as a yellow solid. LC/MS ESI m/z: 459 (M+H)⁺. ¹H NMR (400 MHZ, CDCl₃) δ 9.35 (s, 1H), 8.57 (d, J=5.0 Hz, 1H), 8.45 (s, 1H), 7.58 (s, 1H), 7.32-7.27 (m, 1H), 5.06 (d, J=8.0 Hz, 1H), 4.56 (m, 1H), 4.26 (m, 1H), 4.05-3.69 (m, 1H), 3.64-3.31 (m, 2H), 3.21 (m, 2H), 3.11-2.75 (m, 4H), 2.03-1.90 (m, 4H), 1.25-0.99 (m, 9H).

The following compound was prepared by the procedure similar to the synthesis of compound 144 from the corresponding acids. The acid (4,4,4-trifluoro-3,3-dimethylbutanoic acid) for compound 140 was prepared following the procedure outlined in the experimental of compound 141

| Cmpd | | |
|---|---|---|
| No. | Chemical Name | LCMS and ¹H NMR |
| 148 | (R)-2-(4-(4-isobutyryl-3- | LC/MS ESI m/z: 459.5 (M + H)⁺. ¹H NMR (400 MHz, |
| | methylpiperazin-1-yl)-5- | CDCl₃) δ 9.36 (s, 1H), 8.58 (d, J = 5.0 Hz, 1H), 8.44 (s, |
| | (pyrrolidin-1-yl)-7H- | 1H), 7.58 (s, 1H), 7.30 (d, J = 5.0 Hz, 1H), 4.88-4.81 (m, |
| | pyrrolo[2,3-d]pyrimidin-7- | 1H), 4.60-4.30 (m, 2H), 3.93-3.51 (m, 2H), 3.30-3.22 |
| | yl)isonicotinonitrile | (m, 3H), 2.97-2.75 (m, 4H), 2.00-1.96 (m, 4H), 1.29- |
| | | 1.22 (m, 2H), 1.19-1.12 (m, 7H). |

### Example 18. Synthesis of 2-(5-(2-fluorophenyl)-4-(4-isobutyryl-4,7-diazaspiro[2.5]octan-7-yl)-7H-pyrrolo[2,3-d]pyrimidin-7-yl)isonicotinonitrile (Compound 145)

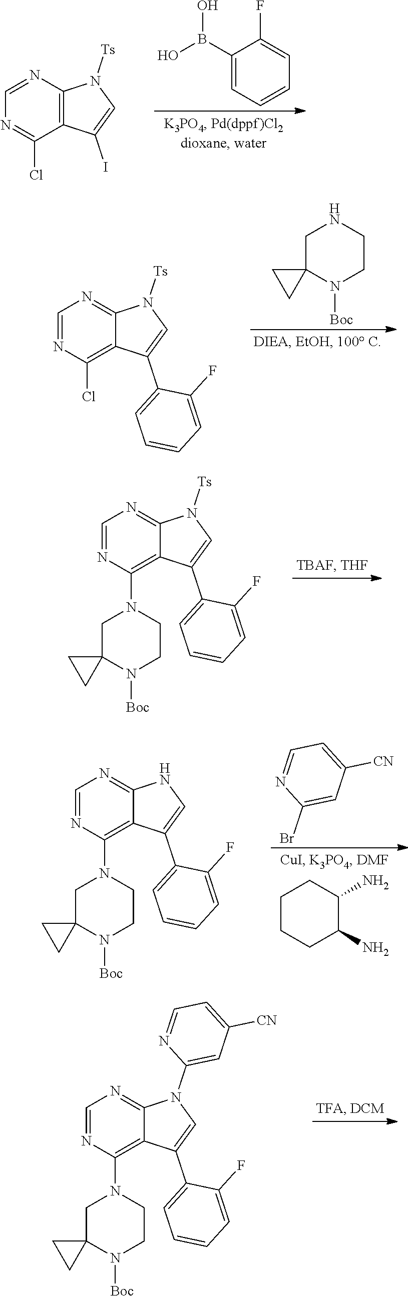

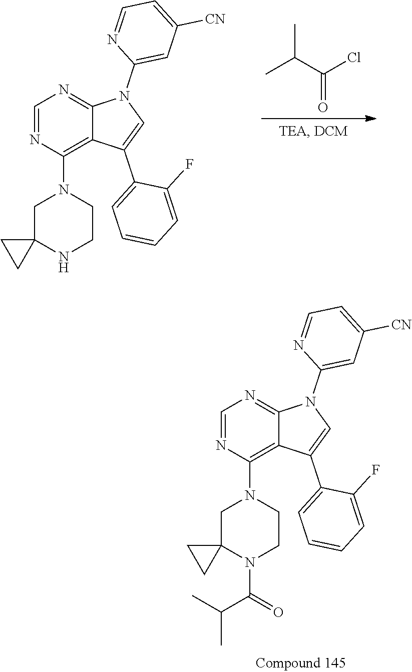

### Step 1. 4-chloro-5-(2-fluorophenyl)-7-tosyl-7H-pyrrolo[2,3-d]pyrimidine

To a solution of 4-chloro-5-iodo-7-tosyl-7H-pyrrolo[2,3-d]pyrimidine (10 g, 23 mmol, prepared following the procedure of compound 128) in dioxane-H₂O (100 mL, 5:1 v/v) were added (2-fluorophenyl) boronic acid (3.3 g, 23 mmol), K₃PO₄ (9.8 g, 46 mmol) and Pd(dppf)Cl₂ (1.7 g, 2.3 mmol). The resulting mixture was heated to 60° C. overnight. After cooling to room temperature, the reaction was filtered. The filtrate was partitioned between EtOAc and water. The aqueous phase was extracted twice with EtOAc. The combined organic layers were washed with brine, dried over Na₂SO₄, filtered and concentrated. The residue was purified by flash column chromatography (silica gel, 0˜30% EtOAc in petroleum ether) to give 4-chloro-5-(2-fluorophenyl)-7-tosyl-7H-pyrrolo[2,3-d]pyrimidine (7.0 g, 75% yield) as a solid. LC/MS ESI m/z: 402 (M+H)⁺.

### Step 2. tert-butyl 7-(5-(2-fluorophenyl)-7-tosyl-7H-pyrrolo[2,3-d]pyrimidin-4-yl)-4,7-diazaspiro[2.5]octane-4-carboxylate

To the solution of 4-chloro-5-(2-fluorophenyl)-7-tosyl-7H-pyrrolo[2,3-d]pyrimidine (550 mg, 2.2 mmol) in EtOH (6 mL) were added DIEA (0.66 mL, 4.0 mmol) and tert-butyl 4,7-diazaspiro[2.5]octane-4-carboxylate (440 mg, 2.1 mmol) respectively. The resulting reaction mixture was stirred at 100° C. under N₂ atmosphere overnight. After cooling to room temperature, solvent was removed. The crude product was purified by flash column chromatography (silica gel, 0˜20%, ethyl acetate in petroleum ether) to afford tert-butyl 7-(5-(2-fluorophenyl)-7-tosyl-7H-pyrrolo[2,3-d]pyrimidin-4-yl)-4,7-diazaspiro[2.5]octane-4-carboxylate (660 mg, 70%) as white solid. LC/MS ESI m/z: 578 (M+H)⁺.

### Step 3. tert-butyl 7-(5-(2-fluorophenyl)-7H-pyrrolo[2,3-d]pyrimidin-4-yl)-4,7-diazaspiro[2.5]octane-4-carboxylate

To the solution of tert-butyl 7-(5-(2-fluorophenyl)-7-tosyl-7H-pyrrolo[2,3-d]pyrimidin-4-yl)-4,7-diazaspiro[2.5]octane-4-carboxylate (660 mg, 1.6 mmol) in THF (10 mL) was added TBAF (6.5 mL, 1.0M in THF). The resulting reaction mixture was stirred at rt under N₂ atmosphere overnight. The reaction was quenched with ice water and extracted twice with EtOAc. The combined organic layers were washed with water, dried over Na₂SO₄, filtered, and concentrated under reduced pressure. The crude product was purified by flash column chromatography (silica gel, 0˜70%, ethyl acetate in petroleum ether) to obtain tert-butyl 7-(5-(2-fluorophenyl)-7H-pyrrolo[2,3-d]pyrimidin-4-yl)-4,7-diazaspiro[2.5]octane-4-carboxylate (410 mg, 62%) as a white solid. LC/MS ESI m/z: 424 (M+H)⁺.

### Step 4. tert-butyl 7-(7-(4-cyanopyridin-2-yl)-5-(2-fluorophenyl)-7H-pyrrolo[2,3-d]pyrimidin-4-yl)-4,7-diazaspiro[2.5]octane-4-carboxylate

To a solution of tert-butyl 7-(5-(2-fluorophenyl)-7H-pyrrolo[2,3-d]pyrimidin-4-yl)-4,7-diazaspiro[2.5]octane-4-carboxylate (400 mg, 0.95 mmol) in DMF (5 mL) were added CuI (90 mg, 0.47 mmol), K₃PO₄ (600 mg, 2.9 mmol), trans-cyclohexane-1,2-diamine (0.030 mL, 0.28 mmol) and 2-bromoisonicotinonitrile (350 mg, 1.9 mmol) respectively. The resulting reaction mixture was stirred at 80° C. under N₂ atmosphere overnight. After cooling to room temperature, the reaction was partitioned between EtOAc and water. The aqueous layer was extracted twice with EtOAc. The combined organic layers were washed with brine, dried over Na₂SO₄, filtered, and concentrated under reduced pressure. The crude product was purified by flash column chromatography (silica gel, 0˜20%, ethyl acetate in petroleum ether) to afford tert-butyl 7-(7-(4-cyanopyridin-2-yl)-5-(2-fluorophenyl)-7H-pyrrolo[2,3-d]pyrimidin-4-yl)-4,7-diazaspiro[2.5]octane-4-carboxylate (450 mg, 91%) as a white solid. LC/MS ESI m/z: 526 (M+H)⁺. ¹H NMR (400 MHZ, CDCl₃) δ 9.40 (s, 1H), 8.63-8.60 (m, 1H), 8.54 (s, 1H), 8.28 (s, 1H), 7.50-7.46 (m, 1H), 7.41-7.35 (m, 2H), 7.28-7.18 (m, 2H), 3.28 (s, 2H), 3.21 (s, 2H), 3.12 (s, 2H), 1.42 (s, 9H), 0.93-0.88 (m, 2H), 0.74 (s, 2H).

### Step 5. 2-(5-(2-fluorophenyl)-4-(4,7-diazaspiro[2.5]octan-7-yl)-7H-pyrrolo[2,3-d]pyrimidin-7-yl)isonicotinonitrile

To a solution of tert-butyl 7-(7-(4-cyanopyridin-2-yl)-5-(2-fluorophenyl)-7H-pyrrolo[2,3-d]pyrimidin-4-yl)-4,7-diazaspiro[2.5]octane-4-carboxylate (70 mg, 0.11 mmol) in DCM (1 mL) at 0° C. was added TFA (1.0 mL). The reaction was stirred at 0° C. for 1 h. The resulting mixture was basified with NaHCO₃ (aq.) and extracted twice with DCM. The combined organic layers were washed with NaHCO₃ (aq.), dried over Na₂SO₄, filtered and concentrated. The residue was used in the next step directly. LC/MS ESI m/z: 426 (M+H)⁺.

### Step 6. 2-(5-(2-fluorophenyl)-4-(4-isobutyryl-4,7-diazaspiro[2.5]octan-7-yl)-7H-pyrrolo[2,3-d]pyrimidin-7-yl)isonicotinonitrile

To a solution of 2-(5-(2-fluorophenyl)-4-(4,7-diazaspiro[2.5]octan-7-yl)-7H-pyrrolo[2,3-d]pyrimidin-7-yl)isonicotinonitrile (50 mg, 0.12 mmol) in DCM (2 mL) at 0° C. was added TEA (0.050 mL, 0.36 mmol), followed by isobutyryl chloride (0.020 mL, 0.24 mmol). The resulting mixture was stirred at room temperature for 20 minutes. The reaction was quenched with ice water and extracted twice with DCM. The combined organic layers were washed with NaHCO₃ (aq.), dried over Na₂SO₄, filtered, concentrated, the residue was purified by Prep-HPLC to afford 2-(5-(2-fluorophenyl)-4-(4-isobutyryl-4,7-diazaspiro[2.5]octan-7-yl)-7H-pyrrolo[2,3-d]pyrimidin-7-yl)isonicotinonitrile (32 mg, 53%) as a white solid. LC/MS ESI m/z: 496 (M+H)⁺. ¹H NMR (400 MHZ, CDCl₃) δ 9.40 (s, 1H), 8.63 (d, J=5.0 Hz, 1H), 8.54 (s, 1H), 8.29 (s, 1H), 7.53-7.46 (m, 1H), 7.42-7.35 (m, 2H), 7.29-7.18 (m, 2H), 3.61-2.48 (m, 7H), 1.12-0.70 (m, 10H).

### Example 19. Synthesis of (S)-1-(4-(7-(3-chlorophenyl)-5-(diethylamino)-7H-pyrrolo[2,3-d]pyrimidin-4-yl)-3-methylpiperazin-1-yl)-2-methylpropan-1-one (Compound 147)

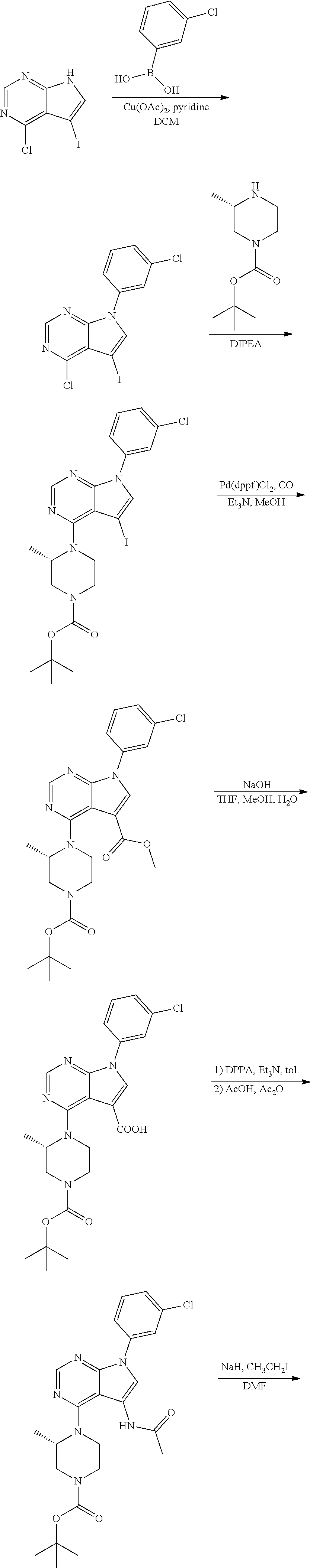

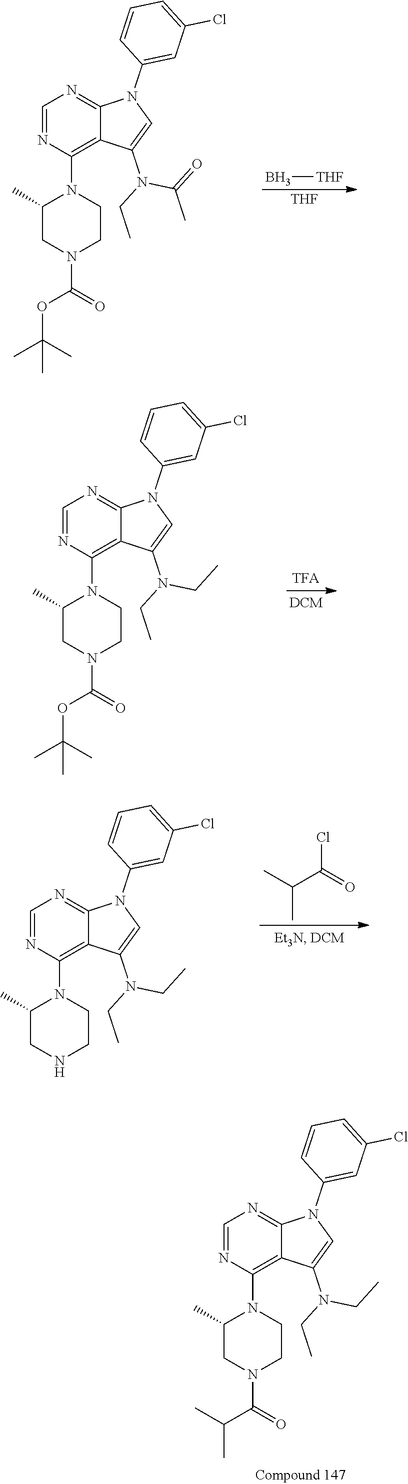

### Step 1. 4-chloro-7-(3-chlorophenyl)-5-iodo-7H-pyrrolo[2,3-d]pyrimidine

To a mixture of 4-chloro-5-iodo-7H-pyrrolo[2,3-d]pyrimidine (10 g, 36 mmol), (3-chlorophenyl) boronic acid (11 g, 72 mmol) and Cu(OAc)₂ (10 g, 55 mmol) in DCM (300 mL) were added pyridine (18 mL, 220 mmol) and 4 Å MS. The reaction was stirred under O₂ atmosphere at room temperature for 2 days. The resultant mixture was then treated with NH₄OH (aq., 25%) and filtered through Celite. The filtrate was washed with water, dried over Na₂SO₄ and concentrated to dryness. The crude solid product was triturated with EtOAc/petroleum ether (1:5, v/v) to give 4-chloro-7-(3-chlorophenyl)-5-iodo-7H-pyrrolo[2,3-d]pyrimidine (12 g, ˜75% purity, 64%) as a brown solid. LC/MS ESI (m/z): 390 (M+H)⁺.

### Step 2. tert-butyl (S)-4-(7-(3-chlorophenyl)-5-iodo-7H-pyrrolo[2,3-d]pyrimidin-4-yl)-3-methylpiperazine-1-carboxylate

A mixture of 4-chloro-7-(3-chlorophenyl)-5-iodo-7H-pyrrolo[2,3-d]pyrimidine (3.0 g, ˜75% purity, 5.8 mmol) and tert-butyl (S)-3-methylpiperazine-1-carboxylate (3.0 g, 15 mmol) in DIPEA (6.5 mL, 39 mmol) was stirred at 140° C. for 2.5 h. DIPEA was removed by rotary evaporation. The residue was purified by flash column chromatography (silica gel, 0˜16% EtOAc in petroleum ether) to provide tert-butyl (S)-4-(7-(3-chlorophenyl)-5-iodo-7H-pyrrolo[2,3-d]pyrimidin-4-yl)-3-methylpiperazine-1-carboxylate (2.6 g, 82%) as a light brown foam. LC/MS ESI (m/z): 554 (M+H)⁺.

### Step 3. methyl(S)-4-(4-(tert-butoxycarbonyl)-2-methylpiperazin-1-yl)-7-(3-chlorophenyl)-7H-pyrrolo[2,3-d]pyrimidine-5-carboxylate

To tert-butyl (S)-4-(7-(3-chlorophenyl)-5-iodo-7H-pyrrolo[2,3-d]pyrimidin-4-yl)-3-methylpiperazine-1-carboxylate (2.6 g, 4.7 mmol) in MeOH (20 mL) were added Pd(dppf)Cl₂ (0.29 g, 0.40 mmol) and Et₃N (2.0 mL, 14 mmol). The reaction was stirred under CO atmosphere at 70° C. for 4 h. The resulting mixture was concentrated and purified by flash column chromatography (silica gel, 0˜23% EtOAc in petroleum ether) to give methyl(S)-4-(4-(tert-butoxycarbonyl)-2-methylpiperazin-1-yl)-7-(3-chlorophenyl)-7H-pyrrolo[2,3-d]pyrimidine-5-carboxylate (2.1 g, 89%) as a light brown foam. LC/MS ESI (m/z): 486 (M+H)⁺.

### Step 4. (S)-4-(4-(tert-butoxycarbonyl)-2-methylpiperazin-1-yl)-7-(3-chlorophenyl)-7H-pyrrolo[2,3-d]pyrimidine-5-carboxylic acid

A solution of methyl(S)-4-(4-(tert-butoxycarbonyl)-2-methylpiperazin-1-yl)-7-(3-chlorophenyl)-7H-pyrrolo[2,3-d]pyrimidine-5-carboxylate (2.1 g, 4.2 mmol) in THF (8 mL)/MeOH (8 mL)/H₂O (4 mL) was treated with NaOH (0.68 g, 17 mmol) at 50° C. for 1 h. The volatile solvents were removed by rotary evaporation. The residue was diluted with H₂O, acidified with 1 N HCl (aq.) under ice-cooling and extracted with EtOAc. The organic layer was dried over Na₂SO₄ and concentrated to provide(S)-4-(4-(tert-butoxycarbonyl)-2-methylpiperazin-1-yl)-7-(3-chlorophenyl)-7H-pyrrolo[2,3-d]pyrimidine-5-carboxylic acid (1.8 g, 90%) as a pale yellow foam. LC/MS ESI (m/z): 472 (M+H)⁺.

### Step 5. tert-butyl (S)-4-(5-acetamido-7-(3-chlorophenyl)-7H-pyrrolo[2,3-d]pyrimidin-4-yl)-3-methylpiperazine-1-carboxylate

To(S)-4-(4-(tert-butoxycarbonyl)-2-methylpiperazin-1-yl)-7-(3-chlorophenyl)-7H-pyrrolo[2,3-d]pyrimidine-5-carboxylic acid (200 mg, 0.42 mmol) in toluene (6 mL) were added DPPA (140 mg, 0.51 mmol) and Et₃N (86 mg, 0.85 mmol). The mixture was degassed and backfilled with N₂ and stirred at rt for 2.5 h. AcOH (1 mL) and Ac₂O (1 mL) were then added, and the mixture was stirred at 60° C. for 1 h. The reaction was quenched with NaHCO₃ (aq.) and extracted with EtOAc. The organic layer was dried over Na₂SO₄, filtered and concentrated. The residue was purified by flash column chromatography (silica gel, 0˜50% EtOAc in petroleum ether) to provide tert-butyl (S)-4-(5-acetamido-7-(3-chlorophenyl)-7H-pyrrolo[2,3-d]pyrimidin-4-yl)-3-methylpiperazine-1-carboxylate (170 mg, 82%) as a light yellow foam. LC/MS ESI (m/z): 485 (M+H)⁺.

### Step 6. tert-butyl (S)-4-(7-(3-chlorophenyl)-5-(N-ethylacetamido)-7H-pyrrolo[2,3-d]pyrimidin-4-yl)-3-methylpiperazine-1-carboxylate

To tert-butyl (S)-4-(5-acetamido-7-(3-chlorophenyl)-7H-pyrrolo[2,3-d]pyrimidin-4-yl)-3-methylpiperazine-1-carboxylate (160 mg, 0.33 mmol) in dry DMF (2 mL) at 0° C. under N₂ atmosphere was added NaH (80 mg, 60% in mineral oil, 2.0 mmol). The mixture was stirred at room temperature for 15 min. Then iodoethane (100 mg, 0.66 mmol) was added. The reaction was stirred at room temperature for 1 h and was then carefully poured into crushed ice. The resultant mixture was extracted with EtOAc, washed with LiCl (5% aq.) and brine, dried over Na₂SO₄, filtered and concentrated. Purification by flash column chromatography (silica gel, 0˜30% EtOAc in petroleum ether) gave tert-butyl (S)-4-(7-(3-chlorophenyl)-5-(N-ethylacetamido)-7H-pyrrolo[2,3-d]pyrimidin-4-yl)-3-methylpiperazine-1-carboxylate (140 mg, 79%) as a light yellow foam. LC/MS ESI (m/z): 513 (M+H)⁺.

### Step 7. tert-butyl (S)-4-(7-(3-chlorophenyl)-5-(diethylamino)-7H-pyrrolo[2,3-d]pyrimidin-4-yl)-3-methylpiperazine-1-carboxylate

To tert-butyl (S)-4-(7-(3-chlorophenyl)-5-(N-ethylacetamido)-7H-pyrrolo[2,3-d]pyrimidin-4-yl)-3-methylpiperazine-1-carboxylate (140 mg, 0.26 mmol) in dry THF (2 mL) at 0° C. under N₂ atmosphere was added BH₃-THF (5.0 mL, 1.0 M in THF) dropwise. The mixture was stirred at 0° C. for 25 min and was then carefully quenched with MeOH. The solvent was removed by rotary evaporation. The residue was partitioned between EtOAc and brine. The organic layer was dried over Na₂SO₄, filtered and concentrated. The residue was purified by prep-TLC (petroleum ether/EtOAc=5:1, v/v) to provide tert-butyl (S)-4-(7-(3-chlorophenyl)-5-(diethylamino)-7H-pyrrolo[2,3-d]pyrimidin-4-yl)-3-methylpiperazine-1-carboxylate (100 mg, 76%) as a pale yellow oil. LC/MS ESI (m/z): 499 (M+H)⁺.

### Step 8. (S)-7-(3-chlorophenyl)-N,N-diethyl-4-(2-methylpiperazin-1-yl)-7H-pyrrolo[2,3-d]pyrimidin-5-amine

A solution of tert-butyl (S)-4-(7-(3-chlorophenyl)-5-(diethylamino)-7H-pyrrolo[2,3-d]pyrimidin-4-yl)-3-methylpiperazine-1-carboxylate (100 mg, 0.20 mmol) in DCM (3 mL) was treated with TFA (1.0 mL) at room temperature for 1 h. The mixture was quenched with NaHCO₃ (aq.) and extracted with DCM. The organic layer was dried over Na₂SO₄, filtered and concentrated to dryness to provide(S)-7-(3-chlorophenyl)-N,N-diethyl-4-(2-methylpiperazin-1-yl)-7H-pyrrolo[2,3-d]pyrimidin-5-amine, which was used directly. LC/MS ESI (m/z): 399 (M+H)⁺.

### Step 9. (S)-1-(4-(7-(3-chlorophenyl)-5-(diethylamino)-7H-pyrrolo[2,3-d]pyrimidin-4-yl)-3-methylpiperazin-1-yl)-2-methylpropan-1-one

To (S)-7-(3-chlorophenyl)-N,N-diethyl-4-(2-methylpiperazin-1-yl)-7H-pyrrolo[2,3-d]pyrimidin-5-amine (80 mg, 0.20 mmol) in dry DCM (2.5 mL) was added Et₃N (61 mg, 0.60 mmol). The mixture was cooled at 0° C. under N₂ atmosphere for 10 min. Isobutyryl chloride (26 mg, 0.24 mmol) was then added. The reaction was stirred at 0° C. for 45 min. The resultant mixture was diluted with DCM and washed with NaHCO₃ (aq.). The organic layer was dried over Na₂SO₄, filtered and concentrated. The residue was purified by prep-HPLC to give (S)-1-(4-(7-(3-chlorophenyl)-5-(diethylamino)-7H-pyrrolo[2,3-d]pyrimidin-4-yl)-3-methylpiperazin-1-yl)-2-methylpropan-1-one (67 mg, 71%) as a white solid. LC/MS ESI (m/z): 469 (M+H)⁺. ¹H NMR (400 MHZ, CDCl₃) δ 8.38 (s, 1H), 7.73 (s, 1H), 7.67 (d, J=8.1 Hz, 1H), 7.43 (t, J=8.1 Hz, 1H), 7.28 (d, J=9.0 Hz, 1H), 6.79 (s, 1H), 5.25 (pair of br. s, 1H, rotamers), 4.50 (m, 2H), 3.86 (m, 1H), 3.64-3.31 (m, 2H), 3.24-2.78 (m, 6H), 1.16 (m, 9H), 1.03 (t, J=7.0 Hz, 6H).

### Example 20. Synthesis of (S)-2-(5-cyclobutyl-4-(4-isobutyryl-2-methylpiperazin-1-yl)-7H-pyrrolo[2,3-d]pyrimidin-7-yl)isonicotinonitrile (Compound 150)

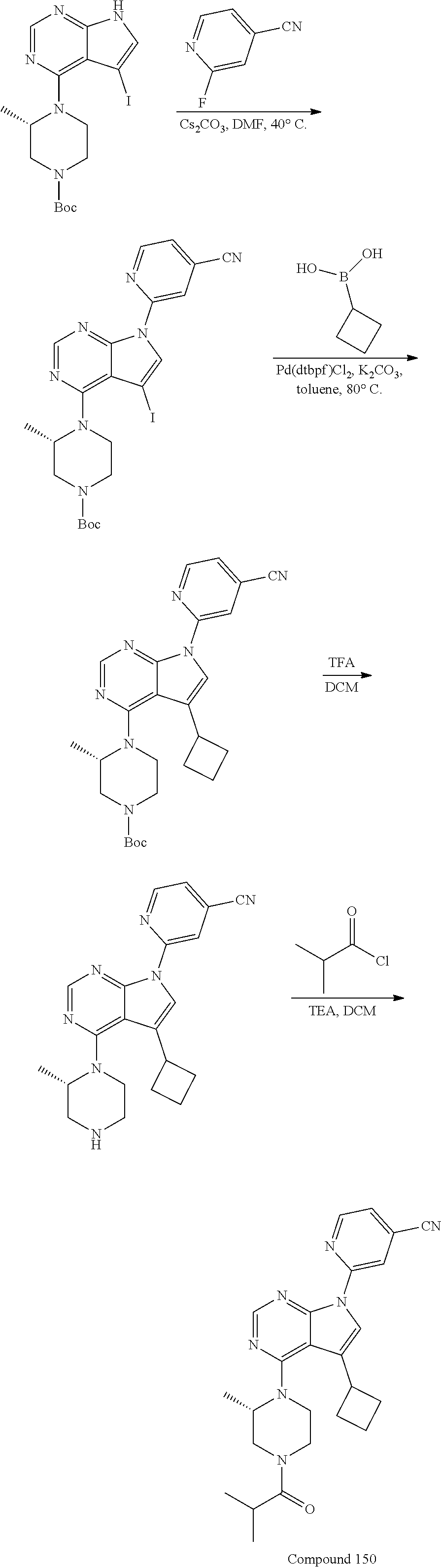

### Step 1. tert-butyl (S)-4-(7-(4-cyanopyridin-2-yl)-5-iodo-7H-pyrrolo[2,3-d]pyrimidin-4-yl)-3-methylpiperazine-1-carboxylate

To a solution of tert-butyl 4-{5-cyclopropyl-7H-pyrrolo[2,3-d]pyrimidin-4-yl}piperidine-1-carboxylate (4.5 g, 10 mmol, prepared following the procedure described for compound 144) and 2-fluoropyridine-4-carbonitrile (1.8 g, 15 mmol) in DMF (60 mL) was added Cs₂CO₃ (16 g, 50 mmol). The reaction was stirred at 40° C. for 18 h. After cooling to room temperature, the resulting mixture was filtered. The filtrate was diluted with ice water and extracted twice with EtOAc. The combined organic layers were washed with brine, dried over Na₂SO₄, filtered and concentrated. The residue was purified by column chromatography on silica gel (0˜40% ethyl acetate in petroleum ether) to give tert-butyl (S)-4-(7-(4-cyanopyridin-2-yl)-5-iodo-7H-pyrrolo[2,3-d]pyrimidin-4-yl)-3-methylpiperazine-1-carboxylate (3.6 g, 66%). LC/MS (ESI) m/z: 546 (M+H)⁺

### Step 2. tert-butyl (S)-4-(7-(4-cyanopyridin-2-yl)-5-cyclobutyl-7H-pyrrolo[2,3-d]pyrimidin-4-yl)-3-methylpiperazine-1-carboxylate

A mixture of tert-butyl (S)-4-(7-(4-cyanopyridin-2-yl)-5-iodo-7H-pyrrolo[2,3-d]pyrimidin-4-yl)-3-methylpiperazine-1-carboxylate (550 mg, 1.0 mmol), cyclobutylboronic acid (0.19 mL, 2.0 mmol), K₂CO₃ (2.7 mg, 19 mmol) and Pd(dtbpf)Cl₂ (66 mg, 0.10 mmol) in toluene (50 mL) was stirred at 80° C. under N₂ atmosphere overnight. After cooling to room temperature, solvent was removed. The residue was purified by flash column chromatography (0˜50% ethyl acetate in petroleum ether) to give tert-butyl (S)-4-(7-(4-cyanopyridin-2-yl)-5-cyclobutyl-7H-pyrrolo[2,3-d]pyrimidin-4-yl)-3-methylpiperazine-1-carboxylate (100 mg, 21%) as a white solid. LC/MS ESI m/z: 474 (M+H)⁺

### Step 3. (S)-2-(5-cyclobutyl-4-(2-methylpiperazin-1-yl)-7H-pyrrolo[2,3-d]pyrimidin-7-yl)isonicotinonitrile

To a solution of tert-butyl (S)-4-(7-(4-cyanopyridin-2-yl)-5-cyclobutyl-7H-pyrrolo[2,3-d]pyrimidin-4-yl)-3-methylpiperazine-1-carboxylate (100 mg, 0.21 mmol) in DCM (2.0 mL) at 0° C. was added TFA (0.31 mL, 4.2 mmol). The reaction was stirred at 0° C. for 3 h. The mixture was basified with NaHCO₃ (aq.) and extracted twice with DCM. The combined organic layers were washed with NaHCO₃ (aq.), dried over Na₂SO₄, filtered and concentrated. The residue was used directly in next step. LC/MS ESI m/z: 374 (M+H)⁺

### Step 4. (S)-2-(5-cyclobutyl-4-(4-isobutyryl-2-methylpiperazin-1-yl)-7H-pyrrolo[2,3-d]pyrimidin-7-yl)isonicotinonitrile

To a solution of (S)-2-(5-cyclobutyl-4-(2-methylpiperazin-1-yl)-7H-pyrrolo[2,3-d]pyrimidin-7-yl)isonicotinonitrile (40 mg, 0.11 mmol) and TEA (0.070 mL, 0.53 mmol) in DCM (2 mL) was added a solution of 2-methylpropanoyl chloride (0.020 mL, 0.21 mmol) in DCM. The mixture was stirred at 0° C. for 1 h. The reaction was quenched with NaHCO₃ (aq.) and extracted twice with DCM. The combined organic phases were washed with brine, dried over anhydrous Na₂SO₄, filtered and concentrated. The residue was purified by Prep-HPLC to give (S)-2-(5-cyclobutyl-4-(4-isobutyryl-2-methylpiperazin-1-yl)-7H-pyrrolo[2,3-d]pyrimidin-7-yl)isonicotinonitrile (20 mg, 42%) as a white solid. LC/MS (ESI) m/z: 444 (M+H)⁺. ¹H NMR (400 MHZ, CDCl₃) δ 9.34 (s, 1H), 8.62 (d, J=4.9 Hz, 1H), 8.54 (s, 1H), 8.13 (d, J=10.4 Hz, 1H), 7.36 (d, J=4.9 Hz, 1H), 4.45-4.15 (m, 2H), 3.95-3.43 (m, 5H), 3.37-3.18 (m, 1H), 2.95-2.78 (m, 1H), 2.45 (m, 2H), 2.29-1.91 (m, 4H), 1.32-1.09 (m, 9H).

### Example 21. Synthesis of (S)-6-(5-(2-fluorophenyl)-4-(4-isobutyryl-2-methylpiperazin-1-yl)-7H-pyrrolo[2,3-d]pyrimidin-7-yl)pyrimidine-4-carbonitrile (Compound 152)

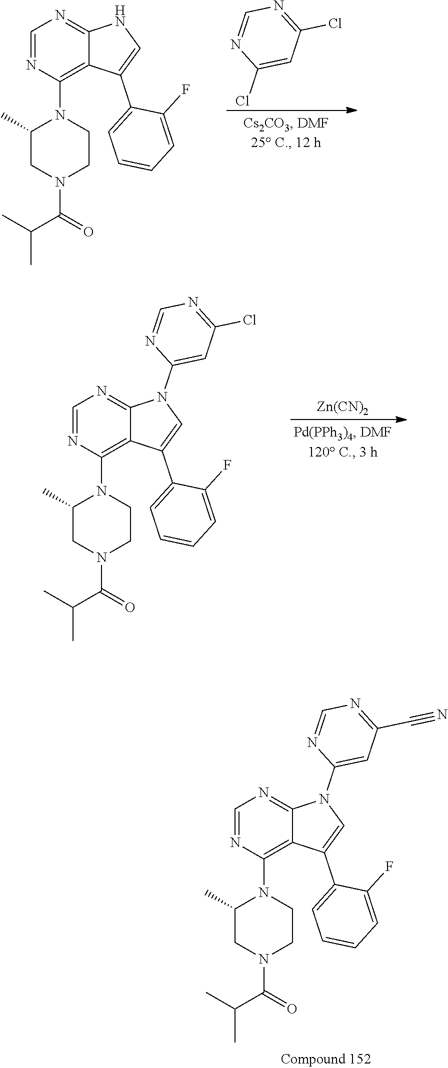 [SMILES: CC(C)C(=O)N1CCN(c2ncnc3[nH]cc(-c4ccccc4F)c23)[C@@H](C)C1 ; Clc1cc(Cl)ncn1 ; CC(C)C(=O)N1CCN(c2ncnc3c2c(-c2ccccc2F)cn3-c2cc(Cl)ncn2)[C@@H](C)C1 ; "Zn(CN)2" ; CC(C)C(=O)N1CCN(c2ncnc3c2c(-c2ccccc2F)cn3-c2cc(C#N)ncn2)[C@@H](C)C1]

### Step 1. (S)-1-(4-(7-(6-chloropyrimidin-4-yl)-5-(2-fluorophenyl)-7H-pyrrolo[2,3-d]pyrimidin-4-yl)-3-methylpiperazin-1-yl)-2-methylpropan-1-one

To a solution of (S)-1-(4-(5-(2-fluorophenyl)-7H-pyrrolo[2,3-d]pyrimidin-4-yl)-3-methylpiperazin-1-yl)-2-methylpropan-1-one (38 mg, 0.10 mmol, prepared following the procedure described for compound 142) in DMF (1 mL) were added 4,6-dichloropyrimidine (30 mg, 0.20 mmol) and Cs₂CO₃ (65 mg, 0.20 mmol) at 25° C. After 3 h, the reaction mixture was poured into H₂O and extracted twice with EtOAc. The combined organic phases were washed with brine, dried over Na₂SO₄, filtered, and concentrated under vacuo. The crude product was purification by silica gel column (SiO₂, petroleum ether:EtOAc=100:0 to 70:30). (S)-1-(4-(7-(6-chloropyrimidin-4-yl)-5-(2-fluorophenyl)-7H-pyrrolo[2,3-d]pyrimidin-4-yl)-3-methylpiperazin-1-yl)-2-methylpropan-1-one (38 mg, 77%) was obtained as a yellow solid. LC/MS ESI (m/z): 494 (M+H)⁺.

### Step 2. (S)-6-(5-(2-fluorophenyl)-4-(4-isobutyryl-2-methylpiperazin-1-yl)-7H-pyrrolo[2,3-d]pyrimidin-7-yl)pyrimidine-4-carbonitrile

To a solution of (S)-1-(4-(7-(6-chloropyrimidin-4-yl)-5-(2-fluorophenyl)-7H-pyrrolo[2,3-d]pyrimidin-4-yl)-3-methylpiperazin-1-yl)-2-methylpropan-1-one (38 mg, 0.077 mmol) in DMF (1 mL) were added Zn(CN)₂ (70 mg, 0.60 mmol) and Pd(PPh₃)⁺ (120 mg, 0.10 mmol) at 25° C. The mixture was degassed 3 times with N₂, and then heated at 120° C. for 2 h. After cooling to 25° C., the mixture was poured into H₂O and extracted twice with EtOAc. The combined organic phases were washed with brine, dried over Na₂SO₄, filtered, and concentrated in vacuo. The residue was purified by Prep-HPLC (0.1% formic acid as additive) to afford (S)-6-(5-(2-fluorophenyl)-4-(4-isobutyryl-2-methylpiperazin-1-yl)-7H-pyrrolo[2,3-d]pyrimidin-7-yl)pyrimidine-4-carbonitrile (16 mg, 43%) as a light yellow solid. LC/MS ESI (m/z): 485 (M+H)⁺. ¹H NMR (400 MHZ, CDCl₃) δ 9.64 (s, 1H), 9.11 (s, 1H), 8.59 (d, J=4.7 Hz, 1H), 8.30 (d, J=5.8 Hz, 1H), 7.48-7.38 (m, 2H), 7.26-7.17 (m, 2H), 4.53-3.99 (m, 2H), 3.75-3.56 (m, 1H), 3.55-3.34 (m, 1H), 3.30-2.91 (m, 2H), 2.83-2.63 (m, 1H), 2.61-2.42 (m, 1H), 1.19-0.85 (m, 9H).

### Example 22. Synthesis of 2-(5-cyclopropyl-4-((2S,5R)-4-(3,3-dimethylbutanoyl)-2,5-dimethylpiperazin-1-yl)-7H-pyrrolo[2,3-d]pyrimidin-7-yl)isonicotinonitrile (Compound 153)

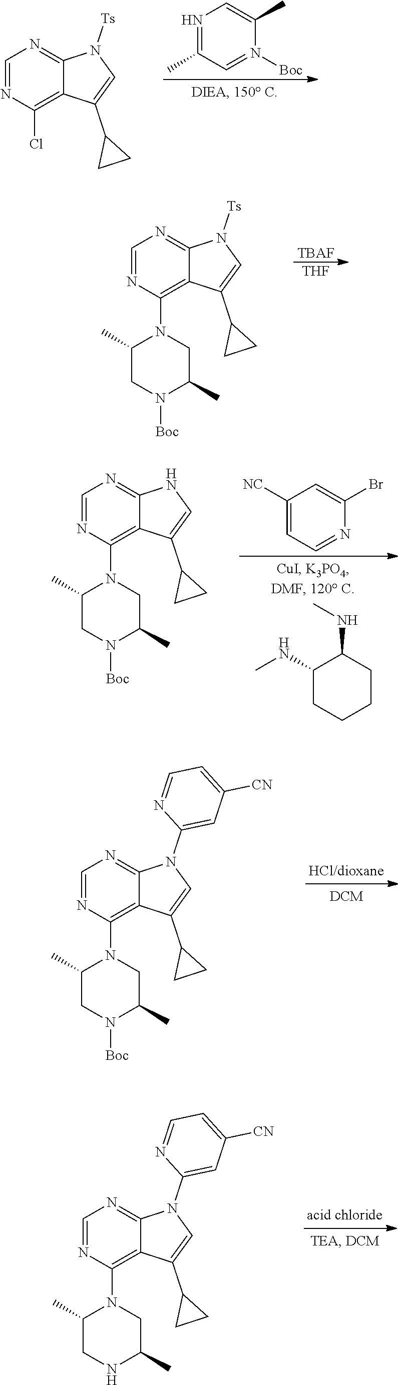

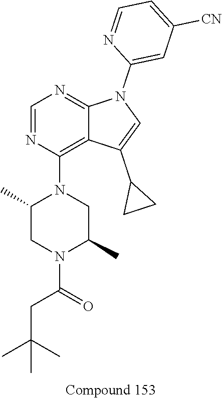 [SMILES: C[C@@H]1CN(c2ncnc3c2c(C2CC2)cn3-c2cc(C#N)ccn2)[C@@H](C)CN1C(=O)CC(C)(C)C]

### Step 1. tert-butyl (2R,5S)-4-(5-cyclopropyl-7-tosyl-7H-pyrrolo[2,3-d]pyrimidin-4-yl)-2,5-dimethylpiperazine-1-carboxylate

To a solution of 4-chloro-5-cyclopropyl-7-tosyl-7H-pyrrolo[2,3-d]pyrimidine (500 mg, 1.4 mmol, prepared following the procedure described for compound 129) in DIEA (1.0 mL) was added tert-butyl (2R,5S)-2,5-dimethylpiperazine-1-carboxylate (1.2 g, 5.8 mmol). The resulting mixture was stirred at 150° C. for 3 h. After cooling to room temperature, solvent was removed. The residue was purified by flash column chromatography (silica gel, 0˜40%, ethyl acetate in petroleum ether) to afford tert-butyl (2R,5S)-4-(5-cyclopropyl-7-tosyl-7H-pyrrolo[2,3-d]pyrimidin-4-yl)-2,5-dimethylpiperazine-1-carboxylate (600 mg, 80%) as a light yellow solid. LC/MS ESI m/z: 526 (M+H)⁺.

### Step 2. tert-butyl (2R,5S)-4-(5-cyclopropyl-7H-pyrrolo[2,3-d]pyrimidin-4-yl)-2,5-dimethylpiperazine-1-carboxylate

To a solution of tert-butyl (2R,5S)-4-(5-cyclopropyl-7-tosyl-7H-pyrrolo[2,3-d]pyrimidin-4-yl)-2,5-dimethylpiperazine-1-carboxylate (600 mg, 1.1 mmol) in THF (3 mL) was added TBAF (5.0 mL, 1.0M in THF). The resulting mixture was stirred at room temperature overnight. The reaction was quenched with water and extracted twice with EtOAc. The combined organic layers were washed with brine, dried over Na₂SO₄, filtered and concentrated. The residue was purified by flash column chromatography (silica gel, 0˜60%, ethyl acetate in petroleum ether) to afford tert-butyl (2R,5S)-4-(5-cyclopropyl-7H-pyrrolo[2,3-d]pyrimidin-4-yl)-2,5-dimethylpiperazine-1-carboxylate (400 mg, 92%) as a white solid. LC/MS ESI m/z: 372 (M+H)⁺.

### Step 3. tert-butyl (2R,5S)-4-(7-(4-cyanopyridin-2-yl)-5-cyclopropyl-7H-pyrrolo[2,3-d]pyrimidin-4-yl)-2,5-dimethylpiperazine-1-carboxylate

To a solution of tert-butyl (2R,5S)-4-(5-cyclopropyl-7H-pyrrolo[2,3-d]pyrimidin-4-yl)-2,5-dimethylpiperazine-1-carboxylate (400 mg, 1.1 mmol) in DMF (5 mL) were added 2-bromoisonicotinonitrile (390 mg, 2.2 mmol), trans-N,N′-dimethylcyclohexane-1,2-diamine (46 mg, 0.32 mmol), CuI (120 mg, 0.65 mmol) and K₃PO₄ (690 mg, 3.2 mmol). The reaction was stirred at 120° C. overnight. After cooled down to room temperature, the mixture was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to afford tert-butyl (2R,5S)-4-(7-(4-cyanopyridin-2-yl)-5-cyclopropyl-7H-pyrrolo[2,3-d]pyrimidin-4-yl)-2,5-dimethylpiperazine-1-carboxylate (350 mg, 68%) as a yellow solid. LC/MS ESI m/z: 474 (M+H)⁺.

### Step 4. 2-(5-cyclopropyl-4-((2S,5R)-2,5-dimethylpiperazin-1-yl)-7H-pyrrolo[2,3-d]pyrimidin-7-yl)isonicotinonitrile

To a solution of tert-butyl (2R,5S)-4-(7-(4-cyanopyridin-2-yl)-5-cyclopropyl-7H-pyrrolo[2,3-d]pyrimidin-4-yl)-2,5-dimethylpiperazine-1-carboxylate (350 mg, 0.74 mmol) in DCM (5 mL) at 0° C. was added HCl (1.0 mL, 4.0M in dioxane). The resulting mixture was stirred at room temperature for 3 h. The reaction was then quenched with NaHCO₃ (aq.) and extracted twice with DCM. The combined organic layers were washed with brine, dried over Na₂SO₄, filtered and concentrated. The residue was used in the next step directly. LC/MS ESI m/z: 374 (M+H)⁺.

### Step 5. 2-(5-cyclopropyl-4-((2S,5R)-4-(3,3-dimethylbutanoyl)-2,5-dimethylpiperazin-1-yl)-7H-pyrrolo[2,3-d]pyrimidin-7-yl)isonicotinonitrile

To a solution of 2-(5-cyclopropyl-4-((2S,5R)-2,5-dimethylpiperazin-1-yl)-7H-pyrrolo[2,3-d]pyrimidin-7-yl)isonicotinonitrile (50 mg, 0.13 mmol) in DCM (5 mL) were added TEA (40 mg, 0.40 mmol) and 3,3-dimethylbutanoyl chloride (36 mg, 0.27 mmol). The resulting mixture was stirred at room temperature for 1 h. The reaction was quenched with water, extracted twice with DCM. The combined organic layers were washed with NaHCO₃ (aq.), dried over Na₂SO₄, filtered and concentrated. The residue was purified by flash column chromatography (silica gel, 0˜50%, ethyl acetate in petroleum ether) to give crude product which was further purified by Prep-HPLC to afford 2-(5-cyclopropyl-4-((2S,5R)-4-(3,3-dimethylbutanoyl)-2,5-dimethylpiperazin-1-yl)-7H-pyrrolo[2,3-d]pyrimidin-7-yl)isonicotinonitrile (35 mg, 60%) as a white solid. LC/MS ESI m/z: 472 (M+H)⁺. ¹H NMR (400 MHZ, CDCl₃) δ 9.31 (s, 1H), 8.59 (d, J=4.9 Hz, 1H), 8.51 (d, J=6.5 Hz, 1H), 7.82 (s, 1H), 7.37-7.33 (m, 1H), 4.96 (s, 1.5H, rotamers), 4.43 (d, J=13.3 Hz, 0.5H, rotamers), 4.30 (s, 0.5H, rotamers), 3.89-3.62 (m, 3H), 3.38 (dd, J=13.2, 2.3 Hz, 0.5H, rotamers), 2.52 (d, J=13.7 Hz, 0.5H, rotamers), 2.39 (d, J=14.0 Hz, 0.5H, rotamers), 2.19 (t, J=14.1 Hz, 1H), 2.05-1.95 (m, 1H), 1.32 (d, J=6.6 Hz, 1.5H, rotamers), 1.24 (d, J=6.4 Hz, 1.5H, rotamers), 1.13-1.03 (m, 14H), 0.98-0.91 (m, 1H), 0.74-0.68 (m, 1H).

The following compound was prepared by the procedures analogous to the synthesis of compound 153 using the corresponding acid chloride.

| Cmpd | | |
|---|---|---|
| No. | Chemical Name | LCMS and ¹H NMR |
| 154 | 2-(5-cyclopropyl-4-((2S,5R)-2,5- | LC/MS ESI (m/z): 458 (M + H)⁺. ¹H NMR (400 MHz, |
| | dimethyl-4-(3- | CDCl₃) δ 9.32 (s, 1H), 8.59 (d, J = 4.9 Hz, 1H), 8.50 (d, J = |
| | methylbutanoyl)piperazin-1-yl)- | 5.1 Hz, 1H), 7.82 (s, 1H), 7.35 (d, J = 4.1 Hz, 1H), 5.00- |
| | 7H-pyrrolo[2,3-d]pyrimidin-7- | 4.91 (m, 1.5H, rotamers), 4.40 (d, J = 13.2 Hz, 0.5H, |
| | yl)isonicotinonitrile | rotamers), 4.25 (s, 0.5H, rotamers), 3.88-3.71 (m, 2.5H, |
| | | rotamers), 3.58 (d, J = 13.0 Hz, 0.5H, rotamers), 3.40 (dd, |
| | | J = 13.2, 3.0 Hz, 0.5H, rotamers), 2.45-2.38 (m, 0.5H, |
| | | rotamers), 2.26-2.16 (m, 2.5H, rotamers), 2.05-1.96 (m, |
| | | 1H), 1.32 (d, J = 6.7 Hz, 1.5H, rotamers), 1.23 (d, J = 6.5 |
| | | Hz, 1.5H, rotamers), 1.13-0.97 (m, 12H), 0.74-0.68 (m, |
| | | 1H). |

### Example 23. Synthesis of 2-(5-(2-fluorophenyl)-4-(7-isobutyryl-4,7-diazaspiro[2.5]octan-4-yl)-7H-pyrrolo[2,3-d]pyrimidin-7-yl)isonicotinonitrile (Compound 155)

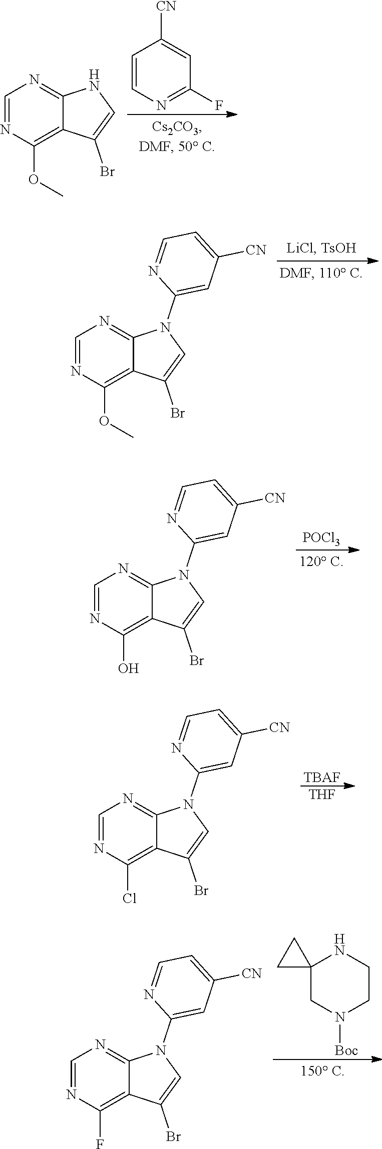

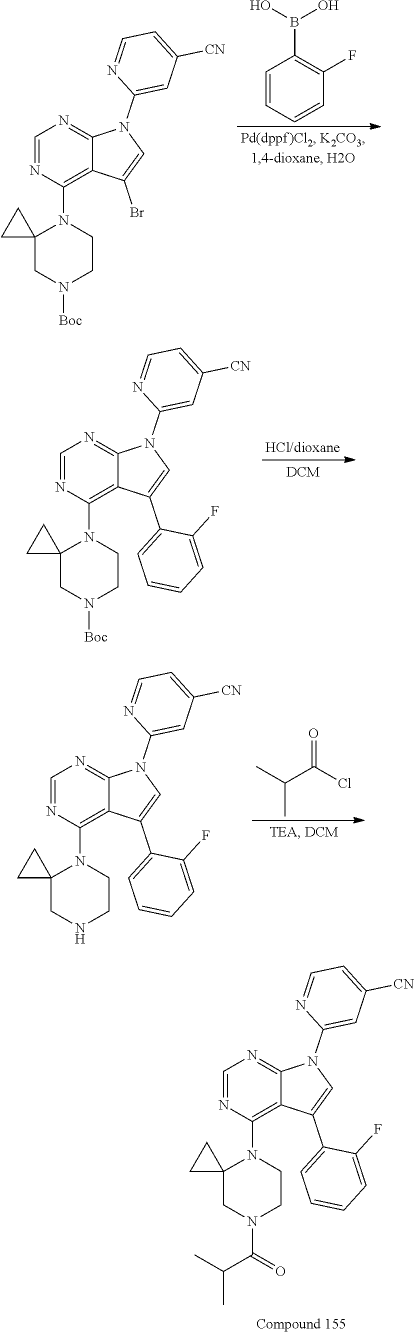

### Step 1. 2-(5-bromo-4-methoxy-7H-pyrrolo[2,3-d]pyrimidin-7-yl)isonicotinonitrile

To a solution of 5-bromo-4-methoxy-7H-pyrrolo[2,3-d]pyrimidine (5.0 g, 22 mmol, prepared from MeONa and 5-bromo-4-chloro-7H-pyrrolo[2,3-d]pyrimidine) in DMF (25 mL) were added 2-fluoroisonicotinonitrile (5.4 g, 44 mmol) and Cs₂CO₃ (36 g, 110 mmol). The resulting mixture was stirred at 50° C. for 5 h. The reaction was quenched with ice water and extracted twice with EtOAc. The combined organic layers were washed with brine, dried over Na₂SO₄, filtered and concentrated. The residue was purified by flash column chromatography (silica gel, 0-50%, ethyl acetate in petroleum ether) to afford 2-(5-bromo-4-methoxy-7H-pyrrolo[2,3-d]pyrimidin-7-yl)isonicotinonitrile (6.0 g, 83%) as a yellow solid. LC/MS ESI m/z: 330, 332 (M+H)⁺.

### Step 2. 2-(5-bromo-4-hydroxy-7H-pyrrolo[2,3-d]pyrimidin-7-yl)isonicotinonitrile

To a solution of 2-(5-bromo-4-methoxy-7H-pyrrolo[2,3-d]pyrimidin-7-yl)isonicotinonitrile (6.0 g, 18 mmol) in DMF (20 mL) were added 4-methylbenzenesulfonic acid (31 g, 180 mmol) and LiOH (7.7 g, 180 mmol). The resulting mixture was stirred at 110° C. for 2 h. The reaction was quenched with water and extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to afford 2-(5-bromo-4-hydroxy-7H-pyrrolo[2,3-d]pyrimidin-7-yl)isonicotinonitrile (5.5 g, 96%) as a yellow solid. LC/MS ESI m/z: 316, 318 (M+H)⁺.

### Step 3. 2-(5-bromo-4-chloro-7H-pyrrolo[2,3-d]pyrimidin-7-yl)isonicotinonitrile

To a solution of POCl₃ (20 mL) was added 2-(5-bromo-4-hydroxy-7H-pyrrolo[2,3-d]pyrimidin-7-yl)isonicotinonitrile (5.5 g, 17 mmol). The resulting mixture was stirred at 120° C. under N₂ atmosphere overnight. The solvent was removed, the residue was poured into water and extracted twice with EtOAc. The combined organic layers were dried over Na₂SO₄, filtered and concentrated, the residue was purified by flash chromatography (silica gel, 0˜30% EtOAc in petroleum ether) to afford 2-(5-bromo-4-chloro-7H-pyrrolo[2,3-d]pyrimidin-7-yl)isonicotinonitrile (5.0 g, 86%) as a light yellow solid. LC/MS ESI (m/z): 334,336 (M+H)⁺.

### Step 4. 2-(5-bromo-4-fluoro-7H-pyrrolo[2,3-d]pyrimidin-7-yl)isonicotinonitrile

To a solution of 2-(5-bromo-4-chloro-7H-pyrrolo[2,3-d]pyrimidin-7-yl)isonicotinonitrile (500 mg, 1.5 mmol) in THF (5 mL) were added TBAF (5.0 ml, 1.0M in THF) at 0° C. The resulting mixture was stirred at room temperature overnight. The reaction was quenched with ice water and extracted twice with EtOAc. The combined organic layers were dried over Na₂SO₄, filtered and concentrated. The residue was purified by flash column chromatography (silica gel, 0˜50%, ethyl acetate in petroleum ether) to afford 2-(5-bromo-4-fluoro-7H-pyrrolo[2,3-d]pyrimidin-7-yl)isonicotinonitrile (100 mg, 25%) as a light yellow solid. LC/MS ESI (m/z): 318, 320 (M+H)⁺.

### Step 5. tert-butyl 4-(5-bromo-7-(4-cyanopyridin-2-yl)-7H-pyrrolo[2,3-d]pyrimidin-4-yl)-4,7-diazaspiro[2.5]octane-7-carboxylate

A mixture of 2-(5-bromo-4-fluoro-7H-pyrrolo[2,3-d]pyrimidin-7-yl)isonicotinonitrile (100 mg, 0.31 mmol) and tert-butyl 4,7-diazaspiro[2.5]octane-7-carboxylate (270 mg, 1.3 mmol) was stirred at 150° C. under N₂ atmosphere for 3 h. After cooling to room temperature, the reaction was purified by flash column chromatography (silica gel, 0˜50%, ethyl acetate in petroleum ether) to afford tert-butyl 4-(5-bromo-7-(4-cyanopyridin-2-yl)-7H-pyrrolo[2,3-d]pyrimidin-4-yl)-4,7-diazaspiro[2.5]octane-7-carboxylate (80 mg, 50%) as a yellow solid. LC/MS ESI (m/z): 510, 512 (M+H)⁺.

### Step 6. tert-butyl 4-(7-(4-cyanopyridin-2-yl)-5-(2-fluorophenyl)-7H-pyrrolo[2,3-d]pyrimidin-4-yl)-4,7-diazaspiro[2.5]octane-7-carboxylate

To a solution of tert-butyl 4-(5-bromo-7-(4-cyanopyridin-2-yl)-7H-pyrrolo[2,3-d]pyrimidin-4-yl)-4,7-diazaspiro[2.5]octane-7-carboxylate (80 mg, 0.16 mmol) in 1.4-dioxane (5 mL) and H₂O (1 ml) were added (2-fluorophenyl) boronic acid (44 mg, 0.31 mmol), K₂CO₃ (65 mg, 0.47 mmol) and Pd(dppf)Cl₂ (4.0 mg, 0.020 mmol). The resulting mixture was heated to 90° C. overnight. After cooling to room temperature, solvent was removed and the residue was purified by flash column chromatography (silica gel, 0˜50%, ethyl acetate in petroleum ether) to give crude product (100 mg) which was further purified by Prep-HPLC to afford tert-butyl 4-(7-(4-cyanopyridin-2-yl)-5-(2-fluorophenyl)-7H-pyrrolo[2,3-d]pyrimidin-4-yl)-4,7-diazaspiro[2.5]octane-7-carboxylate (70 mg, 80%) as a white solid. LC/MS ESI m/z: 526 (M+H)⁺. ¹HNMR (400 MHZ, CDCl₃) δ 9.33 (s, 1H), 8.60-8.54 (m, 2H), 8.23 (s, 1H), 7.35-7.27 (m, 3H), 7.15-7.09 (m, 2H), 3.55-3.37 (m, 2H), 3.32-2.94 (m, 2H), 2.93-2.82 (m, 2H), 1.34 (s, 9H), 0.89-0.76 (m, 4H).

### Step 7. 2-(5-(2-fluorophenyl)-4-(4,7-diazaspiro[2.5]octan-4-yl)-7H-pyrrolo[2,3-d]pyrimidin-7-yl)isonicotinonitrile

To a solution of tert-butyl 4-(7-(4-cyanopyridin-2-yl)-5-(2-fluorophenyl)-7H-pyrrolo[2,3-d]pyrimidin-4-yl)-4,7-diazaspiro[2.5]octane-7-carboxylate (70 mg, 0.13 mmol) in DCM (4 mL) at 0° C. was added HCl (0.50 mL, 4.0M in dioxane). The resulting mixture was stirred at 0° C. for 1 h. The reaction was quenched with NaHCO₃ (aq.) and extracted twice with DCM. The combined organic layers were washed with brine, dried over Na₂SO₄, filtered and concentrated. The residue was used in the next step directly. LC/MS ESI m/z: 372 (M+H)⁺.

### Step 8. 2-(5-(2-fluorophenyl)-4-(7-isobutyryl-4,7-diazaspiro[2.5]octan-4-yl)-7H-pyrrolo[2,3-d]pyrimidin-7-yl)isonicotinonitrile

To a solution of 2-(5-(2-fluorophenyl)-4-(4,7-diazaspiro[2.5]octan-4-yl)-7H-pyrrolo[2,3-d]pyrimidin-7-yl)isonicotinonitrile (0.13 mmol) in DCM (3 mL) at 0° C. were added TEA (49 mg, 0.49 mmol) and isobutyryl chloride (26 mg, 0.24 mmol) dropwise. The resulting mixture was stirred at room temperature for 20 min. The reaction was quenched with NaHCO₃ (aq.) and extracted twice with DCM. The combined organic layers were washed with brine, dried over Na₂SO₄, filtered and concentrated. The residue was purified by Prep-HPLC to afford 2-(5-(2-fluorophenyl)-4-(7-isobutyryl-4,7-diazaspiro[2.5]octan-4-yl)-7H-pyrrolo[2,3-d]pyrimidin-7-yl)isonicotinonitrile (20 mg, 35% over 2 steps) as a white solid. LC/MS ESI m/z: 496 (M+H)⁺. ¹HNMR (400 MHZ, CDCl₃) δ 9.33 (s, 1H), 8.61-8.55 (m, 2H), 8.26-8.21 (m, 1H), 7.35-7.27 (m, 3H), 7.15-7.07 (m, 2H), 3.77-3.07 (m, 4H), 2.99-2.83 (m, 2H), 2.62-2.55 (m, 1H), 1.02-0.97 (m, 6H), 0.88-0.72 (m, 4H).

The following compounds were prepared by the procedures analogous to the synthesis of compound 155 using the corresponding amine (tert-butyl 3,3-dimethylpiperazine-1-carboxylate)

| Cmpd | | |
|---|---|---|
| No. | Chemical Name | LCMS and ¹H NMR |
| 149 | 2-(5-(2-fluorophenyl)-4-(4- | LC/MS ESI m/z: 498 (M + H)⁺ ¹H NMR(400 MHz, CD₃OD) |
| | isobutyryl-2,2- | δ 9.31 (s, 1H), 8.70 (d, J = 5.1 Hz, 1H), 8.67 (s, 1H), 8.31 |
| | dimethylpiperazin-1-yl)-7H- | (d, J = 5.0 Hz, 1H), 7.62-7.55 (m, 2H), 7.51-7.45 (m, |
| | pyrrolo[2,3-d]pyrimidin-7- | 1H), 7.37-7.27 (m, 2H), 3.34 (d, J = 4.1 Hz, 2H), 3.08- |
| | yl)isonicotinonitrile | 2.97 (m, 2H), 2.94-2.69 (m, 1H), 2.67-2.59 (m, 2H), |
| | | 1.54 (pair of s, 6H, rotamers), 1.03 (pair of d, J = 6.7 Hz, |
| | | 6H, rotamers). |

### Example 24. Synthesis of (S)-2-(5-(2-fluorophenyl)-4-(4-isobutyryl-2-methylpiperazin-1-yl)-7H-pyrrolo[2,3-d]pyrimidin-7-yl)pyrimidine-4-carbonitrile (Compound 156)

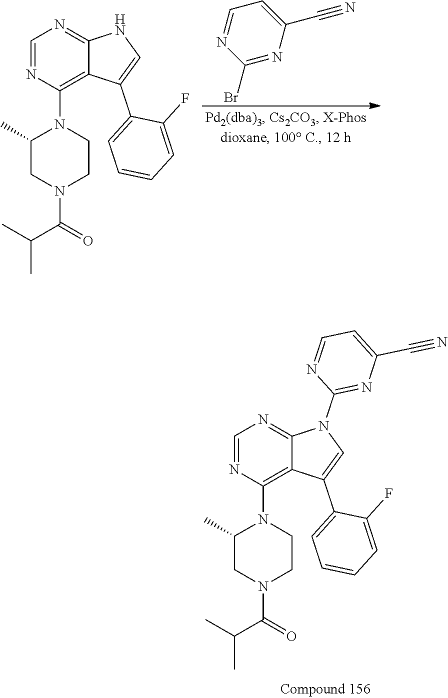 [SMILES: CC(C)C(=O)N1CCN(c2ncnc3[nH]cc(-c4ccccc4F)c23)[C@@H](C)C1 ; N#Cc1ccnc(Br)n1 ; CC(C)C(=O)N1CCN(c2ncnc3c2c(-c2ccccc2F)cn3-c2nccc(C#N)n2)[C@@H](C)C1]

### Step 1. (S)-2-(5-(2-fluorophenyl)-4-(4-isobutyryl-2-methylpiperazin-1-yl)-7H-pyrrolo[2,3-d]pyrimidin-7-yl)pyrimidine-4-carbonitrile

To a solution of (S)-1-(4-(5-(2-fluorophenyl)-7H-pyrrolo[2,3-d]pyrimidin-4-yl)-3-methylpiperazin-1-yl)-2-methylpropan-1-one (38 mg, 0.10 mmol, prepared following the procedure described for compound 142) in dioxane (1 mL) were added 2-bromopyrimidine-4-carbonitrile (28 mg, 0.15 mmol), Pd₂(dba)₃ (18 mg, 0.020 mmol), X-Phos (23 mg, 0.040 mmol) and Cs₂CO₃ (65 mg, 0.20 mmol) at 25° C. The mixture was degassed 3 times with N₂ and then stirred at 100° C. for 2 h. After cooling to room temperature, the reaction mixture was poured into H₂O and extracted twice with EtOAc. The combined organic phases were washed with brine, dried over Na₂SO₄, filtered and concentrated under vacuo. The crude product was purification by Prep-HPLC (0.1% formic acid as additive) to afford (S)-2-(5-(2-fluorophenyl)-4-(4-isobutyryl-2-methylpiperazin-1-yl)-7H-pyrrolo[2,3-d]pyrimidin-7-yl)pyrimidine-4-carbonitrile (31 mg, 66%) was obtained as a solid. LC/MS ESI (m/z): 485 (M+H)⁺. ¹H NMR (400 MHZ, CDCl₃) δ 9.17 (d, J=4.8 Hz, 1H), 8.72 (s, 1H), 8.17 (d, J=4.3 Hz, 1H), 7.54 (d, J=4.7 Hz, 1H), 7.52-7.44 (m, 1H), 7.44-7.35 (m, 1H), 7.26-7.16 (m, 2H), 4.50-3.87 (m, 2H), 3.81-3.54 (m, 1H), 3.53-3.31 (m, 1H), 3.28-2.87 (m, 2H), 2.86-2.63 (m, 1H), 2.63-2.37 (m, 1H), 1.26-0.73 (m, 9H).

### Example 25. Synthesis of (S)-4-(5-(2-fluorophenyl)-4-(4-isobutyryl-2-methylpiperazin-1-yl)-7H-pyrrolo[2,3-d]pyrimidin-7-yl)-1-methyl-1H-pyrrole-2-carbonitrile (Compound 157)

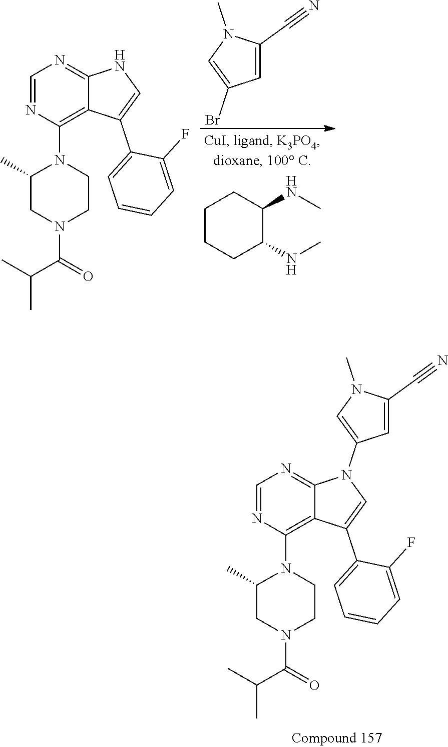

### Step 1. (S)-4-(5-(2-fluorophenyl)-4-(4-isobutyryl-2-methylpiperazin-1-yl)-7H-pyrrolo[2,3-d]pyrimidin-7-yl)-1-methyl-1H-pyrrole-2-carbonitrile

To a solution of (S)-1-(4-(5-(2-fluorophenyl)-7H-pyrrolo[2,3-d]pyrimidin-4-yl)-3-methylpiperazin-1-yl)-2-methylpropan-1-one (95 mg, 0.25 mmol, prepared following the procedure of compound 142) and 4-bromo-1-methyl-1H-pyrrole-2-carbonitrile (92 mg, 0.50 mmol, prepared in three steps from commercially available methyl 4-bromo-1-methyl-1H-pyrrole-2-carboxylate by hydrolysis of ester mediated by sodium hydroxide, followed by conversion to a primary amide using ammonium chloride and dehydration to the nitrile using trifluoroacetic anhydride) in DMF (2 mL) were added CuI (47 mg, 0.25 mmol), trans-N,N′-dimethylcyclohexane-1,2-diamine (35 mg, 0.25 mmol) and K₃PO₄ (530 mg, 2.5 mmol). The resulting mixture was stirred at 120° C. under N₂ atmosphere overnight. After cooling to room temperature, the reaction was partitioned between EtOAc and water. The aqueous layer was extracted with EtOAc twice, the combined organic layers were washed with brine, dried over Na₂SO₄, filtered and concentrated. The residue was purified by flash column chromatography (silica gel, 0˜50%, ethyl acetate in petroleum ether) to give crude product which was further purified by prep-HPLC to afford (S)-4-(5-(2-fluorophenyl)-4-(4-isobutyryl-2-methylpiperazin-1-yl)-7H-pyrrolo[2,3-d]pyrimidin-7-yl)-1-methyl-1H-pyrrole-2-carbonitrile (23 mg, 19% yield) as a white solid. LC/MS ESI (m/z): 486 (M+H)⁺. ¹H NMR (400 MHZ, CDCl₃) δ 8.51 (d, J=2.5 Hz, 1H), 7.57 (s, 1H), 7.51-7.40 (m, 1H), 7.39-7.29 (m, 2H), 7.26-7.15 (m, 2H), 7.03 (s, 1H), 4.46-3.96 (m, 2H), 3.88 (s, 3H), 3.66 (t, J=11.6 Hz, 1H), 3.45 (d, J=13.1 Hz, 1H), 3.28-2.94 (m, 2H), 2.83-2.65 (m, 1H), 2.64-2.49 (m, 1H), 1.18-0.83 (m, 9H).

### Example 26. Synthesis of tert-butyl (S)-4-(1-(4-cyanopyridin-2-yl)-3-(2-fluorophenyl)-1H-pyrrolo[3,2-c]pyridin-4-yl)-3-methylpiperazine-1-carboxylate (Compound 161)

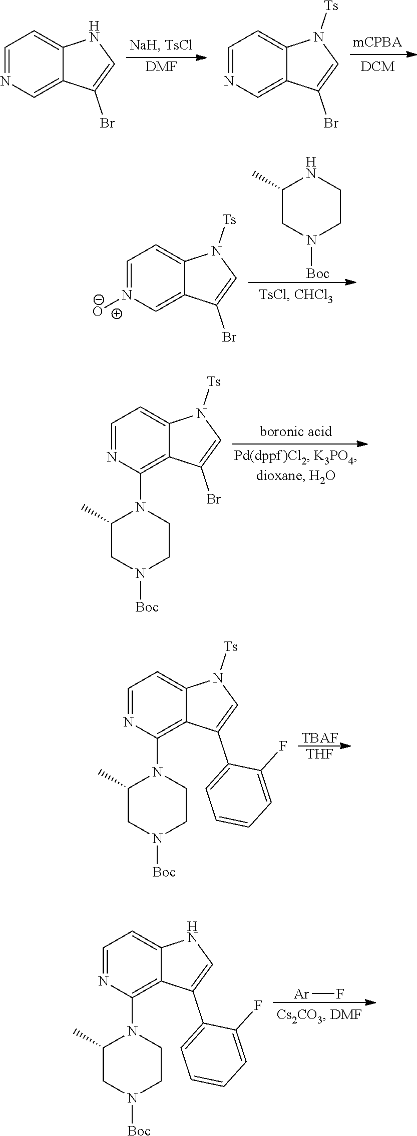

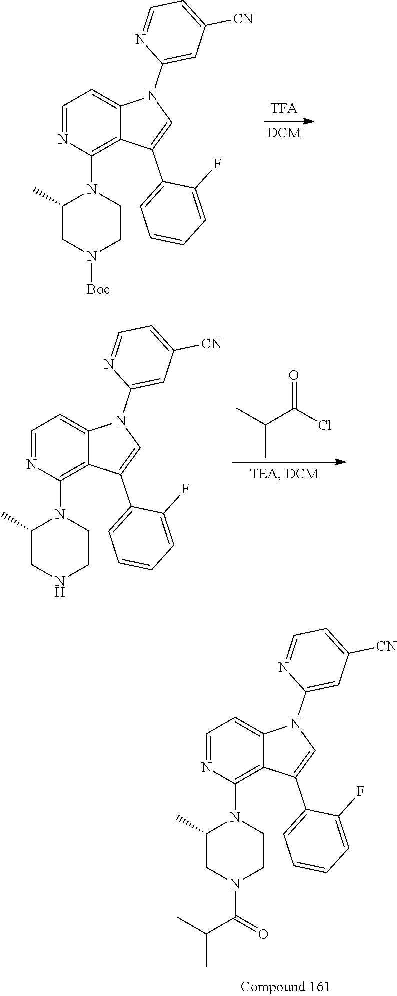

### Step 1. 3-bromo-1-tosyl-1H-pyrrolo[3,2-c]pyridine

To a suspension of NaH (1.2 g, 30 mmol) in DMF (50 mL) at 0° C. were added 3-bromo-1H-pyrrolo[3,2-c]pyridine (5.0 g, 25 mmol). After stirring at 0° C. for 15 min, TsCl (5.3 g, 28 mmol) was added in portions. The resulting mixture was stirred at room temperature overnight under N₂ atmosphere. The mixture was quenched with ice water and was filtered. The filter cake was washed with water and dried under vacuum to provide 3-bromo-1-tosyl-1H-pyrrolo[3,2-c]pyridine (7.5 g, 84%) as a white solid. LC/MS ESI (m/z): 351 (M+H)⁺.

### Step 2. 3-bromo-1-tosyl-1H-pyrrolo[3,2-c]pyridine 5-oxide

To a solution of 3-bromo-1 3-bromo-1-tosyl-1H-pyrrolo[3,2-c]pyridine (7.0 g, 20 mmol) in DCM (70 mL) at 0° C. was added 3-chloroperoxybenzoic acid (5.2 g, 30 mmol) in portions. The resulting mixture was stirred at room temperature overnight. The reaction was quenched with NaHCO₃ (aq.) and extracted twice with DCM. The combined organic layers were washed with brine, dried over Na₂SO₄, filtered and concentrated. The residue was purified by flash column chromatography (silica gel, 0˜20%, MeOH in DCM) to provide 3-bromo-1-tosyl-1H-pyrrolo[3,2-c]pyridine 5-oxide (4.0 g, 54%) as a light yellow solid. LC/MS ESI (m/z): 367, 369 (M+H)⁺.

### Step 3. tert-butyl (S)-4-(3-bromo-1-tosyl-1H-pyrrolo[3,2-c]pyridin-4-yl)-3-methylpiperazine-1-carboxylate

To a solution of 3-bromo-1-tosyl-1H-pyrrolo[3,2-c]pyridine 5-oxide (300 mg, 0.80 mmol) in CHCl₃ (5 mL) at 0° C. were added tert-butyl (S)-3-methylpiperazine-1-carboxylate (390 mg, 1.9 mmol). After stirring at 0° C. for 10 min, TsCl (180 mg, 0.96 mmol) was added. The resulting mixture was stirred at room temperature overnight under N₂ atmosphere. The reaction was quenched with NaHCO₃ (aq.) and extracted twice with DCM. The combined organic layers were washed with brine, dried over Na₂SO₄, filtered and concentrated. The residue was purified by flash column chromatography (silica gel, 0˜40%, ethyl acetate in petroleum ether) to provide ter-butyl (S)-4-(3-bromo-1-tosyl-1H-pyrrolo[3,2-c]pyridin-4-yl)-3-methylpiperazine-1-carboxylate (47 mg, 10%) as a light yellow solid. LC/MS ESI (m/z): 549, 551 (M+H)⁺

### Step 4. tert-butyl (S)-4-(3-(2-fluorophenyl)-1-tosyl-1H-pyrrolo[3,2-c]pyridin-4-yl)-3-methylpiperazine-1-carboxylate

To a solution of tert-butyl (S)-4-(3-bromo-1-tosyl-1H-pyrrolo[3,2-c]pyridin-4-yl)-3-methylpiperazine-1-carboxylate (62 mg, 0.10 mmol) in dioxane (5 mL) and H₂O (1 mL) were added (2-fluorophenyl) boronic acid (16 mg, 0.11 mmol), K₃PO₄ (43 mg, 0.20 mmol) and Pd(dppf)Cl₂ (8.0 mg, 0.010 mmol). The resulting mixture was heated to 90° C. overnight. After cooling to room temperature, solvent was removed and the residue was purified by flash column chromatography (silica gel, 0˜40%, ethyl acetate in petroleum ether) to afford tert-butyl (S)-4-(3-(2-fluorophenyl)-1-tosyl-1H-pyrrolo[3,2-c]pyridin-4-yl)-3-methylpiperazine-1-carboxylate (51 mg, 80%) as a white solid. LC/MS ESI m/z: 565 (M+H)⁺.

### Step 5. tert-butyl (S)-4-(3-(2-fluorophenyl)-1H-pyrrolo[3,2-c]pyridin-4-yl)-3-methylpiperazine-1-carboxylate

To a solution of tert-butyl (S)-4-(3-(2-fluorophenyl)-1-tosyl-1H-pyrrolo[3,2-c]pyridin-4-yl)-3-methylpiperazine-1-carboxylate (50 mg, 0.090 mmol) in THF (1 mL) was added TBAF (1.0 mL, 1.0M in THF). The resulting mixture was stirred at room temperature overnight under N₂ atmosphere. The reaction was quenched with water and extracted twice with EtOAc. The combined organic layers were washed with brine, dried over Na₂SO₄, filtered and concentrated. The residue was purified by flash column chromatography (silica gel, 0˜60%, ethyl acetate in petroleum ether) to provide tert-butyl (S)-4-(3-(2-fluorophenyl)-1H-pyrrolo[3,2-c]pyridin-4-yl)-3-methylpiperazine-1-carboxylate (35 mg, 96%) as a yellow solid. LC/MS ESI m/z: 411 (M+H)⁺.

### Step 6. tert-butyl (S)-4-(1-(4-cyanopyridin-2-yl)-3-(2-fluorophenyl)-1H-pyrrolo[3,2-c]pyridin-4-yl)-3-methylpiperazine-1-carboxylate

To a solution of tert-butyl (S)-4-(3-(2-fluorophenyl)-1H-pyrrolo[3,2-c]pyridin-4-yl)-3-methylpiperazine-1-carboxylate (35 mg, 0.080 mmol) in DMF (5 mL) were added 2-fluoroisonicotinonitrile (20 mg, 0.16 mmol), and Cs₂CO₃ (1.2 g, 8.3 mmol). The resulting mixture was heated to 50° C. overnight. After cooling to room temperature, solvent was removed and the residue was purified by flash column chromatography (silica gel, ethyl acetate in petroleum ether) to give crude product which was further purified by prep-HPLC to afford tert-butyl (S)-4-(1-(4-cyanopyridin-2-yl)-3-(2-fluorophenyl)-1H-pyrrolo[3,2-c]pyridin-4-yl)-3-methylpiperazine-1-carboxylate (51 mg, 80%) as a white solid. LC/MS ESI m/z: 513 (M+H)⁺. ¹H NMR (400 MHZ, CDCl₃) δ 8.77 (d, J=4.9 Hz, 1H), 8.23 (d, J=5.9 Hz, 1H), 7.88 (d, J=5.8 Hz, 1H), 7.71 (d, J=13.5 Hz, 2H), 7.53-7.44 (m, 2H), 7.42-7.35 (m, 1H), 7.24-7.17 (m, 2H), 3.47 (s, 1H), 3.12-2.86 (m, 6H), 1.42 (s, 9H), 0.91 (d, J=4.7 Hz, 3H).

### Step 7. (S)-2-(3-(2-fluorophenyl)-4-(2-methylpiperazin-1-yl)-1H-pyrrolo[3,2-c]pyridin-1-yl)isonicotinonitrile

To a solution of tert-butyl (S)-4-(1-(4-cyanopyridin-2-yl)-3-(2-fluorophenyl)-1H-pyrrolo[3,2-c]pyridin-4-yl)-3-methylpiperazine-1-carboxylate (130 mg, 0.24 mmol) in DCM (2 mL) at 0° C. was added TFA (1 mL). The resulting mixture was stirred at 0° C. for 1.5 h. The reaction was quenched with NaHCO₃ (aq.). The aqueous layer was extracted with DCM twice. The combined organic layers were washed with brine, dried over Na₂SO₄, filtered and concentrated. The residue was used in the next step directly. LC/MS ESI m/z: 413 (M+H)⁺.

### Step 8. (S)-2-(3-(2-fluorophenyl)-4-(4-isobutyryl-2-methylpiperazin-1-yl)-1H-pyrrolo[3,2-c]pyridin-1-yl)isonicotinonitrile

To a solution of (S)-2-(3-(2-fluorophenyl)-4-(2-methylpiperazin-1-yl)-1H-pyrrolo[3,2-c]pyridin-1-yl)isonicotinonitrile (98 mg, 0.24 mmol) in DCM (3 mL) at 0° C. were added dropwise TEA (0.060 mL, 0.48 mmol) and isobutyryl chloride (0.040 mL, 0.36 mmol). The resulting mixture was stirred at room temperature for 1.5 h. The reaction was quenched with ice water and extracted twice with DCM. The combined organic layers were washed with NaHCO₃ (aq.), dried over Na₂SO₄, filtered and concentrated. The residue was purified by flash column chromatography (silica gel, ethyl acetate in petroleum ether) to give crude product which was further purified by prep-HPLC to afford (S)-2-(3-(2-fluorophenyl)-4-(4-isobutyryl-2-methylpiperazin-1-yl)-1H-pyrrolo[3,2-c]pyridin-1-yl)isonicotinonitrile (43 mg, 36%) as a white solid. LC/MS ESI m/z: 483 (M+H)⁺. ¹HNMR (400 MHZ, CDCl₃) δ 8.77 (d, J=4.9 Hz, 1H), 8.25-8.20 (m, 1H), 7.90 (s, 1H), 7.72 (d, J=11.9 Hz, 2H), 7.48 (dd, J=21.6, 5.9 Hz, 2H), 7.42-7.36 (m, 1H), 7.20 (dd, J=17.9, 8.3 Hz, 2H), 3.57-2.71 (m, 8H), 1.13-1.06 (m, 6H), 1.03 (d, J=6.1 Hz, 1.5H, rotamers), 0.83 (d, J=6.3 Hz, 1.5H, rotamers).

### Example 27. Synthesis of (S)-2-(5-(azetidin-1-yl)-4-(4-(2-hydroxy-2-methylpropanoyl)-2-methylpiperazin-1-yl)-7H-pyrrolo[2,3-d]pyrimidin-7-yl)isonicotinonitrile (Compound 162)

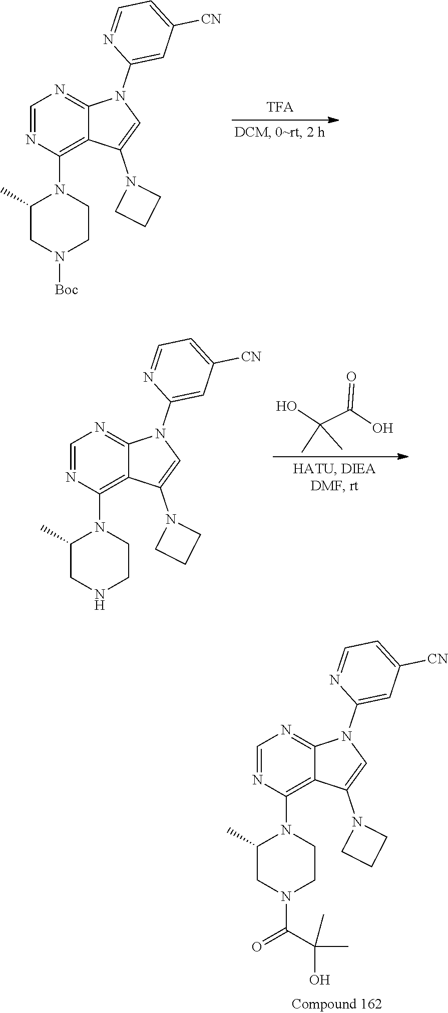

### Step 1. (S)-2-(5-(azetidin-1-yl)-4-(2-methylpiperazin-1-yl)-7H-pyrrolo[2,3-d]pyrimidin-7-yl)isonicotinonitrile

To a solution of tert-butyl (S)-4-(5-(azetidin-1-yl)-7-(4-cyanopyridin-2-yl)-7H-pyrrolo[2,3-d]pyrimidin-4-yl)-3-methylpiperazine-1-carboxylate (100 mg, 0.21 mmol, prepared following the procedure of compound 144, substituting azetidin-2-one for pyrrolidin-2-one in the third step) in DCM (2 mL) at 0° C. was added TFA (0.47 mL, 6.3 mmol). The reaction was stirred at 0° C. for 3 h. The reaction was quenched with saturated of NaHCO₃ solution and extracted twice with EtOAc. The combined organic phases were washed with brine, dried over anhydrous Na₂SO₄, filtered and concentrated. The residue was used directly in next step without further purification. LC/MS ESI m/z: 375 (M+H)⁺.

### Step 2. (S)-2-(5-(azetidin-1-yl)-4-(4-(2-hydroxy-2-methylpropanoyl)-2-methylpiperazin-1-yl)-7H-pyrrolo[2,3-d]pyrimidin-7-yl)isonicotinonitrile

A mixture of 2-hydroxy-2-methylpropanoic acid (21 mg, 0.20 mmol), HATU (85 mg, 0.22 mmol) and DIEA (0.15 mL, 0.93 mmol) in DMF (3 mL) was stirred at room temperature for 0.5 h. (S)-2-(5-(azetidin-1-yl)-4-(2-methylpiperazin-1-yl)-7H-pyrrolo[2,3-d]pyrimidin-7-yl)isonicotinonitrile (70 mg, 0.18 mmol) was then added. After 2.5 h, the reaction mixture was quenched with NaHCO₃ (aq.) and extracted twice with EtOAc. The combined organic phases were washed with brine, dried over anhydrous Na₂SO₄, filtered and concentrated. The residue was purified by prep-HPLC to obtain(S)-2-(5-(azetidin-1-yl)-4-(4-(2-hydroxy-2-methylpropanoyl)-2-methylpiperazin-1-yl)-7H-pyrrolo[2,3-d]pyrimidin-7-yl)isonicotinonitrile (30 mg, 35%) as a yellow solid. LC/MS ESI m/z: 461 (M+H)⁺. ¹H NMR (400 MHZ, CDCl₃) δ 9.32 (s, 1H), 8.57 (d, J=5.0 Hz, 1H), 8.45 (s, 1H), 7.49 (s, 1H), 7.30 (d, J=4.9 Hz, 1H), 5.06 (s, 1H), 4.34 (d, J=19.5 Hz, 3H), 4.18 (d, J=13.5 Hz, 1H), 3.83 (q, J=6.9 Hz, 2H), 3.66 (q, J=6.9 Hz, 2H), 3.60-3.38 (m, 2H), 3.22 (s, 1H), 2.29 (p, J=6.9 Hz, 2H), 1.55 (d, J=8.3 Hz, 6H), 1.16 (d, J=4.8 Hz, 3H).

### Example 28. Synthesis of (S)-2-(5-(ethyl(propyl)amino)-4-(4-isobutyryl-2-methylpiperazin-1-yl)-7H-pyrrolo[2,3-d]pyrimidin-7-yl)isonicotinonitrile (Compound 163)

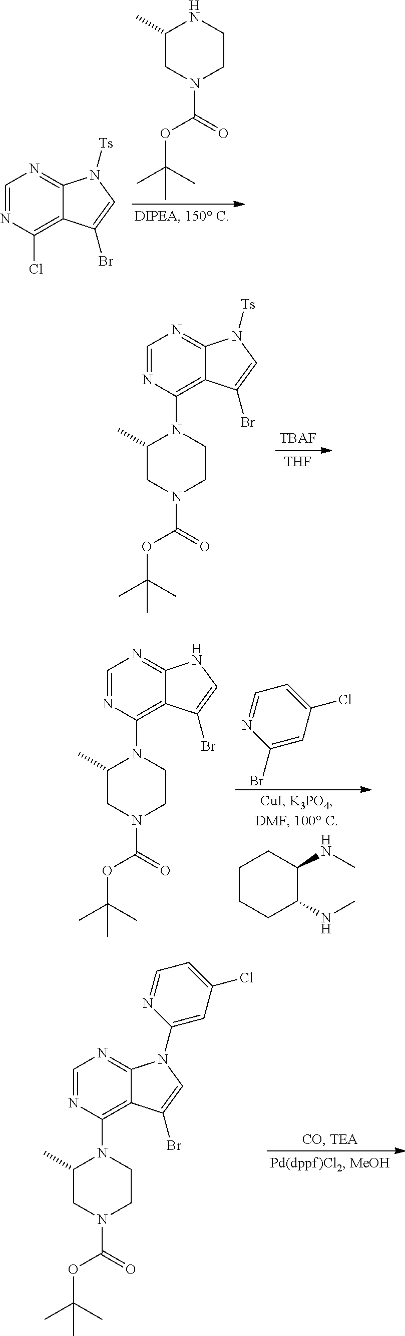

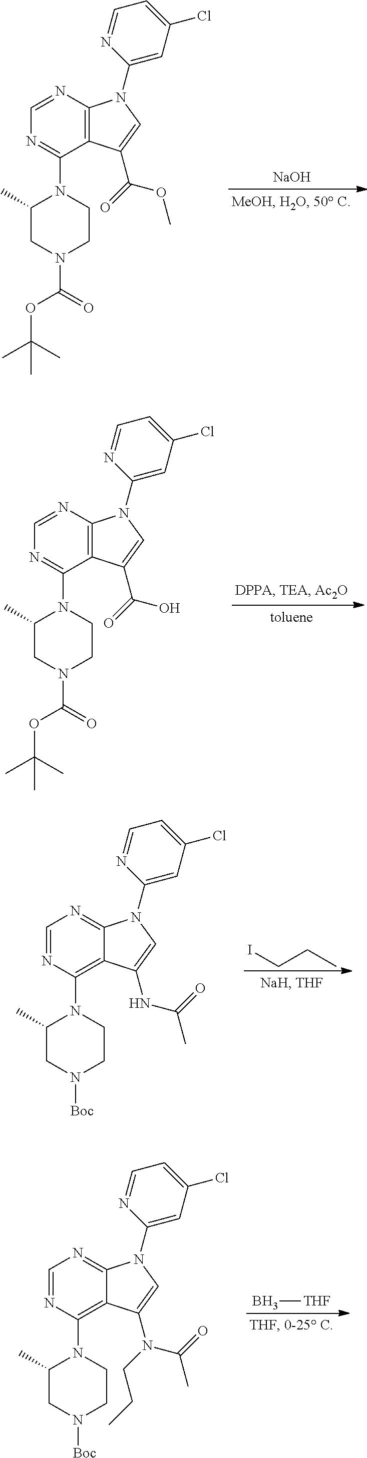

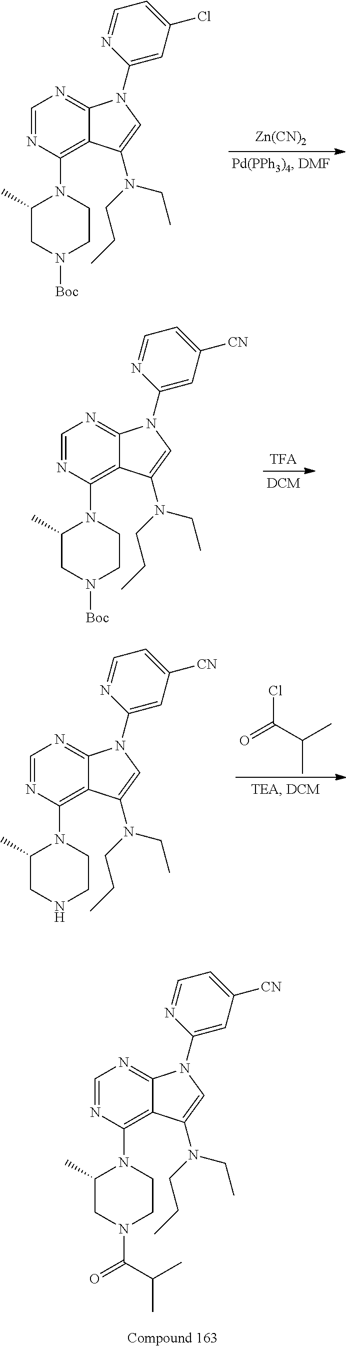

### Step 1. tert-butyl (S)-4-(5-bromo-7-tosyl-7H-pyrrolo[2,3-d]pyrimidin-4-yl)-3-methylpiperazine-1-carboxylate

To a solution of 5-bromo-4-chloro-7-tosyl-7H-pyrrolo[2,3-d]pyrimidine (17 g, 43 mmol, prepared following a similar procedure described for compound 128) in DIPEA (30 mL) was added tert-butyl (S)-3-methylpiperazine-1-carboxylate (22 g, 110 mmol). The resulting mixture was stirred at 150° C. for 2.5 h. After cooling to room temperature, solvent was removed and the residue was purified by flash column chromatography (silica gel, 0˜20%, ethyl acetate in petroleum ether) to afford tert-butyl (S)-4-(5-bromo-7-tosyl-7H-pyrrolo[2,3-d]pyrimidin-4-yl)-3-methylpiperazine-1-carboxylate (20 g, 86%) as a white solid. LC/MS ESI m/z: 550, 552 (M+H)⁺.

### Step 2. tert-butyl (S)-4-(5-bromo-7H-pyrrolo[2,3-d]pyrimidin-4-yl)-3-methylpiperazine-1-carboxylate

To a solution of tert-butyl (S)-4-(5-bromo-7-tosyl-7H-pyrrolo[2,3-d]pyrimidin-4-yl)-3-methylpiperazine-1-carboxylate (15 g, 27 mmol) in THF (30 mL) was added TBAF (64 mL, 1.0M in THF). The resulting mixture was stirred at room temperature for 4 h. The reaction was quenched with water and extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to afford tert-butyl (S)-4-(5-bromo-7H-pyrrolo[2,3-d]pyrimidin-4-yl)-3-methylpiperazine-1-carboxylate (9.3 g, 86%) as a white solid. LC/MS ESI m/z: 396, 398 (M+H)⁺.

### Step 3. tert-butyl (S)-4-(5-bromo-7-(4-chloropyridin-2-yl)-7H-pyrrolo[2,3-d]pyrimidin-4-yl)-3-methylpiperazine-1-carboxylate

To a solution of tert-butyl (S)-4-(5-bromo-7H-pyrrolo[2,3-d]pyrimidin-4-yl)-3-methylpiperazine-1-carboxylate (9.3 g, 23 mmol) in DMF (100 mL) were added 2-bromo-4-chloropyridine (9.0 g, 47 mmol), CuI (4.5 g, 23 mmol), trans-N,N′-dimethylcyclohexane-1,2-diamine (5.0 g, 35 mmol) and K₃PO₄ (10 g, 47 mmol). The resulting mixture was heated to 100° C. for 3 h. After cooling to room temperature, the reaction was quenched with water and extracted twice with EtOAc. The combined organic layers were washed with brine, dried over Na₂SO₄, filtered and concentrated. The residue was purified by flash column chromatography (silica gel, 0˜15%, ethyl acetate in petroleum ether) to afford tert-butyl (S)-4-(5-bromo-7-(4-chloropyridin-2-yl)-7H-pyrrolo[2,3-d]pyrimidin-4-yl)-3-methylpiperazine-1-carboxylate (9.8 g, 82%) as a white solid. LC/MS ESI m/z: 507, 509 (M+H)⁺.

### Step 4. methyl(S)-4-(4-(tert-butoxycarbonyl)-2-methylpiperazin-1-yl)-7-(4-chloropyridin-2-yl)-7H-pyrrolo[2,3-d]pyrimidine-5-carboxylate

To a solution of tert-butyl (S)-4-(5-bromo-7-(4-chloropyridin-2-yl)-7H-pyrrolo[2,3-d]pyrimidin-4-yl)-3-methylpiperazine-1-carboxylate (9.8 g, 19 mmol) in MeOH (80 mL) were added Pd(dppf)Cl₂ (1.4 g, 1.9 mmol) and TEA (5.3 mL, 39 mmol). The resulting mixture was stirred at 50° C. overnight under CO atmosphere. After cooling to room temperature, solvent was removed and the residue was purified by flash column chromatography (silica gel, 0˜15%, ethyl acetate in petroleum ether) to afford methyl(S)-4-(4-(tert-butoxycarbonyl)-2-methylpiperazin-1-yl)-7-(4-chloropyridin-2-yl)-7H-pyrrolo[2,3-d]pyrimidine-5-carboxylate (8.7 g, 92%) as a white solid. LC/MS ESI m/z: 487 (M+H)⁺.

### Step 5. (S)-4-(4-(tert-butoxycarbonyl)-2-methylpiperazin-1-yl)-7-(4-chloropyridin-2-yl)-7H-pyrrolo[2,3-d]pyrimidine-5-carboxylic acid

To a solution of methyl(S)-4-(4-(tert-butoxycarbonyl)-2-methylpiperazin-1-yl)-7-(4-chloropyridin-2-yl)-7H-pyrrolo[2,3-d]pyrimidine-5-carboxylate (8.7 g, 18 mmol) in MeOH (90 mL) and H₂O (10 mL) was added NaOH (1.4 g, 36 mmol). The resulting mixture was stirred at 80° C. overnight. After cooling to room temperature, the reaction was concentrated. The residue was treated with H₂O and acidified to pH 5 with HCl (aq.). The precipitate was collected by filtration to afford (S)-4-(4-(tert-butoxycarbonyl)-2-methylpiperazin-1-yl)-7-(4-chloropyridin-2-yl)-7H-pyrrolo[2,3-d]pyrimidine-5-carboxylic acid (5.1 g, 60%) as a white solid. LC/MS ESI m/z: 473 (M+H)⁺.

### Step 6. tert-butyl (S)-4-(5-acetamido-7-(4-chloropyridin-2-yl)-7H-pyrrolo[2,3-d]pyrimidin-4-yl)-3-methylpiperazine-1-carboxylate

To a solution of (S)-4-(4-(tert-butoxycarbonyl)-2-methylpiperazin-1-yl)-7-(4-chloropyridin-2-yl)-7H-pyrrolo[2,3-d]pyrimidine-5-carboxylic acid (400 mg, 0.84 mmol) in toluene (8 mL) were added TEA (0.40 mL, 2.5 mmol) and DPPA (460 mg, 1.7 mmol). The resulting mixture was stirred at room temperature for 2 h. Ac₂O (4 mL) was added, and the resulting mixture was heated to 60° C. for 2 h. After cooling to room temperature, the reaction was quenched with NaHCO₃ (aq.) and extracted twice with EtOAc. The combined organic layers were washed with brine, dried over Na₂SO₄, filtered and concentrated. The residue was purified by flash column chromatography (silica gel, ethyl acetate in petroleum ether) to afford tert-butyl (S)-4-(5-acetamido-7-(4-chloropyridin-2-yl)-7H-pyrrolo[2,3-d]pyrimidin-4-yl)-3-methylpiperazine-1-carboxylate (350 mg, 86%) as a light yellow solid. LC/MS ESI m/z: 486 (M+H)⁺.

### Step 7. tert-butyl (S)-4-(7-(4-chloropyridin-2-yl)-5-(N-propylacetamido)-7H-pyrrolo[2,3-d]pyrimidin-4-yl)-3-methylpiperazine-1-carboxylate

To a suspension of NaH (40 mg, 1.0 mmol) in THF (8 mL) at 0° C. were added tert-butyl (S)-4-(5-acetamido-7-(4-chloropyridin-2-yl)-7H-pyrrolo[2,3-d]pyrimidin-4-yl)-3-methylpiperazine-1-carboxylate (250 mg, 0.50 mmol). After stirring at 0° C. for 15 min, 1-iodopropane (13 mg, 0.75 mmol) was added. The resulting mixture was stirred at room temperature overnight under N₂ atmosphere. The reaction was quenched with ice water and extracted twice with EtOAc. The combined organic layers were washed with brine, dried over Na₂SO₄, filtered and concentrated. The residue was purified by flash column chromatography (silica gel, ethyl acetate in petroleum ether) to afford tert-butyl (S)-4-(7-(4-chloropyridin-2-yl)-5-(N-propylacetamido)-7H-pyrrolo[2,3-d]pyrimidin-4-yl)-3-methylpiperazine-1-carboxylate (190 mg, 70%) as a light yellow solid. LC/MS ESI m/z: 528 (M+H)⁺.

### Step 8. tert-butyl (S)-4-(7-(4-chloropyridin-2-yl)-5-(ethyl(propyl)amino)-7H-pyrrolo[2,3-d]pyrimidin-4-yl)-3-methylpiperazine-1-carboxylate

To a solution of tert-butyl (S)-4-(7-(4-chloropyridin-2-yl)-5-(N-propylacetamido)-7H-pyrrolo[2,3-d]pyrimidin-4-yl)-3-methylpiperazine-1-carboxylate (190 mg, 0.36 mmol) in THF (1 mL) at 0° C. were added BH₃-THF (3.0 mL, 1.0 M in THF) dropwise. After stirring at room temperature for 1 h, the reaction was quenched with ice water and extracted twice with EtOAc. The combined organic layers were washed with brine, dried over Na₂SO₄, filtered and concentrated. The residue was purified by flash column chromatography (silica gel, 0˜35%, ethyl acetate in petroleum ether) to afford tert-butyl (S)-4-(7-(4-chloropyridin-2-yl)-5-(ethyl(propyl)amino)-7H-pyrrolo[2,3-d]pyrimidin-4-yl)-3-methylpiperazine-1-carboxylate (110 mg, 57%) as a colorless oil. LC/MS ESI m/z: 514 (M+H)⁺.

### Step 9. tert-butyl (S)-4-(7-(4-cyanopyridin-2-yl)-5-(ethyl(propyl)amino)-7H-pyrrolo[2,3-d]pyrimidin-4-yl)-3-methylpiperazine-1-carboxylate

To a solution of tert-butyl (S)-4-(7-(4-chloropyridin-2-yl)-5-(ethyl(propyl)amino)-7H-pyrrolo[2,3-d]pyrimidin-4-yl)-3-methylpiperazine-1-carboxylate (110 mg, 0.20 mmol) in DMF (5 mL) were added Zn(CN)₂ (140 mg, 1.2 mmol) and Pd(PPh₃)⁺ (120 mg, 0.10 mmol). The resulting reaction mixture was stirred at 120° C. under N₂ atmosphere overnight. After cooling to room temperature, the reaction mixture was concentrated under reduced pressure. The residue was purified by flash column chromatography (silica gel, ethyl acetate in petroleum ether) and to afford tert-butyl (S)-4-(7-(4-cyanopyridin-2-yl)-5-(ethyl(propyl)amino)-7H-pyrrolo[2,3-d]pyrimidin-4-yl)-3-methylpiperazine-1-carboxylate (100 mg, 97%) as a yellow solid. LC/MS ESI m/z: 505 (M+H)⁺.

### Step 10. (S)-2-(5-(ethyl(propyl)amino)-4-(2-methylpiperazin-1-yl)-7H-pyrrolo[2,3-d]pyrimidin-7-yl)isonicotinonitrile

To a solution of tert-butyl (S)-4-(7-(4-cyanopyridin-2-yl)-5-(ethyl(propyl)amino)-7H-pyrrolo[2,3-d]pyrimidin-4-yl)-3-methylpiperazine-1-carboxylate (100 mg, 0.20 mmol) in DCM (1 mL) at 0° C. was added TFA (1 mL). The resulting mixture was stirred at the same temperature for 1.5 h. The reaction was quenched with NaHCO₃ (aq.). The aqueous layer was extracted twice with DCM. The combined organic layers were washed with brine, dried over Na₂SO₄, filtered and concentrated. The residue was used in the next step directly. LC/MS ESI m/z: 405 (M+H)⁺.

### Step 11. (S)-2-(5-(ethyl(propyl)amino)-4-(4-isobutyryl-2-methylpiperazin-1-yl)-7H-pyrrolo[2,3-d]pyrimidin-7-yl)isonicotinonitrile

To a solution of (S)-2-(5-(ethyl(propyl)amino)-4-(2-methylpiperazin-1-yl)-7H-pyrrolo[2,3-d]pyrimidin-7-yl)isonicotinonitrile (80 mg, 0.20 mmol) in DCM (3 mL) at 0° C. were added dropwise TEA (0.060 mL, 0.46 mmol) and isobutyryl chloride (0.030 mL, 0.30 mmol). The resulting mixture was stirred at room temperature for 30 minutes. The reaction was quenched with ice water and extracted twice with DCM. The combined organic layers were washed with NaHCO₃ (aq.) and brine, dried over Na₂SO₄, filtered and concentrated. The residue was purified by flash column chromatography (silica gel, ethyl acetate in petroleum ether) to give crude product which was further purified by prep-HPLC to afford (S)-2-(5-(ethyl(propyl)amino)-4-(4-isobutyryl-2-methylpiperazin-1-yl)-7H-pyrrolo[2,3-d]pyrimidin-7-yl)isonicotinonitrile (19 mg, 41%) as a yellow solid. LC/MS ESI m/z: 475 (M+H)⁺. ¹H NMR (400 MHZ, CDCl₃) δ 9.38 (s, 1H), 8.58 (d, J=4.9 Hz, 1H), 8.44 (s, 1H), 7.62 (s, 1H), 7.30 (d, J=4.7 Hz, 1H), 5.23 (d, J=44.4 Hz, 1H), 4.69-4.30 (m, 2H), 3.97 (d, J=10.8 Hz, 0.5H, rotamers), 3.76 (d, J=13.7 Hz, 0.5H, rotamers), 3.59 (d, J=11.8 Hz, 0.5H, rotamers), 3.42-3.01 (m, 6H), 2.92-2.78 (m, 1.5H), 1.57-1.50 (m, 2H), 1.25-1.13 (m, 8H), 1.06 (d, J=4.9 Hz, 1H), 0.98-0.88 (m, 6H).

The following compounds were prepared by procedures analogous to the synthesis of compound 163 from the corresponding acid chloride or acid anhydride.

| Cmpd | | |
|---|---|---|
| No. | Chemical Name | LCMS and ¹H NMR |
| 134 | (S)-2-(5-(ethyl(propyl)amino)-4- | LC/MS ESI (m/z): 489 (M + H)⁺. ¹H NMR (400 MHz, |
| | (2-methyl-4-pivaloylpiperazin-1- | CDCl₃) δ 9.38 (s, 1H), 8.58 (d, J = 4.9 Hz, 1H), 8.44 (S, |
| | yl)-7H-pyrrolo[2,3-d]pyrimidin- | 1H), 7.62 (s, 1H), 7.30 (d, J = 4.9 Hz, 1H), 5.25 (s, 1H), |
| | 7-yl)isonicotinonitrile | 4.36 (t, J = 13.0 Hz, 3H), 3.44 (t, J = 11.8 Hz, 1H), 3.31- |
| | | 3.02 (m, 6H), 1.56-1.51 (m, 2H), 1.34 (s, 9H), 1.12 (d, J = |
| | | 5.8 Hz, 3H), 0.97-0.88 (m, 6H). |
| 166 | (S)-2-(4-(4-(3,3- | LC/MS ESI (m/z): 503 (M + H)⁺. ¹H NMR (400 MHz, |
| | dimethylbutanoyl)-2- | CDCl₃) δ 9.38 (s, 1H), 8.58 (d, J = 5.0 Hz, 1H), 8.44 (d, J = |
| | methylpiperazin-1-yl)-5- | 4.6 Hz, 1H), 7.62 (d, J = 6.3 Hz, 1H), 7.30 (d, J = 5.0 Hz, |
| | (ethyl(propyl)amino)-7H- | 1H), 5.35-5.27 (m, 0.5H, rotamers), 5.19-5.05 (m, 0.5H, |
| | pyrrolo [2,3-d]pyrimidin-7- | rotamers), 4.72-4.61 (m, 0.5H, rotamers), 4.52 (d, J = 13.1 |
| | yl)isonicotinonitrile | Hz, 1H), 4.37-4.24 (m, 0.5H, rotamers), 4.03-3.95 (m, |
| | | 0.5H, rotamers), 3.79-3.71 (m, 0.5H, rotamers), 3.62- |
| | | 3.52 (m, 0.5H, rotamers), 3.40-3.00 (m, 6H), 2.91-2.82 |
| | | (m, 0.5H, rotamers), 2.42-2.30 (m, 1.5H), 2.23-2.15 (m, |
| | | 0.5H, rotamers), 1.56-1.51 (m, 2H), 1.22 (d, J = 6.6 Hz, |
| | | 1H), 1.09 (d, J = 6.7 Hz, 11H), 0.97-0.87 (m, 6H). |
| 151 | (S)-2-(5-(diethylamino)-4-(4- | LC/MS ESI (m/z): 461 (M + H)⁺. ¹H NMR (400 MHz, |
| | isobutyryl-2-methylpiperazin-1- | CDCl₃) δ 9.38 (s, 1H), 8.58 (d, J = 4.9 Hz, 1H), 8.44 (s, |
| | yl)-7H-pyrrolo[2,3-d]pyrimidin- | 1H), 7.64 (s, 1H), 7.30 (d, J = 4.9 Hz, 1H), 5.21 (d, J = 43.0 |
| | 7-yl)isonicotinonitrile | Hz, 1H), 4.73-4.26 (m, 2H), 3.87 (dd, J = 84.3, 11.6 Hz, |
| | | 1H), 3.63-3.33 (m, 2H), 3.26-2.80 (m, 6H), 1.26-1.07 |
| | | (m, 9H), 1.03 (t, J = 7.0 Hz, 6H). |

### Example 29. Synthesis of 2-(4-((S)-4-(3,3-dimethylbutanoyl)-2-methylpiperazin-1-yl)-5-((R)-2-methylmorpholino)-7H-pyrrolo[2,3-d]pyrimidin-7-yl)isonicotinonitrile (Compound 165)

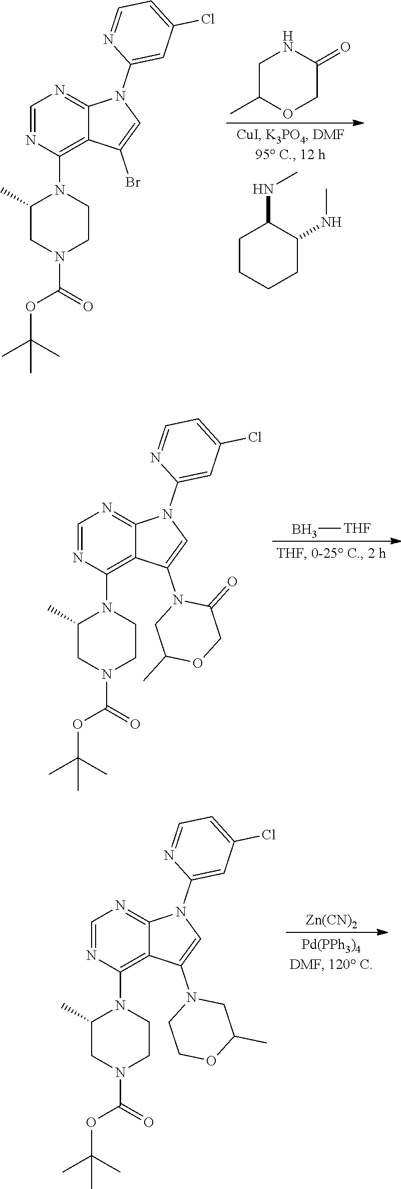

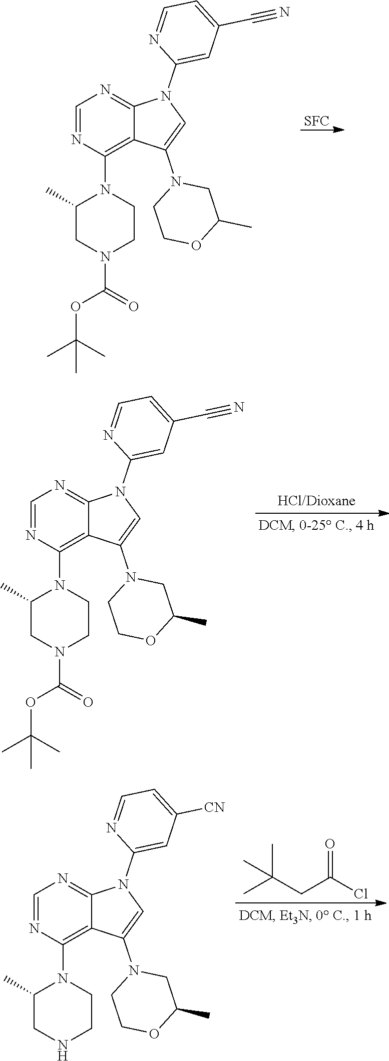

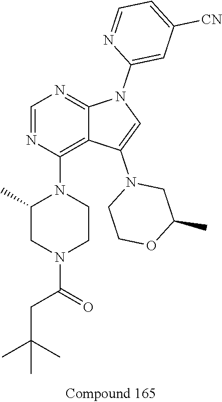 [SMILES: C[C@@H]1CN(c2cn(-c3cc(C#N)ccn3)c3ncnc(N4CCN(C(=O)CC(C)(C)C)C[C@@H]4C)c23)CCO1]

### Step 1. tert-butyl (S)-4-(5-bromo-7-(4-chloropyridin-2-yl)-7H-pyrrolo[2,3-d]pyrimidin-4-yl)-3-methylpiperazine-1-carboxylate

To a solution of tert-butyl (S)-4-(5-bromo-7-(4-chloropyridin-2-yl)-7H-pyrrolo[2,3-d]pyrimidin-4-yl)-3-methylpiperazine-1-carboxylate (600 mg, 1.2 mmol, prepared following the procedure described for compound 163) in DMF (15 mL) were added CuI (110 mg, 0.59 mmol), 6-methylmorpholin-3-one (410 mg, 3.5 mmol), K₃PO₄ (500 mg, 2.4 mmol) and trans-N,N′-dimethylcyclohexane-1,2-diamine (340 mg, 2.4 mmol) at 25° C. The mixture was degassed 3 times with N₂ and then stirred at 95° C. for 12 h. The mixture was cooled to 25° C., poured into H₂O and extracted twice with EtOAc. The combined organic phases were washed with brine, dried over Na₂SO₄, filtered, and concentrated under vacuo. The crude product was purified by silica gel column (SiO₂, petroleum ether:EtOAc=5:1 to 4:1) to afford tert-butyl (3S)-4-(7-(4-chloropyridin-2-yl)-5-(2-methyl-5-oxomorpholino)-7H-pyrrolo[2,3-d]pyrimidin-4-yl)-3-methylpiperazine-1-carboxylate (60 mg, 9%) as a solid. LC/MS ESI (m/z): 542 (M+H)⁺.

### Step 2. tert-butyl (3S)-4-(7-(4-chloropyridin-2-yl)-5-(2-methylmorpholino)-7H-pyrrolo[2,3-d]pyrimidin-4-yl)-3-methylpiperazine-1-carboxylate

To a solution of tert-butyl (3S)-4-(7-(4-chloropyridin-2-yl)-5-(2-methyl-5-oxomorpholino)-7H-pyrrolo[2,3-d]pyrimidin-4-yl)-3-methylpiperazine-1-carboxylate (60 mg, 0.11 mmol) in THF (5 mL) was added BH₃-THF (1.0 mL, 1.0M in THF) at 0° C. under N₂ atmosphere. The mixture was warmed to 25° C. and stirred for 2 h. The reaction was quenched by addition of MeOH (10 mL) at 0° C. and concentrated in vacuo. The crude product was purification by silica gel column (SiO₂, petroleum ether:EtOAc=50:1 to 40:1) to afford tert-butyl (3S)-4-(7-(4-chloropyridin-2-yl)-5-(2-methylmorpholino)-7H-pyrrolo[2,3-d]pyrimidin-4-yl)-3-methylpiperazine-1-carboxylate (40 mg, 68%) as a solid. LC/MS ESI (m/z): 528 (M+H)⁺.

### Step 3. tert-butyl (3S)-4-(7-(4-cyanopyridin-2-yl)-5-(2-methylmorpholino)-7H-pyrrolo[2,3-d]pyrimidin-4-yl)-3-methylpiperazine-1-carboxylate

To a solution of tert-butyl (3S)-4-(7-(4-chloropyridin-2-yl)-5-(2-methylmorpholino)-7H-pyrrolo[2,3-d]pyrimidin-4-yl)-3-methylpiperazine-1-carboxylate (40 mg, 0.076 mmol) in DMF (3 mL) were added Zn(CN)₂ (53 mg, 0.45 mmol) and Pd(PPh₃)⁺ (44 mg, 0.038 mmol) at 25° C. The mixture was degassed 3 times with N₂ and then stirred at 120° C. for 3 h. The mixture was poured into H₂O and extracted twice with EtOAc. The combined organic phases were washed with brine, dried over Na₂SO₄, filtered, and concentrated in vacuo. The crude product was purification by silica gel column (SiO₂, petroleum ether:EtOAc=50:1 to 30:1) and Prep-HPLC (with 0.1% formic acid) to afford tert-butyl (3S)-4-(7-(4-cyanopyridin-2-yl)-5-(2-methylmorpholino)-7H-pyrrolo[2,3-d]pyrimidin-4-yl)-3-methylpiperazine-1-carboxylate (10 mg, 25%) as a yellow solid. LC/MS ESI (m/z): 519 (M+H)⁺.

### Step 4. tert-butyl (S)-4-(7-(4-cyanopyridin-2-yl)-5-((R)-2-methylmorpholino)-7H-pyrrolo[2,3-d]pyrimidin-4-yl)-3-methylpiperazine-1-carboxylate

The racemate tert-butyl (3S)-4-(7-(4-cyanopyridin-2-yl)-5-(2-methylmorpholino)-7H-pyrrolo[2,3-d]pyrimidin-4-yl)-3-methylpiperazine-1-carboxylate (10 mg, 0.019 mmol) was separate by chiral Prep-HPLC (IA-H 4.6*250 mm IPA+0.05% DEA 40% 8 min). The longer retention time peak labeled as tert-butyl (S)-4-(7-(4-cyanopyridin-2-yl)-5-((R)-2-methylmorpholino)-7H-pyrrolo[2,3-d]pyrimidin-4-yl)-3-methylpiperazine-1-carboxylate (2.0 mg, 0.004 mmol) was obtained as a white solid. LC/MS ESI (m/z): 519 (M+H)⁺. ¹H NMR (400 MHZ, CDCl₃) δ 9.30 (s, 1H), 8.52 (d, J=4.8 Hz, 1H), 8.39 (s, 1H), 7.57 (s, 1H), 7.25 (d, J=5.0 Hz, 1H), 4.98 (s, 1H), 4.29-4.04 (m, 2H), 3.93-3.69 (m, 4H), 3.43-3.30 (m, 2H), 3.28-3.11 (m, 2H), 2.80 (m, 1H), 2.55-2.33 (m, 2H), 1.43 (s, 9H), 1.10-1.05 (m, 3H), 0.83-0.76 (m, 3H).

Scale-up using 2.0 g of tert-butyl (S)-4-(5-bromo-7-(4-chloropyridin-2-yl)-7H-pyrrolo[2,3-d]pyrimidin-4-yl)-3-methylpiperazine-1-carboxylate following the synthetic route described above gave 30 mg of tert-butyl (S)-4-(7-(4-cyanopyridin-2-yl)-5-((R)-2-methylmorpholino)-7H-pyrrolo[2,3-d]pyrimidin-4-yl)-3-methylpiperazine-1-carboxylate.

### Step 5. 2-(5-((R)-2-methylmorpholino)-4-((S)-2-methylpiperazin-1-yl)-7H-pyrrolo[2,3-d]pyrimidin-7-yl)isonicotinonitrile

To a solution of tert-butyl (S)-4-(7-(4-cyanopyridin-2-yl)-5-((R)-2-methylmorpholino)-7H-pyrrolo[2,3-d]pyrimidin-4-yl)-3-methylpiperazine-1-carboxylate (30 mg, 0.058 mmol) in DCM (5 mL) at 0° C. was added HCl (0.072 mL, 4.0M in dioxane). The mixture was warmed up to 25° C. and stirred for 4 h. The mixture was quenched with NaHCO₃ (aq.) and extracted twice with EtOAc. The combined organic phases were washed with brine, dried over Na₂SO₄, filtered and concentrated in vacuo. 2-(5-((R)-2-methylmorpholino)-4-((S)-2-methylpiperazin-1-yl)-7H-pyrrolo[2,3-d]pyrimidin-7-yl)isonicotinonitrile (20 mg, 83%) was obtained as a yellow solid. LC/MS ESI m/z: 419 (M+H)⁺.

### Step 6. 2-(4-((S)-4-(3,3-dimethylbutanoyl)-2-methylpiperazin-1-yl)-5-((R)-2-methylmorpholino)-7H-pyrrolo[2,3-d]pyrimidin-7-yl)isonicotinonitrile

To a solution of 2-(5-((R)-2-methylmorpholino)-4-((S)-2-methylpiperazin-1-yl)-7H-pyrrolo[2,3-d]pyrimidin-7-yl)isonicotinonitrile (20 mg, 0.048 mmol) in DCM (4 mL) at 0° C. was added Et₃N (9.7 mg, 0.096 mmol), followed by 3,3-dimethylbutanoyl chloride (8.0 mg, 0.053 mmol). The mixture was stirred at 0° C. for 1 h. The reaction was quenched with NaHCO₃ (aq.) and extracted twice with EtOAc. The combined organic phases were washed with brine, dried over Na₂SO₄, filtered and concentrated in vacuo. The crude product was purified by Prep-HPLC to afford 2-(4-((S)-4-(3,3-dimethylbutanoyl)-2-methylpiperazin-1-yl)-5-((R)-2-methylmorpholino)-7H-pyrrolo[2,3-d]pyrimidin-7-yl)isonicotinonitrile (10 mg, 40%) as a yellow solid. LC/MS ESI m/z: 517 (M+H)⁺. ¹H NMR (400 MHZ, CDCl₃) δ 9.30 (s, 1H), 8.52 (d, J=5.0 Hz, 1H), 8.40 (d, J=6.7 Hz, 1H), 7.59 (d, J=8.4 Hz, 1H), 7.26 (d, J=5.0 Hz, 1H), 5.06 (d, J=48.9 Hz, 1H), 4.51 (dd, J=35.9, 13.4 Hz, 1H), 4.34-4.07 (m, 1H), 3.98-3.87 (m, 1.5H), 3.85-3.65 (m, 2.5H, rotamers), 3.53 (dd, J=13.3, 3.1 Hz, 0.5H), 3.47-3.30 (m, 2H), 3.25-3.12 (m, 1.5H, rotamers), 3.03 (dd, J=12.9, 2.6 Hz, 0.5H, rotamers), 2.74-2.60 (m, 0.5H, rotamers), 2.56-2.36 (m, 2H), 2.34-2.22 (m, 1.5H, rotamers), 2.16-2.09 (m, 0.5H, rotamers), 1.28-1.20 (m, 3H), 1.20-1.11 (m, 3H), 1.02 (d, J=5.9 Hz, 9H).

### Example 30. Synthesis of 1-((2R,5S)-4-(5-(2-fluorophenyl)-7-(4-fluoropyridin-2-yl)-7H-pyrrolo[2,3-d]pyrimidin-4-yl)-2,5-dimethylpiperazin-1-yl)-2-methylpropan-1-one (Compound 167)

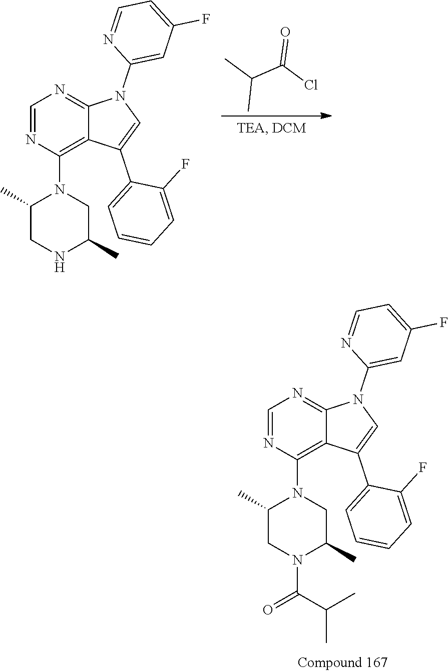

### Step 1. 1-((2R,5S)-4-(5-(2-fluorophenyl)-7-(4-fluoropyridin-2-yl)-7H-pyrrolo[2,3-d]pyrimidin-4-yl)-2,5-dimethylpiperazin-1-yl)-2-methylpropan-1-one

To a solution of 4-((2S,5R)-2,5-dimethylpiperazin-1-yl)-5-(2-fluorophenyl)-7-(4-fluoropyridin-2-yl)-7H-pyrrolo[2,3-d]pyrimidine (100 mg, 0.24 mmol, prepared following the procedure described for compound 169) and TEA (0.19 mL, 1.4 mmol) in DCM (5 mL) was added a solution of 2-methylpropanoyl chloride (0.060 mL, 0.59 mmol) in DCM. The resulting mixture was stirred at 0° C. for 1 h. The reaction was quenched with NaHCO₃ (aq.) and extracted twice with DCM. The combined organic phases were washed with brine, dried over anhydrous Na₂SO₄, filtered and concentrated. The residue was purified by flash chromatography (0˜50% ethyl acetate in petroleum ether) to give 1-[(2R,5S)-4-[5-(2-fluorophenyl)-7-(4-fluoropyridin-2-yl)-7H-pyrrolo[2,3-d]pyrimidin-4-yl]-2,5-dimethylpiperazin-1-yl]-2-methylpropan-1-one (25 mg, 21%) as a white solid. LC/MS ESI m/z: 491 (M+H)⁺. ¹H NMR (400 MHz, CDCl₃) δ 8.80 (d, J=10.5 Hz, 1H), 8.56 (d, J=4.7 Hz, 1H), 8.43 (dd, J=8.5, 5.7 Hz, 1H), 8.31 (d, J=3.3 Hz, 1H), 7.55-7.45 (m, 1H), 7.37 (dd, J=13.0, 6.1 Hz, 1H), 7.21 (dd, J=18.2, 8.6 Hz, 2H), 6.96 (t, J=5.5 Hz, 1H), 4.74-4.29 (m, 1H), 4.06 (dd, J=39.9, 5.2 Hz, 1H), 3.68 (m, 1H), 3.35 (d, J=13.3 Hz, 1H), 3.21-2.73 (m, 2H), 2.58 (dt, J=16.7, 8.6 Hz, 1H), 1.27 (d, J=6.6 Hz, 1.5H, rotamers), 1.16-1.07 (m, 5.5H, rotamers), 1.04-0.80 (m, 5H).

### Example 31. Synthesis of 1-((2R,5S)-4-(7-(4-chloropyridin-2-yl)-5-(2-fluorophenyl)-7H-pyrrolo[2,3-d]pyrimidin-4-yl)-2,5-dimethylpiperazin-1-yl)-2-methylpropan-1-one (Compound 168)

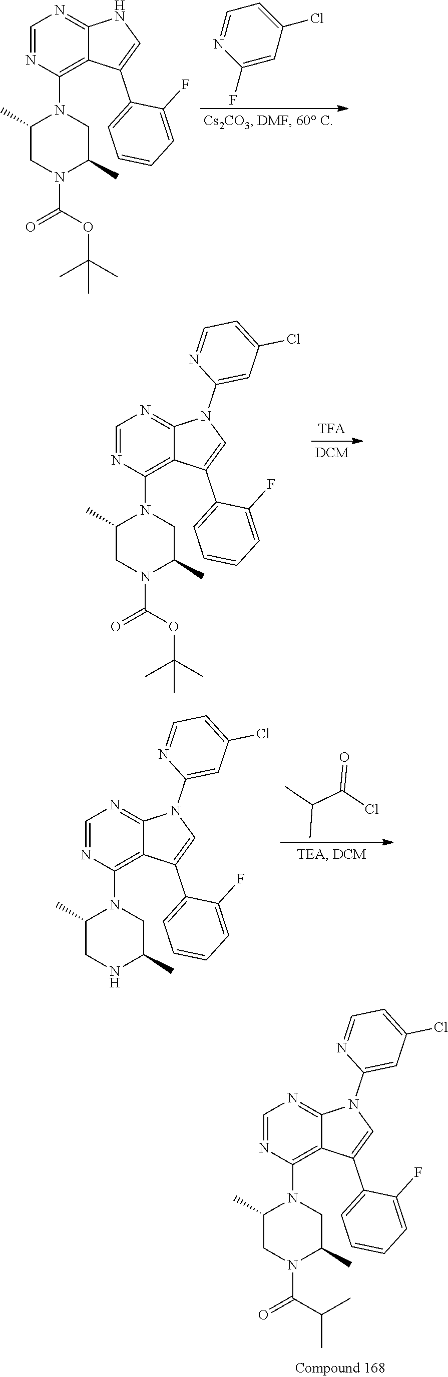

### Step 1. tert-butyl (2R,5S)-4-(7-(4-chloropyridin-2-yl)-5-(2-fluorophenyl)-7H-pyrrolo[2,3-d]pyrimidin-4-yl)-2,5-dimethylpiperazine-1-carboxylate

To a solution of tert-butyl (2R,5S)-4-(5-(2-fluorophenyl)-7H-pyrrolo[2,3-d]pyrimidin-4-yl)-2,5-dimethylpiperazine-1-carboxylate (340 mg, 0.80 mmol, prepared following the procedure outlined in compound 169) and 4-chloro-2-fluoropyridine (210 mg, 1.6 mmol) in DMF (5 mL) was added Cs₂CO₃ (1.0 g, 3.2 mmol). The resulting mixture was stirred at 60° C. for 18 h. After cooling to room temperature, the reaction mixture was poured into water and extracted twice with EtOAc. The combined organic phases were washed with brine, dried over anhydrous Na₂SO₄, filtered and concentrated. The residue was purified by column chromatography on silica gel (0˜40% EtOAc in petroleum ether) to give tert-butyl (2R,5S)-4-(7-(4-chloropyridin-2-yl)-5-(2-fluorophenyl)-7H-pyrrolo[2,3-d]pyrimidin-4-yl)-2,5-dimethylpiperazine-1-carboxylate (370 mg, 86%) as a white solid. LC/MS (ESI) m/z: 537 (M+H)⁺

### Step 2. 7-(4-chloropyridin-2-yl)-4-((2S,5R)-2,5-dimethylpiperazin-1-yl)-5-(2-fluorophenyl)-7H-pyrrolo[2,3-d]pyrimidine

To a solution of tert-butyl (2R,5S)-4-(7-(4-chloropyridin-2-yl)-5-(2-fluorophenyl)-7H-pyrrolo[2,3-d]pyrimidin-4-yl)-2,5-dimethylpiperazine-1-carboxylate (300 mg, 0.56 mmol) in DCM (3 mL) was added TFA (1.0 mL, 14 mmol). The resulting mixture was stirred at 0° C. for 3 h. After removal of solvent, the residue was used directly to next step without further purification. LC/MS ESI m/z: 437 (M+H)⁺.

### Step 3. 1-((2R,5S)-4-(7-(4-chloropyridin-2-yl)-5-(2-fluorophenyl)-7H-pyrrolo[2,3-d]pyrimidin-4-yl)-2,5-dimethylpiperazin-1-yl)-2-methylpropan-1-one

To a solution of 7-(4-chloropyridin-2-yl)-4-((2S,5R)-2,5-dimethylpiperazin-1-yl)-5-(2-fluorophenyl)-7H-pyrrolo[2,3-d]pyrimidine (240 mg, 0.55 mmol) and TEA (0.46 mL, 3.3 mmol) in DCM (5.0 mL) at 0° C. was added a solution of 2-methylpropanoyl chloride (0.14 mL, 1.4 mmol) in DCM. The resulting mixture was stirred at the same temperature for 1 h. The mixture was quenched with NaHCO₃ (aq.) and extracted twice with DCM. The combined organic phases were washed with brine, dried over anhydrous Na₂SO₄, filtered and concentrated. The residue was purified by Prep-HPLC to give 1-((2R,5S)-4-(7-(4-chloropyridin-2-yl)-5-(2-fluorophenyl)-7H-pyrrolo[2,3-d]pyrimidin-4-yl)-2,5-dimethylpiperazin-1-yl)-2-methylpropan-1-one (70 mg, 25%) as a white solid. LC/MS (ESI) m/z: 507 (M+H)⁺, ¹H NMR (400 MHZ, CDCl₃) δ 9.04 (s, 1H), 8.57 (d, J=4.9 Hz, 1H), 8.37 (d, J=5.3 Hz, 1H), 8.27 (d, J=3.4 Hz, 1H), 7.49 (dd, J=13.2, 6.3 Hz, 1H), 7.36 (dd, J=12.9, 6.1 Hz, 1H), 7.20 (dt, J=12.0, 8.5 Hz, 3H), 4.72-4.31 (m, 1H), 4.06 (m, 1H), 3.68 (m, 1H), 3.35 (d, J=13.5 Hz, 1H), 3.22-2.73 (m, 2H), 2.64-2.50 (m, 1H), 1.30-1.07 (m, 6H), 1.05-0.80 (m, 6H).

### Example 32. Synthesis of 1-((2R,5S)-4-(5-(2-fluorophenyl)-7-(4-fluoropyridin-2-yl)-7H-pyrrolo[2,3-d]pyrimidin-4-yl)-2,5-dimethylpiperazin-1-yl)-2-hydroxy-2-methylpropan-1-one (Compound 169)

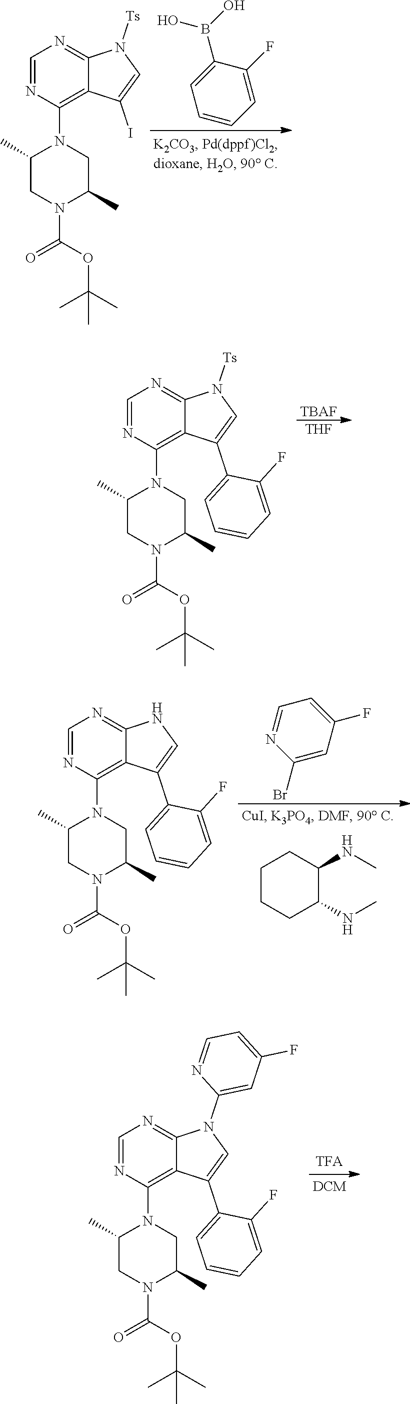

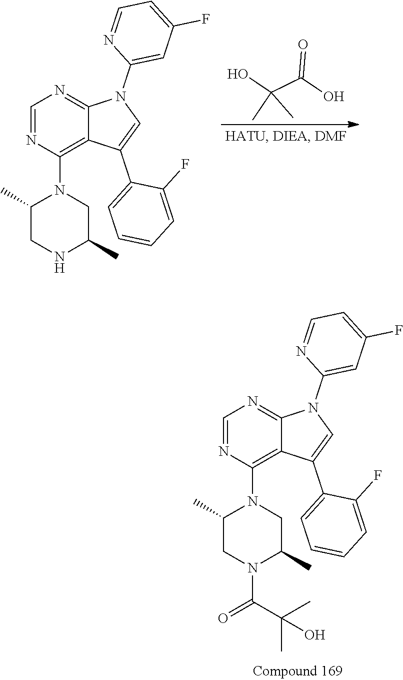

### Step 1. tert-butyl (2R,5S)-4-(5-(2-fluorophenyl)-7-tosyl-7H-pyrrolo[2,3-d]pyrimidin-4-yl)-2,5-dimethylpiperazine-1-carboxylate

A mixture of tert-butyl (2R,5S)-4-(5-iodo-7-tosyl-7H-pyrrolo[2,3-d]pyrimidin-4-yl)-2,5-dimethylpiperazine-1-carboxylate (3.1 g, 5.0 mmol, prepared following a similar procedure described for compound 128), (2-fluorophenyl) boronic acid (0.84 g, 6.0 mmol), K₂CO₃ (1.4 g, 10 mmol) and Pd(dppf)Cl₂ (0.36 g, 0.50 mmol) in dioxane-water (30 mL, 5:1 v/v) was stirred at 90° C. under N₂ atmosphere for 18 h. After cooling to room temperature, the reaction was partitioned between EtOAc and water. The aqueous layer was extracted twice with EtOAc. The combined organic phases were washed with brine, dried over anhydrous Na₂SO₄, filtered and concentrated. The residue was purified by column chromatography on silica gel (0˜30% EtOAc in petroleum ether) to give tert-butyl (2R,5S)-4-(5-(2-fluorophenyl)-7-tosyl-7H-pyrrolo[2,3-d]pyrimidin-4-yl)-2,5-dimethylpiperazine-1-carboxylate (2.7 g, 93%) as a white solid. LC/MS ESI m/z: 580 (M+H)⁺

### Step 2. tert-butyl (2R,5S)-4-(5-(2-fluorophenyl)-7H-pyrrolo[2,3-d]pyrimidin-4-yl)-2,5-dimethylpiperazine-1-carboxylate

To a solution of tert-butyl (2R,5S)-4-(5-(2-fluorophenyl)-7-tosyl-7H-pyrrolo[2,3-d]pyrimidin-4-yl)-2,5-dimethylpiperazine-1-carboxylate (2.7 g, 4.7 mmol) in THF (20 mL) was added TBAF (12 mL, 1.0M in THF). The resulting mixture was stirred at room temperature overnight. The reaction was poured into water and extracted twice with EtOAc. The combined organic phases were washed water and brine, dried over anhydrous Na₂SO₄, filtered and concentrated. The residue was purified by column chromatography on silica gel (0˜70% ethyl acetate in petroleum ether) to give tert-butyl (2R,5S)-4-(5-(2-fluorophenyl)-7H-pyrrolo[2,3-d]pyrimidin-4-yl)-2,5-dimethylpiperazine-1-carboxylate (1.8 g, 91%) as a white solid. LC/MS ESI m/z: 426 (M+H)⁺

### Step 3. tert-butyl (2R,5S)-4-(5-(2-fluorophenyl)-7-(4-fluoropyridin-2-yl)-7H-pyrrolo[2,3-d]pyrimidin-4-yl)-2,5-dimethylpiperazine-1-carboxylate

A mixture of tert-butyl (2R,5S)-4-(5-(2-fluorophenyl)-7H-pyrrolo[2,3-d]pyrimidin-4-yl)-2,5-dimethylpiperazine-1-carboxylate (550 mg, 1.3 mmol), 2-bromo-4-fluoropyridine (680 mg, 3.9 mmol), K₃PO₄ (820 mg, 3.9 mmol), CuI (120 mg, 0.64 mmol) and trans-N,N′-dimethylcyclohexane-1,2-diamine (280 mg, 1.9 mmol) in DMF (20 mL) was stirred at 90° C. under N₂ atmosphere overnight. After cooling to room temperature, the reaction was partitioned between EtOAc and water. The aqueous layer was extracted twice with EtOAc. The combined organic phases were washed with brine, dried over anhydrous Na₂SO₄, filtered and concentrated. The residue was purified by column chromatography on silica gel (0˜40% ethyl acetate in petroleum ether) to give ter-butyl (2R,5S)-4-(5-(2-fluorophenyl)-7-(4-fluoropyridin-2-yl)-7H-pyrrolo[2,3-d]pyrimidin-4-yl)-2,5-dimethylpiperazine-1-carboxylate (580 mg, 86%) as a white solid. LC/MS ESI m/z: 521 (M+H)⁺.

### Step 4. 4-((2S,5R)-2,5-dimethylpiperazin-1-yl)-5-(2-fluorophenyl)-7-(4-fluoropyridin-2-yl)-7H-pyrrolo[2,3-d]pyrimidine

To a solution of tert-butyl (2R,5S)-4-(5-(2-fluorophenyl)-7-(4-fluoropyridin-2-yl)-7H-pyrrolo[2,3-d]pyrimidin-4-yl)-2,5-dimethylpiperazine-1-carboxylate (580 mg, 1.1 mmol) in DCM (5 mL) was added TFA (1.7 mL, 22 mmol), the reaction was stirred at 0° C. for 2 h. After removal of solvent, the residue was used directly in next step. LC/MS ESI m/z: 421 (M+H)⁺.

### Step 5. 1-((2R,5S)-4-(5-(2-fluorophenyl)-7-(4-fluoropyridin-2-yl)-7H-pyrrolo[2,3-d]pyrimidin-4-yl)-2,5-dimethylpiperazin-1-yl)-2-hydroxy-2-methylpropan-1-one

To a solution of 4-((2S,5R)-2,5-dimethylpiperazin-1-yl)-5-(2-fluorophenyl)-7-(4-fluoropyridin-2-yl)-7H-pyrrolo[2,3-d]pyrimidine (210 mg, 0.50 mmol) in DMF (5.0 mL) were added DIEA (0.33 mL, 2.0 mmol), 2-hydroxy-2-methylpropanoic acid (62 mg, 0.60 mmol) and HATU (230 mg, 0.60 mmol). The resulting mixture was stirred at room temperature for 30 minutes. The reaction was quenched with NaHCO₃ (aq.) and extracted twice with EtOAc. The combined organic phases were washed with brine, dried over anhydrous Na₂SO₄, filtered and concentrated. The residue was purified by column chromatography on silica gel (0˜50% ethyl acetate in petroleum ether) and Prep-HPLC to give 1-((2R,5S)-4-(5-(2-fluorophenyl)-7-(4-fluoropyridin-2-yl)-7H-pyrrolo[2,3-d]pyrimidin-4-yl)-2,5-dimethylpiperazin-1-yl)-2-hydroxy-2-methylpropan-1-one (70 mg, 28%) as a white solid. LC/MS ESI m/z: 507 (M+H)⁺. ¹H NMR (400 MHZ, CDCl₃) δ 8.80 (dd, J=10.9, 1.9 Hz, 1H), 8.57 (s, 1H), 8.43 (dd, J=8.6, 5.7 Hz, 1H), 8.32 (s, 1H), 7.51 (t, J=6.3 Hz, 1H), 7.40-7.33 (m, 1H), 7.27-7.13 (m, 2H), 7.01-6.87 (m, 1H), 4.46 (m, 2H), 4.06-3.81 (m, 1H), 3.58-2.52 (m, 3H), 1.48 (m, 9H), 0.92 (pair of s, 3H, rotamers).

### Example 33. Synthesis of (S)-6-(5-(2-fluorophenyl)-4-(4-isobutyryl-2-methylpiperazin-1-yl)-7H-pyrrolo[2,3-d]pyrimidin-7-yl)pyridazine-4-carbonitrile (Compound 171)

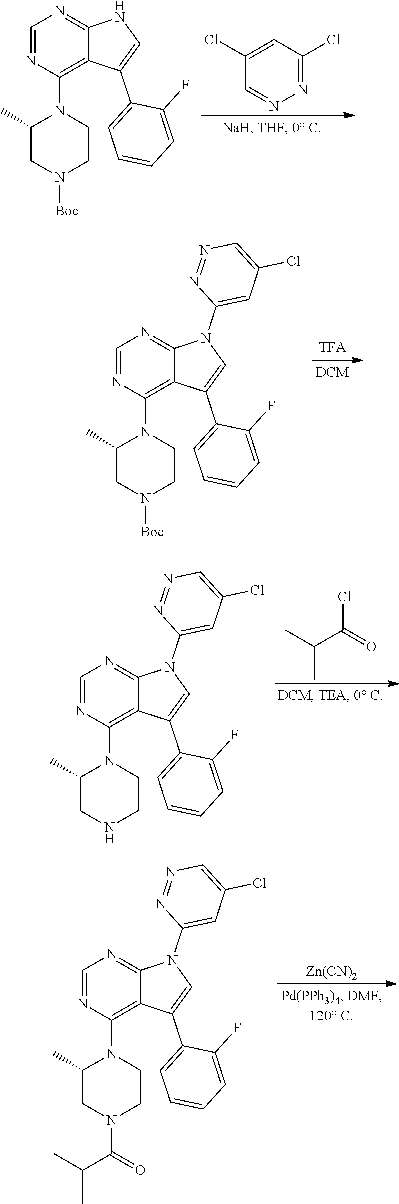

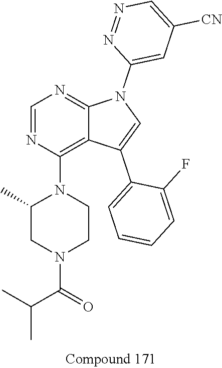 [SMILES: CC(C)C(=O)N1CCN(c2ncnc3c2c(-c2ccccc2F)cn3-c2cc(C#N)cnn2)[C@@H](C)C1]

### Step 1. tert-butyl (S)-4-(7-(5-chloropyridazin-3-yl)-5-(2-fluorophenyl)-7H-pyrrolo[2,3-d]pyrimidin-4-yl)-3-methylpiperazine-1-carboxylate

To a solution of tert-butyl (S)-4-(5-(2-fluorophenyl)-7H-pyrrolo[2,3-d]pyrimidin-4-yl)-3-methylpiperazine-1-carboxylate (160 mg, 0.40 mmol, prepared following a similar procedure described for compound 128) in THF (2 mL) was added NaH (64 mg, 0.16 mmol, 60% wt) at 0° C. The resulting mixture was stirred for 20 min under N₂ atmosphere before 3,5-dichloropyridazine (120 mg, 0.80 mmol) was added. After stirring at room temperature overnight, the reaction was quenched with NH₄Cl (aq.) and diluted with EtOAc. The organic layer was washed with brine, dried over Na₂SO₄, filter and concentrated. The residue was purified by flash column chromatography (silica gel, 0˜40% EtOAc in petroleum ether) to afford tert-butyl (S)-4-(7-(5-chloropyridazin-3-yl)-5-(2-fluorophenyl)-7H-pyrrolo[2,3-d]pyrimidin-4-yl)-3-methylpiperazine-1-carboxylate (72 mg, 34% yield) as a white solid. LC/MS ESI (m/z): 524 (M+H)⁺. The regioisomer tert-butyl (S)-4-(7-(6-chloropyridazin-4-yl)-5-(2-fluorophenyl)-7H-pyrrolo[2,3-d]pyrimidin-4-yl)-3-methylpiperazine-1-carboxylate (72 mg, 34%) was also obtained as a white solid. LC/MS ESI (m/z): 524 (M+H)⁺.

### Step 2. (S)-7-(5-chloropyridazin-3-yl)-5-(2-fluorophenyl)-4-(2-methylpiperazin-1-yl)-7H-pyrrolo[2,3-d]pyrimidine

To a solution of tert-butyl (S)-4-(7-(5-chloropyridazin-3-yl)-5-(2-fluorophenyl)-7H-pyrrolo[2,3-d]pyrimidin-4-yl)-3-methylpiperazine-1-carboxylate (72 mg, 0.14 mmol) in DCM (5 mL) at 0° C. was added TFA (1.0 mL). The resulting mixture was stirred at the room temperature for 1 h. The reaction mixture was basified with NaHCO₃ (aq.) and extracted twice with DCM. The combined organic layers were washed with NaHCO₃ (aq.), dried over Na₂SO₄ and concentrated to afford the(S)-7-(5-chloropyridazin-3-yl)-5-(2-fluorophenyl)-4-(2-methylpiperazin-1-yl)-7H-pyrrolo[2,3-d]pyrimidine (50 mg, 88%) as a yellow solid which was used in the next step without further purification. LC/MS ESI m/z: 424 (M+H)⁺

### Step 3. (S)-1-(4-(7-(5-chloropyridazin-3-yl)-5-(2-fluorophenyl)-7H-pyrrolo[2,3-d]pyrimidin-4-yl)-3-methylpiperazin-1-yl)-2-methylpropan-1-one

To a solution of (S)-7-(5-chloropyridazin-3-yl)-5-(2-fluorophenyl)-4-(2-methylpiperazin-1-yl)-7H-pyrrolo[2,3-d]pyrimidine (50 mg, 0.11 mmol) and TEA (36 mg, 0.30 mmol) in DCM (5 mL) at 0° C. was added isobutyryl chloride (25 mg, 0.24 mmol). The resulting mixture was stirred at room temperature for 20 minutes. The reaction was quenched with ice water and extracted twice with DCM. The combined layers were washed with NaHCO₃ (aq.) and brine, dried over Na₂SO₄ and concentrated. The residue was purified by column chromatography on silica gel (0˜40% EtOAc in petroleum ether, V/V) to give (S)-1-(4-(7-(5-chloropyridazin-3-yl)-5-(2-fluorophenyl)-7H-pyrrolo[2,3-d]pyrimidin-4-yl)-3-methylpiperazin-1-yl)-2-methylpropan-1-one (51 mg, 90%) as a yellow solid LC/MS ESI m/z: 494 (M+H)⁺

### Step 4. (S)-6-(5-(2-fluorophenyl)-4-(4-isobutyryl-2-methylpiperazin-1-yl)-7H-pyrrolo[2,3-d]pyrimidin-7-yl)pyridazine-4-carbonitrile

To a solution of (S)-1-(4-(7-(5-chloropyridazin-3-yl)-5-(2-fluorophenyl)-7H-pyrrolo[2,3-d]pyrimidin-4-yl)-3-methylpiperazin-1-yl)-2-methylpropan-1-one (51 mg, 0.10 mmol) in DMF (5 mL) was added Zn(CN)₂ (49 mg, 0.40 mmol) and Pd(PPh₃)⁺ (53 mg, 0.040 mmol). The mixture was stirred at 120° C. under N₂ atmosphere for 3 h. After cooling to room temperature, the reaction mixture was diluted with EtOAc, washed with brine, dried over Na₂SO₄, filter and concentrated. The residue was purified by flash column chromatography (silica gel, 0˜40% EtOAc in petroleum ether) to provide a crude product which was further purified by Prep-HPLC to afford (S)-6-(5-(2-fluorophenyl)-4-(4-isobutyryl-2-methylpiperazin-1-yl)-7H-pyrrolo[2,3-d]pyrimidin-7-yl)pyridazine-4-carbonitrile (20 mg, 40% yield) as a yellow solid. LC/MS ESI (m/z): 485 (M+H)⁺. ¹H NMR (400 MHZ, CDCl₃) δ 9.77 (s, 1H), 9.23 (s, 1H), 8.58-8.53 (m, 1H), 8.51-8.47 (m, 1H), 7.51-7.46 (m, 1H), 7.42-7.38 (m, 1H), 7.22-7.18 (m, 2H), 4.48-4.04 (m, 2H), 3.69-3.61 (m, 1H), 3.51-3.43 (m, 1H), 3.25-3.02 (m, 2H), 2.77-2.66 (m, 1H), 2.58-2.48 (m, 1H), 1.11-0.91 (m, 9H).

### Example 34. Synthesis of 2-(5-(2-fluorophenyl)-4-(7-(2-hydroxy-2-methylpropanoyl)-4,7-diazaspiro[2.5]octan-4-yl)-7H-pyrrolo[2,3-d]pyrimidin-7-yl)isonicotinonitrile (Compound 174)

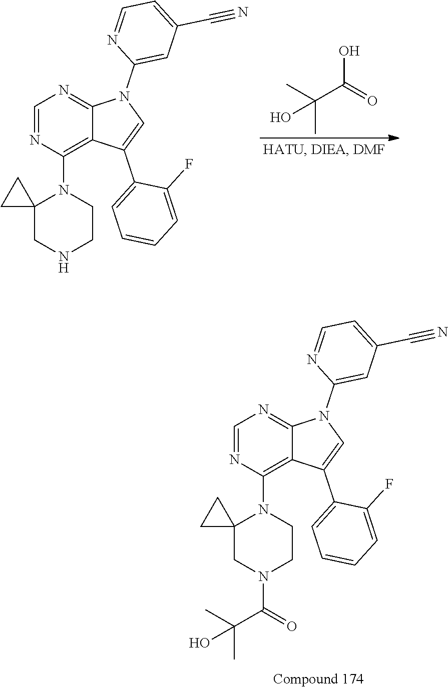

### Step 1. 2-(5-(2-fluorophenyl)-4-(7-(2-hydroxy-2-methylpropanoyl)-4,7-diazaspiro[2.5]octan-4-yl)-7H-pyrrolo[2,3-d]pyrimidin-7-yl)isonicotinonitrile

To a solution of 2-(5-(2-fluorophenyl)-4-(4,7-diazaspiro[2.5]octan-4-yl)-7H-pyrrolo[2,3-d]pyrimidin-7-yl)isonicotinonitrile (60 mg, 0.16 mmol, prepared following the procedure described for compound 155) in DMF (4 mL) were added 2-hydroxy-2-methylpropanoic acid (29 mg, 0.28 mmol), DIEA (55 mg, 0.42 mmol) and HATU (84 mg, 0.23 mmol). The resulting mixture was stirred at room temperature overnight. The reaction was quenched with NaHCO₃ (aq.) and extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to give crude product which was further purified by prep-HPLC to afford 2-(5-(2-fluorophenyl)-4-(7-(2-hydroxy-2-methylpropanoyl)-4,7-diazaspiro[2.5]octan-4-yl)-7H-pyrrolo[2,3-d]pyrimidin-7-yl)isonicotinonitrile (20 mg, 38%) as a white solid. LC/MS ESI m/z: 512 (M+H)⁺. ¹HNMR (400 MHZ, CDCl₃) δ 9.32 (s, 1H), 8.61 (s, 1H), 8.57 (d, J=4.9 Hz, 1H), 8.26 (s, 1H), 7.37-7.34 (m, 1H), 7.33-7.27 (m, 2H), 7.15-7.08 (m, 2H), 4.06-3.90 (m, 1H), 3.81-3.63 (m, 2H), 3.44-3.25 (m, 1H), 3.02-2.86 (m, 2H), 1.34 (s, 6H), 0.94-0.70 (m, 4H).

The following compounds were prepared by the procedures similar to the synthesis of compound 174 from the corresponding amine (tert-butyl 3,3-dimethylpiperazine-1-carboxylate).

| Cmpd | | |
|---|---|---|
| No. | Chemical Name | LCMS and ¹H NMR |
| 170 | 2-(5-(2-fluorophenyl)-4-(4-(2- | LC/MS ESI m/z: 514 (M + H)⁺ ¹H NMR(400 MHz, CDCl₃) |
| | hydroxy-2-methylpropanoyl)- | δ 9.33 (s, 1H), 8.61 (s, 1H), 8.55 (d, J = 5.0 Hz, 1H), 8.21 |
| | 2,2-dimethylpiperazin-1-yl)-7H- | (s, 1H), 7.43-7.39 (m, 1H), 7.36-7.31 (m, 2H), 7.16- |
| | pyrrolo[2,3-d]pyrimidin-7- | 7.11 (m, 2H), 4.17 (s, 1H), 3.38-3.29 (m, 2H), 2.99-2.94 |
| | yl)isonicotinonitrile | (m, 2H), 2.68-2.58 (m, 2H), 1.51 (s, 6H), 1.34 (s, 6H) |

### Example 35. Synthesis of 1-((2R,5S)-4-(7-(3,5-difluorophenyl)-5-(3-methylpyrazin-2-yl)-7H-pyrrolo[2,3-d]pyrimidin-4-yl)-2,5-dimethylpiperazin-1-yl)-3-methylbutan-1-one (Compound 177)

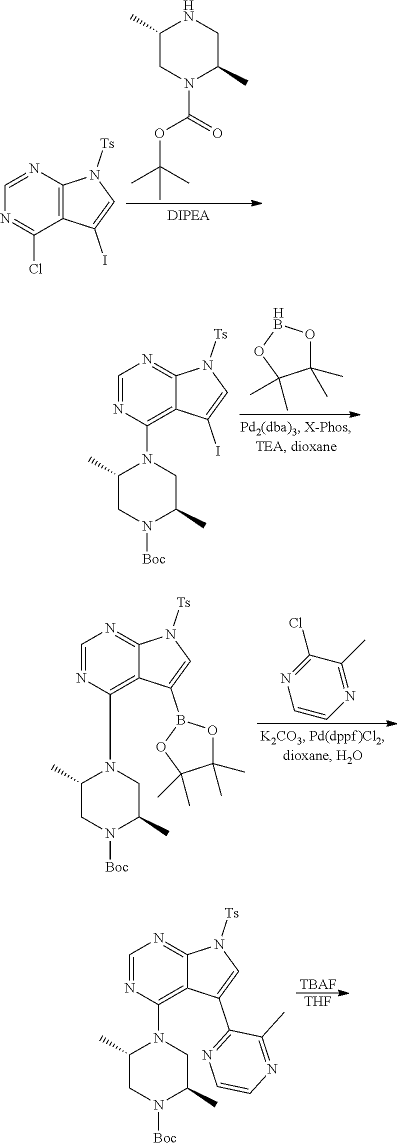

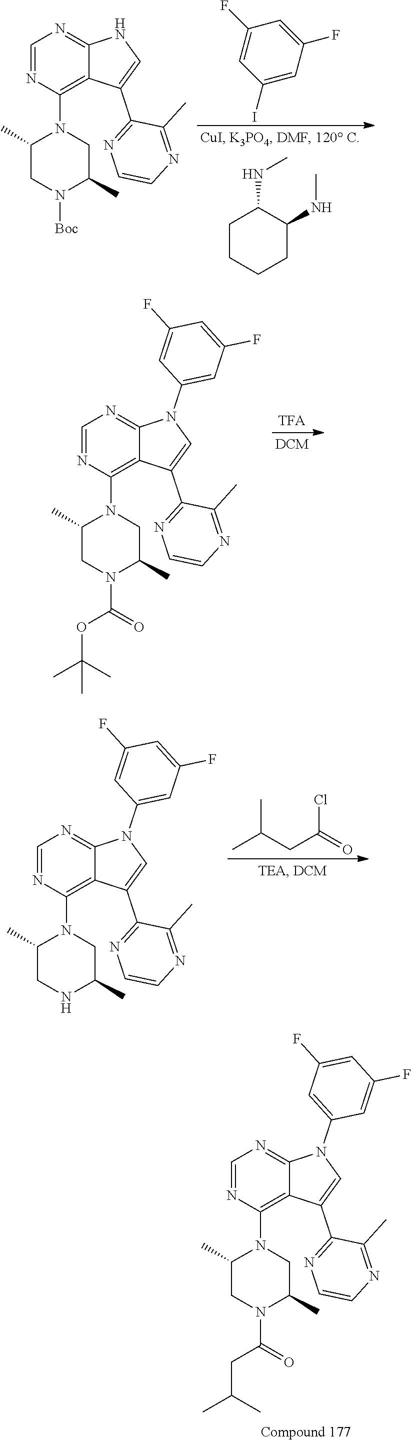

### Step 1. tert-butyl (2R,5S)-4-(5-iodo-7-tosyl-7H-pyrrolo[2,3-d]pyrimidin-4-yl)-2,5-dimethylpiperazine-1-carboxylate

To a solution of 4-chloro-5-iodo-7-tosyl-7H-pyrrolo[2,3-d]pyrimidine (12 g, 28 mmol, prepared following the procedure described for compound 128) in DIPEA (15 mL) was added tert-butyl (2R,5S)-2,5-dimethylpiperazine-1-carboxylate (9.0 g, 42 mmol). The resulting mixture was heated to 150° C. for 3 h under N₂ atmosphere. After cooling to room temperature, solvent was removed and the residue was purified by flash column chromatography (silica gel, 0˜30%, ethyl acetate in petroleum ether) to afford tert-butyl (2R,5S)-4-(5-iodo-7-tosyl-7H-pyrrolo[2,3-d]pyrimidin-4-yl)-2,5-dimethylpiperazine-1-carboxylate (16 g, 94%) as a light yellow solid. LC/MS ESI m/z: 612 (M+H)⁺.

### Step 2. tert-butyl (2R,5S)-2,5-dimethyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (2.0 g, 3.2 mmol) in dioxane (20 mL) were added 4,4,5,5-tetramethyl-1,3,2-dioxaborolane (1.7 g, 13 mmol), TEA (2.2 mL, 16 mmol), X-Phos (0.31 g, 0.65 mmol) and Pd₂(dba)₃ (0.30 g, 0.32 mmol). The resulting mixture was stirred at 95° C. overnight. After cooling to room temperature, the reaction was quenched with water and extracted twice with DCM. The combined organic layers were washed with brine, dried over Na₂SO₄, filtered and concentrated to afford crude tert-butyl (2R,5S)-2,5-dimethyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate as a brown oil, which was used in the next step directly. LC/MS ESI m/z: 612 (M+H)⁺.

### Step 3. tert-butyl (2R,5S)-2,5-dimethyl-4-(5-(3-methylpyrazin-2-yl)-7-tosyl-7H-pyrrolo[2,3-d]pyrimidin-4-yl)piperazine-1-carboxylate

To a solution of tert-butyl (2R,5S)-2,5-dimethyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate (2.0 g, 3.2 mmol, theoretical) in dioxane (20 mL) and H₂O (4 mL) were added 2-chloro-3-methylpyrazine (0.92 g, 7.2 mmol), K₂CO₃ (2.5 g, 18 mmol) and Pd(dppf)Cl₂ (0.26 g, 0.36 mmol). The resulting mixture was heated to 90° C. overnight. After cooling to room temperature, solvent was removed and the residue was purified by flash column chromatography (silica gel, 0˜50%, ethyl acetate in petroleum ether) to afford tert-butyl (2R,5S)-2,5-dimethyl-4-(5-(3-methylpyrazin-2-yl)-7-tosyl-7H-pyrrolo[2,3-d]pyrimidin-4-yl)piperazine-1-carboxylate (1.3 g, 62%) as a yellow solid. LC/MS ESI m/z: 578 (M+H)⁺.

### Step 4. tert-butyl (2R,5S)-2,5-dimethyl-4-(5-(3-methylpyrazin-2-yl)-7H-pyrrolo[2,3-d]pyrimidin-4-yl)piperazine-1-carboxylate

To a solution of tert-butyl (2R,5S)-2,5-dimethyl-4-(5-(3-methylpyrazin-2-yl)-7-tosyl-7H-pyrrolo[2,3-d]pyrimidin-4-yl)piperazine-1-carboxylate (1.3 g, 2.2 mmol) in THF (10 mL) was added TBAF (10 mL, 1.0M in THF). The resulting mixture was stirred at room temperature for 2 h. The reaction was quenched with water and extracted twice with EtOAc. The combined organic layers were washed with NH₄Cl (aq.), dried over Na₂SO₄, filtered and concentrated to afford crude tert-butyl (2R,5S)-2,5-dimethyl-4-(5-(3-methylpyrazin-2-yl)-7H-pyrrolo[2,3-d]pyrimidin-4-yl)piperazine-1-carboxylate (950 mg, 99%) as a brown solid. LC/MS ESI m/z: 424 (M+H)⁺.

### Step 5. tert-butyl (R)-4-(7-(3,5-difluorophenyl)-5-(pyridin-2-yl)-7H-pyrrolo[2,3-d]pyrimidin-4-yl)-2-methylpiperazine-1-carboxylate

To a solution of tert-butyl (2R,5S)-2,5-dimethyl-4-(5-(3-methylpyrazin-2-yl)-7H-pyrrolo[2,3-d]pyrimidin-4-yl)piperazine-1-carboxylate (480 mg, 1.1 mmol) in DMF (5 mL) were added 1,3-difluoro-5-iodobenzene (310 mg, 1.3 mmol), trans-N,N′-dimethylcyclohexane-1,2-diamine (71 mg, 0.50 mmol), CuI (210 mg, 1.1 mmol) and K₃PO₄ (700 mg, 3.3 mmol). The resulting mixture was heated to 120° C. overnight. After cooling to room temperature, the reaction was quenched with water and extracted twice with EtOAc. The combined organic layers were washed with brine, dried over Na₂SO₄, filtered and concentrated. The residue was purified by flash column chromatography (silica gel, 0˜30%, ethyl acetate in petroleum ether) to give crude product which was further purified by Prep-HPLC to afford tert-butyl (2R,5S)-4-(7-(3,5-difluorophenyl)-5-(3-methylpyrazin-2-yl)-7H-pyrrolo[2,3-d]pyrimidin-4-yl)-2,5-dimethylpiperazine-1-carboxylate (780 mg, 84%) as a white solid. LC/MS ESI m/z: 536 (M+H)⁺. ¹H NMR (400 MHZ, CDCl₃) δ 8.57-8.51 (m, 3H), 7.49-7.43 (m, 3H), 6.84 (t, J=8.7 Hz, 1H), 4.09 (m, 2H), 3.31 (m, 2H), 3.09 (s, 1H), 2.95-2.66 (m, 1H), 2.53 (s, 3H), 1.43 (s, 9H), 1.02-0.97 (m, 6H).

### Step 6. 7-(3,5-difluorophenyl)-4-((2S,5R)-2,5-dimethylpiperazin-1-yl)-5-(3-methylpyrazin-2-yl)-7H-pyrrolo[2,3-d]pyrimidine

To a solution of tert-butyl (2R,5S)-4-(7-(3,5-difluorophenyl)-5-(3-methylpyrazin-2-yl)-7H-pyrrolo[2,3-d]pyrimidin-4-yl)-2,5-dimethylpiperazine-1-carboxylate (650 mg, 1.2 mmol) in DCM (3 mL) at 0° C. was added TFA (2.0 mL). The resulting mixture was stirred at the room temperature for 1.5 h. The reaction was quenched with NaHCO₃ (aq.). The aqueous layer was extracted with DCM twice. The combined organic layers were washed with brine, dried over Na₂SO₄, filtered and concentrated. The residue was used in the next step directly. LC/MS ESI m/z: 436 (M+H)⁺.

### Step 7. 1-((2R,5S)-4-(7-(3,5-difluorophenyl)-5-(3-methylpyrazin-2-yl)-7H-pyrrolo[2,3-d]pyrimidin-4-yl)-2,5-dimethylpiperazin-1-yl)-3-methylbutan-1-one

To a solution of 7-(3,5-difluorophenyl)-4-((2S,5R)-2,5-dimethylpiperazin-1-yl)-5-(3-methylpyrazin-2-yl)-7H-pyrrolo[2,3-d]pyrimidine (100 mg, 0.23 mmol) in DCM (3 mL) at 0° C. were added dropwise TEA (0.060 mL, 0.46 mmol) and 3-methylbutanoyl chloride (0.040 mL, 0.34 mmol). The resulting mixture was stirred at room temperature for 1.5 h. The reaction was quenched with ice water and extracted twice with DCM. The combined organic layers were washed with NaHCO₃ (aq.) and brine, dried over Na₂SO₄, filtered and concentrated. The residue was purified by flash column chromatography (silica gel, ethyl acetate in petroleum ether) to give crude product which was further purified by Prep-HPLC to afford 1-((2R,5S)-4-(7-(3,5-difluorophenyl)-5-(3-methylpyrazin-2-yl)-7H-pyrrolo[2,3-d]pyrimidin-4-yl)-2,5-dimethylpiperazin-1-yl)-3-methylbutan-1-one (33 mg, 27%) as a white solid. LC/MS ESI m/z: 520 (M+H)⁺. ¹H NMR (400 MHZ, CDCl₃) δ 8.58-8.50 (m, 3H), 7.47 (t, J=7.1 Hz, 3H), 6.85 (t, J=8.6 Hz, 1H), 4.52 (pair of s, 1H, rotamers), 3.98 (s, 1H), 3.88 (d, J=13.5 Hz, 0.5H, rotamers), 3.50 (d, J=13.1 Hz, 0.5H, rotamers), 3.27-3.19 (m, 2H), 2.98-2.92 (m, 0.5H, rotamers), 2.54 (s, 3H), 2.39 (d, J=14.7 Hz, 0.5H, rotamers), 2.32-2.25 (m, 0.5H, rotamers), 2.13-2.03 (m, 2.5H, rotamers), 1.18 (d, J=6.6 Hz, 1.5H, rotamers), 1.08 (d, J=6.6 Hz, 1.5H, rotamers), 0.98-0.91 (m, 7.5H, rotamers), 0.86 (d, J=6.6 Hz, 1.5H, rotamers).

The following compounds were prepared by the procedures analogous to the synthesis of compound 177 from the corresponding acid chlorides and aryl halides.

| Cmpd | | |
|---|---|---|
| No. | Chemical Name | LCMS and ¹H NMR |
| 175 | 1-((2R,5S)-4-(7-(3- | LC/MS ESI (m/z): 504 (M + H)⁺. ¹H NMR (400 MHz, |
| | chlorophenyl)-5-(3- | CDCl₃) δ 8.57-8.51 (m, 3H), 7.81 (s, 1H), 7.69 (d, J = 7.9 |
| | methylpyrazin-2-yl)-7H- | Hz, 1H), 7.49 (t, J = 8.0 Hz, 2H), 7.39 (d, J = 7.9 Hz, 1H), |
| | pyrrolo[2,3-d]pyrimidin-4-yl)- | 4.60-4.45 (m, 1H), 4.09-4.03 (m, 0.5H, rotamers), 4.00- |
| | 2,5-dimethylpiperazin-1-yl)-2- | 3.93 (m, 0.5H, rotamers), 3.88 (d, J = 13.3 Hz, 0.5H, |
| | methylpropan-1-one | rotamers), 3.52 (d, J = 13.5 Hz, 0.5H, rotamers), 3.31- |
| | | 3.22 (m, 2H), 3.00-2.94 (m, 0.5H, rotamers), 2.78-2.71 |
| | | (m, 0.5H, rotamers), 2.65-2.60 (m, 0.5H, rotamers), 2.55 |
| | | (s, 3H), 2.41 (d, J = 12.6 Hz, 0.5H, rotamers), 1.21 (d, J = |
| | | 6.6 Hz, 1.5H, rotamers), 1.15-1.09 (m, 6H), 1.03 (d, J = |
| | | 6.7 Hz, 1.5H, rotamers), 0.94 (d, J = 6.7 Hz, 1.5H, |
| | | rotamers), 0.85 (d, J = 6.5 Hz, 1.5H, rotamers). |
| 176 | 1-((2R,5S)-4-(7-(3- | LC/MS ESI (m/z): 518 (M + H)⁺. ¹H NMR (400 MHz, |
| | chlorophenyl)-5-(3- | CDCl₃) δ 8.57-8.50 (m, 3H), 7.81 (d, J = 1.8 Hz, 1H), |
| | methylpyrazin-2-yl)-7H- | 7.69 (d, J = 8.1 Hz, 1H), 7.52-7.46 (m, 2H), 7.39 (d, J = |
| | pyrrolo[2,3-d]pyrimidin-4-yl)- | 8.0 Hz, 1H), 4.62-4.56 (m, 0.5H, rotamers), 4.51-4.41 |
| | 2,5-dimethylpiperazin-1-yl)-3- | (m, 0.5H, rotamers), 4.04-3.94 (m, 1H), 3.88 (d, J = 13.9 |
| | methylbutan-1-one | Hz, 0.5H, rotamers), 3.51 (d, J = 13.4 Hz, 0.5H, rotamers), |
| | | 3.29-3.19 (m, 2H), 2.97 (dd, J = 13.5, 3.3 Hz, 0.5H, |
| | | rotamers), 2.55 (s, 3H), 2.40 (d, J = 11.9 Hz, 0.5H, |
| | | rotamers), 2.30-2.25 (m, 0.5H, rotamers), 2.13-2.05 (m, |
| | | 2.5H, rotamers), 1.19 (d, J = 6.7 Hz, 1.5H, rotamers), 1.08 |
| | | (d, J = 6.6 Hz, 1.5H, rotamers), 0.95 (m, 7.5H, rotamers), |
| | | 0.86 (d, J = 6.5 Hz, 1.5H, rotamers). |

### Example 36. Synthesis of 2-(5-(2-fluorophenyl)-4-((2R,5S)-4-isobutyryl-2,5-dimethylpiperazin-1-yl)-7H-pyrrolo[2,3-d]pyrimidin-7-yl)isonicotinonitrile (Compound 201)

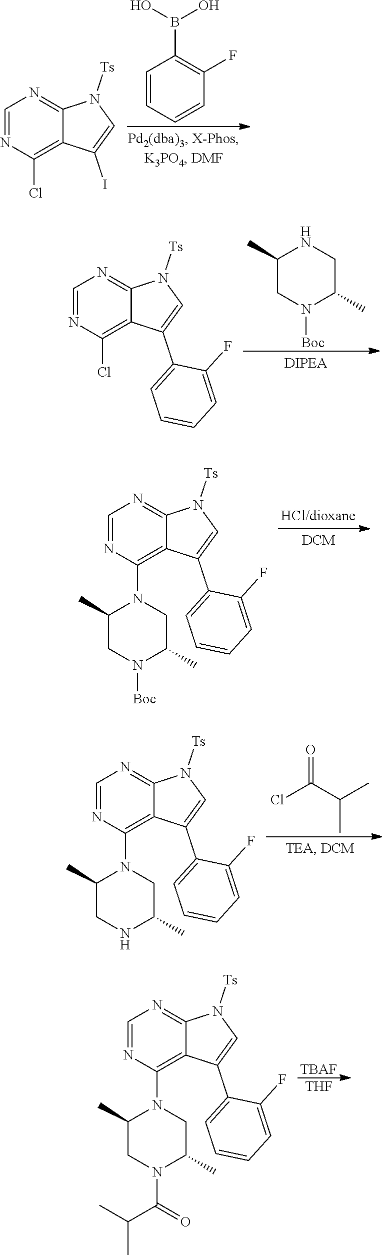

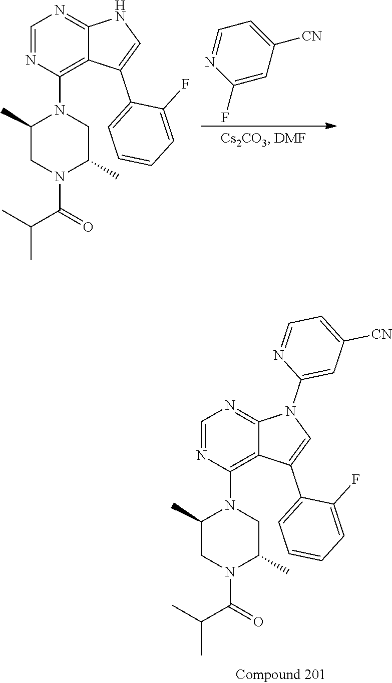

### Step 1. 4-Chloro-5-(2-fluorophenyl)-7-tosyl-7H-pyrrolo[2,3-d]pyrimidine

To a solution of 4-chloro-5-iodo-7-tosyl-7H-pyrrolo[2,3-d]pyrimidine (1.1 g, 2.5 mmol, prepared following the procedure outlined for compound 230) in DMF (15 mL) and water (0.5 mL) were added (2-fluorophenyl) boronic acid (420 mg, 0.60 mmol), X-Phos (180 mg, 0.37 mmol), K₃PO₄ (1.6 g, 7.5 mmol) and Pd₂(dba)₃ (230 mg, 0.25 mmol). The resulting mixture was heated at 60° C. overnight. After cooling to room temperature, the reaction mixture was filtered, and the filtrate was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜50% ethyl acetate in petroleum ether) to afford 4-chloro-5-(2-fluorophenyl)-7-tosyl-7H-pyrrolo[2,3-d]pyrimidine (600 mg, 60%). LC/MS ESI (m/z): 402 (M+H)⁺.

### Step 2. tert-Butyl (2S,5R)-4-(5-(2-fluorophenyl)-7-tosyl-7H-pyrrolo[2,3-d]pyrimidin-4-yl)-2,5-dimethylpiperazine-1-carboxylate

To a solution of 4-chloro-5-(2-fluorophenyl)-7-tosyl-7H-pyrrolo[2,3-d]pyrimidine (300 mg, 0.74 mmol) in DIPEA (0.40 mL, 2.2 mmol) was added tert-butyl (2S,5R)-2,5-dimethylpiperazine-1-carboxylate (240 mg, 1.1 mmol). The resulting mixture was heated at 150° C. for 2 h. After cooling to room temperature, the solvent was removed and the residue was purified by flash column chromatography (silica gel, 0˜20% ethyl acetate in petroleum ether) to afford tert-butyl (2S,5R)-4-(5-(2-fluorophenyl)-7-tosyl-7H-pyrrolo[2,3-d]pyrimidin-4-yl)-2,5-dimethylpiperazine-1-carboxylate (320 mg, 74%) as a white solid. LC/MS ESI (m/z): 580 (M+H)⁺.

### Step 3. 4-((2R,5S)-2,5-Dimethylpiperazin-1-yl)-5-(2-fluorophenyl)-7-tosyl-7H-pyrrolo[2,3-d]pyrimidine

To a solution of tert-butyl (2S,5R)-4-(5-(2-fluorophenyl)-7-tosyl-7H-pyrrolo[2,3-d]pyrimidin-4-yl)-2,5-dimethylpiperazine-1-carboxylate (320 mg, 0.55 mmol) in DCM (3 mL) was added HCl (1.0 mL, 4.0 M in dioxane). The resulting mixture was stirred at room temperature for 2 h. After removal of solvent, the residue was used in the next step directly. LC/MS ESI (m/z): 480 (M+H)⁺.

### Step 4. 1-((2S,5R)-4-(5-(2-Fluorophenyl)-7-tosyl-7H-pyrrolo[2,3-d]pyrimidin-4-yl)-2,5-dimethylpiperazin-1-yl)-2-methylpropan-1-one

To a solution of 4-((2R,5S)-2,5-dimethylpiperazin-1-yl)-5-(2-fluorophenyl)-7-tosyl-7H-pyrrolo[2,3-d]pyrimidine (260 mg, 0.54 mmol) and TEA (0.38 mL, 2.7 mmol) in DCM (3 mL) at 0° C. was added isobutyryl chloride (0.12 mL, 1.1 mmol). After 1 h, the reaction was partitioned between DCM and water. The aqueous layer was extracted twice with DCM. The combined organic layers were washed with brine, dried over Na₂SO₄, filtered and concentrated. The residue was purified by flash column chromatography (silica gel, 0˜50% ethyl acetate in petroleum ether) to afford 1-((2S,5R)-4-(5-(2-fluorophenyl)-7-tosyl-7H-pyrrolo[2,3-d]pyrimidin-4-yl)-2,5-dimethylpiperazin-1-yl)-2-methylpropan-1-one (290 mg, 95%) as a white solid. LC/MS ESI (m/z): 550 (M+H)⁺.

### Step 5. 1-((2S,5R)-4-(5-(2-Fluorophenyl)-7H-pyrrolo[2,3-d]pyrimidin-4-yl)-2,5-dimethylpiperazin-1-yl)-2-methylpropan-1-one

To a solution of 1-((2S,5R)-4-(5-(2-fluorophenyl)-7-tosyl-7H-pyrrolo[2,3-d]pyrimidin-4-yl)-2,5-dimethylpiperazin-1-yl)-2-methylpropan-1-one (290 mg, 0.52 mmol) in THF (3 mL) was added TBAF (2.1 mL, 1.0 M in THF). After 2 h, the reaction was diluted with water and extracted twice with EtOAc. The combined organic layers were washed with brine, dried over Na₂SO₄, filtered and concentrated. The residue was purified by flash column chromatography (silica gel, 0˜60% ethyl acetate in petroleum ether) to afford 1-((2S,5R)-4-(5-(2-fluorophenyl)-7H-pyrrolo[2,3-d]pyrimidin-4-yl)-2,5-dimethylpiperazin-1-yl)-2-methylpropan-1-one (200 mg, 97%) as a white solid. LC/MS ESI (m/z): 396 (M+H)⁺.

### Step 6. 2-(5-(2-Fluorophenyl)-4-((2R,5S)-4-isobutyryl-2,5-dimethylpiperazin-1-yl)-7H-pyrrolo[2,3-d]pyrimidin-7-yl)isonicotinonitrile

To a solution of 1-((2S,5R)-4-(5-(2-fluorophenyl)-7H-pyrrolo[2,3-d]pyrimidin-4-yl)-2,5-dimethylpiperazin-1-yl)-2-methylpropan-1-one (200 mg, 0.50 mmol) in DMF (10 mL) were added 2-fluoroisonicotinonitrile (120 mg, 1.0 mmol) and Cs₂CO₃ (1.6 g, 5.1 mmol). The resulting mixture was stirred at 50° C. for 1 h. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜50% ethyl acetate in petroleum ether) to give crude product which was further purified by prep-HPLC to afford 2-(5-(2-fluorophenyl)-4-((2R,5S)-4-isobutyryl-2,5-dimethylpiperazin-1-yl)-7H-pyrrolo[2,3-d]pyrimidin-7-yl)isonicotinonitrile (27 mg, 10%) as a white solid. LC/MS ESI (m/z): 498 (M+H)⁺. ¹H NMR (400 MHZ, CDCl₃) δ 9.34 (s, 1H), 8.56 (d, J=4.9 Hz, 1H), 8.51 (d, J=6.0 Hz, 1H), 8.23 (d, J=4.6 Hz, 1H), 7.43 (dd, J=13.9, 6.9 Hz, 1H), 7.35-7.28 (m, 2H), 7.20-7.10 (m, 2H), 4.60 (br. s, ˜0.5H), 4.34 (br. s, ˜0.5H), 4.05 (br. s, ˜0.5H), 3.95 (br. s, ˜0.5H), 3.78 (d, J=16 Hz, ˜0.5H), 3.48 (d, J=16 Hz, ˜0.5H), 3.27 (m, 1H), 3.16-3.02 (m, 1H), 2.80-2.37 (m, 2H), 1.21 (d, J=6.6 Hz, 2H), 1.10-1.01 (m, 5H), 0.99-0.89 (m, 4H), 0.76 (d, J=6.6 Hz, 1H); several peaks and their integrations are fractions of protons on account of presence of rotomers.

### Example 37. Synthesis of 2-(5-cyclopropyl-4-((S)-4-((R)-2-fluoro-3,3-dimethylbutanoyl)-2-methylpiperazin-1-yl)-7H-pyrrolo[2,3-d]pyrimidin-7-yl)isonicotinonitrile (Compound 202) and 2-(5-cyclopropyl-4-((S)-4-((S)-2-fluoro-3,3-dimethylbutanoyl)-2-methylpiperazin-1-yl)-7H-pyrrolo[2,3-d]pyrimidin-7-yl)isonicotinonitrile (Compound 203)

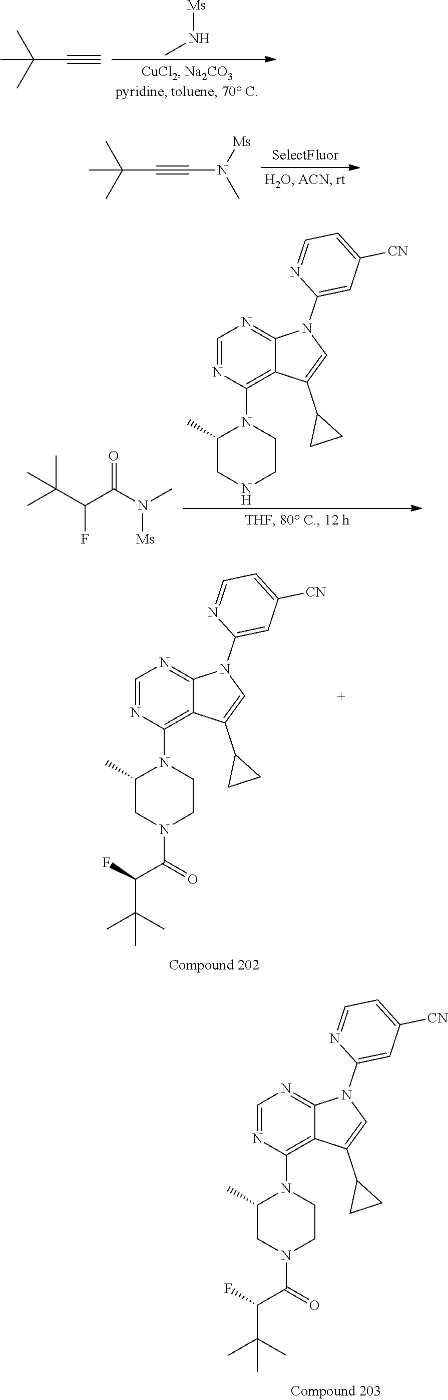

### Step 1. N-(3,3-Dimethylbut-1-yn-1-yl)-N-methylmethanesulfonamide

To a suspension of CuCl₂ (65 mg, 0.49 mmol), N-methylmethanesulfonamide (660 mg, 6.1 mmol) and Na₂CO₃ (520 mg, 4.9 mmol) in toluene (12 mL) under O₂ atmosphere was added pyridine (430 mg, 4.9 mmol). After stirring at 70° C. for 15 min, a solution of 3,3-dimethylbut-1-yne (200 mg, 2.4 mmol) in toluene (12 mL) was added dropwise. After stirring at 70° C. for 4 h, the reaction was cooled to room temperature and the solvent was removed. The residue was purified by flash chromatography (silica gel, ethyl acetate in petroleum ether) to afford N-(3,3-dimethylbut-1-yn-1-yl)-N-methylmethanesulfonamide (230 mg, 50%).

### Step 2. 2-Fluoro-N,3,3-trimethyl-N-(methylsulfonyl) butanamide

To a solution of N-(3,3-dimethylbut-1-yn-1-yl)-N-methylmethanesulfonamide (180 mg, 1.0 mmol) in MeCN (15 mL) and water (5 mL) was added SelectFluor (710 mg, 2.0 mmol). After stirring at room temperature for 2 h, the solvent was removed and the residue was purified by flash chromatography (silica gel) to afford 2-fluoro-N,3,3-trimethyl-N-(methylsulfonyl) butanamide (80 mg, 36%).

### Step 3. 2-(5-Cyclopropyl-4-((S)-4-((R)-2-fluoro-3,3-dimethylbutanoyl)-2-methylpiperazin-1-yl)-7H-pyrrolo[2,3-d]pyrimidin-7-yl)isonicotinonitrile and 2-(5-cyclopropyl-4-((S)-4-((S)-2-fluoro-3,3-dimethylbutanoyl)-2-methylpiperazin-1-yl)-7H-pyrrolo[2,3-d]pyrimidin-7-yl)isonicotinonitrile

A mixture of (S)-2-(5-cyclopropyl-4-(2-methylpiperazin-1-yl)-7H-pyrrolo[2,3-d]pyrimidin-7-yl)isonicotinonitrile (110 mg, 0.31 mmol, prepared following the procedure outlined for compound 264) and 2-fluoro-N,3,3-trimethyl-N-(methylsulfonyl) butanamide (70 mg, 0.31 mmol) in THF (5 mL) was stirred at 80° C. for 12 h. After cooling to room temperature, the solvent was removed and the residue was purified by flash chromatography (silica gel, 0˜50% ethyl acetate in petroleum ether) and prep-HPLC to afford two isomers: Peak 1 was assigned as 2-(5-cyclopropyl-4-((S)-4-((R)-2-fluoro-3,3-dimethylbutanoyl)-2-methylpiperazin-1-yl)-7H-pyrrolo[2,3-d]pyrimidin-7-yl)isonicotinonitrile compound 202 (5.0 mg, 3.0%) without additional confirmation. The material was isolated as a white solid. LC/MS ESI (m/z): 476 (M+H)⁺. ¹H NMR (400 MHZ, CDCl₃) δ 9.32 (s, 1H), 8.58 (d, J=4.9 Hz, 1H), 8.52 (s, 1H), 7.80 (d, J=0.8 Hz, 1H), 7.35 (dd, J=5.0, 1.3 Hz, 1H), 5.00-4.83 (m, 1H), 4.83-4.61 (m, 1H), 4.44-4.37 (m, 1H), 4.22 (d, J=12.8 Hz, ˜0.5H), 4.02-3.88 (m, 1H), 3.75-3.54 (m, 2H), 3.45-3.38 (m, ˜0.5H), 3.25-3.17 (m, 1H), 2.05-2.01 (m, 1H), 1.32 (d, J=6.6 Hz, ˜1.5H), 1.20 (d, J=6.7 Hz, ˜1.5H), 1.11 and 1.10 (pair of s, 9H), 1.06-1.02 (m, 2H), 0.89-0.85 (m, 1H), 0.80-0.74 (m, 1H); several peaks and their integrations are fractions of protons on account of presence of rotomers.

Peak 2 was assigned as 2-(5-cyclopropyl-4-((S)-4-((S)-2-fluoro-3,3-dimethylbutanoyl)-2-methylpiperazin-1-yl)-7H-pyrrolo[2,3-d]pyrimidin-7-yl)isonicotinonitrile compound 203 (5.0 mg, 0.3%) without additional confirmation. The material was isolated as a white solid. LC/MS ESI (m/z): 476 (M+H)⁺. ¹H NMR (400 MHZ, CDCl₃) δ 9.32 (s, 1H), 8.58 (d, J=5.0 Hz, 1H), 8.51 (d, J=2.2 Hz, 1H), 7.80 (d, J=2.5 Hz, 1H), 7.35 (m, 1H), 4.98-4.67 (m, 2H), 4.49-4.39 (m, 1H), 4.20-4.10 (m, 1H), 4.05-3.92 (m, 1H), 3.63-3.45 (m, 2H), 3.23-3.10 (m, 1H), 2.05-1.99 (m, 1H), 1.30 (d, J=6.7 Hz, ˜1.5H), 1.21 (d, J=6.7 Hz, ˜1.5H), 1.13 and 1.09 (pair of s, 9H), 1.06-1.02 (m, 2H), 0.89-0.86 (m, 1H), 0.80-0.75 (m, 1H); several peaks and their integrations are fractions of protons on account of presence of rotomers.

### Example 38. Synthesis of 1-((2R,5S)-4-(7-(3-chlorophenyl)-5-(trifluoromethyl)-7H-pyrrolo[2,3-d]pyrimidin-4-yl)-2,5-dimethylpiperazin-1-yl)-2,2-dimethylpropan-1-one (Compound 204)

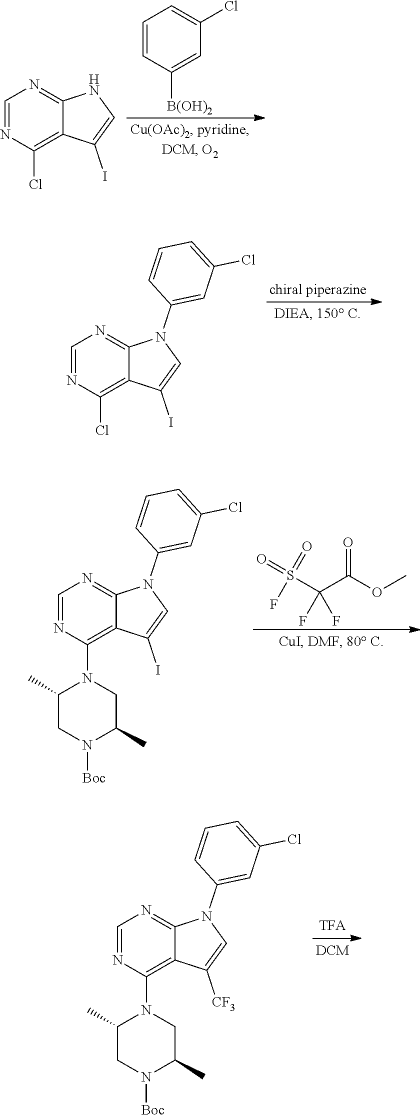 [SMILES: Clc1ncnc2[nH]cc(I)c12 ; OB(O)c1cccc(Cl)c1 ; Clc1cccc(-n2cc(I)c3c(Cl)ncnc32)c1 ; C[C@@H]1CN(c2ncnc3c2c(I)cn3-c2cccc(Cl)c2)[C@@H](C)CN1C(=O)OC(C)(C)C ; COC(=O)C(F)(F)S(=O)(=O)F ; C[C@@H]1CN(c2ncnc3c2c(C(F)(F)F)cn3-c2cccc(Cl)c2)[C@@H](C)CN1C(=O)OC(C)(C)C]

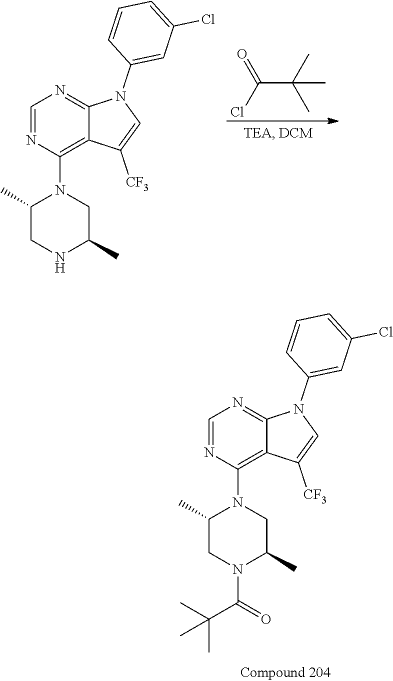

### Step 1. 4-Chloro-7-(3-chlorophenyl)-5-iodo-7H-pyrrolo[2,3-d]pyrimidine

To a solution of 4-chloro-5-iodo-7H-pyrrolo[2,3-d]pyrimidine (10 g, 36 mmol) in DCM (400 mL) were added (3-chlorophenyl) boronic acid (8.7 g, 72 mmol), 4 Å molecular sieves (5.0 g), Cu(OAc)₂ (16 g, 89 mmol) and pyridine (17 mL, 210 mmol). The resulting mixture was stirred at room temperature under O₂ atmosphere for 48 hours. The reaction was then quenched with aq. NH₄OH (30 mL) in an ice water bath and filtered. The filtrate was extracted twice with DCM twice. The combined organic layers were dried over Na₂SO₄, filtered and concentrated. The residue was purified by flash column chromatography (silica gel, 0˜30% ethyl acetate in petroleum ether) to afford 4-chloro-7-(3-chlorophenyl)-5-iodo-7H-pyrrolo[2,3-d]pyrimidine (7.5 g, 54%) as a white solid. LC/MS ESI (m/z): 390 (M+H)⁺.

### Step 2. tert-Butyl (2R,5S)-4-(7-(3-chlorophenyl)-5-iodo-7H-pyrrolo[2,3-d]pyrimidin-4-yl)-2,5-dimethylpiperazine-1-carboxylate

To a solution of 4-chloro-7-(3-chlorophenyl)-5-iodo-7H-pyrrolo[2,3-d]pyrimidine (5.0 g, 13 mmol) in DIPEA (50 mL) was added tert-butyl (2R,5S)-2,5-dimethylpiperazine-1-carboxylate (5.5 g, 26 mmol). After stirring at 150° C. for 2 h under N₂, the reaction was concentrated and purified by flash column chromatography (silica gel, 0˜20% ethyl acetate in petroleum ether) to afford tert-butyl (2R,5S)-4-(7-(3-chlorophenyl)-5-iodo-7H-pyrrolo[2,3-d]pyrimidin-4-yl)-2,5-dimethylpiperazine-1-carboxylate (7.0 g, 89%) as a white solid. LC/MS ESI (m/z): 568 (M+H)⁺.

### Step 3. tert-Butyl (2R,5S)-4-(7-(3-chlorophenyl)-5-(trifluoromethyl)-7H-pyrrolo[2,3-d]pyrimidin-4-yl)-2,5-dimethylpiperazine-1-carboxylate

To a solution of tert-butyl (2R,5S)-4-(7-(3-chlorophenyl)-5-iodo-7H-pyrrolo[2,3-d]pyrimidin-4-yl)-2,5-dimethylpiperazine-1-carboxylate (1.0 g, 1.8 mmol) in DMF (10 mL) were added methyl 2,2-difluoro-2-(fluorosulfonyl)acetate (1.1 g, 5.6 mmol) and CuI (38 mg, 0.20 mmol). The resulting mixture was heated at 80° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜30% ethyl acetate in petroleum ether) to afford tert-butyl (2R,5S)-4-(7-(3-chlorophenyl)-5-(trifluoromethyl)-7H-pyrrolo[2,3-d]pyrimidin-4-yl)-2,5-dimethylpiperazine-1-carboxylate (600 mg, 67%) as a white solid. LC/MS ESI (m/z): 510 (M+H)⁺.

### Step 4. 7-(3-Chlorophenyl)-4-((2S,5R)-2,5-dimethylpiperazin-1-yl)-5-(trifluoromethyl)-7H-pyrrolo[2,3-d]pyrimidine

To a solution of tert-butyl (2R,5S)-4-(7-(3-chlorophenyl)-5-(trifluoromethyl)-7H-pyrrolo[2,3-d]pyrimidin-4-yl)-2,5-dimethylpiperazine-1-carboxylate (480 mg, 0.94 mmol) in DCM (2 mL) was added TFA (2 mL). The resulting mixture was stirred at room temperature for 2 h. After removal of solvent, the residue was diluted with DCM and washed with NaHCO₃ (aq.). The organic layer was washed with brine, dried over Na₂SO₄, filtered and concentrated. The residue was used in the next step directly. LC/MS ESI (m/z): 410 (M+H)⁺.

### Step 5. tert-Butyl (2R,5S)-4-(7-(3-chlorophenyl)-5-(trifluoromethyl)-7H-pyrrolo[2,3-d]pyrimidin-4-yl)-2,5-dimethylpiperazine-1-carboxylate

To a solution of 7-(3-chlorophenyl)-4-((2S,5R)-2,5-dimethylpiperazin-1-yl)-5-(trifluoromethyl)-7H-pyrrolo[2,3-d]pyrimidine (180 mg, 0.44 mmol) in DCM (10 mL) at 0° C. were added TEA (55 mg, 0.54 mmol) and pivaloyl chloride (130 mg, 1.2 mmol). The resulting mixture was stirred at room temperature for 2 h. The reaction was diluted with DCM and washed with brine. The aqueous layer was extracted twice with DCM. The combined organic layers were washed with brine, dried over Na₂SO₄, filtered and concentrated. The residue was purified by flash column chromatography (silica gel, 0˜50% ethyl acetate in petroleum ether) to give crude product which was further purified by prep-HPLC to afford 1-((2R,5S)-4-(7-(3-chlorophenyl)-5-(trifluoromethyl)-7H-pyrrolo[2,3-d]pyrimidin-4-yl)-2,5-dimethylpiperazin-1-yl)-2,2-dimethylpropan-1-one (92 mg, 43%) as a white solid. LC/MS ESI (m/z): 494 (M+H)⁺. ¹H NMR (400 MHz, CDCl₃) δ 8.53 (s, 1H), 7.76-7.69 (m, 2H), 7.61-7.56 (m, 1H), 7.52-7.41 (m, 2H), 4.78-4.54 (m, 2H), 4.40-3.91 (m, 1H), 3.83-3.49 (m, 3H), 1.34 (s, 9H), 1.28-1.05 (m, 6H).

### Example 39. Synthesis of 1-((2R,5S)-4-(5-cyclopropyl-7-(3,5-difluorophenyl)-7H-pyrrolo[2,3-d]pyrimidin-4-yl)-2,5-dimethylpiperazin-1-yl)-2,2-dimethylpropan-1-one (Compound 205)

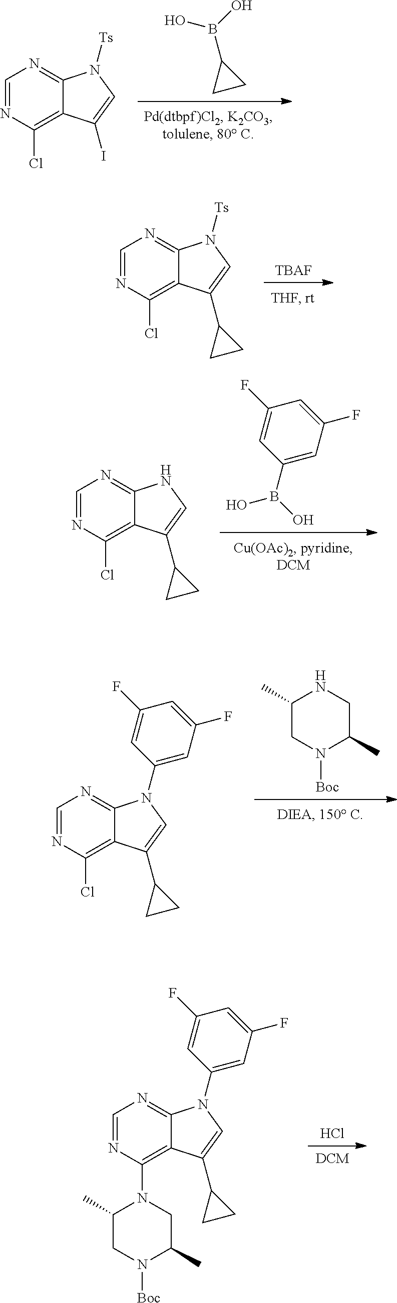

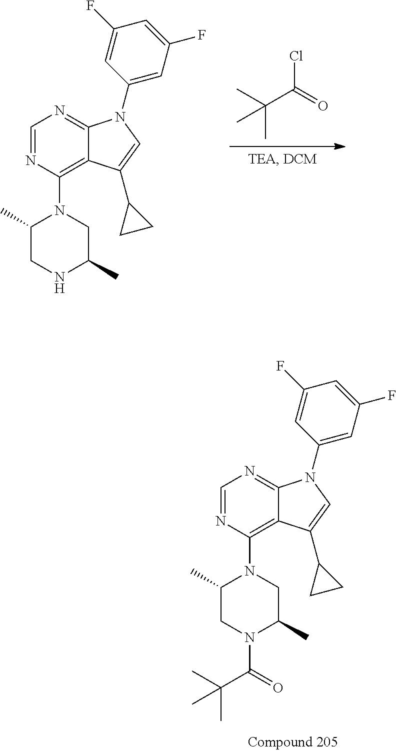

### Step 1. 4-Chloro-5-cyclopropyl-7-tosyl-7H-pyrrolo[2,3-d]pyrimidine

To a solution of 4-chloro-5-iodo-7-tosyl-7H-pyrrolo[2,3-d]pyrimidine (5.8 g, 13 mmol, prepared following the procedure outlined for compound 230) in toluene (50 mL) were added cyclopropylboronic acid (1.1 g, 13 mmol), K₂CO₃ (24 g, 170 mmol) and Pd(dtbpf)Cl₂ (880 mg, 1.3 mmol). The resulting mixture was heated at 80° C. overnight. After cooling to room temperature, the reaction mixture was filtered and concentrated. Purification by flash column chromatography (silica gel, 0˜30% ethyl acetate in petroleum ether) afforded 4-chloro-5-cyclopropyl-7-tosyl-7H-pyrrolo[2,3-d]pyrimidine (3.6 g, 77% yield) as a solid. LC/MS ESI (m/z): 348 (M+H)⁺.

### Step 2. 4-Chloro-5-cyclopropyl-7H-pyrrolo[2,3-d]pyrimidine

To a solution of 4-chloro-5-cyclopropyl-7-tosyl-7H-pyrrolo[2,3-d]pyrimidine (3.6 g, 10 mmol) in THF (50 mL) at 0° C. was added TBAF (20 mL, 1.0 M in THF). After stirring at 0° C. for 5 h, the reaction was diluted with water and extracted twice with EtOAc. The combined organic layers were washed with brine, dried over Na₂SO₄, filtered and concentrated. The residue was purified by flash column chromatography (silica gel, 0˜10% methanol in dichloromethane) to afford 4-chloro-5-cyclopropyl-7H-pyrrolo[2,3-d]pyrimidine (1.8 g, 90%) as a white solid. LC/MS ESI (m/z): 194 (M+H)⁺.

### Step 3. 4-Chloro-5-cyclopropyl-7-(3,5-difluorophenyl)-7H-pyrrolo[2,3-d]pyrimidine

To a solution of 4-chloro-5-cyclopropyl-7H-pyrrolo[2,3-d]pyrimidine (780 mg, 4.0 mmol) in DCM (20 mL) were added (3,5-difluorophenyl) boronic acid (1.3 g, 8.0 mmol), pyridine (1.6 mL, 20 mmol) and Cu(OAc)₂ (3.2 g, 16 mmol). The resulting mixture was stirred at room temperature under O₂ atmosphere for 48 h. The reaction was quenched with aq. NH₄OH and filtered. The filtrate was washed with water and brine, dried over Na₂SO₄, filtered and concentrated. The residue was purified by flash column chromatography (silica gel, 0˜30% ethyl acetate in petroleum ether) to afford 4-chloro-5-cyclopropyl-7-(3,5-difluorophenyl)-7H-pyrrolo[2,3-d]pyrimidine (400 mg, 33%) as a solid. LC/MS ESI (m/z): 306 (M+H)⁺.

### Step 4. tert-Butyl (2R,5S)-4-(5-cyclopropyl-7-(3,5-difluorophenyl)-7H-pyrrolo[2,3-d]pyrimidin-4-yl)-2,5-dimethylpiperazine-1-carboxylate

To a solution of tert-butyl (2R,5S)-2,5-dimethylpiperazine-1-carboxylate (220 mg, 1.0 mmol) in DIEA (0.33 mL, 2.0 mmol) was added tert-butyl (2R,5S)-4-(5-cyclopropyl-7-(3,5-difluorophenyl)-7H-pyrrolo[2,3-d]pyrimidin-4-yl)-2,5-dimethylpiperazine-1-carboxylate (150 mg, 0.50 mmol). The resulting mixture was stirred at 150° C. for 1 h. After cooling to room temperature, DIEA was removed and the residue was purified by column chromatography (silica gel, 0˜30% ethyl acetate in petroleum ether) to give tert-butyl (2R,5S)-4-(5-cyclopropyl-7-(3,5-difluorophenyl)-7H-pyrrolo[2,3-d]pyrimidin-4-yl)-2,5-dimethylpiperazine-1-carboxylate (220 mg, 91%) as a white solid. LC/MS ESI (m/z): 484 (M+H)⁺.

### Step 5. 5-Cyclopropyl-7-(3,5-difluorophenyl)-4-((2S,5R)-2,5-dimethylpiperazin-1-yl)-7H-pyrrolo[2,3-d]pyrimidine

To a solution of tert-butyl (2R,5S)-4-(5-cyclopropyl-7-(3,5-difluorophenyl)-7H-pyrrolo[2,3-d]pyrimidin-4-yl)-2,5-dimethylpiperazine-1-carboxylate (220 mg, 0.45 mmol) in DCM (3.0 mL) was added HCl (2.3 mL, 4.0 M in dioxane). After 2 h, the mixture was concentrated, and the residue was used directly into the next step. LC/MS ESI (m/z): 384 (M+H)⁺.

### Step 6. 1-((2R,5S)-4-(5-Cyclopropyl-7-(3,5-difluorophenyl)-7H-pyrrolo[2,3-d]pyrimidin-4-yl)-2,5-dimethylpiperazin-1-yl)-2,2-dimethylpropan-1-one

To a solution of 5-cyclopropyl-7-(3,5-difluorophenyl)-4-((2S,5R)-2,5-dimethylpiperazin-1-yl)-7H-pyrrolo[2,3-d]pyrimidine (80 mg, 0.21 mmol) and TEA (0.14 mL, 1.0 mmol) in DCM (2.0 mL) in an ice water bath was added 2,2-dimethylpropanoyl chloride (0.05 mL, 0.42 mmol). After 1 h, the reaction was quenched with water and extracted twice with DCM. The combined organic layers were dried over anhydrous Na₂SO₄, filtered and concentrated. The residue was purified by prep-HPLC to give 1-((2R,5S)-4-(5-cyclopropyl-7-(3,5-difluorophenyl)-7H-pyrrolo[2,3-d]pyrimidin-4-yl)-2,5-dimethylpiperazin-1-yl)-2,2-dimethylpropan-1-one (50 mg, 51%) as a white solid. LC/MS ESI (m/z): 468 (M+H)⁺. ¹H NMR (400 MHZ, CDCl₃) δ 8.44 (s, 1H), 7.39-7.32 (m, 2H), 6.94 (d, J=0.8 Hz, 1H), 6.77 (m, 1H), 5.02-4.91 (m, 1H), 4.71 (br. s, 1H), 4.37-4.04 (br. s, 1H), 3.86-3.72 (m, 2H), 3.69-3.41 (m, 1H), 2.07-1.98 (m, 1H), 1.34 (s, 9H), 1.25 (m, 3H), 1.14 (m, 3H), 1.07-1.02 (m, 2H), 0.91-0.84 (m, 1H), 0.69-0.63 (m, 1H).

The following compound was prepared by a procedure analogous to the synthesis of compound 205 from the corresponding arylboronic acid.

| Cmpd | | |
|---|---|---|
| No. | Chemical Name | LCMS and ¹H NMR |
| 206 | 1-((2R,5S)-4-(7-(3- | LC/MS ESI (m/z): 466 (M + H)⁺. ¹H NMR (400 MHz, |
| | chlorophenyl)-5-cyclopropyl- | CDCl₃) δ 8.44 (s, 1H), 7.68 (t, J = 2.0 Hz, 1H), 7.61 (m, |
| | 7H-pyrrolo[2,3-d]pyrimidin-4- | 1H), 7.42 (t, J = 8.1 Hz, 1H), 7.31 (m, 1H), 6.95 (d, J = 0.8 |
| | yl)-2,5-dimethylpiperazin-1-yl)- | Hz, 1H), 5.03-4.91 (m, 1H), 4.71 (br. s, 1H), 4.37-4.05 |
| | 2,2-dimethylpropan-1-one | (br. s, 1H), 3.84-3.71 (m, 2H), 3.58 (m, 1H), 2.09-1.97 |
| | | (m, 1H), 1.34 (s, 9H), 1.26 (m, 3H), 1.13 (m, 3H), 1.06- |
| | | 0.99 (m, 2H), 0.92-0.83 (m, 1H), 0.70-0.60 (m, 1H). |

### Example 40. Synthesis of 2-(4-((2S,5R)-2,5-dimethyl-4-pivaloylpiperazin-1-yl)-5-(trifluoromethyl)-7H-pyrrolo[2,3-d]pyrimidin-7-yl)isonicotinonitrile (Compound 207)

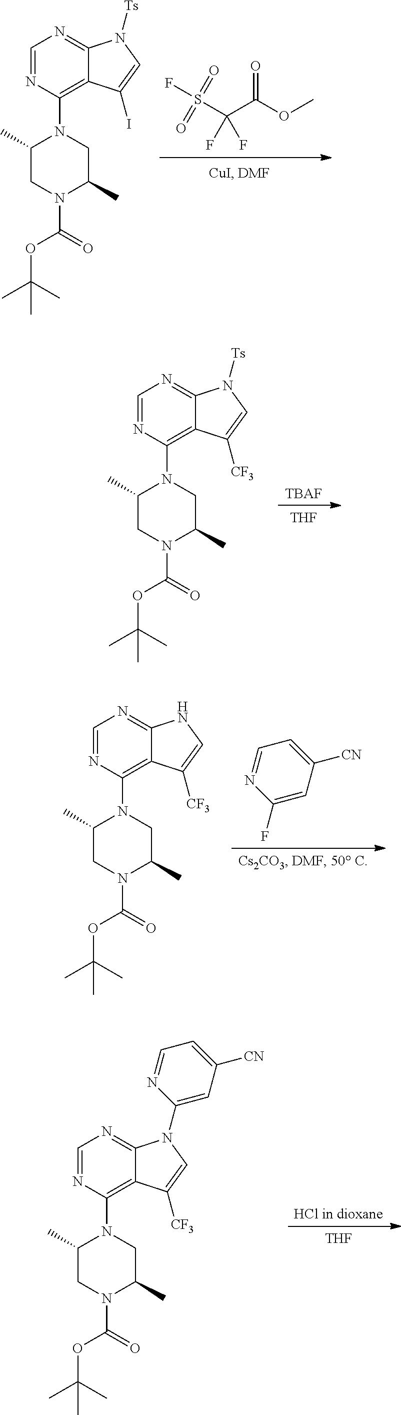

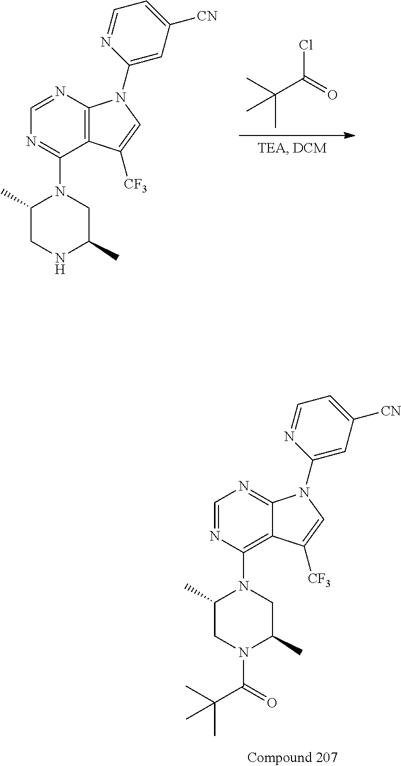

### Step 1. tert-Butyl (2R,5S)-2,5-dimethyl-4-(7-tosyl-5-(trifluoromethyl)-7H-pyrrolo[2,3-d]pyrimidin-4-yl)piperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (26 g, 43 mmol, prepared following the procedure outlined for compound 230) in DMF (50 mL) were added methyl 2,2-difluoro-2-(fluorosulfonyl)acetate (24 g, 130 mmol) and CuI (8.0 g, 43 mmol). The resulting mixture was heated at 80° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜30% ethyl acetate in petroleum ether) to afford tert-butyl (2R,5S)-2,5-dimethyl-4-(7-tosyl-5-(trifluoromethyl)-7H-pyrrolo[2,3-d]pyrimidin-4-yl)piperazine-1-carboxylate (13 g, 55%) as a yellow oil. LC/MS ESI (m/z): 554 (M+H)⁺.

### Step 2. tert-Butyl (2R,5S)-2,5-dimethyl-4-(5-(trifluoromethyl)-7H-pyrrolo[2,3-d]pyrimidin-4-yl)piperazine-1-carboxylate

To a solution of tert-butyl (2R,5S)-2,5-dimethyl-4-(7-tosyl-5-(trifluoromethyl)-7H-pyrrolo[2,3-d]pyrimidin-4-yl)piperazine-1-carboxylate (12 g, 22 mmol) in THF (10 mL) was added TBAF (42 mL, 1.0 M in THF). After 2 h, the reaction was diluted with water and extracted twice with EtOAc. The combined organic layers were washed with brine, dried over Na₂SO₄, filtered and concentrated. The residue was purified by flash column chromatography (silica gel, 0˜50% ethyl acetate in petroleum ether) to afford tert-butyl (2R,5S)-2,5-dimethyl-4-(5-(trifluoromethyl)-7H-pyrrolo[2,3-d]pyrimidin-4-yl)piperazine-1-carboxylate (7.5 g, 87%) as a yellow oil. LC/MS ESI (m/z): 400 (M+H)⁺.

### Step 3. tert-Butyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate

To a solution of tert-butyl (2R,5S)-2,5-dimethyl-4-(5-(trifluoromethyl)-7H-pyrrolo[2,3-d]pyrimidin-4-yl)piperazine-1-carboxylate (1.2 g, 3.0 mmol) in DMF (200 ml) were added Cs₂CO₃ (2.0 g, 6.0 mmol) and 2-fluoroisonicotinonitrile (1.1 g, 9.0 mmol) at 25° C. The resulting mixture was heated to 50° C. and stirred for 2 h. After cooling to room temperature, the reaction mixture was poured into H₂O (100 ml) and extracted with EtOAc (3×100 mL). The combined organic phases were washed twice with NH₄Cl (aq.) and brine, dried over Na₂SO₄, filtered, and concentrated in vacuo. The crude product was purified by flash column chromatography (silica gel, 0˜30% ethyl acetate in petroleum ether) to afford tert-butyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate (1.4 g, 92%) as a white solid. LC/MS ESI (m/z): 502 (M+H)⁺. ¹H NMR (400 MHz, CDCl₃) δ 9.27 (d, J=0.9 Hz, 1H), 8.60 (d, J=5.0 Hz, 1H), 8.57 (s, 1H), 8.50 (s, 1H), 7.43-7.39 (m, 1H), 4.52 (s, 1H), 4.31 (s, 1H), 3.76-3.63 (m, 2H), 3.59-3.52 (m, 1H), 3.41 (d, J=13.3 Hz, 1H), 1.42 (s, 9H), 1.09 (d, J=6.6 Hz, 3H), 1.03 (d, J=6.8 Hz, 3H).

### Step 4. 2-(4-((2S,5R)-2,5-Dimethylpiperazin-1-yl)-5-(trifluoromethyl)-7H-pyrrolo[2,3-d]pyrimidin-7-yl)isonicotinonitrile

To a solution of tert-butyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate (1.4 g, 2.8 mmol) in THF (30 mL) was added HCl in dioxane (4.0 mL, 4.0 M in dioxane). After 16 h, the reaction mixture was concentrated in vacuo to give 1.5 g of 2-(4-((2S,5R)-2,5-dimethylpiperazin-1-yl)-5-(trifluoromethyl)-7H-pyrrolo[2,3-d]pyrimidin-7-yl)isonicotinonitrile as a HCl salt which was used in the next step without further purification. LC/MS ESI (m/z): 402 (M+H)⁺.

### Step 5. 2-(4-((2S,5R)-2,5-Dimethyl-4-pivaloylpiperazin-1-yl)-5-(trifluoromethyl)-7H-pyrrolo[2,3-d]pyrimidin-7-yl)isonicotinonitrile

To a solution of 2-(4-((2S,5R)-2,5-dimethylpiperazin-1-yl)-5-(trifluoromethyl)-7H-pyrrolo[2,3-d]pyrimidin-7-yl)isonicotinonitrile (60 mg, 0.15 mmol) and TEA (0.10 ml, 0.75 mmol) in DCM (10 mL) at 0° C. was added pivaloyl chloride (22 mg, 0.18 mmol). After 20 minutes, the reaction was quenched with ice water and extracted twice with DCM. The combined layers were washed with brine, dried over Na₂SO₄, filtered and concentrated. The residue was purified by column chromatography on silica gel (0˜30% ethyl acetate in petroleum ether) to give the crude product. The crude product was further purified by prep-HPLC to give the 2-(4-((2S,5R)-2,5-dimethyl-4-pivaloylpiperazin-1-yl)-5-(trifluoromethyl)-7H-pyrrolo[2,3-d]pyrimidin-7-yl)isonicotinonitrile (45 mg, 62%) as a white solid. LC/MS ESI (m/z): 486 (M+H)⁺. ¹H NMR (400 MHZ, CDCl₃) δ 9.34 (s, 1H), 8.72-8.65 (m, 2H), 8.59 (s, 1H), 7.49 (dd, J=5.0, 1.3 Hz, 1H), 4.76-4.56 (m, 2H), 4.16 (br. s, 1H), 3.86-3.57 (m, 2H), 3.52 (d, J=13.4 Hz, 1H), 1.34 (s, 9H), 1.24-1.07 (m, 6H).

The following compounds were prepared by procedures analogous to the synthesis of compound 207 from the corresponding carboxylic acid chloride.

| Cmpd | | |
|---|---|---|
| No. | Chemical Name | LCMS and ¹H NMR |
| 208 | 2-(4-((2S,5R)-4-(2-methoxy-2- | LC/MS ESI (m/z): 502 (M + H)⁺. ¹H NMR (400 MHz, |
| | methylpropanoyl)-2,5- | DMSO-d6) δ 9.04 (s, 1H), 8.86 (dd, J = 5.0, 0.7 Hz, 1H), |
| | dimethylpiperazin-1-yl)-5- | 8.75 (s, 1H), 8.66 (s, 1H), 7.96 (dd, J = 5.0, 1.3 Hz, 1H), |
| | (trifluoromethyl)-7H- | 5.13 (br. s, ~0.4H), 4.62 (br. s, ~0.6H), 4.52 (br. s, 1H), |
| | pyrrolo[2,3-d]pyrimidin-7- | 4.31 (m, 1H), 3.98-3.43 (m, 3H), 3.25 (s, 1H), 3.20 (s, |
| | yl)isonicotinonitrile | 2H), 1.46-1.33 (m, 6H), 1.22 (d, J = 4 Hz, 1H), 1.15 (d, |
| | | J = 8 Hz, 2H), 0.99 (m, 3H); several peaks and their |
| | | integrations are fractions of protons on account of presence |
| | | of rotomers. |
| 209 | 2-(4-((2S,5R)-4-(3,3- | LC/MS ESI (m/z): 500 (M + H)⁺. ¹H NMR(400 MHz, |
| | dimethylbutanoyl)-2,5- | CDCl3) δ 9.34 (s, 1H), 8.69-8.65 (m, 2H), 8.59 (d, J = 7.4 |
| | dimethylpiperazin-1-yl)-5- | Hz, 1H), 7.49 (d, J = 4.2 Hz, 1H), 4.94 (br. s, ~0.5H), 4.65 |
| | (trifluoromethyl)-7H- | (br. s, ~0.5 H), 4.59 (br. s, ~0.5H), 4.37 (d, J = 12 |
| | pyrrolo[2,3-d]pyrimidin-7- | Hz, ~0.5H), 4.25 (br. s, ~0.5H), 3.88 (m, ~0.5H), 3.77 (m, 1H), |
| | yl)isonicotinonitrile | 3.63-3.45 (m, 2H), 2.49 (d, J = 12 Hz, ~0.5H), 2.38 (d, J = |
| | | 16 Hz, ~0.5 H), 2.18 (m, 1H), 1.22 (m, 3H), 1.12-1.07 (m, |
| | | 12H); several peaks and their integrations are fractions of |
| | | protons on account of presence of rotomers. |
| 210 | (S)-2-(4-(4-(2-methoxy-2- | LC/MS ESI (m/z): 488 (M + H)⁺. ¹H NMR(400 MHz, |
| | methylpropanoyl)-2- | CD3OD) δ 9.20 (s, 1H), 8.81-8.60 (m, 3H), 7.70 (dd, J = |
| | methylpiperazin-1-yl)-5- | 5.0, 1.2 Hz, 1H), 4.72-4.22 (m, 2H), 4.12-3.93 (m, 1H), |
| | (trifluoromethyl)-7H- | 3.86-3.52 (m, 3H), 3.50-3.36 (m, 1H), 3.29 (s, 3H), 1.48 |
| | pyrrolo[2,3-d]pyrimidin-7- | (s, 3H), 1.45 (s, 3H), 1.17 (d, J = 5.8 Hz, 3H). |
| | yl)isonicotinonitrile | |

### Example 41. Synthesis of (S)-2-(4-(4-isobutyryl-2-methylpiperazin-1-yl)-5-(pyridin-2-yl)-5H-pyrrolo[3,2-d]pyrimidin-7-yl)isonicotinonitrile (Compound 211)

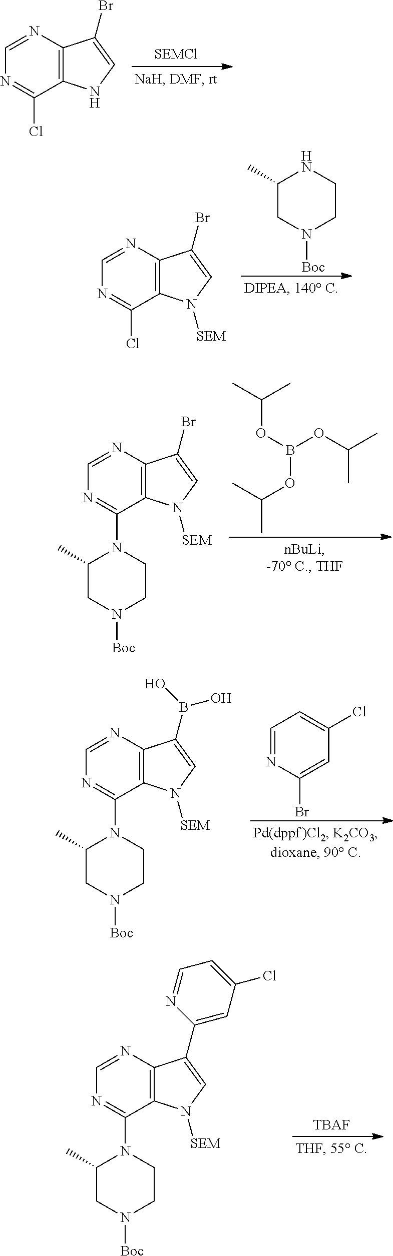

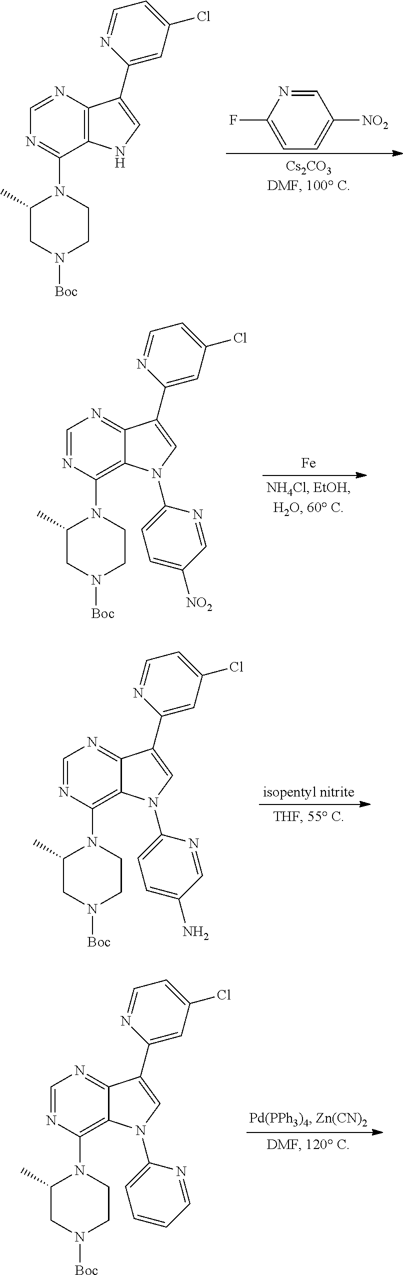

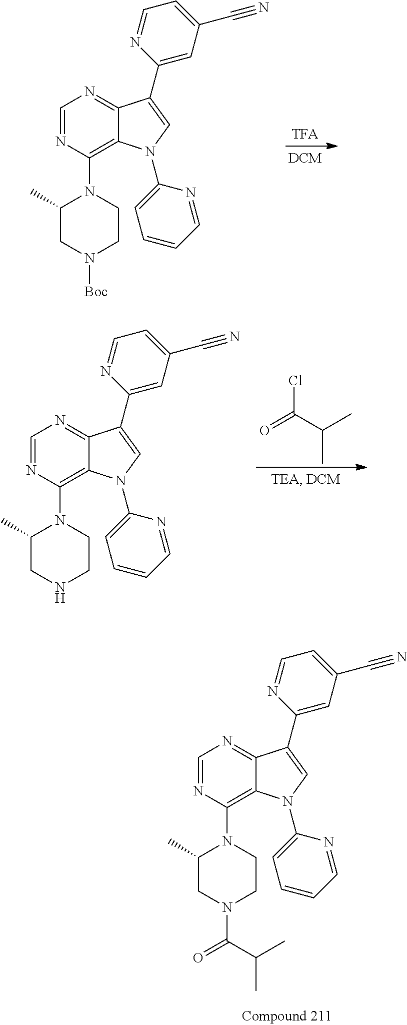

### Step 1. 7-Bromo-4-chloro-5-((2-(trimethylsilyl)ethoxy)methyl)-5H-pyrrolo[3,2-d]pyrimidine

To a solution of 7-bromo-4-chloro-5H-pyrrolo[3,2-d]pyrimidine (2.0 g, 8.6 mmol) in DMF (20 mL) at 0° C. were added NaH (410 mg, 17 mmol, 60 wt % in mineral oil) in portions followed by SEM-C₁ (1.6 g, 9.5 mmol) dropwise. The resulting mixture was stirred at 0° C. for 30 min and then at room temperature for 2 h. The reaction was then quenched with ice water and extracted twice with EtOAc. The combined organic layers were dried over Na₂SO₄, filtered and concentrated. The residue was purified by flash column chromatography (0˜10% ethyl acetate in petroleum ether) to afford 7-bromo-4-chloro-5-((2-(trimethylsilyl)ethoxy)methyl)-5H-pyrrolo[3,2-d]pyrimidine (2.8 g, 90%) as a solid. LC/MS ESI (m/z): 362, 364 (M+H)⁺.

### Step 2. tert-Butyl (S)-4-(7-bromo-5-((2-(trimethylsilyl)ethoxy)methyl)-5H-pyrrolo[3,2-d]pyrimidin-4-yl)-3-methylpiperazine-1-carboxylate

To a solution of 7-bromo-4-chloro-5-((2-(trimethylsilyl)ethoxy)methyl)-5H-pyrrolo[3,2-d]pyrimidine (2.8 g, 7.8 mmol) in DIPEA (5 mL) was added tert-butyl (S)-3-methylpiperazine-1-carboxylate (3.1 g, 16 mmol). The resulting mixture was heated at 140° C. for 1.5 h. After cooling to room temperature, solvent was removed and the residue was purified by flash column chromatography (silica gel, 0˜15% ethyl acetate in petroleum ether) to afford tert-butyl (S)-4-(7-bromo-5-((2-(trimethylsilyl)ethoxy)methyl)-5H-pyrrolo[3,2-d]pyrimidin-4-yl)-3-methylpiperazine-1-carboxylate (2.9 g, 72%) as a solid. LC/MS ESI (m/z): 526, 528 (M+H)⁺.

### Step 3. (S)-(4-(4-(tert-Butoxycarbonyl)-2-methylpiperazin-1-yl)-5-((2-(trimethylsilyl)ethoxy)methyl)-5H-pyrrolo[3,2-d]pyrimidin-7-yl) boronic acid

To a solution of tert-butyl (S)-4-(7-bromo-5-((2-(trimethylsilyl)ethoxy)methyl)-5H-pyrrolo[3,2-d]pyrimidin-4-yl)-3-methylpiperazine-1-carboxylate (2.9 g, 5.5 mmol) in THF (15 mL) at −70° C. were added triisopropyl borate (4.2 g, 23 mmol) followed by n-butyllithium (9.0 ml, 2.5 M in hexane) dropwise. The resulting mixture was stirred at −70° C. for 1 h. The reaction was then quenched with aq. NH₄Cl solution carefully and extracted twice with EtOAc. The combined organic layers were dried over Na₂SO₄, filtered, and concentrated to afford the crude product which was used in the next step directly.

### Step 4. tert-Butyl (S)-4-(7-(4-chloropyridin-2-yl)-5-((2-(trimethylsilyl)ethoxy)methyl)-5H-pyrrolo[3,2-d]pyrimidin-4-yl)-3-methylpiperazine-1-carboxylate

To a solution of (S)-(4-(4-(tert-butoxycarbonyl)-2-methylpiperazin-1-yl)-5-((2-(trimethylsilyl)ethoxy)methyl)-5H-pyrrolo[3,2-d]pyrimidin-7-yl) boronic acid (from step 3, 5.5 mmol) in dioxane (13 mL) and H₂O (2 mL) were added 2-bromo-4-chloropyridine (1.3 g, 6.6 mmol), K₂CO₃ (6.5 g, 47 mmol) and Pd(dppf)Cl₂ (570 mg, 0.78 mmol). The resulting mixture was heated at 90° C. overnight under N₂. After cooling to room temperature, solvent was removed and the residue was purified by flash column chromatography (silica gel, 0˜40% ethyl acetate in petroleum ether) to afford tert-butyl (S)-4-(7-(4-chloropyridin-2-yl)-5-((2-(trimethylsilyl)ethoxy)methyl)-5H-pyrrolo[3,2-d]pyrimidin-4-yl)-3-methylpiperazine-1-carboxylate (1.0 g, 32% over two steps) as an oil. LC/MS ESI (m/z): 559 (M+H)⁺.

### Step 5. tert-Butyl (S)-4-(7-(4-chloropyridin-2-yl)-5H-pyrrolo[3,2-d]pyrimidin-4-yl)-3-methylpiperazine-1-carboxylate

To a solution of tert-butyl (S)-4-(7-(4-chloropyridin-2-yl)-5-((2-(trimethylsilyl)ethoxy)methyl)-5H-pyrrolo[3,2-d]pyrimidin-4-yl)-3-methylpiperazine-1-carboxylate (1.0 g, 1.8 mmol) in THF (10 mL) was added TBAF (20 mL, 1.0 M in THF). The resulting mixture was heated at 55° C. for 2 h. The reaction was then quenched with water and extracted twice with EtOAc. The combined organic layers were washed with brine, dried over Na₂SO₄, filtered, and concentrated to afford tert-butyl (S)-4-(7-(4-chloropyridin-2-yl)-5H-pyrrolo[3,2-d]pyrimidin-4-yl)-3-methylpiperazine-1-carboxylate (700 mg, 91%) as a solid. LC/MS ESI (m/z): 429 (M+H)⁺.

### Step 6. tert-Butyl (S)-4-(7-(4-chloropyridin-2-yl)-5-(5-nitropyridin-2-yl)-5H-pyrrolo[3,2-d]pyrimidin-4-yl)-3-methylpiperazine-1-carboxylate

To a solution of tert-butyl (S)-4-(7-(4-chloropyridin-2-yl)-5H-pyrrolo[3,2-d]pyrimidin-4-yl)-3-methylpiperazine-1-carboxylate (700 mg, 1.6 mmol) in DMF (10 mL) were added 2-fluoro-5-nitropyridine (690 mg, 4.9 mmol) and Cs₂CO₃ (2.1 g, 6.5 mmol). The resulting mixture was heated at 100° C. for 2 h. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜60%, ethyl acetate in petroleum ether) to afford tert-butyl (S)-4-(7-(4-chloropyridin-2-yl)-5-(5-nitropyridin-2-yl)-5H-pyrrolo[3,2-d]pyrimidin-4-yl)-3-methylpiperazine-1-carboxylate (800 mg, 89%) as a solid. LC/MS ESI (m/z): 551 (M+H)⁺.

### Step 7. tert-Butyl (S)-4-(5-(5-aminopyridin-2-yl)-7-(4-chloropyridin-2-yl)-5H-pyrrolo[3,2-d]pyrimidin-4-yl)-3-methylpiperazine-1-carboxylate

To a solution of tert-butyl (S)-4-(7-(4-chloropyridin-2-yl)-5-(5-nitropyridin-2-yl)-5H-pyrrolo[3,2-d]pyrimidin-4-yl)-3-methylpiperazine-1-carboxylate (700 mg, 1.3 mmol) in EtOH (10 mL) and H₂O (5 mL) were added NH₄Cl (680 mg, 13 mmol) and Fe (710 mg, 13 mmol). The resulting mixture was heated at 60° C. overnight. After cooling to room temperature, the reaction was filtered, and the filtrate was partitioned between EtOAc and water. The aqueous layer was extracted twice with EtOAc. The combined organic layers were dried over Na₂SO₄, filtered and concentrated. The residue was purified by flash column chromatography (silica gel, 0˜80% ethyl acetate in petroleum ether) to afford tert-butyl (S)-4-(5-(5-aminopyridin-2-yl)-7-(4-chloropyridin-2-yl)-5H-pyrrolo[3,2-d]pyrimidin-4-yl)-3-methylpiperazine-1-carboxylate (490 mg, 74%) as a solid. LC/MS ESI (m/z): 521 (M+H)⁺.

### Step 8. tert-Butyl (S)-4-(7-(4-chloropyridin-2-yl)-5-(pyridin-2-yl)-5H-pyrrolo[3,2-d]pyrimidin-4-yl)-3-methylpiperazine-1-carboxylate

To a solution of tert-butyl (S)-4-(5-(5-aminopyridin-2-yl)-7-(4-chloropyridin-2-yl)-5H-pyrrolo[3,2-d]pyrimidin-4-yl)-3-methylpiperazine-1-carboxylate (300 mg, 0.58 mmol) in THF (10 mL) was added isopentyl nitrite (270 mg, 2.3 mmol). The resulting mixture was heated at 55° C. for overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜70%, ethyl acetate in petroleum ether) to afford tert-butyl (S)-4-(7-(4-chloropyridin-2-yl)-5-(pyridin-2-yl)-5H-pyrrolo[3,2-d]pyrimidin-4-yl)-3-methylpiperazine-1-carboxylate (200 mg, 68%) as a solid. LC/MS ESI (m/z): 506 (M+H)⁺.

### Step 9. tert-Butyl (S)-4-(7-(4-cyanopyridin-2-yl)-5-(pyridin-2-yl)-5H-pyrrolo[3,2-d]pyrimidin-4-yl)-3-methylpiperazine-1-carboxylate

To a solution of tert-butyl (S)-4-(7-(4-chloropyridin-2-yl)-5-(pyridin-2-yl)-5H-pyrrolo[3,2-d]pyrimidin-4-yl)-3-methylpiperazine-1-carboxylate (150 mg, 0.30 mmol) in DMF (10 mL) were added Pd(PPh₃)⁺ (170 mg, 0.15 mmol) and Zn(CN)₂ (250 mg, 2.1 mmol). The resulting mixture was heated at 120° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜60% ethyl acetate in petroleum ether) to afford tert-butyl (S)-4-(7-(4-cyanopyridin-2-yl)-5-(pyridin-2-yl)-5H-pyrrolo[3,2-d]pyrimidin-4-yl)-3-methylpiperazine-1-carboxylate (120 mg, 81%) as a solid. LC/MS ESI (m/z): 497 (M+H)⁺.

### Step 10. (S)-2-(4-(2-Methylpiperazin-1-yl)-5-(pyridin-2-yl)-5H-pyrrolo[3,2-d]pyrimidin-7-yl)isonicotinonitrile

To a solution of tert-butyl (S)-4-(7-(4-cyanopyridin-2-yl)-5-(pyridin-2-yl)-5H-pyrrolo[3,2-d]pyrimidin-4-yl)-3-methylpiperazine-1-carboxylate (30 mg, 0.06 mmol) in DCM (3 mL) was added TFA (0.3 mL). The resulting mixture was stirred at room temperature for 1 h. The reaction was quenched with NaHCO₃ (aq.) and extracted twice with DCM. The combined organic layers were washed with brine, dried over Na₂SO₄, filtered and concentrated. The residue was used in the next step directly. LC/MS ESI (m/z): 397 (M+H)⁺.

### Step 11. (S)-2-(4-(4-Isobutyryl-2-methylpiperazin-1-yl)-5-(pyridin-2-yl)-5H-pyrrolo[3,2-d]pyrimidin-7-yl)isonicotinonitrile

To a solution of (S)-2-(4-(2-methylpiperazin-1-yl)-5-(pyridin-2-yl)-5H-pyrrolo[3,2-d]pyrimidin-7-yl)isonicotinonitrile (20 mg, 0.05 mmol) in DCM (3 mL) at 0° C. were added TEA (0.10 mL) and isobutyryl chloride (20 mg, 0.19 mmol). After 1 h, the reaction was partitioned between DCM and water. The aqueous layer was extracted twice with DCM. The combined organic layers were washed with brine, dried over Na₂SO₄, filtered and concentrated. The residue was purified by prep-HPLC to afford (S)-2-(4-(4-isobutyryl-2-methylpiperazin-1-yl)-5-(pyridin-2-yl)-5H-pyrrolo[3,2-d]pyrimidin-7-yl)isonicotinonitrile (10 mg, 43%) as a white solid. LC/MS ESI (m/z): 467 (M+H)⁺. ¹H NMR (400 MHZ, CDCl₃) δ 9.04 (s, 1H), 8.70 (s, 1H), 8.68 (d, J=5.0 Hz, 1H), 8.63 (d, J=7.5 Hz, 1H), 8.57 (br. s, 1H), 7.82 (m, 1H), 7.33-7.29 (m, 2H), 7.20 (m, 1H), 4.28 (br. s, ˜0.5H), 4.14 (d, J=16 Hz, ˜0.5H), 3.97 (d, J=12 Hz, ˜0.5H), 3.86 (br. s, ˜0.5H), 3.65 (d, J=12 Hz, ˜0.5H), 3.56 (d, J=12 Hz, ˜0.5H), 3.43 (d, J=12 Hz, ˜0.5H), 3.28 (d, J=16 Hz, ˜0.5H), 3.10 (m, 1H), 3.05-2.70 (m, 1H), 2.74-2.59 (m, 1H), 2.42 (m, 1H), 1.02 (m, 7.5H), 0.80 (d, J=6.5 Hz, 1.5H); several peaks and their integrations are fractions of protons on account of presence of rotomers.

The following compounds were prepared by the procedure analogous to the synthesis of compound 211 from the corresponding aryl fluoride (1,2-difluoro-4-nitrobenzene) and chiral amine.

| Cmpd | | |
|---|---|---|
| No. | Chemical Name | LCMS and ¹H NMR |
| 212 | (S)-2-(5-(2-fluorophenyl)-4-(4- | LC/MS ESI (m/z): 484 (M + H)⁺. ¹H NMR (400 MHz, |
| | isobutyryl-2-methylpiperazin-1- | CD₃OD) δ 9.02 (s, 1H), 8.76-8.70 (m, 2H), 8.38 (s, 1H), |
| | yl)-5H-pyrrolo[3,2-d]pyrimidin- | 7.76-7.52 (m, 3H), 7.45 (m, 2H), 3.94-3.34 (m, ~3.5H), |
| | 7-yl)isonicotinonitrile | 3.20-2.98 (m, ~2.5H), 2.95-2.76 (m, ~1.5H), 2.49 (br. |
| | | s, ~0.5H), 1.09-1.00 (m, ~7.5H), 0.87 (d, J = 6.4 Hz, ~1.5H); |
| | | several peaks and their integrations are fractions of |
| | | protons on account of presence of rotomers. |
| 213 | 2-(5-(2-fluorophenyl)-4- | LC/MS ESI (m/z): 498 (M + H)⁺. ¹H NMR (400 MHz, |
| | ((2S,5R)-4-isobutyryl-2,5- | CDCl₃) δ 9.13 (s, 1H), 8.74 (br. s, 1H), 8.70 (d, J = 4.9 Hz, |
| | dimethylpiperazin-1-yl)-5H- | 1H), 8.27 (s, 1H), 7.55-7.45 (m, 1H), 7.44-7.25 (m, 4H), |
| | pyrrolo [3,2-d]pyrimidin-7- | 4.65 (br. s, ~0.5H), 4.13 (s, 1H), 3.85 (d, J = 13.3 Hz, ~0.5H), |
| | yl)isonicotinonitrile | 3.69 (br. s, ~0.5H), 3.38 (br. s, 1H), 3.27-2.86 (m, |
| | | 2H), 2.76 (br. s, ~0.5H), 2.58 (br. s, ~0.5H), 2.18-2.01 |
| | | (m, ~0.5H), 1.59-1.05 (m, ~6.5H), 0.98 (m, 4H), 0.77 (br. |
| | | s, ~1.5H); several peaks and their integrations are fractions of |
| | | protons on account of presence of rotomers. |

### Example 42. Synthesis of 1-((2R,5S)-4-(5-cyclopropyl-7-(3-fluorophenyl)-7H-pyrrolo[2,3-d]pyrimidin-4-yl)-2,5-dimethylpiperazin-1-yl)-2,2-dimethylpropan-1-one (Compound 214)

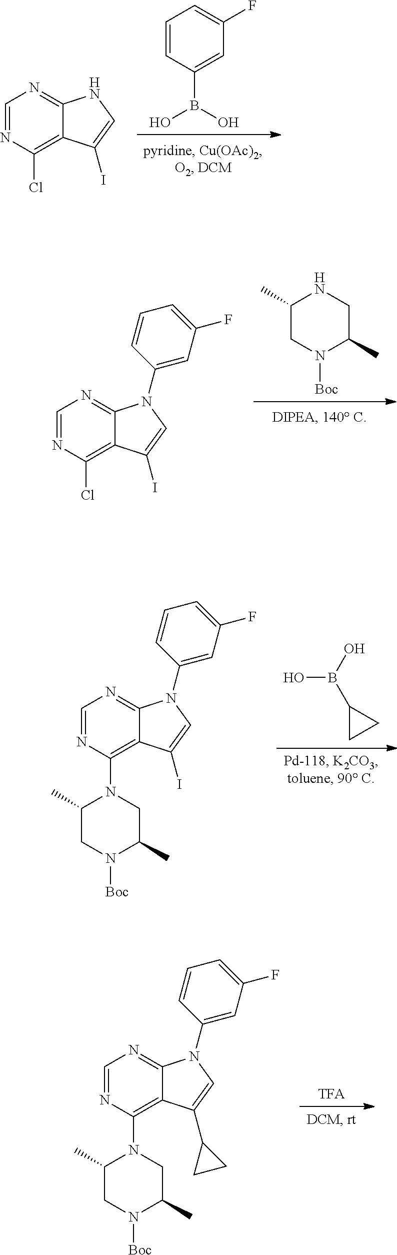

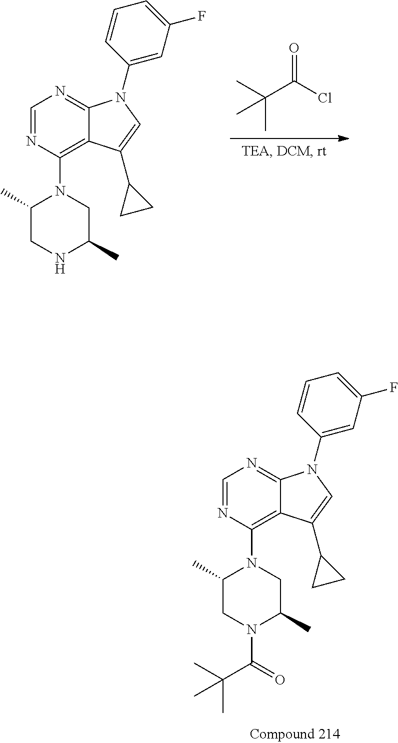

### Step 1. 4-Chloro-7-(3-fluorophenyl)-5-iodo-7H-pyrrolo[2,3-d]pyrimidine

To a solution of 4-chloro-5-iodo-7H-pyrrolo[2,3-d]pyrimidine (6.0 g, 22 mmol) in DCM (60 mL) were added (3-fluorophenyl) boronic acid (6.0 g, 43 mmol), 4 Å molecular sieves (3.0 g), Cu(OAc)₂ (16 g, 86 mmol) and pyridine (10 g, 130 mmol). The resulting mixture was stirred at 30° C. under O₂ atmosphere for 2 days. The reaction was then quenched with aq. NH₄OH (10 mL) in an ice water bath and filtered. The filtrate was extracted twice with DCM. The combined organic layers were dried over Na₂SO₄, filtered and concentrated. The residue was triturated with petroleum ether to afford 4-chloro-7-(3-fluorophenyl)-5-iodo-7H-pyrrolo[2,3-d]pyrimidine (5.0 g, 62%) as a solid. LC/MS ESI (m/z): 374 (M+H)⁺.

### Step 2. tert-Butyl (2R,5S)-4-(7-(3-fluorophenyl)-5-iodo-7H-pyrrolo[2,3-d]pyrimidin-4-yl)-2,5-dimethylpiperazine-1-carboxylate

To a solution of 4-chloro-7-(3-fluorophenyl)-5-iodo-7H-pyrrolo[2,3-d]pyrimidine (1.0 g, 2.7 mmol) in DIPEA (3 mL) was added tert-butyl (2R,5S)-2,5-dimethylpiperazine-1-carboxylate (1.1 g, 5.4 mmol). The resulting reaction mixture was heated at 140° C. under N₂ for 2 h. After cooling to room temperature, solvent was removed and the residue was purified by flash column chromatography (silica gel, 0˜30% ethyl acetate in petroleum ether) to afford tert-butyl (2R,5S)-4-(7-(3-fluorophenyl)-5-iodo-7H-pyrrolo[2,3-d]pyrimidin-4-yl)-2,5-dimethylpiperazine-1-carboxylate (1.0 g, 68%) as a solid. LC/MS ESI (m/z): 552 (M+H)⁺.

### Step 3. tert-Butyl (2R,5S)-4-(5-cyclopropyl-7-(3-fluorophenyl)-7H-pyrrolo[2,3-d]pyrimidin-4-yl)-2,5-dimethylpiperazine-1-carboxylate

To a solution of tert-butyl (2R,5S)-4-(7-(3-fluorophenyl)-5-iodo-7H-pyrrolo[2,3-d]pyrimidin-4-yl)-2,5-dimethylpiperazine-1-carboxylate (250 mg, 0.45 mmol) in toluene (5 mL) were added cyclopropylboronic acid (57 mg, 0.67 mmol), K₂CO₃ (1.2 g, 9.0 mmol) and PdCl₂ (dtbpf) (14 mg, 0.02 mmol). The resulting mixture was heated at 90° C. overnight. After cooling to room temperature, solvents were removed and the residue was purified by flash column chromatography (silica gel, 0˜40% ethyl acetate in petroleum ether) to afford tert-butyl (2R,5S)-4-(5-cyclopropyl-7-(3-fluorophenyl)-7H-pyrrolo[2,3-d]pyrimidin-4-yl)-2,5-dimethylpiperazine-1 carboxylate (170 mg, 81%) as a white solid. LC/MS ESI (m/z): 466 (M+H)⁺.

### Step 4. 5-Cyclopropyl-4-((2S,5R)-2,5-dimethylpiperazin-1-yl)-7-(3-fluorophenyl)-7H-pyrrolo[2,3-d]pyrimidine

To a solution of tert-butyl (2R,5S)-4-(5-cyclopropyl-7-(3-fluorophenyl)-7H-pyrrolo[2,3-d]pyrimidin-4-yl)-2,5-dimethylpiperazine-1-carboxylate (170 mg, 0.36 mmol) in DCM (3 mL) was added TFA (1.0 mL). The resulting mixture was stirred at room temperature for 2 h. After the removal of solvent, the residue was used in the next step directly. LC/MS ESI (m/z): 366 (M+H)⁺.

### Step 5. 1-((2R,5S)-4-(5-Cyclopropyl-7-(3-fluorophenyl)-7H-pyrrolo[2,3-d]pyrimidin-4-yl)-2,5-dimethylpiperazin-1-yl)-2,2-dimethylpropan-1-one

To a solution of 5-cyclopropyl-4-((2S,5R)-2,5-dimethylpiperazin-1-yl)-7-(3-fluorophenyl)-7H-pyrrolo[2,3-d]pyrimidine (130 mg, 0.36 mmol) and TEA (0.10 mL, 1.1 mmol) in DCM (2 mL) at 0° C. was added pivaloyl chloride (0.10 mL, 0.72 mmol). After stirring at room temperature for 1 h, the reaction was partitioned between DCM and water. The aqueous layer was extracted twice with DCM. The combined organic layers were washed with brine, dried over Na₂SO₄, filtered and concentrated. The residue was purified by prep-HPLC to afford 1-((2R,5S)-4-(5-cyclopropyl-7-(3-fluorophenyl)-7H-pyrrolo[2,3-d]pyrimidin-4-yl)-2,5-dimethylpiperazin-1-yl)-2,2-dimethylpropan-1-one (51 mg, 31%) as a white solid. LC/MS ESI (m/z): 450 (M+H)⁺. ¹H NMR (400 MHZ, CDCl₃) δ 8.37 (s, 1H), 7.43-7.37 (m, 3H), 6.99-6.94 (m, 1H), 6.90 (s, 1H), 4.93-4.88 (m, 1H), 4.65 (br. s, 1H), 4.17 (br. s, 1H), 3.74-3.49 (m, 3H), 2.00-1.94 (m, 1H), 1.28 (s, 9H), 1.21 (br. s, 3H), 1.07 (br. s, 3H), 0.98-0.94 (m, 2H), 0.83-0.78 (m, 1H), 0.63-0.58 (m, 1H).

The following compound was prepared by a procedure analogous to the synthesis of compound 214 from the corresponding chiral amine.

| Cmpd | | |
|---|---|---|
| No. | Chemical Name | LCMS and ¹H NMR |
| 215 | (S)-1-(4-(5-cyclopropyl-7-(3- | LC/MS ESI (m/z): 436 (M + H)⁺. ¹H NMR (400 MHz, |
| | fluorophenyl)-7H-pyrrolo[2,3- | CDCl₃) δ 8.39 (s, 1H), 7.42-7.36 (m, 3H), 6.99-6.93 (m, |
| | d]pyrimidin-4-yl)-3- | 1H), 6.87 (s, 1H), 4.72-4.65 (m, 1H), 4.23-4.14 (m, 2H), |
| | methylpiperazin-1-yl)-2,2- | 3.83 (d, J = 13.2 Hz, 1H), 3.57-3.49 (m, 1H), 3.38-3.26 |
| | dimethylpropan-1-one | (m, 2H), 2.00 (m, 1H), 1.28 (s, 9H), 1.15 (d, J = 6.6 Hz, |
| | | 3H), 0.96 (dd, J = 8.2, 1.7 Hz, 2H), 0.76 (m, 1H), 0.66- |
| | | 0.60 (m, 1H). |

### Example. Synthesis of (S)-1-(4-(5-(azetidin-1-yl)-7-(3-fluorophenyl)-7H-pyrrolo[2,3-d]pyrimidin-4-yl)-3-methylpiperazin-1-yl)-2,2-dimethylpropan-1-one (Compound 216)

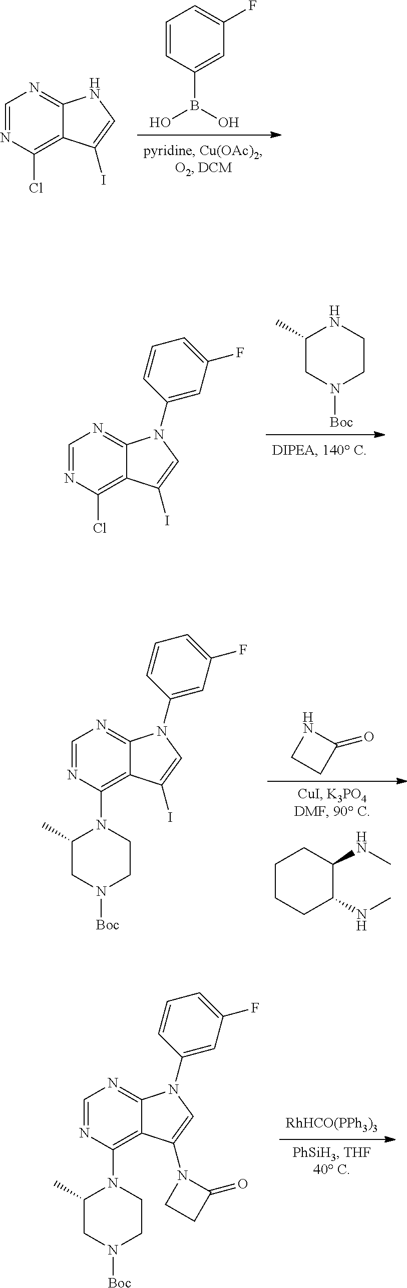

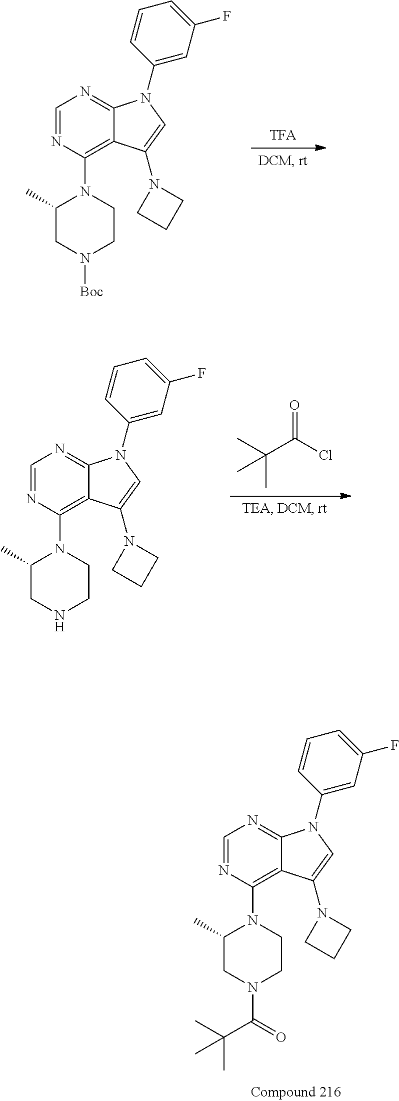

### Step 1. 4-Chloro-7-(3-fluorophenyl)-5-iodo-7H-pyrrolo[2,3-d]pyrimidine

To a solution of 4-chloro-5-iodo-7H-pyrrolo[2,3-d]pyrimidine (6.0 g, 22 mmol) in DCM (60 mL) were added (3-fluorophenyl) boronic acid (6.0 g, 43 mmol), 4 Å molecular sieves (3.0 g), Cu(OAc)₂ (16 g, 86 mmol) and pyridine (10 g, 130 mmol). The resulting mixture was heated at 30° C. under O₂ atmosphere for 2 days. The reaction was quenched with aq. NH₄OH (10 mL) in an ice water bath and filtered. The filtrate was extracted twice with DCM. The combined organic layers were dried over Na₂SO₄, filtered and concentrated. The residue was triturated with petroleum ether to afford 4-chloro-7-(3-fluorophenyl)-5-iodo-7H-pyrrolo[2,3-d]pyrimidine (5.0 g, 62%) as a solid. LC/MS ESI (m/z): 374 (M+H)⁺.

### Step 2. tert-Butyl (S)-4-(7-(3-fluorophenyl)-5-iodo-7H-pyrrolo[2,3-d]pyrimidin-4-yl)-3-methylpiperazine-1-carboxylate

To a solution of 4-chloro-7-(3-fluorophenyl)-5-iodo-7H-pyrrolo[2,3-d]pyrimidine (1.0 g, 2.7 mmol) in DIPEA (3 mL) was added tert-butyl (S)-3-methylpiperazine-1-carboxylate (1.1 g, 5.4 mmol). The resulting reaction mixture was heated at 140° C. under N₂ for 2 h. After cooling to room temperature, solvent was removed and the residue was purified by flash column chromatography (silica gel, 0˜30% ethyl acetate in petroleum ether) to afford tert-butyl (S)-4-(7-(3-fluorophenyl)-5-iodo-7H-pyrrolo[2,3-d]pyrimidin-4-yl)-3-methylpiperazine-1-carboxylate (800 mg, 68%) as a solid. LC/MS ESI (m/z): 538 (M+H)⁺.

### Step 3. tert-Butyl (S)-4-(7-(3-fluorophenyl)-5-(2-oxoazetidin-1-yl)-7H-pyrrolo[2,3-d]pyrimidin-4-yl)-3-methylpiperazine-1-carboxylate

To a solution of tert-butyl (S)-4-(7-(3-fluorophenyl)-5-iodo-7H-pyrrolo[2,3-d]pyrimidin-4-yl)-3-methylpiperazine-1-carboxylate (600 mg, 1.1 mmol) in DMF (5 mL) were added azetidin-2-one (320 mg, 1.1 mmol), trans-N,N-dimethylcyclohexane-1,2-diamine (160 mg, 1.1 mmol), CuI (210 mg, 1.1 mmol) and K₃PO₄ (710 mg, 3.4 mmol). The resulting mixture was heated at 90° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜70% ethyl acetate in petroleum ether) to afford tert-butyl (S)-4-(7-(3-fluorophenyl)-5-(2-oxoazetidin-1-yl)-7H-pyrrolo[2,3-d]pyrimidin-4-yl)-3-methylpiperazine-1-carboxylate (300 mg, 56%) as a solid. LC/MS ESI (m/z): 481 (M+H)⁺.

### Step 4. tert-Butyl (S)-4-(5-(azetidin-1-yl)-7-(3-fluorophenyl)-7H-pyrrolo[2,3-d]pyrimidin-4-yl)-3-methylpiperazine-1-carboxylate

To a solution of tert-butyl (S)-4-(7-(3-fluorophenyl)-5-(2-oxoazetidin-1-yl)-7H-pyrrolo[2,3-d]pyrimidin-4-yl)-3-methylpiperazine-1-carboxylate (250 mg, 0.52 mmol) in THF (5 mL) were added RhHCO(PPh₃)₃ (130 mg, 0.14 mmol) and PhSiH₃ (220 mg, 2.1 mmol) dropwise. The resulting mixture was heated at 40° C. for 1 h. After cooling to room temperature, the reaction was quenched in an ice water bath and extracted twice with EtOAc. The combined organic layers were dried over Na₂SO₄, filtered and concentrated. The residue was purified by flash column chromatography (silica gel, 0˜70% ethyl acetate in petroleum ether) to afford tert-butyl (S)-4-(5-(azetidin-1-yl)-7-(3-fluorophenyl)-7H-pyrrolo[2,3-d]pyrimidin-4-yl)-3-methylpiperazine-1-carboxylate (200 mg, 68%) as a solid. LC/MS ESI (m/z): 467 (M+H)⁺.

### Step 5. (S)-5-(Azetidin-1-yl)-7-(3-fluorophenyl)-4-(2-methylpiperazin-1-yl)-7H-pyrrolo[2,3-d]pyrimidine

To a solution of tert-butyl (S)-4-(5-(azetidin-1-yl)-7-(3-fluorophenyl)-7H-pyrrolo[2,3-d]pyrimidin-4-yl)-3-methylpiperazine-1-carboxylate (80 mg, 0.17 mmol) in DCM (5 mL) was added TFA (0.50 mL). The resulting mixture was stirred at room temperature for 1 h. The reaction was quenched with NaHCO₃ (aq.) and extracted twice with DCM. The combined organic layers were dried over Na₂SO₄, filtered, and concentrated to afford (S)-5-(azetidin-1-yl)-7-(3-fluorophenyl)-4-(2-methylpiperazin-1-yl)-7H-pyrrolo[2,3-d]pyrimidine (60 mg, 97%) as a solid. LC/MS ESI (m/z): 367 (M+H)⁺.

### (S)-1-(4-(5-(Azetidin-1-yl)-7-(3-fluorophenyl)-7H-pyrrolo[2,3-d]pyrimidin-4-yl)-3-methylpiperazin-1-yl)-2,2-dimethylpropan-1-one

To a solution of (S)-5-(azetidin-1-yl)-7-(3-fluorophenyl)-4-(2-methylpiperazin-1-yl)-7H-pyrrolo[2,3-d]pyrimidine (60 mg, 0.16 mmol) in DCM (5 mL) at 0° C. were added TEA (0.15 mL) and pivaloyl chloride (58 mg, 0.48 mmol). The resulting mixture was stirred at room temperature for 1 h. The reaction was then partitioned between DCM and water. The aqueous layer was extracted twice with DCM. The combined organic layers were washed with brine, dried over Na₂SO₄, filtered and concentrated. The residue was purified by flash column chromatography (silica gel, 0˜30%, ethyl acetate in petroleum ether) to give crude product which was further purified by prep-HPLC to afford (S)-1-(4-(5-(azetidin-1-yl)-7-(3-fluorophenyl)-7H-pyrrolo[2,3-d]pyrimidin-4-yl)-3-methylpiperazin-1-yl)-2,2-dimethylpropan-1-one (40 mg, 55%) as a solid. LC/MS ESI (m/z): 451 (M+H)⁺. ¹H NMR (400 MHZ, CDCl₃) δ 8.33 (s, 1H), 7.49-7.44 (m, 2H), 7.41-7.35 (m, 1H), 6.96-6.90 (m, 1H), 6.57 (s, 1H), 5.00-4.92 (m, 1H), 4.27 (d, J=13.2 Hz, 2H), 4.03 (d, J=12 Hz, 1H), 3.74 (app. q, J=6.9 Hz, 2H), 3.57 (app. q, J=6.8 Hz, 2H), 3.52-3.45 (m, 1H), 3.40-3.32 (m, 1H), 3.18-3.09 (m, 1H), 2.27-2.19 (m, 2H), 1.27 (s, 9H), 1.07 (d, J=6.7 Hz, 3H).

The following compounds were prepared by procedures analogous to the synthesis of compound 216 from the corresponding boronic acids, chiral amine, and acid chlorides.

| Cmpd | | |
|---|---|---|
| No. | Chemical Name | LCMS and ¹H NMR |
| 217 | 1-((2R,5S)-4-(5-(azetidin-1-yl)- | LC/MS ESI (m/z): 483 (M + H)⁺. ¹H NMR (400 MHz, |
| | 7-(3,5 difluorophenyl)-7H- | CDCl₃) δ 8.38 (s, 1H), 7.45-7.41 (m, 2H), 6.76-6.70 (m, |
| | pyrrolo[2,3-d]pyrimidin-4-yl) | 1H), 6.59 (s, 1H), 5.19 (br. s, 1H), 4.67 (br. s, 1H), 4.35- |
| | 2,5-dimethylpiperazin-1-yl)-2,2- | 4.09 (m, 1H), 4.03 (m, 1H), 3.85-3.80 (m, 2H), 3.73- |
| | dimethylpropan-1-one | 3.58 (m, 4H), 2.34-2.28 (m, 2H), 1.33 (s, 9H), 1.17-1.03 |
| | | (m, 6H). |
| 218 | 1-((2R,5S)-4-(5-(azetidin-1-yl)- | LC/MS ESI (m/z): 469 (M + H)⁺. ¹H NMR (400 MHz, |
| | 7-(3,5-difluorophenyl)-7H- | CDCl₃) δ 8.38 (d, J = 5.6 Hz, 1H), 7.46-7.41 (m, 2H), |
| | pyrrolo[2,3-d]pyrimidin-4-yl)- | 6.77-6.70 (m, 1H), 6.60 (d, J = 8.6 Hz, 1H), 5.19 (s, 1H), |
| | 2,5-dimethylpiperazin-1-yl)-2- | 4.90-4.84 (m, ~0.5H), 4.44-4.36 (m, ~0.5H), 4.27-4.20 |
| | methylpropan-1-one | (m, ~0.5H), 4.20-4.00 (m, 1H), 3.94-3.89 (m, ~0.5H), |
| | | 3.87-3.71 (m, 3H), 3.64-3.58 (m, ~2.5H), 3.53-3.46 |
| | | (m, ~0.5H), 2.89-2.77 (m, 1H), 2.34-2.27 (m, 2H), 1.24- |
| | | 1.16 (m, ~7.5H), 1.12 (d, J = 6.8 Hz, ~1.5H), 1.06 (d, J = |
| | | 6.7 Hz, ~1.5H), 0.96 (d, J = 6.8 Hz, ~1.5H); several peaks |
| | | and their integrations are fractions of protons on account of |
| | | presence of rotomers. |
| 219 | 1-((2R,5S)-4-(5-(azetidin-1-yl)- | LC/MS ESI (m/z): 465 (M + H)⁺. 1 H NMR (400 MHz, |
| | 7-(3-fluorophenyl)-7H- | CDCl₃) δ 8.31 (s, 1H), 7.50-7.45 (m, 2H), 7.40-7.34 (m, |
| | pyrrolo[2,3-d]pyrimidin-4-yl) | 1H), 6.96-6.90 (m, 1H), 6.56 (s, 1H), 5.14 (br. s, 1H), 4.60 |
| | 2,5-dimethylpiperazin-1-yl)-2,2- | (br. s, 1H), 4.33-3.91 (m, 2H), 3.78-3.52 (m, 6H), 2.27- |
| | dimethylpropan-1-one | 2.20 (m, 2H), 1.27 (s, 9H), 1.13-0.97 (m, 6H). |
| 220 | 1-((2R,5S)-4-(5-(azetidin-1-yl)- | LC/MS ESI (m/z): 481 (M + H)⁺. ¹H NMR (400 MHz, |
| | 7-(3-chlorophenyl)-7H- | CDCl₃) δ 8.37 (s, 1H), 7.75 (t, J = 1.9 Hz, 1H), 7.70-7.66 |
| | pyrrolo[2,3-d]pyrimidin-4-yl)- | (m, 1H), 7.42 (t, J = 8.1 Hz, 1H), 7.28 (m, 1H), 6.62 (s, |
| | 2,5-dimethylpiperazin-1-yl)-2,2- | 1H), 5.21 (br. s, 1H), 4.68 (br. s, 1H), 4.36-4.12 (m, 1H), |
| | dimethylpropan-1-one | 4.04 (d, J = 12.9 Hz, 1H), 3.83 (app. q, J = 6.9 Hz, 2H), |
| | | 3.75-3.58 (m, 4H), 2.31 (m, 2H), 1.34 (s, 9H), 1.20-1.02 |
| | | (m, 6H). |
| 221 | 1-((2R,5S)-4-(5-(azetidin-1-yl)- | LC/MS ESI (m/z): 467 (M + H)⁺. ¹H NMR (400 MHz, |
| | 7-(3-chlorophenyl)-7H- | CDCl₃) δ 8.37 (d, J = 5.4 Hz, 1H), 7.75 (t, J = 2.0 Hz, 1H), |
| | pyrrolo[2,3-d]pyrimidin-4-yl)- | 7.68 (d, J = 8.0 Hz, 1H), 7.42 (t, J = 8.1 Hz, 1H), 7.28 (s, |
| | 2,5-dimethylpiperazin-1-yl)-2- | 1H), 6.63 (d, J = 8.4 Hz, 1H), 5.20 (br. s, 1H), 4.87 (br. |
| | methylpropan-1-one | s, ~0.5H), 4.40 (d, J = 16 Hz, ~0.5H), 4.23 (br. s, ~0.5H), 4.14 |
| | | (d, J = 16 Hz, ~0.5H), 4.05 (d, J = 16 Hz, ~0.5H), 3.95- |
| | | 3.47 (m, 6.5 H), 2.89-2.77 (m, 1H), 2.34-2.26 (m, 2H), |
| | | 1.24-0.96 (m, 12H); several peaks and their integrations |
| | | are fractions of protons on account of presence of rotomers. |

### Example 43. Synthesis of 3-(4-((2S,5R)-2,5-dimethyl-4-pivaloylpiperazin-1-yl)-5-(trifluoromethyl)-7H-pyrrolo[2,3-d]pyrimidin-7-yl)benzonitrile (Compound 222)

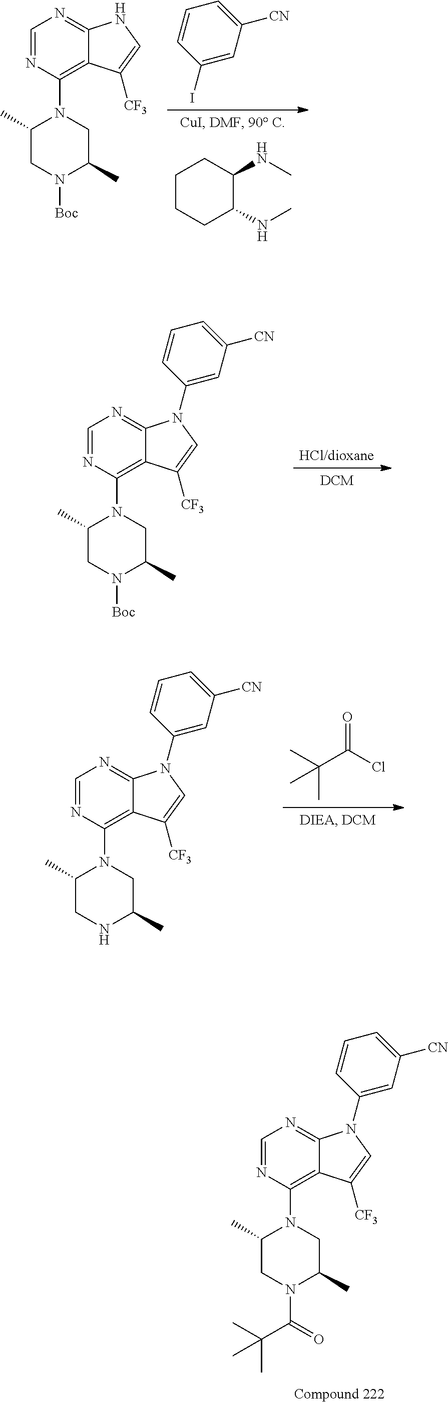

### Step 1. 3-(4-((2S,5R)-4-Isobutyryl-2,5-dimethylpiperazin-1-yl)-5-(trifluoromethyl)-7H-pyrrolo[2,3-d]pyrimidin-7-yl)benzonitrile

To a solution of tert-butyl (2R,5S)-2,5-dimethyl-4-(5-(trifluoromethyl)-7H-pyrrolo[2,3-d]pyrimidin-4-yl)piperazine-1-carboxylate (1.0 g, 2.5 mmol, prepared following the procedure outlined for compound 207) in DMF (10 mL) were added 3-iodobenzonitrile (1.2 g, 5.0 mmol), CuI (480 mg, 2.5 mmol), K₃PO₄ (1.6 g, 7.5 mmol) and trans-N,N′-dimethylcyclohexane-1,2-diamine (71 mg, 0.50 mmol). The mixture was degassed 3 times with N₂ and was then heated at 90° C. for 12 h. The mixture was cooled to room temperature, poured into H₂O (50 mL) and extracted with EtOAc (100 mL). The organic phase was washed with brine, dried over Na₂SO₄, filtered, and concentrated in vacuo. The crude product was purified by column chromatography (silica gel, 0˜20% ethyl acetate in petroleum ether) to give tert-butyl (2R,5S)-4-(7-(3-cyanophenyl)-5-(trifluoromethyl)-7H-pyrrolo[2,3-d]pyrimidin-4-yl)-2,5-dimethylpiperazine-1-carboxylate (600 mg, 48%) as an white solid. LC/MS ESI (m/z): 501 (M+H)⁺.

### Step 2. 3-(4-((2S,5R)-2,5-Dimethylpiperazin-1-yl)-5-(trifluoromethyl)-7H-pyrrolo[2,3-d]pyrimidin-7-yl)benzonitrile

To a solution of tert-butyl (2R,5S)-4-(7-(3-cyanophenyl)-5-(trifluoromethyl)-7H-pyrrolo[2,3-d]pyrimidin-4-yl)-2,5-dimethylpiperazine-1-carboxylate (600 mg, 1.2 mmol) in DCM (5 mL) at 0° C. was added HCl (1.0 mL, 4.0 M in dioxane). The resulting mixture was stirred at room temperature for 2 h. The reaction was then basified with NaHCO₃ (aq.) to pH 8 and extracted twice with DCM. The combined organic layers were dried over Na₂SO₄, filtered, and concentrated in vacuo. The crude product was used in the next step directly. LC/MS ESI (m/z): 401 (M+H)⁺.

### Step 3. 3-(4-((2S,5R)-2,5-Dimethyl-4-pivaloylpiperazin-1-yl)-5-(trifluoromethyl)-7H-pyrrolo[2,3-d]pyrimidin-7-yl)benzonitrile

To a solution of 3-(4-((2S,5R)-2,5-dimethylpiperazin-1-yl)-5-(trifluoromethyl)-7H-pyrrolo[2,3-d]pyrimidin-7-yl)benzonitrile (200 mg, 0.50 mmol) in DCM (10.0 mL) at 0° C. was added DIEA (0.16 mL, 1.0 mmol) and pivaloyl chloride (92 mg, 0.76 mmol). After 1 h, the reaction was quenched with water and extracted twice with DCM. The combined organic phases were washed with brine, dried over Na₂SO₄, filtered, and concentrated in vacuo. The crude product was purified by prep-HPLC to give 3-(4-((2S,5R)-2,5-dimethyl-4-pivaloylpiperazin-1-yl)-5-(trifluoromethyl)-7H-pyrrolo[2,3-d]pyrimidin-7-yl)benzonitrile (130 mg, 54%). LC/MS ESI (m/z): 485 (M+H)⁺. ¹H NMR (400 MHZ, CDCl₃) δ 8.52 (s, 1H), 8.07 (m, 1H), 7.96 (m, 1H), 7.76 (m, 1H), 7.74-7.65 (m, 2H), 4.79-4.54 (m, 2H), 4.15 (br. s, 1H), 3.78 (dd, J=16, 4.0 Hz, 1H), 3.68 (br. s, 1H), 3.54 (d, J=13.4 Hz, 1H), 1.34 (s, 9H), 1.24-1.07 (m, 6H).

The following compounds were prepared by procedures analogous to the synthesis of compound 222 from the corresponding aryl halides and carboxylic acid.

| Cmpd | | |
|---|---|---|
| No. | Chemical Name | LCMS and ¹H NMR |
| 223 | 1-((2R,5S)-4-(7-(3- | LC/MS ESI (m/z): 494 (M + H)⁺. ¹H NMR (400 MHz, |
| | fluorophenyl)-5- | CD₃OD) δ 8.46 (d, J = 5.0 Hz, 1H), 8.23 (d, J = 7.3 Hz, |
| | (trifluoromethyl)-7H- | 1H), 7.64 (m, 1H), 7.61-7.55 (m, 2H), 7.24 (m, 1H), 5.28 |
| | pyrrolo[2,3-d]pyrimidin-4-yl)- | (br. s, ~0.5H), 4.66 (m, ~1.5H), 4.49-4.31 (m, 1H), 4.13 |
| | 2,5-dimethylpiperazin-1-yl)-2- | (m, ~0.5H), 3.95-3.75 (m, 1H), 3.70-3.50 (m, ~1.5H), |
| | methoxy-2-methylpropan-1-one | 3.35 (s, 1H), 3.26 (s, 2H), 1.47 (m, 6H), 1.28 (m, 3H), 1.08 |
| | | (m, 3H); several peaks and their integrations are fractions of |
| | | protons on account of presence of rotomers. |
| 224 | 5-(4-((2S,5R)-2,5-dimethyl-4- | LC/MS ESI (m/z): 486 (M + H)⁺. ¹H NMR (400 MHz, |
| | pivaloylpiperazin-1-yl)-5- | CDCl₃) δ 9.09 (d, J = 2.5 Hz, 1H), 8.85 (d, J = 1.8 Hz, 1H), |
| | (trifluoromethyl)-7H- | 8.52 (m, 1H), 8.45 (s, 1H), 7.73 (s, 1H), 4.70-4.54 (m, |
| | pyrrolo[2,3-d]pyrimidin-7- | 2H), 4.24-3.96 (br. s, 1H), 3.73 (m, 1H), 3.66-3.45 (m, |
| | yl)nicotinonitrile | 2H), 1.27 (s, 9H), 1.16-1.06 (m, 6H). |
| 225 | 1-((2R,5S)-4-(7-(3,5- | |
| | difluorophenyl)-5- | LC/MS ESI (m/z): 496 (M + H)⁺. ¹H NMR (400 MHz, |
| | (trifluoromethyl)-7H- | CDCl₃) δ 8.46 (s, 1H), 7.66 (s, 1H), 7.28 (m, 2H), 6.83 (m, |
| | pyrrolo[2,3-d]pyrimidin-4-yl)- | 1H), 4.63 (br. s, 1H), 4.53 (br. s, 1H), 4.28-3.86 (br. s, |
| | 2,5-dimethylpiperazin-1-yl)-2,2- | 1H), 3.73-3.43 (m, 3H), 1.27 (s, 9H), 1.17-1.03 (m, 6H). |
| | dimethylpropan-1-one | |

### Example 44. Synthesis of 1-((2R,5S)-4-(3-(azetidin-1-yl)-1-(3-fluorophenyl)-1H-pyrrolo[3,2-c]pyridin-4-yl)-2,5-dimethylpiperazin-1-yl)-2,2-dimethylpropan-1-one (Compound 226)

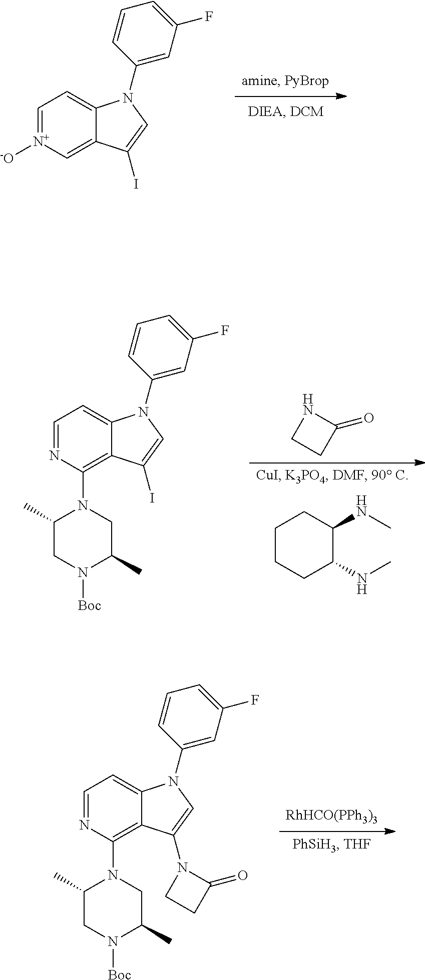

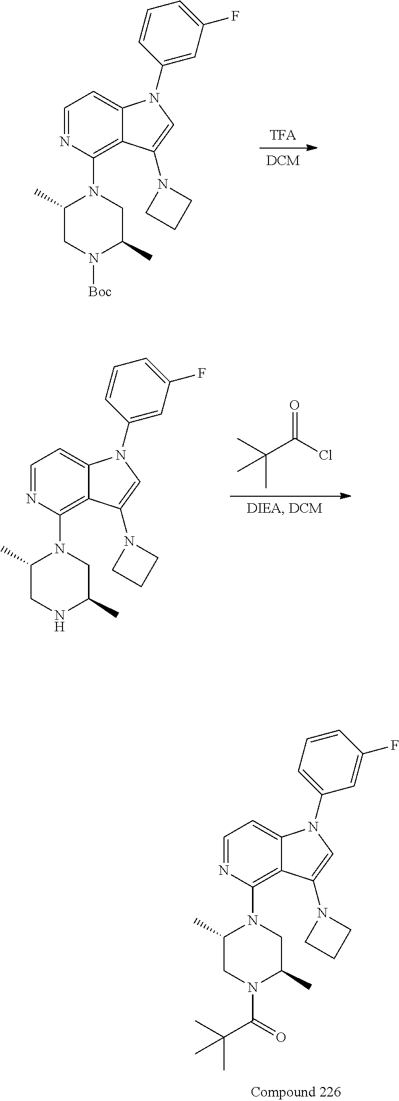

### Step 1. tert-Butyl (2R,5S)-4-(1-(3-fluorophenyl)-3-iodo-1H-pyrrolo[3,2-c]pyridin-4-yl)-2,5-dimethylpiperazine-1-carboxylate

To a solution of 1-(3-fluorophenyl)-3-iodo-1H-pyrrolo[3,2-c]pyridine 5-oxide (450 mg, 1.3 mmol, prepared following the procedure outlined for compound 228) in DCM (10 mL) were added tert-butyl (2R,5S)-2,5-dimethylpiperazine-1-carboxylate (1.3 g, 6.4 mmol), DIPEA (0.90 mL, 5.1 mmol) and PyBrop (1.2 g, 2.5 mmol). After stirring at room temperature over multiple days, the reaction was partitioned between DCM and water. The aqueous layer was extracted twice with DCM. The combined organic layers were washed with brine, dried over Na₂SO₄, filtered and concentrated, the residue was purified by flash column chromatography (silica gel, 0˜20% ethyl acetate in petroleum ether) to afford tert-butyl (2R,5S)-4-(1-(3-fluorophenyl)-3-iodo-1H-pyrrolo[3,2-c]pyridin-4-yl)-2,5-dimethylpiperazine-1-carboxylate (250 mg, 36%) as a yellow solid. LC/MS ESI (m/z): 551 (M+H)⁺.

### Step 2. tert-Butyl (2R,5S)-4-(1-(3-fluorophenyl)-3-(2-oxoazetidin-1-yl)-1H-pyrrolo[3,2-c]pyridin-4-yl)-2,5-dimethylpiperazine-1-carboxylate

To a solution of tert-butyl (2R,5S)-4-(1-(3-fluorophenyl)-3-iodo-1H-pyrrolo[3,2-c]pyridin-4-yl)-2,5-dimethylpiperazine-1-carboxylate (200 mg, 0.36 mmol) in DMF (5 mL) were added azetidin-2-one (100 mg, 1.4 mmol), CuI (34 mg, 0.18 mmol), trans-N,N′-dimethylcyclohexane-1,2-diamine (51 mg, 0.36 mmol) and K₃PO₄ (150 mg, 0.72 mmol). The resulting mixture was stirred at 90° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜55% ethyl acetate in petroleum ether) to afford tert-butyl (2R,5S)-4-(1-(3-fluorophenyl)-3-(2-oxoazetidin-1-yl)-1H-pyrrolo[3,2-c]pyridin-4-yl)-2,5-dimethylpiperazine-1-carboxylate (160 mg, 90%) as a yellow solid. LC/MS ESI (m/z): 494 (M+H)⁺.

### Step 3. tert-Butyl (2R,5S)-4-(3-(azetidin-1-yl)-1-(3-fluorophenyl)-1H-pyrrolo[3,2-c]pyridin-4-yl)-2,5-dimethylpiperazine-1-carboxylate

To a solution of tert-butyl (2R,5S)-4-(1-(3-fluorophenyl)-3-(2-oxoazetidin-1-yl)-1H-pyrrolo[3,2-c]pyridin-4-yl)-2,5-dimethylpiperazine-1-carboxylate (160 mg, 0.32 mmol) in THF (5 mL) at 0° C. were added RhHCO(PPh₃)₃ (29 mg, 0.032 mmol) and PhSiH₃ (0.20 mL, 1.6 mmol) dropwise. After stirring at room temperature for 1 h under N₂, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜50% ethyl acetate in petroleum ether) to give crude product which was further purified by prep-HPLC to afford tert-butyl (2R,5S)-4-(3-(azetidin-1-yl)-1-(3-fluorophenyl)-1H-pyrrolo[3,2-c]pyridin-4-yl)-2,5-dimethylpiperazine-1-carboxylate (85 mg, 55%) as a white solid. LC/MS ESI (m/z): 480 (M+H)⁺.

### Step 4. tert-Butyl (2R,5S)-4-(3-(azetidin-1-yl)-1-(3-fluorophenyl)-1H-pyrrolo[3,2-c]pyridin-4-yl)-2,5-dimethylpiperazine-1-carboxylate

To a solution of tert-butyl (2R,5S)-4-(3-(azetidin-1-yl)-1-(3-fluorophenyl)-1H-pyrrolo[3,2-c]pyridin-4-yl)-2,5-dimethylpiperazine-1-carboxylate (33 mg, 0.070 mmol) in DCM (2 mL) at 0° C. was added TFA (1 mL). After stirring at 0° C. for 1 h, the reaction was quenched with NaHCO₃ (aq.) and extracted twice with DCM. The combined organic layers were dried over Na₂SO₄, filtered and concentrated. The residue was used in the next step directly. LC/MS ESI (m/z): 380 (M+H)⁺.

### Step 5. 1-((2R,5S)-4-(3-(Azetidin-1-yl)-1-(3-fluorophenyl)-1H-pyrrolo[3,2-c]pyridin-4-yl)-2,5-dimethylpiperazin-1-yl)-2,2-dimethylpropan-1-one

To a solution of 3-(azetidin-1-yl)-4-((2S,5R)-2,5-dimethylpiperazin-1-yl)-1-(3-fluorophenyl)-1H-pyrrolo[3,2-c]pyridine (26 mg, 0.070 mmol) and TEA (0.10 mL, 0.21 mmol) in DCM (2 mL) at 0° C. was added pivaloyl chloride (0.10 mL, 0.14 mmol) dropwise. After stirring at 0° C. for 1 h, the reaction was partitioned between DCM and water. The aqueous layer was extracted twice with DCM. The combined organic layers were washed with brine, dried over Na₂SO₄, filtered and concentrated. The residue was purified by prep-HPLC to afford 1-((2R,5S)-4-(3-(azetidin-1-yl)-1-(3-fluorophenyl)-1H-pyrrolo[3,2-c]pyridin-4-yl)-2,5-dimethylpiperazin-1-yl)-2,2-dimethylpropan-1-one (7.8 mg, 22%) as a yellow solid. LC/MS ESI (m/z): 464 (M+H)⁺. ¹H NMR (400 MHZ, CDCl₃) δ 7.88 (d, J=5.9 Hz, 1H), 7.50-7.43 (m, 1H), 7.27 (m, 1H), 7.19 (dt, J=9.8, 2.2 Hz, 1H), 7.05 (td, J=8.2, 2.0 Hz, 1H), 6.98 (d, J=5.9 Hz, 1H), 6.62 (s, 1H), 4.96 (br. s, 1H), 4.68 (br. s, 1H), 4.24 (br. s, 1H), 3.97 (q, J=6.8 Hz, 2H), 3.77-3.48 (m, 5H), 2.29 (app. p, J=6.9 Hz, 2H), 1.35 (s, 9H), 1.30-1.18 (m, 3H), 1.03-0.87 (br. s, 3H).

The following compound was prepared by a procedure analogous to the synthesis of compound 226 from the corresponding chiral amine.

| Cmpd | | |
|---|---|---|
| No. | Chemical Name | LCMS and ¹H NMR |
| 227 | (S)-1-(4-(3-(azetidin-1-yl)-1-(3- | LC/MS ESI (m/z): 450 (M + H)⁺. ¹H NMR (400 MHz, |
| | fluorophenyl)-1H-pyrrolo[3,2- | CDCl₃) δ 7.95 (d, J = 6.0 Hz, 1H), 7.47 (m, 1H), 7.26 (m, |
| | c]pyridin-4-yl)-3- | 1H), 7.18 (dt, J = 9.7, 2.2 Hz, 1H), 7.09-7.01 (m, 2H), |
| | methylpiperazin-1-yl)-2,2- | 6.63 (s, 1H), 4.40 (br. s, 1H), 4.09-3.89 (m, 4H), 3.80 (d, |
| | dimethylpropan-1-one | J = 10.6 Hz, 1H), 3.72-3.57 (m, 4H), 3.32 (m, 1H), 2.28 |
| | | (app. p, J = 7.0 Hz, 2H), 1.34 (s, 9H), 1.03 (d, J = 6.5 Hz, |
| | | 3H). |

### Example 45. Synthesis of (S)-1-(4-(3-cyclopropyl-1-(3-fluorophenyl)-1H-pyrrolo[3,2-c]pyridin-4-yl)-3-methylpiperazin-1-yl)-2,2-dimethylpropan-1-one (Compound 228)

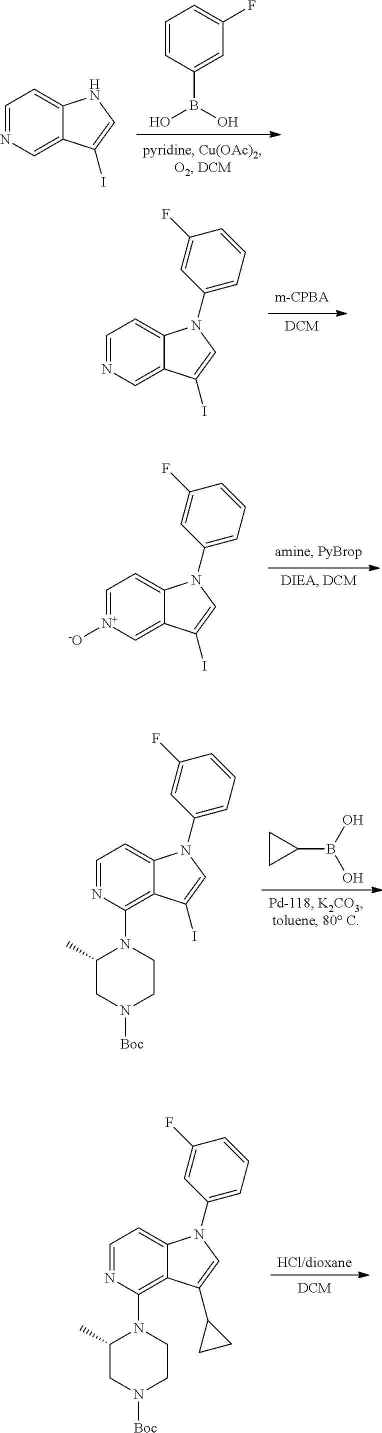

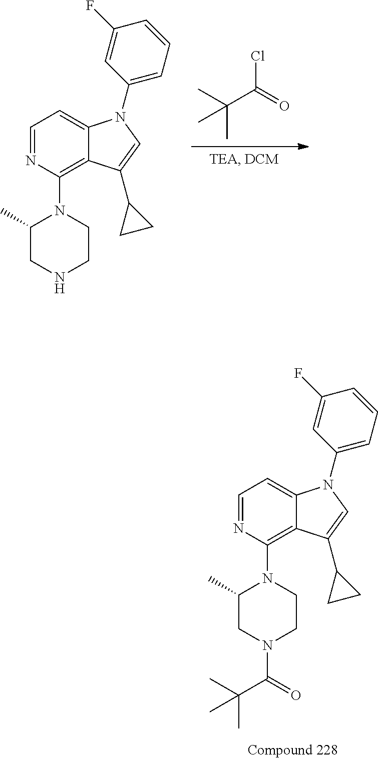

### Step 1. 1-(3-Fluorophenyl)-3-iodo-1H-pyrrolo[3,2-c]pyridine

A mixture of 3-iodo-1H-pyrrolo[3,2-c]pyridine (2.9 g, 12 mmol), (3-fluorophenyl) boronic acid (4.9 g, 35 mmol), Cu(OAc)₂ (5.8 g, 30 mmol) and pyridine (5.7 mL, 71 mmol) in DCM (50 mL) was stirred at room temperature for 48 h under an atmosphere of oxygen. The reaction mixture was then treated with aq. ammonium hydroxide and filtered. The filtrate was extracted twice with DCM. The combined layers were washed with brine, dried over Na₂SO₄, filtered and concentrated. The residue was purified by column chromatography on silica gel (0˜30% ethyl acetate in petroleum ether) to give 1-(3-fluorophenyl)-3-iodo-1H-pyrrolo[3,2-c]pyridine (1.5 g, 37%) as a solid. LC/MS ESI (m/z): 339 (M+H)⁺.

### Step 2. 1-(3-Fluorophenyl)-3-iodo-1H-pyrrolo[3,2-c]pyridine 5-oxide

To a solution of 1-(3-fluorophenyl)-3-iodo-1H-pyrrolo[3,2-c]pyridine (1.5 g, 4.4 mmol) in DCM (20 mL) at 0° C. was added 3-chloroperoxybenzoic acid (1.1 g, 6.6 mmol) in portions. The resulting mixture was stirred at room temperature overnight. The reaction was quenched with NaHCO₃ (aq.) and extracted twice with DCM. The combined organic layers were washed with brine, dried over Na₂SO₄, filtered and concentrated. The residue was purified by flash column chromatography (silica gel, 0˜20% MeOH in DCM) to provide 1-(3-fluorophenyl)-3-iodo-1H-pyrrolo[3,2-c]pyridine 5-oxide (920 mg, 61%) as a light yellow solid. LC/MS ESI (m/z): 355 (M+H)⁺.

### Step 3. tert-Butyl (S)-4-(1-(3-fluorophenyl)-3-iodo-1H-pyrrolo[3,2-c]pyridin-4-yl)-3-methylpiperazine-1-carboxylate

To a solution of 1-(3-fluorophenyl)-3-iodo-1H-pyrrolo[3,2-c]pyridine 5-oxide (450 mg, 1.3 mmol) in DCM (10 mL) were added tert-butyl (S)-3-methylpiperazine-1-carboxylate (1.3 g, 6.4 mmol), DIPEA (0.90 mL, 5.1 mmol) and PyBrop (1.2 g, 2.5 mmol). After stirring for 3 days, the reaction was partitioned between DCM and water. The aqueous layer was extracted twice with DCM. The combined organic layers were washed with brine, dried over Na₂SO₄, filtered and concentrated. The residue was purified by flash column chromatography (silica gel, 0˜20% ethyl acetate in petroleum ether) to afford tert-butyl (S)-4-(1-(3-fluorophenyl)-3-iodo-1H-pyrrolo[3,2-c]pyridin-4-yl)-3-methylpiperazine-1-carboxylate (250 mg, 36%) as a yellow solid. LC/MS ESI (m/z): 537 (M+H)⁺.

### Step 4. tert-Butyl (S)-4-(3-cyclopropyl-1-(3-fluorophenyl)-1H-pyrrolo[3,2-c]pyridin-4-yl)-3-methylpiperazine-1-carboxylate

To a solution of tert-butyl (S)-4-(1-(3-fluorophenyl)-3-iodo-1H-pyrrolo[3,2-c]pyridin-4-yl)-3-methylpiperazine-1-carboxylate (50 mg, 0.093 mmol) in toluene (5 mL) were added cyclopropylboronic acid (32 mg, 0.37 mmol), K₂CO₃ (260 mg, 1.9 mmol) and Pd-118 (6.0 mg, 0.01 mmol). The resulting mixture was stirred at 80° C. overnight. After cooling to room temperature, the reaction was concentrated and the residue was purified by flash column chromatography (silica gel, 0˜20% ethyl acetate in petroleum ether) to afford tert-butyl (S)-4-(3-cyclopropyl-1-(3-fluorophenyl)-1H-pyrrolo[3,2-c]pyridin-4-yl)-3-methylpiperazine-1-carboxylate (30 mg, 71%) as a yellow oil. LC/MS ESI (m/z): 451 (M+H)⁺.

### Step 5. (S)-3-Cyclopropyl-1-(3-fluorophenyl)-4-(2-methylpiperazin-1-yl)-1H-pyrrolo[3,2-c]pyridine

To a solution of tert-butyl (S)-4-(3-cyclopropyl-1-(3-fluorophenyl)-1H-pyrrolo[3,2-c]pyridin-4-yl)-3-methylpiperazine-1-carboxylate (30 mg, 0.067 mmol) in DCM (1 mL) was added HCl (0.50 mL, 4.0 M in dioxane). The resulting mixture was stirred at room temperature for 2 h. After removal of solvent, the residue was used in the next step directly. LC/MS ESI (m/z): 351 (M+H)⁺.

### Step 6. (S)-1-(4-(3-Cyclopropyl-1-(3-fluorophenyl)-1H-pyrrolo[3,2-c]pyridin-4-yl)-3-methylpiperazin-1-yl)-2,2-dimethylpropan-1-one

To a solution of (S)-3-cyclopropyl-1-(3-fluorophenyl)-4-(2-methylpiperazin-1-yl)-1H-pyrrolo[3,2-c]pyridine (23 mg, 0.066 mmol) and TEA (0.050 mL, 0.32 mmol) in DCM (2 mL) at 0° C. was added pivaloyl chloride (0.020 mL, 0.13 mmol). After stirring at room temperature for 1 h, the reaction was partitioned between DCM and water. The aqueous layer was extracted twice with DCM. The combined organic layers were washed with brine, dried over Na₂SO₄, filtered and concentrated. The residue was purified by prep-HPLC to afford (S)-1-(4-(3-cyclopropyl-1-(3-fluorophenyl)-1H-pyrrolo[3,2-c]pyridin-4-yl)-3-methylpiperazin-1-yl)-2,2-dimethylpropan-1-one (6.3 mg, 22%) as a white solid. LC/MS ESI (m/z): 435 (M+H)⁺. ¹H NMR (400 MHZ, CDCl₃) δ 7.98 (d, J=5.9 Hz, 1H), 7.40 (m, 1H), 7.16 (m, 1H), 7.10-7.06 (m, 2H), 6.78 (m, 1H), 6.78 (s, 1H), 3.97-3.80 (m, 3H), 3.65-3.47 (m, 3H), 3.07 (m, 1H), 2.43 (m, 1H), 1.27 (s, 9H), 0.99 (d, J=6.2 Hz, 3H), 0.96-0.91 (m, 2H), 0.77-0.72 (m, 1H), 0.55-0.50 (m, 1H).

The following compound was prepared by the procedure analogous to the synthesis of compound 228 from the corresponding chiral amine.

| Cmpd | | |
|---|---|---|
| No. | Chemical Name | LCMS and ¹H NMR |
| 229 | 1-((2R,5S)-4-(3-cyclopropyl-1- | LC/MS ESI (m/z): 449 (M + H)⁺. ¹H NMR (400 MHz, |
| | (3-fluorophenyl)-1H- | CDCl₃) δ 7.97 (d, J = 5.9 Hz, 1H), 7.47 (m, 1H), 7.23 (d, |
| | pyrrolo[3,2-c]pyridin-4-yl)-2,5- | J = 8.0 Hz, 1H), 7.15 (m, 1H), 7.11-7.04 (m, 2H), 6.85 (s, |
| | dimethylpiperazin-1-yl)-2,2- | 1H), 4.73 (br. s, 1H), 4.50 (br. s, 1H), 4.29 (br. s, 1H), 3.82 |
| | dimethylpropan-1-one | (dd, J = 12.6, 3.2 Hz, 1H), 3.56 (br. s, 1H), 3.24 (d, J = 12.7 |
| | | Hz, 1H), 2.39 (m, 1H), 1.41 (br. s, 3H), 1.35 (s, 9H), 1.07 |
| | | (m, 2H), 0.95 (m, 3H), 0.95 (m, 1H), 0.64 (m, 1H). |

### Example 46. Synthesis of 2-(4-((2S,5R)-2,5-dimethyl-4-pivaloylpiperazin-1-yl)-5-(2-fluorophenyl)-7H-pyrrolo[2,3-d]pyrimidin-7-yl)isonicotinonitrile (Compound 230)

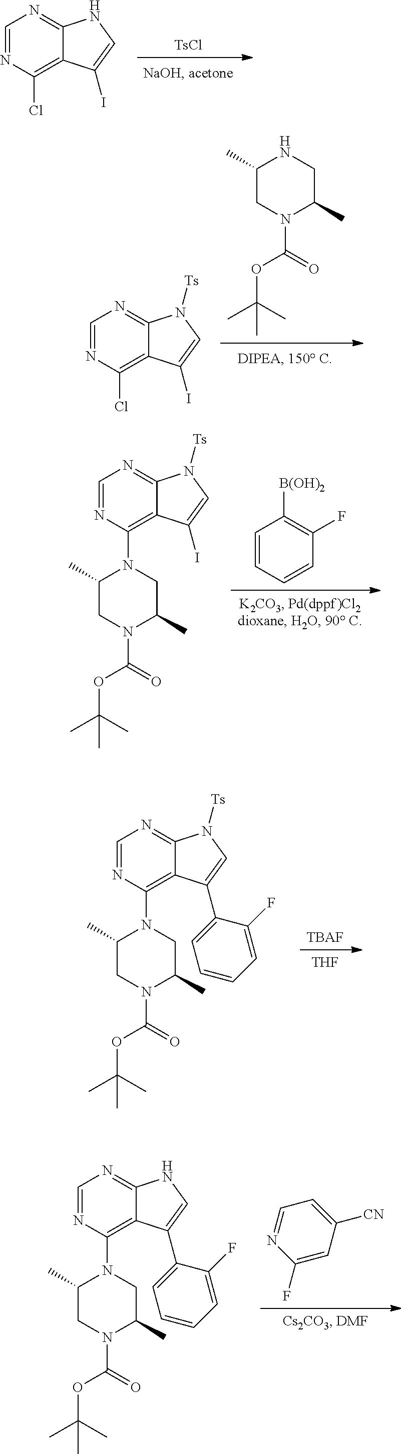

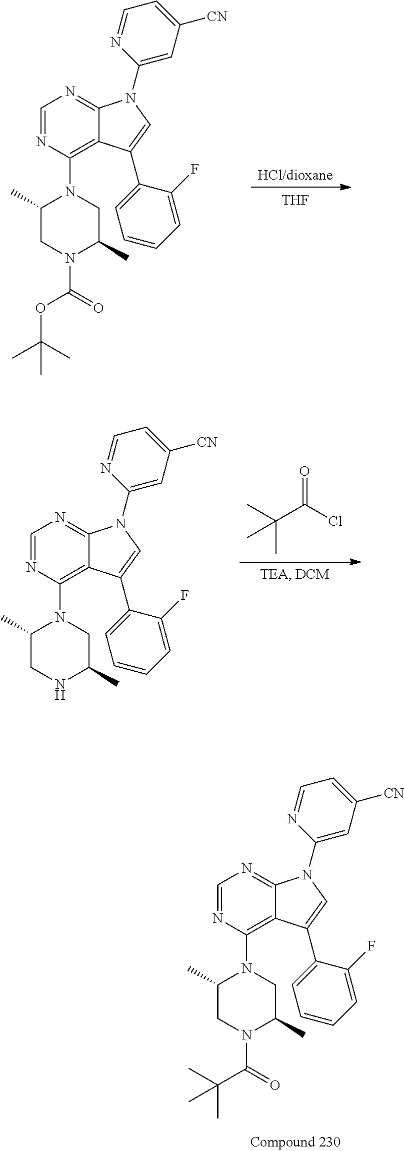

### Step 1. 4-Chloro-5-iodo-7-tosyl-7H-pyrrolo[2,3-d]pyrimidine

To a solution of 4-chloro-5-iodo-7H-pyrrolo[2,3-d]pyrimidine (200 g, 0.71 mol) and 4-methylbenzene-1-sulfonyl chloride (180 g, 0.93 mol) in acetone (2 L) at 0° C. was added 2.0M NaOH (0.53 L) dropwise. After complete addition, the reaction was allowed to warm up to room temperature and stirred for another 3 h. The resulting precipitate was collected by filtration, washed twice with water and dried in vacuo to afford 4-chloro-5-iodo-7-tosyl-7H-pyrrolo[2,3-d]pyrimidine (300 g, 95%) as an off-white solid. LC/MS ESI (m/z): 434 (M+H)⁺.

### Step 2. tert-Butyl (2R,5S)-4-(5-iodo-7-tosyl-7H-pyrrolo[2,3-d]pyrimidin-4-yl)-2,5-dimethylpiperazine-1-carboxylate

To a solution of 4-chloro-5-iodo-7-tosyl-7H-pyrrolo[2,3-d]pyrimidine (10 g, 23 mmol) in DIPEA (100 mL) was added tert-butyl (2R,5S)-2,5-dimethylpiperazine-1-carboxylate (9.9 g, 46 mmol). The resulting mixture was stirred at 150° C. for 2 h. After cooling to room temperature, the reaction was concentrated and purified by flash column chromatography (silica gel, 0˜30% ethyl acetate in petroleum ether) to afford tert-butyl (2R,5S)-4-(5-iodo-7-tosyl-7H-pyrrolo[2,3-d]pyrimidin-4-yl)-2,5-dimethylpiperazine-1-carboxylate (13 g, 92%) as a yellow solid. LC/MS ESI (m/z): 612 (M+H)⁺.

### Step 3. tert-Butyl (2R,5S)-4-(5-(2-fluorophenyl)-7-tosyl-7H-pyrrolo[2,3-d]pyrimidin-4-yl)-2,5-dimethylpiperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (6.0 g, 9.8 mmol) in dioxane (60 mL) and H₂O (10 mL) were added (2-fluorophenyl) boronic acid (1.7 g, 12 mmol), K₂CO₃ (4.1 g, 29 mmol) and Pd(dppf)Cl₂ (0.72 g, 0.98 mmol). The resulting mixture was stirred at 90° C. overnight. After cooling to room temperature, the reaction was concentrated and the residue was purified by flash column chromatography (silica gel, 0˜30% ethyl acetate in petroleum ether) to afford tert-butyl (2R,5S)-4-(5-(2-fluorophenyl)-7-tosyl-7H-pyrrolo[2,3-d]pyrimidin-4-yl)-2,5-dimethylpiperazine-1-carboxylate (5.5 g, 97%) as a yellow solid. LC/MS ESI (m/z): 580 (M+H)⁺.

### Step 4. tert-Butyl (2R,5S)-4-(5-(2-fluorophenyl)-7H-pyrrolo[2,3-d]pyrimidin-4-yl)-2,5-dimethylpiperazine-1-carboxylate

To a solution of tert-butyl (2R,5S)-4-(5-(2-fluorophenyl)-7-tosyl-7H-pyrrolo[2,3-d]pyrimidin-4-yl)-2,5-dimethylpiperazine-1-carboxylate (3.0 g, 5.2 mmol) in THF (15 mL) was added TBAF (31 mL, 1.0 M in THF). After stirring at room temperature for 2 h, the reaction was diluted with water and extracted twice with EtOAc. The combined organic layers were washed with brine, dried over Na₂SO₄, filtered and concentrated. The residue was purified by flash column chromatography (silica gel, 0˜60% ethyl acetate in petroleum ether) to afford tert-butyl (2R,5S)-4-(5-(2-fluorophenyl)-7H-pyrrolo[2,3-d]pyrimidin-4-yl)-2,5-dimethylpiperazine-1-carboxylate (2.0 g, 90%) as a yellow solid. LC/MS ESI (m/z): 426 (M+H)⁺.

### Step 5. tert-Butyl (2R,5S)-4-(7-(4-cyanopyridin-2-yl)-5-(2-fluorophenyl)-7H-pyrrolo[2,3-d]pyrimidin-4-yl)-2,5-dimethylpiperazine-1-carboxylate

To a solution of tert-butyl (2R,5S)-4-(5-(2-fluorophenyl)-7H-pyrrolo[2,3-d]pyrimidin-4-yl)-2,5-dimethylpiperazine-1-carboxylate (300 mg, 0.70 mmol) in DMF (10 mL) were added 2-fluoroisonicotinonitrile (170 mg, 1.4 mmol) and Cs₂CO₃ (690 mg, 2.1 mmol). The resulting mixture was stirred at 50° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜20% ethyl acetate in petroleum ether) to afford tert-butyl (2R,5S)-4-(7-(4-cyanopyridin-2-yl)-5-(2-fluorophenyl)-7H-pyrrolo[2,3-d]pyrimidin-4-yl)-2,5-dimethylpiperazine-1-carboxylate (320 mg, 86%) as a yellow solid. LC/MS ESI (m/z): 528 (M+H)⁺.

### Step 6. 2-(4-((2S,5R)-2,5-Dimethylpiperazin-1-yl)-5-(2-fluorophenyl)-7H-pyrrolo[2,3-d]pyrimidin-7-yl)isonicotinonitrile

To a solution of tert-butyl (2R,5S)-4-(7-(4-cyanopyridin-2-yl)-5-(2-fluorophenyl)-7H-pyrrolo[2,3-d]pyrimidin-4-yl)-2,5-dimethylpiperazine-1-carboxylate (320 mg, 0.60 mmol) in THF (5 mL) was added HCl (3.0 mL, 4.0 M in dioxane). After stirring at room temperature overnight, the reaction was partitioned between DCM and water. The aqueous layer was extracted twice with DCM. The combined organic layers were washed with brine, dried over Na₂SO₄, filtered and concentrated. The residue was purified by flash column chromatography (silica gel, 0˜10% MeOH in DCM) to afford 2-(4-((2S,5R)-2,5-dimethylpiperazin-1-yl)-5-(2-fluorophenyl)-7H-pyrrolo[2,3-d]pyrimidin-7-yl)isonicotinonitrile (190 mg, 73%) as a pink solid. LC/MS ESI (m/z): 428 (M+H)⁺.

### Step 7. 2-(4-((2S,5R)-2,5-Dimethyl-4-pivaloylpiperazin-1-yl)-5-(2-fluorophenyl)-7H-pyrrolo[2,3-d]pyrimidin-7-yl)isonicotinonitrile

To a solution of 2-(4-((2S,5R)-2,5-dimethylpiperazin-1-yl)-5-(2-fluorophenyl)-7H-pyrrolo[2,3-d]pyrimidin-7-yl)isonicotinonitrile (90 mg, 0.21 mmol) and TEA (0.090 mL, 0.63 mmol) in DCM (3 mL) at 0° C. was added pivaloyl chloride (0.050 mL, 0.42 mmol).

After stirring at room temperature for 1 h, the reaction was partitioned between DCM and water. The aqueous layer was extracted twice with DCM. The combined organic layers were washed with brine, dried over Na₂SO₄, filtered and concentrated. The residue was purified by prep-HPLC to afford 2-(4-((2S,5R)-2,5-dimethyl-4-pivaloylpiperazin-1-yl)-5-(2-fluorophenyl)-7H-pyrrolo[2,3-d]pyrimidin-7-yl)isonicotinonitrile (65 mg, 61%) as a white solid. LC/MS ESI (m/z): 512 (M+H)⁺. ¹H NMR (400 MHZ, CDCl₃) δ 9.34 (s, 1H), 8.56 (d, J=5.0 Hz, 1H), 8.51 (s, 1H), 8.23 (s, 1H), 7.43 (m, 1H), 7.34 (dd, J=5.0, 1.3 Hz, 1H), 7.30 (m, 1H), 7.22-7.08 (m, 2H), 4.45 (br. s, 1H), 4.25-3.86 (br. s, 1H), 3.63 (m, 1H), 3.37 (d, J=12.9 Hz, 1H), 3.22 (m, 1H), 3.01-2.35 (br. s, 1H), 1.17 (s, 9H), 1.11 (m, 3H), 0.85 (br. s, 3H).

The following compounds were prepared by procedures analogous to the synthesis of compound 230 from the corresponding boronic acids and acid chlorides. For some analogs, the cyclobutyl group was introduced by coupling of the corresponding aryl bromide with cyclobutylboronic acid (Pd-118, K₂CO₃ (20 eq.), 80° C. in toluene). For compound 239, the corresponding carboxylic acid was synthesized following procedures described in US patent US20190248809).

| Cmpd | | |
|---|---|---|
| No. | Chemical Name | LCMS and ¹H NMR |
| 231 | 2-(4-((2S,5R)-4-(2,2-bis(methyl- | LC/MS ESI (m/z): 521 (M + H)⁺. ¹H NMR (400 MHz, |
| | d₃)propanoyl-3,3,3-d3)-2,5- | CDCl₃) δ 9.34 (s, 1H), 8.56 (d, J = 5.0 Hz, 1H), 8.51 (s, |
| | dimethylpiperazin-1-yl)-5-(2- | 1H), 8.23 (s, 1H), 7.43 (m, 1H), 7.35-7.27 (m, 2H), 7.20 |
| | fluorophenyl)-7H-pyrrolo[2,3- | (m, 1H), 7.15 (m, 1H), 4.44 (br. s, 1H), 4.07 (br. s, 1H), |
| | d]pyrimidin-7- | 3.62 (br. s, 1H), 3.45-3.15 (m, 2H), 3.08-2.30 (br. s, |
| | yl)isonicotinonitrile | 1H), 1.13 (br. s, 3H), 0.85 (br. s, 3H). |
| 232 | 2-(5-(2-fluorophenyl)-4- | LC/MS ESI (m/z): 528 (M + H)⁺. ¹H NMR (400 MHz, |
| | ((2S,5R)-4-(2-methoxy-2- | CDCl₃) δ 9.40 (s, 1H), 8.69-8.53 (m, 2H), 8.30 (d, J = 3.9 |
| | methylpropanoyl)-2,5- | Hz, 1H), 7.50 (m, 1H), 7.45-7.34 (m, 2H), 7.30-7.18 (m, |
| | dimethylpiperazin-1-yl)-7H- | 2H), 5.14 (br. s, ~0.5H), 4.61 (br. s, ~0.5H), 4.31 (br. |
| | pyrrolo[2,3-d]pyrimidin-7- | s, ~0.5H), 4.10 (d, J = 12 Hz, 1H), 3.89 (d, J = 16 Hz, ~0.5H), |
| | yl)isonicotinonitrile | 3.55-3.15 (m, 5.5H), 2.61 (d, J = 16 Hz, ~0.5H), |
| | | 1.50-0.80 (m, 12H); several peaks and their integrations |
| | | are fractions of protons on account of presence of rotomers. |
| 233 | 2-(4-((2S,5R)-2,5-dimethyl-4-(2- | LC/MS ESI (m/z): 505 (M + H)⁺. ¹H NMR (400 MHz, |
| | (methyl-d3)propanoyl-2,3,3,3- | CDCl₃) δ 9.34 (s, 1H), 8.56 (d, J = 4.8 Hz, 1H), 8.51 (d, J = |
| | d4)piperazin-1-yl)-5-(2- | 6.0 Hz, 1H), 8.26-8.20 (m, 1H), 7.43 (m, 1H), 7.35-7.25 |
| | fluorophenyl)-7H-pyrrolo[2,3- | (m, 2H), 7.18-7.10 (m, 2H), 4.60 (br. s, ~0.5H), 4.35 (br. |
| | d]pyrimidin-7- | s, ~0.5H), 4.04 (br. s, ~0.5H), 3.96 (br. s, ~0.5H), 3.79 (d, |
| | yl)isonicotinonitrile | J = 12 Hz, ~0.5H), 3.48 (d, J = 12 Hz, ~0.5H), 3.30-3.05 |
| | | (m, ~2.5H), 2.49 (d, J = 12 Hz, ~0.5H), 1.21 (d, J = 6.8 |
| | | Hz, ~1.5H), 0.98 (d, J = 6.7 Hz, ~1.5H), 0.93 (d, J = 6.8 |
| | | Hz, ~1.5H), 0.76 (d, J = 6.6 Hz, ~1.5H); several peaks and their |
| | | integrations are fractions of protons on account of presence |
| | | of rotomers. |
| 234 | 2-(4-((2S,5R)-2,5-dimethyl-4-(2- | LC/MS ESI (m/z): 504 (M + H)⁺. ¹H NMR (400 MHz, |
| | (methyl-d3)propanoyl-3,3,3- | CDCl₃) δ 9.35 (s, 1H), 8.56 (d, J = 5.0 Hz, 1H), 8.51 (d, J = |
| | d3)piperazin-1-yl)-5-(2- | 6.0 Hz, 1H), 8.23 (d, J = 4.8 Hz, 1H), 7.43 (m, 1H), 7.36- |
| | fluorophenyl)-7H-pyrrolo[2,3- | 7.28 (m, 2H), 7.19-7.09 (m, 2H), 4.60 (br. s, ~0.5H), 4.34 |
| | d]pyrimidin-7- | (br. s, ~0.5H), 4.04 (br. s, ~0.5H), 3.96 (br. s, ~0.5H), 3.80 |
| | yl)isonicotinonitrile | (d, J = 12 Hz, ~0.5H), 3.48 (d, J = 16 Hz, ~0.5H), 3.30- |
| | | 3.05 (m, ~2.5H), 2.67 (s, ~0.5H), 2.49 (m, 1H), 1.20 (d, J = |
| | | 6.8 Hz, ~1.5H), 0.98 (d, J = 6.7 Hz, ~1.5H), 0.93 (d, J = 6.9 |
| | | Hz, ~1.5H), 0.76 (d, J = 6.6 Hz, ~1.5H); several peaks and |
| | | their integrations are fractions of protons on account of |
| | | presence of rotomers. |
| 235 | 2-(4-((2S,5R)-4-(1- | LC/MS ESI (m/z): 514 (M + H)⁺. ¹H NMR (400 MHz, |
| | fluorocyclopropane-1-carbonyl)- | CDCl3) δ 9.34 (s, 1H), 8.56 (d, J = 8.0 Hz, 1H), 8.52 (s, |
| | 2,5-dimethylpiperazin-1-yl)-5- | 1H), 8.24 (s, 1H), 7.44 (m, 1H), 7.37-7.28 (m, 2H), 7.21- |
| | (2-fluorophenyl)-7H- | 7.12 (m, 2H), 4.48 (br. s, 1H), 4.28 (br. s, ~0.5H), 4.04 (br. |
| | pyrrolo[2,3-d]pyrimidin-7- | s, ~0.5H), 3.75-3.42 (m, ~1.5H), 3.38-3.13 (m, 2H), 2.63 |
| | yl)isonicotinonitrile | (d, J = 12.7 Hz, ~0.5H), 1.29-0.76 (m, 10H); several |
| | | peaks and their integrations are fractions of protons on |
| | | account of presence of rotomers. |
| 236 | 2-(5-cyclobutyl-4-((2S,5R)-4-(1- | LC/MS ESI (m/z): 474 (M + H)⁺. ¹H NMR (400 MHz, |
| | fluorocyclopropane-1-carbonyl)- | CDCl3) δ 9.35 (s, 1H), 8.62 (d, J = 5.0 Hz, 1H), 8.51 (s, |
| | 2,5-dimethylpiperazin-1-yl)-7H- | 1H), 8.11 (s, 1H), 7.36 (d, J = 5.0 Hz, 1H), 4.85-4.48 (m, |
| | pyrrolo[2,3-d]pyrimidin-7- | 2H), 4.30-3.90 (m, ~1.5H), 3.82 (m, 1H), 3.74-3.68 |
| | yl)isonicotinonitrile | (m, ~1.5H), 3.53 (d, J = 13.3 Hz, 1H), 2.55-2.37 (m, 2H), 2.27 |
| | | (m, 1H), 2.19-1.92 (m, 3H), 1.33-1.08 (m, 10H); several |
| | | peaks and their integrations are fractions of protons on |
| | | account of presence of rotomers. |
| 237 | 2-(5-cyclobutyl-4-((2S,5R)-2,5- | LC/MS ESI (m/z): 472 (M + H)⁺. ¹H NMR (400 MHz, |
| | dimethyl-4-pivaloylpiperazin-1- | DMSO-d6) δ 9.16 (s, 1H), 8.79 (d, J = 5.1 Hz, 1H), 8.51 (s, |
| | yl)-7H-pyrrolo[2,3-d]pyrimidin- | 1H), 8.09 (s, 1H), 7.80 (dd, J = 5.1, 1.3 Hz, 1H), 4.63- |
| | 7-yl)isonicotinonitrile | 4.44 (m, 2H), 4.13 (m, 1H), 3.78-3.67 (m, 2H), 3.63- |
| | | 3.42 (m, 2H), 2.47-2.28 (m, 2H), 2.22 (m, 1H), 2.05 (m, |
| | | 2H), 1.89 (m, 1H), 1.25 (s, 9H), 1.04 (br. s, 6H). |
| 238 | 2-(5-cyclobutyl-4-((2S,5R)-4- | LC/MS ESI (m/z): 458 (M + H)⁺. ¹H NMR (400 MHz, |
| | isobutyryl-2,5- | CDCl3) δ 9.34 (s, 1H), 8.63 (d, J = 5.0 Hz, 1H), 8.51 (d, J = |
| | dimethylpiperazin-1-yl)-7H- | 4.2 Hz, 1H), 8.11 (d, J = 21.3 Hz, 1H), 7.37 (d, J = 5.1 Hz, |
| | pyrrolo[2,3-d]pyrimidin-7- | 1H), 4.95-4.70 (m, 1H), 4.42-4.24 (m, 1H), 4.07-3.40 |
| | yl)isonicotinonitrile | (m, 5H), 2.96-2.73 (m, 1H), 2.60-2.38 (m, 2H), 2.32- |
| | | 1.95 (m, 4H), 1.31-1.02 (m, 12H); several peaks and their |
| | | integrations are fractions of protons on account of presence |
| | | of rotomers. |
| 239 | 2-(5-cyclobutyl-4-((2S,5R)-2,5- | LC/MS ESI (m/z): 540 (M + H)⁺. ¹H NMR (400 MHz, |
| | dimethyl-4-(4,4,4-trifluoro-3,3- | CDCl3) δ 9.34 (s, 1H), 8.62 (d, J = 5.0 Hz, 1H), 8.50 (d, J = |
| | dimethylbutanoyl)piperazin-1- | 8.5 Hz, 1H), 8.11 (d, J = 22.9 Hz, 1H), 7.37 (d, J = 5.0 Hz, |
| | yl)-7H-pyrrolo[2,3-d]pyrimidin- | 1H), 4.84 (s, ~0.5H), 4.68 (s, ~0.5H), 4.46 (s, ~0.5H), 4.36 |
| | 7-yl)isonicotinonitrile | (d, J = 14.0 Hz, ~0.5H), 4.20 (s, ~0.5H), 3.91 (d, J = 10.5 |
| | | Hz, 0.5H), 3.85-3.63 (m, 2H), 3.61-3.43 (m, 2H), 2.70 |
| | | (m, 1H), 2.55-2.35 (m, 3H), 2.32-1.95 (m, 4H), 1.37- |
| | | 1.20 (m, 9H), 1.05 (m, 3H); several peaks and their |
| | | integrations are fractions of protons on account of presence |
| | | of rotomers. |
| 240 | 2-(5-cyclobutyl-4-((2S,5R)-2,5- | LC/MS ESI (m/z): 526 (M + H)⁺. ¹H NMR (400 MHz, |
| | dimethyl-4-(3,3,3-trifluoro-2,2- | CDCl3) δ 9.34 (s, 1H), 8.62 (d, J = 5.0 Hz, 1H), 8.50 (s, |
| | dimethylpropanoyl)piperazin-1- | 1H), 8.11 (s, 1H), 7.37 (dd, J = 5.0, 1.1 Hz, 1H), 4.61 (m, |
| | yl)-7H-pyrrolo[2,3-d]pyrimidin- | 2H), 4.16 (br. s, 1H), 3.83-3.67 (m, 3H), 3.51 (d, J = 13.2 |
| | 7-yl)isonicotinonitrile | Hz, 1H), 2.51-2.40 (m, 2H), 2.27 (m, 1H), 2.16-1.97 (m, |
| | | 3H), 1.58-1.57 (s, 6H), 1.23-1.11 (m, 6H). |
| 241 | (S)-2-(5-cyclopropyl-4-(4-(2- | LC/MS ESI (m/z): 460 (M + H)⁺. ¹H NMR (400 MHz, |
| | methoxy-2-methylpropanoyl)-2- | CDCl3) δ 9.32 (s, 1H), 8.59 (d, J = 5.0 Hz, 1H), 8.53 (S, |
| | methylpiperazin-1-yl)-7H- | 1H), 7.80 (s, 1H), 7.35 (dd, J = 5.0, 1.3 Hz, 1H), 4.96- |
| | pyrrolo[2,3-d]pyrimidin-7- | 4.21 (m, 3H), 3.90 (d, J = 13.2 Hz, 1H), 3.61 (m, 1H), 3.49 |
| | yl)isonicotinonitrile | (m, 1H), 3.31 (s, 3H), 3.21 (m, 1H), 2.08 (m, 1H), 1.51 (m, |
| | | 6H), 1.32-1.17 (m, 3H), 1.05 (d, J = 8.1 Hz, 2H), 0.91 (m, |
| | | 1H), 0.77 (m, 1H); several peaks and their integrations are |
| | | fractions of protons on account of presence of rotomers. |
| 242 | (S)-2-(5-(2-fluorophenyl)-4-(4- | LC/MS ESI (m/z): 514 (M + H)⁺. ¹H NMR (400 MHz, |
| | (2-methoxy-2- | CDCl3) δ 9.34 (m, 1H), 8.56 (dd, J = 5.0, 0.7 Hz, 1H), 8.52 |
| | methylpropanoyl)-2- | (s, 1H), 8.22 (s, 1H), 7.41 (td, J = 7.5, 1.6 Hz, 1H), 7.34 |
| | methylpiperazin-1-yl)-7H- | (dd, J = 5.0, 1.3 Hz, 1H), 7.30 (m, 1H), 7.21-7.11 (m, |
| | pyrrolo[2,3-d]pyrimidin-7- | 2H), 4.63-3.79 (m, ~3.5H), 3.58-3.26 (m, ~1.5H), 3.13 |
| | yl)isonicotinonitrile | (s, 3H), 3.01 (m, 1H), 2.74-2.44 (m, 1H), 1.34 (m, 6H), |
| | | 1.07 (br. s, ~1H), 0.86 (m, ~2H); several peaks and their |
| | | integrations are fractions of protons on account of presence |
| | | of rotomers. |
| 243 | (S)-2-(5-(2-fluorophenyl)-4-(4- | LC/MS ESI (m/z): 512 (M + H)⁺. 1HNMR(400 MHz, |
| | (1-methoxycyclopropane-1- | CDCl3) δ 9.34 (m, 1H), 8.59-8.49 (m, 2H), 8.22 (s, 1H), |
| | carbonyl)-2-methylpiperazin-1- | 7.41 (t, J = 6.8 Hz, 1H), 7.35-7.27 (m, 2H), 7.20-7.11 |
| | yl)-7H-pyrrolo[2,3-d]pyrimidin- | (m, 2H), 4.32-3.82 (m, 3H), 3.52 (m, 1H), 3.11 (s, 3H), |
| | 7-yl)isonicotinonitrile | 3.10-2.92 (m, 2H), 2.59 (m, 1H), 1.03-0.79 (m, 7H); |
| | | several peaks and their integrations are fractions of protons |
| | | on account of presence of rotomers. |

### Example 47. Synthesis of 1-((2R,5S)-4-(7-(4-fluoropyridin-2-yl)-5-methyl-7H-pyrrolo[2,3-d]pyrimidin-4-yl)-2,5-dimethylpiperazin-1-yl)-2,2-dimethylpropan-1-one (Compound 244)

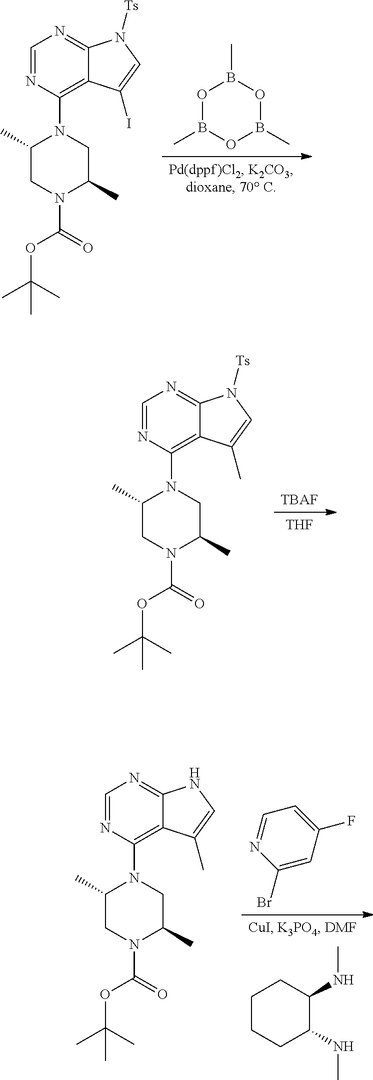

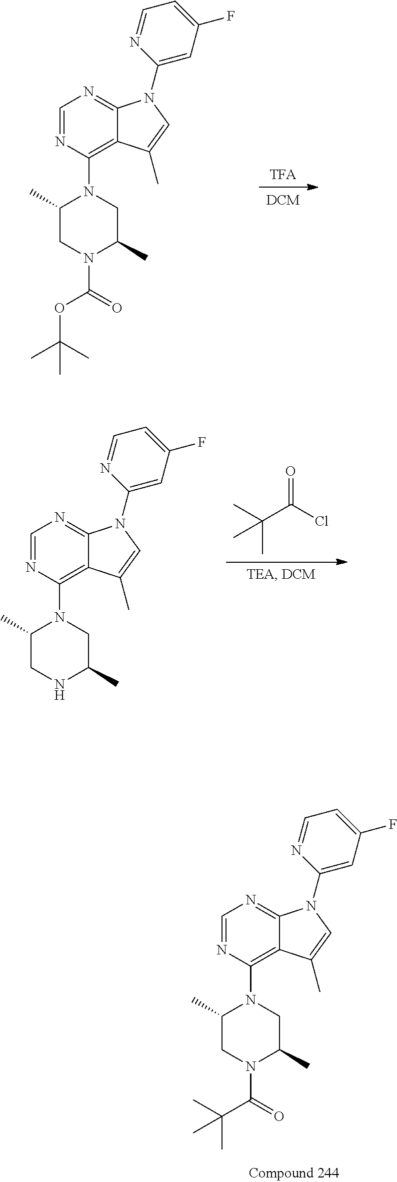

### Step 1. tert-Butyl (2R,5S)-2,5-dimethyl-4-(5-methyl-7-tosyl-7H-pyrrolo[2,3-d]pyrimidin-4-yl)piperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (4.0 g, 6.7 mmol, prepared following the procedure outlined for compound 230) in dioxane (50 mL) and H₂O (10 mL) were added 2,4,6-trimethyl-1,3,5,2,4,6-trioxatriborinane (1.7 g, 13 mmol), K₂CO₃ (2.8 g, 20 mmol) and Pd(dppf)Cl₂ (490 mg, 0.67 mmol). The resulting mixture was heated at 70° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜50% ethyl acetate in petroleum ether) to afford tert-butyl (2R,5S)-2,5-dimethyl-4-(5-methyl-7-tosyl-7H-pyrrolo[2,3-d]pyrimidin-4-yl)piperazine-1-carboxylate (2.0 g, 61%) as a white solid. LC/MS ESI (m/z): 500 (M+H)⁺.

### Step 2. tert-Butyl (2R,5S)-2,5-dimethyl-4-(5-methyl-7H-pyrrolo[2,3-d]pyrimidin-4-yl)piperazine-1-carboxylate

To a solution of tert-butyl (2R,5S)-2,5-dimethyl-4-(5-methyl-7-tosyl-7H-pyrrolo[2,3-d]pyrimidin-4-yl)piperazine-1-carboxylate (2.0 g, 4.0 mmol) in THF (20 mL) was added TBAF (24 mL, 1.0 M in THF). After 5 h, the reaction was diluted with water and extracted twice with EtOAc. The combined organic layers were washed with brine, dried over Na₂SO₄, filtered and concentrated. The residue was purified by flash column chromatography (silica gel, 0˜80% ethyl acetate in petroleum ether) to afford tert-butyl (2R,5S)-2,5-dimethyl-4-(5-methyl-7H-pyrrolo[2,3-d]pyrimidin-4-yl)piperazine-1-carboxylate (1.2 g, 87%) as a white solid. LC/MS ESI (m/z): 346 (M+H)⁺.

### Step 3. tert-Butyl (2R,5S)-4-(7-(4-fluoropyridin-2-yl)-5-methyl-7H-pyrrolo[2,3-d]pyrimidin-4-yl)-2,5-dimethylpiperazine-1-carboxylate

To a solution of tert-butyl (2R,5S)-2,5-dimethyl-4-(5-methyl-7H-pyrrolo[2,3-d]pyrimidin-4-yl)piperazine-1-carboxylate (100 mg, 0.30 mmol) in DMF (5 mL) were added 2-bromo-4-fluoropyridine (67 mg, 0.60 mmol), trans-N,N′-dimethylcyclohexane-1,2-diamine (6.0 mg, 0.04 mmol), CuI (7.6 mg, 0.04 mmol) and K₃PO₄ (190 mg, 0.90 mmol). The resulting mixture was heated at 120° C. overnight. After cooling to room temperature, the reaction was diluted with water and extracted twice with EtOAc. The combined organic layers were washed with brine, dried over Na₂SO₄, filtered and concentrated. The residue was purified by flash column chromatography (silica gel, 0˜50% ethyl acetate in petroleum ether) to afford tert-butyl (2R,5S)-4-(7-(4-fluoropyridin-2-yl)-5-methyl-7H-pyrrolo[2,3-d]pyrimidin-4-yl)-2,5-dimethylpiperazine-1-carboxylate (110 mg, 90%) as a white solid. LC/MS ESI (m/z): 441 (M+H)⁺.

### Step 4. 4-((2S,5R)-2,5-Dimethylpiperazin-1-yl)-7-(4-fluoropyridin-2-yl)-5-methyl-7H-pyrrolo[2,3-d]pyrimidine

tert-Butyl (2R,5S)-4-(7-(4-fluoropyridin-2-yl)-5-methyl-7H-pyrrolo[2,3-d]pyrimidin-4-yl)-2,5-dimethylpiperazine-1-carboxylate (110 mg, 0.25 mmol) in DCM (5 mL) was treated with TFA (2.0 mL). The resulting mixture was stirred at room temperature for 2 h. After removal of solvent, the residue was diluted with DCM and washed with NaHCO₃ (aq.). The organic layer was extracted twice with DCM. The combined organic layers were washed with brine, dried over Na₂SO₄, filtered and concentrated. The residue was used in the next step directly. LC/MS ESI (m/z): 341 (M+H)⁺.

### Step 5. 1-((2R,5S)-4-(7-(4-Fluoropyridin-2-yl)-5-methyl-7H-pyrrolo[2,3-d]pyrimidin-4-yl)-2,5-dimethylpiperazin-1-yl)-2,2-dimethylpropan-1-one

To a solution of 4-((2S,5R)-2,5-dimethylpiperazin-1-yl)-7-(4-fluoropyridin-2-yl)-5-methyl-7H-pyrrolo[2,3-d]pyrimidine (70 mg, 0.21 mmol) in DCM (10 mL) at 0° C. were added TEA (55 mg, 0.54 mmol) and pivaloyl chloride (32 mg, 0.27 mmol). After stirring at room temperature for 2 h, the reaction was diluted with DCM and washed with brine. The aqueous layer was extracted twice with DCM. The combined organic layers were washed with brine, dried over Na₂SO₄, filtered and concentrated. The residue was purified by flash column chromatography (silica gel, 0˜50% ethyl acetate in petroleum ether) to give crude product which was further purified by prep-HPLC to afford 1-((2R,5S)-4-(7-(4-fluoropyridin-2-yl)-5-methyl-7H-pyrrolo[2,3-d]pyrimidin-4-yl)-2,5-dimethylpiperazin-1-yl)-2,2-dimethylpropan-1-one (15 mg, 17%) as a white solid. LC/MS ESI (m/z): 425 (M+H)⁺. ¹H NMR (400 MHZ, CD₃OD) δ 8.68 (d, J=11.2 Hz, 1H), 8.50-8.40 (m, 2H), 8.03 (s, 1H), 7.09 (t, J=5.7 Hz, 1H), 4.72 (m, 1H), 4.57 (m, 1H), 4.39-4.08 (br. s, 1H), 3.86-3.51 (m, 3H), 2.50 (s, 3H), 1.34 (s, 9H), 1.26 (m, 3H), 1.13 (br. s, 3H).

The following compounds were prepared by procedures analogous to the synthesis of compound 244 from the corresponding boronic acids and acid chlorides. In one instance, the cyclopropyl group was introduced by Suzuki coupling with cyclopropylboronic acid (Pd-118, K₂CO₃ (20 eq.), 80° C. in toluene). For some analogs, the cyclobutyl group was introduced by Suzuki coupling of cyclobutylboronic acid and the corresponding aryl bromides. For compound 256, compound 262, compound 258, and compound 251 the corresponding carboxylic acid was synthesized following procedures described in US patent US20190248809).

| Cmpd | | |
|---|---|---|
| No. | Chemical Name | LCMS and ¹H NMR |
| 245 | 1-((2R,5S)-2,5-dimethyl-4-(5- | LC/MS ESI (m/z): 475 (M + H)⁺. ¹H NMR (400 MHz, |
| | methyl-7-(4- | CD₃OD) δ 9.23 (s, 1H), 8.69 (d, J = 5.1 Hz, 1H), 8.45 (s, |
| | (trifluoromethyl)pyridin-2-yl)- | 1H), 8.07 (s, 1H), 7.52 (d, J = 5.0 Hz, 1H), 4.72 (br. s, 1H), |
| | 7H-pyrrolo[2,3-d]pyrimidin-4- | 4.58 (br. s, 1H), 4.23 (br. s, 1H), 3.91-3.47 (m, 3H), 2.51 |
| | yl)piperazin-1-yl)-2,2- | (s, 3H), 1.34 (s, 9H), 1.30-1.02 (m, 6H). |
| | dimethylpropan-1-one | |
| 246 | 1-((2R,5S)-4-(5-cyclopropyl-7- | LC/MS ESI (m/z): 466 (M + H)⁺. ¹H NMR (400 MHz, |
| | (3-fluorophenyl)-7H- | CDCl₃) δ 8.38 (d, J = 3.7 Hz, 1H), 7.43-7.35 (m, 3H), |
| | pyrrolo[2,3-d]pyrimidin-4-yl)- | 6.96 (m, 1H), 6.90 (m, 1H), 5.24 (m, ~0.5H), 4.97-4.86 |
| | 2,5-dimethylpiperazin-1-yl)-2- | (m, 1H), 4.77 (m, ~0.5H), 4.42 (m, 1H), 3.90 (m, ~0.5H), |
| | methoxy-2-methylpropan-1-one | 3.84-3.63 (m, 2H), 3.40 (m, ~0.5H), 3.29 (s, ~1.5H), 3.19 |
| | | (s, ~1.5H), 1.97 (m, 1H), 1.43 (m, 6H), 1.32 (d, J = 6.7 |
| | | Hz, ~1.5H), 1.18 (d, J = 6.6 Hz, ~1.5H), 1.10 (d, J = 6.7 |
| | | Hz, ~1.5H), 1.01 (d, J = 6.7 Hz, ~1.5H), 0.99-0.93 (m, 2H), |
| | | 0.80 (m, 1H), 0.59 (m, 1H); several peaks and their |
| | | integrations are fractions of protons on account of presence |
| | | of rotomers. |
| 247 | (1-fluorocyclopropyl)((2R,5S)- | LC/MS ESI (m/z): 507 (M + H)⁺. ¹H NMR (400 MHz, |
| | 4-(5-(2-fluorophenyl)-7-(4- | CDCl3) δ 8.81 (dd, J = 10.9, 2.2 Hz, 1H), 8.58 (s, 1H), 8.44 |
| | fluoropyridin-2-yl)-7H- | (dd, J = 8.6, 5.7 Hz, 1H), 8.32 (s, 1H), 7.51 (t, J = 7.3 Hz, |
| | pyrrolo[2,3-d]pyrimidin-4-yl)- | 1H), 7.38 (m, 1H), 7.29-7.18 (m, 2H), 6.96 (m, 1H), 4.55 |
| | 2,5-dimethylpiperazin-1- | (br. s, 1H), 4.31 (br. s, ~0.5H), 4.09 (br. s, ~0.5H), 3.77- |
| | yl)methanone | 3.47 (m, ~1.5H), 3.45-3.20 (m, 2H), 2.69 (d, J = 11.8 |
| | | Hz, ~0.5H), 1.36-0.81 (m, 10H); several peaks and their |
| | | integrations are fractions of protons on account of presence |
| | | of rotomers. |
| 248 | 1-((2R,5S)-4-(5-cyclobutyl-7-(3- | LC/MS ESI (m/z): 450 (M + H)⁺. ¹H NMR (400 MHz, |
| | fluorophenyl)-7H-pyrrolo[2,3- | CDCl3) δ 8.38 (m, 1H), 7.49-7.37 (m, 3H), 7.14 (m, 1H), |
| | d]pyrimidin-4-yl)-2,5- | 6.98 (t, J = 8.3 Hz, 1H), 4.77 (br. s, ~0.5H), 4.60 (br. |
| | dimethylpiperazin-1-yl)-2- | s, ~0.5H), 4.40-4.10 (m, ~1.5H), 3.85-3.61 (m, ~2.5H), |
| | methylpropan-1-one | 3.58-3.41 (m, ~2H), 2.88-2.67 (m, 1H), 2.47-2.32 (m, |
| | | 2H), 2.15-1.86 (m, 4H), 1.28-1.11 (m, 8H), 1.07-0.95 |
| | | (m, 4H); several peaks and their integrations are fractions of |
| | | protons on account of presence of rotomers. |
| 249 | ((2R,5S)-4-(5-cyclobutyl-7-(3- | LC/MS ESI (m/z): 466 (M + H)⁺. ¹H NMR (400 MHz, |
| | fluorophenyl)-7H-pyrrolo[2,3- | CDCl3) δ 8.39 (s, 1H), 7.50-7.38 (m, 3H), 7.17 (s, 1H), |
| | d]pyrimidin-4-yl)-2,5- | 6.98 (m, 1H), 4.72-4.42 (m, 2H), 4.21-3.57 (m, 4H), |
| | dimethylpiperazin-1-yl)(1- | 3.47 (d, J = 13.3 Hz, 1H), 2.47-2.34 (m, 2H), 2.16-1.87 |
| | fluorocyclopropyl)methanone | (m, 4H), 1.29-1.01 (m, 10H); several peaks and their |
| | | integrations are fractions of protons on account of presence |
| | | of rotomers. |
| 250 | 1-((2R,5S)-4-(5-cyclobutyl-7-(3- | LC/MS ESI (m/z): 464 (M + H)⁺. ¹H NMR (400 MHz, |
| | fluorophenyl)-7H-pyrrolo[2,3- | CDCl3) δ 8.45 (s, 1H), 7.57-7.44 (m, 3H), 7.24 (s, 1H), |
| | d]pyrimidin-4-yl)-2,5- | 7.05 (m, 1H), 4.67 (br. s, 1H), 4.50 (br. s, 1H), 4.20 (br. s, |
| | dimethylpiperazin-1-yl)-2,2- | 1H), 3.90-3.54 (m, 3H), 3.50 (d, J = 13.3 Hz, 1H), 2.55- |
| | dimethylpropan-1-one | 2.39 (m, 2H), 2.23-1.93 (m, 4H), 1.35 (s, 9H), 1.30-1.08 |
| | | (m, 6H). |
| 251 | 1-((2R,5S)-4-(5-cyclobutyl-7-(3- | LC/MS ESI (m/z): 532 (M + H)⁺. ¹H NMR (400 MHz, |
| | fluorophenyl)-7H-pyrrolo[2,3- | CDCl3) δ 8.38 (d, J = 7.9 Hz, 1H), 7.50-7.36 (m, 3H), |
| | d]pyrimidin-4-yl)-2,5- | 7.14 (m, 1H), 6.98 (t, J = 8.2 Hz, 1H), 4.78 (br. s, ~0.5H), |
| | dimethylpiperazin-1-yl)-4,4,4- | 4.60 (br. s, ~0.5H), 4.44-4.05 (m, ~1.5H), 3.90-3.60 |
| | trifluoro-3,3-dimethylbutan-1- | (m, ~2.5H), 3.55-3.41 (m, 2H), 2.75-2.50 (m, 1H), 2.47- |
| | one | 2.31 (m, 3H), 2.18-1.87 (m, 4H), 1.31-1.28 (m, 4H), |
| | | 1.25-1.13 (m, 5H), 0.99 (m, 3H); several peaks and their |
| | | integrations are fractions of protons on account of presence |
| | | of rotomers. |
| 252 | 1-((2R,5S)-4-(5-cyclobutyl-7-(3- | LC/MS ESI (m/z): 518 (M + H)⁺. ¹H NMR (400 MHz, |
| | fluorophenyl)-7H-pyrrolo[2,3- | CDCl3) δ 8.38 (s, 1H), 7.52-7.36 (m, 3H), 7.17 (s, 1H), |
| | d]pyrimidin-4-yl)-2,5- | 6.98 (t, J = 8.2 Hz, 1H), 4.65-4.39 (m, 2H), 4.10 (br. s, |
| | dimethylpiperazin-1-yl)-3,3,3- | 1H), 3.81-3.54 (m, 3H), 3.45 (d, J = 13.2 Hz, 1H), 2.47- |
| | trifluoro-2,2-dimethylpropan-1- | 2.31 (m, 2H), 2.18-1.87 (m, 4H), 1.51-1.48 (m, 6H), |
| | one | 1.19-1.01 (m, 6H). |
| 253 | 1-((2R,5S)-4-(7-(3- | LC/MS ESI (m/z): 480 (M + H)⁺. ¹H NMR (400 MHz, |
| | chlorophenyl)-5-cyclobutyl-7H- | CDCl3) δ 8.44 (s, 1H), 7.74 (t, J = 2.0 Hz, 1H), 7.66 (m, |
| | pyrrolo[2,3-d]pyrimidin-4-yl)- | 1H), 7.45 (t, J = 8.1 Hz, 1H), 7.33 (m, 1H), 7.23 (s, 1H), |
| | 2,5-dimethylpiperazin-1-yl)-2,2- | 4.58 (m, 2H), 4.22 (br. s, 1H), 3.82-3.62 (m, 3H), 3.50 (d, |
| | dimethylpropan-1-one | J = 13.2 Hz, 1H), 2.47 (m, 2H), 2.22-1.95 (m, 4H), 1.35 |
| | | (s, 9H), 1.29-1.08 (m, 6H). |
| 254 | ((2R,5S)-4-(7-(3-chlorophenyl)- | LC/MS ESI (m/z): 482 (M + H)⁺. ¹H NMR (400 MHz, |
| | 5-cyclobutyl-7H-pyrrolo[2,3- | CDCl3) δ 8.45 (s, 1H), 7.74 (t, J = 2.0 Hz, 1H), 7.67 (m, |
| | d]pyrimidin-4-yl)-2,5- | 1H), 7.45 (t, J = 8.1 Hz, 1H), 7.33 (m, 1H), 7.23 (s, 1H), |
| | dimethylpiperazin-1-yl)(1- | 4.80-4.50 (m, 2H), 4.30-4.10 (m, ~1.5H), 3.85-3.64 |
| | fluorocyclopropyl)methanone | (m, ~2.5H), 3.54 (d, J = 13.4 Hz, 1H), 2.48 (m, 2H), 2.22- |
| | | 1.95 (m, 4H), 1.38-1.10 (m, 10H); several peaks and their |
| | | integrations are fractions of protons on account of presence |
| | | of rotomers. |
| 255 | 1-((2R,5S)-4-(5-cyclobutyl-7- | LC/MS ESI (m/z): 482 (M + H)⁺. ¹H NMR(400 MHz, |
| | (3,5-difluorophenyl)-7H- | CDCl3) δ 8.45 (s, 1H), 7.41 (m, 2H), 7.22 (s, 1H), 6.78 (m, |
| | pyrrolo[2,3-d]pyrimidin-4-yl)- | 1H), 4.70-4.50 (m, 2H), 4.19 (br. s, 1H), 3.85-3.62 (m, |
| | 2,5-dimethylpiperazin-1-yl)-2,2- | 3H), 3.49 (d, J = 13.2 Hz, 1H), 2.47 (m, 2H), 2.22-1.95 |
| | dimethylpropan-1-one | (m, 4H), 1.34 (s, 9H), 1.29-1.08 (m, 6H). |
| 256 | 1-((2R,5S)-4-(5-cyclobutyl-7- | LC/MS ESI (m/z): 550 (M + H)⁺. ¹H NMR (400 MHz, |
| | (3,5-difluorophenyl)-7H- | CDCl3) δ 8.46 (d, J = 8.1 Hz, 1H), 7.41 (d, J = 6.4 Hz, 2H), |
| | pyrrolo[2,3-d]pyrimidin-4-yl)- | 7.22 (d, J = 22.9 Hz, 1H), 6.79 (t, J = 8.7 Hz, 1H), 4.85- |
| | 2,5-dimethylpiperazin-1-yl)- | 4.65 (m, 1H), 4.46 (br. s, ~0.5H), 4.37 (d, J = 13.3 |
| | 4,4,4-trifluoro-3,3- | Hz, ~0.5H), 4.20 (br. s, ~0.5H), 3.92 (d, J = 10.6 Hz, ~0.5H), |
| | dimethylbutan-1-one | 3.83-3.66 (m, 2H), 3.59-3.46 (m, 2H), 2.80-2.60 (m, |
| | | 1H), 2.50-2.36 (m, 3H), 2.24-1.96 (m, 4H), 1.40-1.15 |
| | | (m, 9H), 1.05 (d, J = 6.7 Hz, 3H); several peaks and their |
| | | integrations are fractions of protons on account of presence |
| | | of rotomers. |
| 257 | 1-((2R,5S)-4-(7-(3- | LC/MS ESI (m/z): 534 (M + H)⁺. ¹H NMR (400 MHz, |
| | chlorophenyl)-5-cyclobutyl-7H- | CDCl3) δ 8.44 (s, 1H), 7.74 (t, J = 1.9 Hz, 1H), 7.66 (dd, |
| | pyrrolo[2,3-d]pyrimidin-4-yl)- | J = 8.1, 1.0 Hz, 1H), 7.45 (t, J = 8.1 Hz, 1H), 7.33 (dd, J = |
| | 2,5-dimethylpiperazin-1-yl)- | 8.1, 1.0 Hz, 1H), 7.23 (s, 1H), 4.70-4.50 (m, 2H), 4.17 (br. |
| | 3,3,3-trifluoro-2,2- | s, 1H), 3.83-3.66 (m, 3H), 3.52 (d, J = 13.3 Hz, 1H), 2.46 |
| | dimethylpropan-1-one | (m, 2H), 2.21-1.97 (m, 4H), 1.59 (s, 3H), 1.41-1.07 (m, |
| | | 9H). |
| 258 | 1-((2R,5S)-4-(7-(3- | LC/MS ESI (m/z): 548 (M + H)⁺. ¹H NMR (400 MHz, |
| | chlorophenyl)-5-cyclobutyl-7H- | CDCl3) δ 8.44 (d, J = 7.8 Hz, 1H), 7.74 (m, 1H), 7.66 (d, |
| | pyrrolo[2,3-d]pyrimidin-4-yl)- | J = 8.1 Hz, 1H), 7.45 (t, J = 8.1 Hz, 1H), 7.33 (d, J = 7.9 Hz, |
| | 2,5-dimethylpiperazin-1-yl)- | 1H), 7.23 (d, J = 22.4 Hz, 1H), 4.90-4.65 (m, 1H), 4.53- |
| | 4,4,4-trifluoro-3,3- | 4.33 (m, 1H), 4.20 (br. s, ~0.5H), 3.92 (d, J = 10.2 |
| | dimethylbutan-1-one | Hz, ~0.5H), 3.82-3.68 (m, 2H), 3.60-3.45 (m, 2H), 2.80- |
| | | 2.60 (m, 1H), 2.50-2.37 (m, 3H), 2.21-1.94 (m, 4H), |
| | | 1.40-1.25 (m, ~7.5H), 1.22 (d, J = 6.6 Hz, ~1.5H), 1.06 |
| | | (m, 3H); several peaks and their integrations are fractions of |
| | | protons on account of presence of rotomers. |
| 259 | 1-((2R,5S)-4-(5-cyclobutyl-7- | LC/MS ESI (m/z): 536 (M + H)⁺. ¹H NMR (400 MHz, |
| | (3,5-difluorophenyl)-7H- | CDCl3) δ 8.45 (s, 1H), 7.41 (m, 2H), 7.21 (s, 1H), 6.82- |
| | pyrrolo[2,3-d]pyrimidin-4-yl)- | 6.76 (m, 1H), 4.61 (m, 2H), 4.15 (br. s, 1H), 3.85-3.68 (m, |
| | 2,5-dimethylpiperazin-1-yl)- | 3H), 3.51 (d, J = 13.4 Hz, 1H), 2.47 (m, 2H), 2.22-1.96 |
| | 3,3,3-trifluoro-2,2- | (m, 4H), 1.59 (s, 3H), 1.52-1.07 (m, 9H). |
| | dimethylpropan-1-one | |
| 260 | ((2R,5S)-4-(5-cyclobutyl-7-(3- | LC/MS ESI (m/z): 516 (M + H)⁺. ¹H NMR (400 MHz, |
| | (trifluoromethyl)phenyl)-7H- | CDCl3) δ 8.45 (s, 1H), 8.03-7.95 (m, 2H), 7.68-7.59 (m, |
| | pyrrolo[2,3-d]pyrimidin-4-yl)- | 2H), 7.26 (s, 1H), 4.80-4.50 (m, 2H), 4.41-4.01 (m, ~1.5H), |
| | 2,5-dimethylpiperazin-1-yl)(1- | 3.87-3.62 (m, ~2.5H), 3.55 (d, J = 13.3 Hz, 1H), |
| | fluorocyclopropyl)methanone | 2.49 (m, 2H), 2.27-1.96 (m, 4H), 1.37-1.09 (m, 10H); |
| | | several peaks and their integrations are fractions of protons |
| | | on account of presence of rotomers. |
| 261 | 1-((2R,5S)-4-(5-cyclobutyl-7-(3- | LC/MS ESI (m/z): 514 (M + H)⁺. ¹H NMR (400 MHz, |
| | (trifluoromethyl)phenyl)-7H- | CDCl3) δ 8.44 (s, 1H), 8.02-7.94 (m, 2H), 7.68-7.59 (m, |
| | pyrrolo[2,3-d]pyrimidin-4-yl)- | 2H), 7.26 (s, 1H), 4.75-4.45 (m, 2H), 4.21 (br. s, 1H), 3.85- |
| | 2,5-dimethylpiperazin-1-yl)-2,2- | 3.59 (m, 3H), 3.51 (d, J = 13.1 Hz, 1H), 2.49 (m, 2H), |
| | dimethylpropan-1-one | 2.25-1.96 (m, 4H), 1.35 (s, 9H), 1.28-1.05 (m, 6H). |
| 262 | 1-((2R,5S)-4-(5-cyclobutyl-7-(3- | LC/MS ESI (m/z): 582 (M + H)⁺. ¹H NMR (400 MHz, |
| | (trifluoromethyl)phenyl)-7H- | CDCl3) δ 8.44 (d, J = 7.9 Hz, 1H), 8.02-7.95 (m, 2H), |
| | pyrrolo[2,3-d]pyrimidin-4-yl)- | 7.68-7.59 (m, 2H), 7.29-7.23 (m, 1H), 4.86 (br. s, ~0.5H), |
| | 2,5-dimethylpiperazin-1-yl)- | 4.69 (br. s, ~0.5H), 4.51-4.31 (m, 1H), 4.20 (br. |
| | 4,4,4-trifluoro-3,3- | s, ~0.5H), 3.92 (d, J = 10.3 Hz, ~0.5H), 3.84-3.69 (m, 2H), |
| | dimethylbutan-1-one | 3.60-3.45 (m, 2H), 2.80-2.60 (m, 1H), 2.50-2.35 (m, |
| | | 3H), 2.24-1.97 (m, 4H), 1.40-1.20 (m, 9H), 1.07 (m, |
| | | 3H); several peaks and their integrations are fractions of |
| | | protons on account of presence of rotomers. |
| 263 | 1-((2R,5S)-4-(5-cyclobutyl-7-(3- | LC/MS ESI (m/z): 568 (M + H)⁺. ¹H NMR (400 MHz, |
| | (trifluoromethyl)phenyl)-7H- | CDCl3) δ 8.44 (s, 1H), 8.02-7.94 (m, 2H), 7.68-7.59 (m, |
| | pyrrolo[2,3-d]pyrimidin-4-yl)- | 2H), 7.26 (br. s, 1H), 4.70-4.50 (m, 2H), 4.17 (br. s, 1H), |
| | 2,5-dimethylpiperazin-1-yl)- | 3.85-3.68 (m, 3H), 3.53 (d, J = 13.1 Hz, 1H), 2.48 (m, |
| | 3,3,3-trifluoro-2,2- | 2H), 2.24-1.95 (m, 4H), 1.58 (s, 6H), 1.24-1.06 (m, 6H). |
| | dimethylpropan-1-one | |

### Example 48. Synthesis of 2-(5-cyclopropyl-4-((2S,5R)-4-(2-methoxy-2-methylpropanoyl)-2,5-dimethylpiperazin-1-yl)-7H-pyrrolo[2,3-d]pyrimidin-7-yl)isonicotinonitrile (Compound 264)

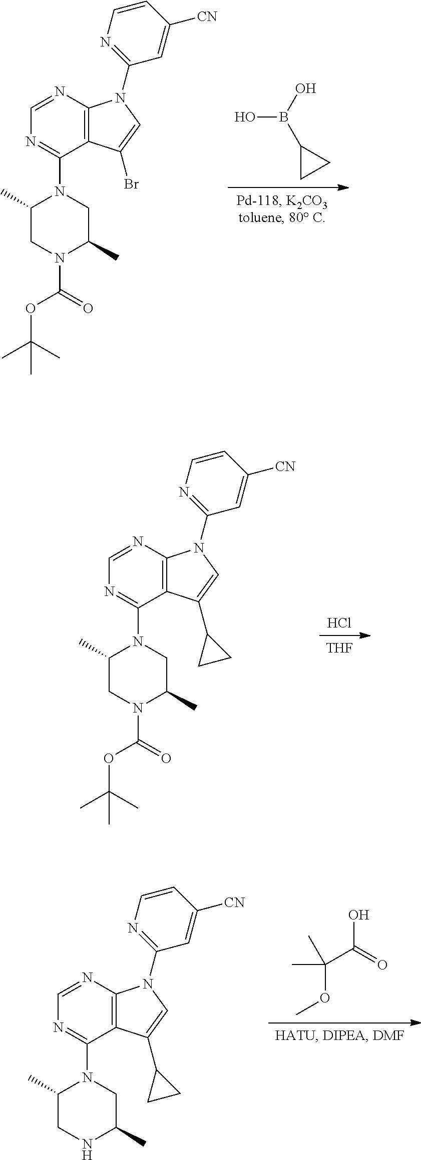

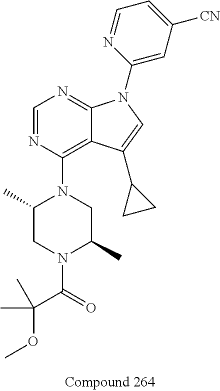 [SMILES: COC(C)(C)C(=O)N1C[C@H](C)N(c2ncnc3c2c(C2CC2)cn3-c2cc(C#N)ccn2)C[C@H]1C]

### Step 1. tert-Butyl (2R,5S)-4-(7-(4-cyanopyridin-2-yl)-5-cyclopropyl-7H-pyrrolo[2,3-d]pyrimidin-4-yl)-2,5-dimethylpiperazine-1-carboxylate

To a solution of tert-butyl (2R,5S)-4-[5-bromo-7-(4-cyanopyridin-2-yl)-7H-pyrrolo[2,3-d]pyrimidin-4-yl]-2,5-dimethylpiperazine-1-carboxylate (1500 mg, 2.9 mmol, prepared following a similar procedure outlined for compound 268 using 2-fluoroisonicotinonitrile instead) in toluene (15 mL) were added cyclopropylboronic acid (500 mg, 5.9 mmol), dipotassium carbonate (8.1 g, 59 mmol) and Pd-118 (380 mg, 0.60 mmol). After heating at 80° C. overnight, the reaction mixture was cooled to room temperature and filtered. The filtrate was concentrated and purified by flash column chromatography to afford tert-butyl (2R,5S)-4-(7-(4-cyanopyridin-2-yl)-5-cyclopropyl-7H-pyrrolo[2,3-d]pyrimidin-4-yl)-2,5-dimethylpiperazine-1-carboxylate (1000 mg, 73%) as a solid. LC/MS ESI (m/z): 474 (M+H)⁺.

### Step 2. 2-(5-Cyclopropyl-4-((2S,5R)-2,5-dimethylpiperazin-1-yl)-7H-pyrrolo[2,3-d]pyrimidin-7-yl)isonicotinonitrile

To a solution of tert-butyl (2R,5S)-4-(7-(4-cyanopyridin-2-yl)-5-cyclopropyl-7H-pyrrolo[2,3-d]pyrimidin-4-yl)-2,5-dimethylpiperazine-1-carboxylate (1000 mg, 2.1 mmol) in THF (8 mL) at 0° C. was added HCl (4.0 mL, 4.0 M in dioxane). The resulting mixture was stirred at room temperature for 2 h. The reaction mixture was then basified with NaHCO₃ (aq.) and extracted twice with DCM. The combined organic layers were washed with NaHCO₃ (aq.), dried over Na₂SO₄, filtered and concentrated. The residue was used in the next step directly. LC/MS ESI (m/z): 374 (M+H)⁺.

### Step 3. 2-(5-Cyclopropyl-4-((2S,5R)-4-(2-methoxy-2-methylpropanoyl)-2,5-dimethylpiperazin-1-yl)-7H-pyrrolo[2,3-d]pyrimidin-7-yl)isonicotinonitrile

To a solution of 2-(5-cyclopropyl-4-((2S,5R)-2,5-dimethylpiperazin-1-yl)-7H-pyrrolo[2,3-d]pyrimidin-7-yl)isonicotinonitrile (100 mg, 0.27 mmol) in DMF (8 mL) at 0° C. were added DIPEA (70 mg, 0.54 mmol), 2-methoxy-2-methylpropanoic acid (38 mL, 0.32 mmol) and HATU (120 mg, 0.32 mol). After stirring at room temperature for 2 h, the reaction was quenched with NaHCO₃ (aq.) and extracted twice with EtOAc. The combined organic layers were washed with brine, dried over Na₂SO₄, filtered, concentrated. The residue was purified by prep-HPLC to afford 2-(5-cyclopropyl-4-((2S,5R)-4-(2-methoxy-2-methylpropanoyl)-2,5-dimethylpiperazin-1-yl)-7H-pyrrolo[2,3-d]pyrimidin-7-yl)isonicotinonitrile (60 mg, 47%) as an off-white solid. LC/MS ESI (m/z): 474 (M+H)⁺. ¹H NMR (400 MHz, CDCl₃) δ 9.25 (s, 1H), 8.52 (d, J=5.0 Hz, 1H), 8.44 (d, J=4.6 Hz, 1H), 7.74 (d, J=3.3 Hz, 1H), 7.28 (d, J=5.0 Hz, 1H), 5.23 (m, ˜0.5H), 4.96-4.82 (m, 1H), 4.76 (m, ˜0.5H), 4.48-4.33 (m, 1H), 3.93-3.64 (m, ˜2.5H), 3.38 (m, ˜0.5H), 3.29 (s, ˜1.5H), 3.19 (s, ˜1.5H), 1.94 (m, 1H), 1.48-1.38 (m, 6H), 1.31-1.17 (m, 3H), 1.08-1.00 (m, 3H), 1.00-0.94 (m, 2H), 0.89 (m, 1H), 0.65 (m, 1H); several peaks and their integrations are fractions of protons on account of presence of rotomers.

The following compounds were prepared by procedures analogous to the synthesis of compound 264 from the corresponding carboxylic acids, chiral amines and boronic acids.

| Cmpd | | |
|---|---|---|
| No. | Chemical Name | LCMS and ¹H NMR |
| 265 | (R)-2-(5-cyclopropyl-4-(4-(2- | LC/MS ESI (m/z): 460 (M + H)⁺. ¹H NMR (400 MHz, |
| | methoxy-2-methylpropanoyl)-3- | CDCl3) δ 9.33 (s, 1H), 8.59 (dd, J = 5.0, 0.7 Hz, 1H), 8.51 |
| | methylpiperazin-1-yl)-7H- | (s, 1H), 7.82 (s, 1H), 7.35 (dd, J = 5.0, 1.3 Hz, 1H), 5.39- |
| | pyrrolo [2,3-d]pyrimidin-7- | 3.63 (m, 5H), 3.42-3.31 (m, 2H), 3.29-3.25 (m, 2H), |
| | yl)isonicotinonitrile | 3.18-2.96 (m, 1H), 2.03 (m, 1H), 1.52-1.37 (m, 7H), |
| | | 1.24 (m, 2H), 1.06 (m, 2H), 1.00-0.93 (m, 1H), 0.75 (m, |
| | | 1H); several peaks and their integrations are fractions of |
| | | protons on account of presence of rotomers. |
| 266 | (R)-2-(5-(2-fluorophenyl)-4-(4- | LC/MS ESI (m/z): 514 (M + H)⁺. ¹H NMR (400 MHz, |
| | (2-methoxy-2- | CDCl3) δ 9.34 (s, 1H), 8.56 (d, J = 5.0 Hz, 1H), 8.51 (s, |
| | methylpropanoyl)-3- | 1H), 8.22 (s, 1H), 7.43 (t, J = 7.5 Hz, 1H), 7.36-7.29 (m, |
| | methylpiperazin-1-yl)-7H- | 2H), 7.21 (m, 1H), 7.16 (m, 1H), 5.20-4.60 (m, 1H), 4.30- |
| | pyrrolo[2,3-d]pyrimidin-7- | 3.52 (m, 3H), 3.17 (s, 1H), 3.05 (s, 2H), 2.94 (m, 1H), |
| | yl)isonicotinonitrile | 2.85-2.23 (m, 2H), 1.40-1.20 (m, 7H), 1.05 (d, J = 6.8 |
| | | Hz, 2H); several peaks and their integrations are fractions |
| | | of protons on account of presence of rotomers. |
| 267 | 2-(5-(2-fluorophenyl)-4- | LC/MS ESI (m/z): 526 (M + H)⁺. ¹H NMR (400 MHz, |
| | ((2S,5R)-4-(1- | CDCl3) δ 9.35 (s, 1H), 8.56 (d, J = 5.0 Hz, 1H), 8.52 (d, J = |
| | methoxycyclopropane-1- | 6.1 Hz, 1H), 8.24 (d, J = 5.4 Hz, 1H), 7.44 (m, 1H), 7.36- |
| | carbonyl)-2,5- | 7.28 (m, 2H), 7.24-7.10 (m, 2H), 4.78 (br. s, ~0.5H), 4.50 |
| | dimethylpiperazin-1-yl)-7H- | (br. s, ~0.5H), 4.31 (br. s, ~0.5H), 4.00-3.70 (m, ~1.5H), |
| | pyrrolo [2,3-d]pyrimidin-7- | 3.48 (d, J = 16 Hz, ~0.5H), 3.30-3.10 (m, 5H), 2.55 (d, J = |
| | yl)isonicotinonitrile | 16 Hz, ~0.5H), 1.27-1.15 (m, 2H), 1.05-0.90 (m, 4H), |
| | | 0.90-0.75 (m, 4H); several peaks and their integrations are |
| | | fractions of protons on account of presence of rotomers. |

### Example 49. Synthesis of 2-(5-(azetidin-1-yl)-4-((2S,5R)-4-(2-methoxy-2-methylpropanoyl)-2,5-dimethylpiperazin-1-yl)-7H-pyrrolo[2,3-d]pyrimidin-7-yl)isonicotinonitrile (Compound 268)

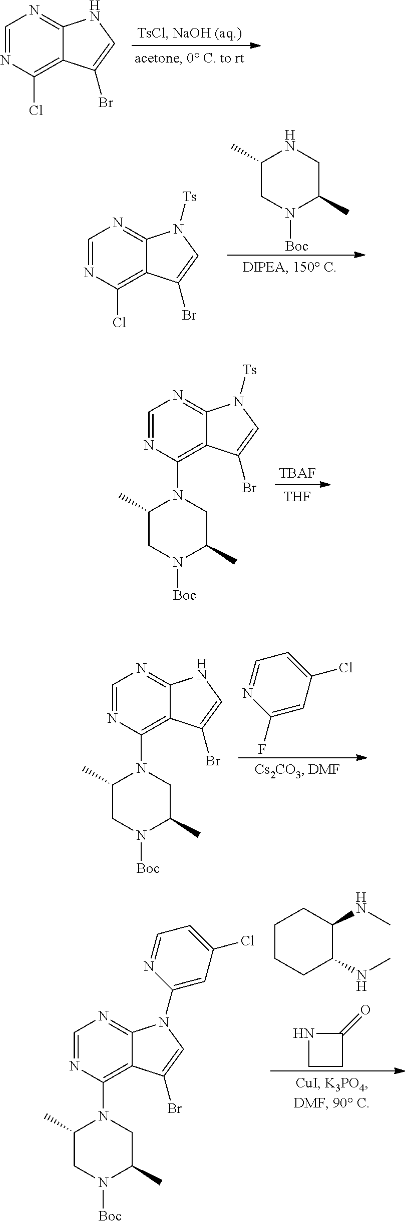

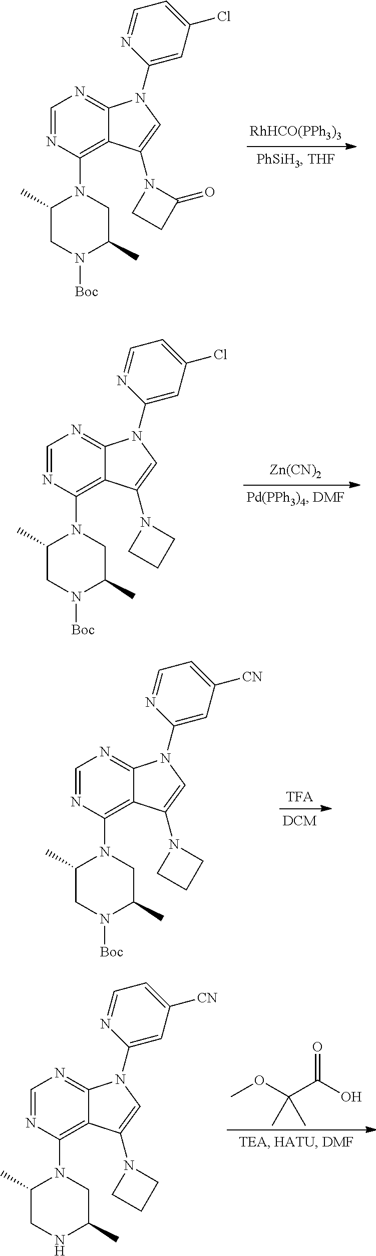

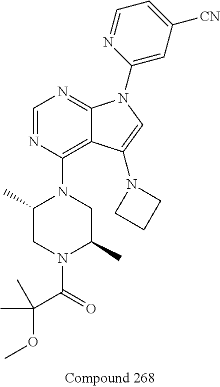 [SMILES: COC(C)(C)C(=O)N1C[C@H](C)N(c2ncnc3c2c(N2CCC2)cn3-c2cc(C#N)ccn2)C[C@H]1C]

### Step 1. 5-Bromo-4-chloro-7-tosyl-7H-pyrrolo[2,3-d]pyrimidine

To a solution of 5-bromo-4-chloro-7H-pyrrolo[2,3-d]pyrimidine (100 g, 0.43 mol) and 4-methylbenzenesulfonyl chloride (120 g, 0.65 mol) in acetone (1.5 L) at 0° C. was added 2.0 M NaOH (0.33 L) dropwise. After the addition, the reaction was allowed to warm up to room temperature and stirred for another 3 h. The resulting precipitate was collected by filtration, washed twice with water twice and dried in vacuo to afford 5-bromo-4-chloro-7-tosyl-7H-pyrrolo[2,3-d]pyrimidine (160 g, 96%) as an off-white solid. LC/MS ESI (m/z): 386, 388 (M+H)⁺.

### Step 2. tert-Butyl (2R,5S)-4-(5-bromo-7-tosyl-7H-pyrrolo[2,3-d]pyrimidin-4-yl)-2,5-dimethylpiperazine-1-carboxylate

To a solution of 5-bromo-4-chloro-7-tosyl-7H-pyrrolo[2,3-d]pyrimidine (17 g, 44 mmol) in DIPEA (200 mL) was added tert-butyl (2R,5S)-2,5-dimethylpiperazine-1-carboxylate (19 g, 88 mmol). The resulting mixture was stirred at 150° C. for 2 h. After cooling to room temperature, the reaction was concentrated and purified by flash column chromatography (silica gel, 0˜30% ethyl acetate in petroleum ether) to afford tert-butyl (2R,5S)-4-(5-bromo-7-tosyl-7H-pyrrolo[2,3-d]pyrimidin-4-yl)-2,5-dimethylpiperazine-1-carboxylate (20 g, 80%) as a white solid. LC/MS ESI (m/z): 564, 566 (M+H)⁺.

### Step 3. tert-Butyl (2R,5S)-4-(5-bromo-7H-pyrrolo[2,3-d]pyrimidin-4-yl)-2,5-dimethylpiperazine-1-carboxylate

To a solution of tert-butyl (2R,5S)-4-(5-bromo-7-tosyl-7H-pyrrolo[2,3-d]pyrimidin-4-yl)-2,5-dimethylpiperazine-1-carboxylate (20 g, 37 mmol) in THF (200 mL) was added TBAF (180 mL, 1.0 M in THF). After 3 h, the reaction was diluted with water and extracted twice with EtOAc. The combined organic layers were washed with brine, dried over Na₂SO₄, filtered and concentrated. The residue was purified by flash column chromatography (silica gel, 0˜80% ethyl acetate in petroleum ether) to afford tert-butyl (2R,5S)-4-(5-bromo-7H-pyrrolo[2,3-d]pyrimidin-4-yl)-2,5-dimethylpiperazine-1-carboxylate (13 g, 90%) as a white solid. LC/MS ESI (m/z): 410, 412 (M+H)⁺.

### Step 4. tert-Butyl (2R,5S)-4-(5-bromo-7-(4-chloropyridin-2-yl)-7H-pyrrolo[2,3-d]pyrimidin-4-yl)-2,5-dimethylpiperazine-1-carboxylate

To a solution of tert-butyl (2R,5S)-4-(5-bromo-7H-pyrrolo[2,3-d]pyrimidin-4-yl)-2,5-dimethylpiperazine-1-carboxylate (13 g, 32 mmol) in DMF (150 mL) were added 4-chloro-2-fluoropyridine (8.3 g, 64 mmol) and Cs₂CO₃ (52 g, 160 mmol). The resulting mixture was heated at 60° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜40% ethyl acetate in petroleum ether) to afford tert-butyl (2R,5S)-4-(5-bromo-7-(4-chloropyridin-2-yl)-7H-pyrrolo[2,3-d]pyrimidin-4-yl)-2,5-dimethylpiperazine-1-carboxylate (7.8 g, 47%) as a white solid. LC/MS ESI (m/z): 521, 523 (M+H)⁺.

### Step 5. tert-Butyl (2R,5S)-4-(7-(4-chloropyridin-2-yl)-5-(2-oxoazetidin-1-yl)-7H-pyrrolo[2,3-d]pyrimidin-4-yl)-2,5-dimethylpiperazine-1-carboxylate

To a solution of tert-butyl (2R,5S)-4-(5-bromo-7-(4-chloropyridin-2-yl)-7H-pyrrolo[2,3-d]pyrimidin-4-yl)-2,5-dimethylpiperazine-1-carboxylate (7.3 g, 14 mmol) in DMF (70 mL) were added azetidin-2-one (2.0 g, 28 mmol), trans-N,N′-dimethylcyclohexane-1,2-diamine (1.6 g, 14 mmol), CuI (2.7 g, 14 mmol) and K₃PO₄ (8.9 g, 42 mmol). The resulting mixture was heated at 90° C. overnight. After cooling to room temperature, the reaction was diluted with water and extracted twice with EtOAc. The combined organic layers were washed with brine, dried over Na₂SO₄, filtered and concentrated. The residue was purified by flash column chromatography (silica gel, 0˜50% ethyl acetate in petroleum ether) to afford tert-butyl (2R,5S)-4-(7-(4-chloropyridin-2-yl)-5-(2-oxoazetidin-1-yl)-7H-pyrrolo[2,3-d]pyrimidin-4-yl)-2,5-dimethylpiperazine-1-carboxylate (4.8 g, 67%) as a white solid. LC/MS ESI (m/z): 512 (M+H)⁺.

### Step 6. tert-Butyl (2R,5S)-4-(5-(azetidin-1-yl)-7-(4-chloropyridin-2-yl)-7H-pyrrolo[2,3-d]pyrimidin-4-yl)-2,5-dimethylpiperazine-1-carboxylate

To a solution of tert-butyl (2R,5S)-4-(7-(4-chloropyridin-2-yl)-5-(2-oxoazetidin-1-yl)-7H-pyrrolo[2,3-d]pyrimidin-4-yl)-2,5-dimethylpiperazine-1-carboxylate (2.0 g, 3.9 mmol) in THF (20 mL) at 0° C. were added RhHCO(PPh₃)₃ (370 mg, 0.40 mmol) and PhSiH₃ (2.2 g, 20 mmol) dropwise. After stirring at room temperature overnight under N₂, the reaction was partitioned between EtOAc and water. The aqueous layer was extracted twice with EtOAc twice. The combined organic layers were washed with brine, dried over Na₂SO₄, filtered and concentrated. The residue was purified by flash column chromatography (silica gel, 0˜35% ethyl acetate in petroleum ether) to afford tert-butyl (2R,5S)-4-(5-(azetidin-1-yl)-7-(4-chloropyridin-2-yl)-7H-pyrrolo[2,3-d]pyrimidin-4-yl)-2,5-dimethylpiperazine-1-carboxylate (1.4 g, 72%) as a yellow solid. LC/MS ESI (m/z): 498 (M+H)⁺.

### Step 7. tert-Butyl (2R,5S)-4-(5-(azetidin-1-yl)-7-(4-cyanopyridin-2-yl)-7H-pyrrolo[2,3-d]pyrimidin-4-yl)-2,5-dimethylpiperazine-1-carboxylate

To a solution of tert-butyl (2R,5S)-4-(5-(azetidin-1-yl)-7-(4-chloropyridin-2-yl)-7H-pyrrolo[2,3-d]pyrimidin-4-yl)-2,5-dimethylpiperazine-1-carboxylate (1.3 g, 2.6 mmol) in DMF (15 mL) were added Zn(CN)₂ (1.5 g, 13 mmol) and Pd(PPh₃)⁺ (1.5 g, 1.3 mmol). The resulting mixture was heated at 120° C. overnight. After cooling to room temperature, the solvent was removed and the residue was purified by flash column chromatography (silica gel, 0˜60% ethyl acetate in petroleum ether) to afford tert-butyl (2R,5S)-4-(5-(azetidin-1-yl)-7-(4-cyanopyridin-2-yl)-7H-pyrrolo[2,3-d]pyrimidin-4-yl)-2,5-dimethylpiperazine-1-carboxylate (710 mg, 55%) as a yellow solid. LC/MS ESI (m/z): 489 (M+H)⁺.

### Step 8. 2-(5-(Azetidin-1-yl)-4-((2S,5R)-2,5-dimethylpiperazin-1-yl)-7H-pyrrolo[2,3-d]pyrimidin-7-yl)isonicotinonitrile

To a solution of tert-butyl (2R,5S)-4-(5-(azetidin-1-yl)-7-(4-cyanopyridin-2-yl)-7H-pyrrolo[2,3-d]pyrimidin-4-yl)-2,5-dimethylpiperazine-1-carboxylate (710 mg, 1.5 mmol) in DCM (10 mL) at 0° C. was added TFA (3.0 mL). The resulting mixture was stirred at 0° C. for 2 h. After removal of solvent, the residue was diluted with DCM and washed with NaHCO₃ (aq.). The aqueous layer was extracted twice with DCM. The combined organic layers were washed with brine, dried over Na₂SO₄, filtered and concentrated. The residue was used in the next step directly. LC/MS ESI (m/z): 389 (M+H)⁺.

### Step 9. 2-(5-(Azetidin-1-yl)-4-((2S,5R)-4-(2-methoxy-2-methylpropanoyl)-2,5-dimethylpiperazin-1-yl)-7H-pyrrolo[2,3-d]pyrimidin-7-yl)isonicotinonitrile

To a solution of 2-(5-(azetidin-1-yl)-4-((2S,5R)-2,5-dimethylpiperazin-1-yl)-7H-pyrrolo[2,3-d]pyrimidin-7-yl)isonicotinonitrile (70 mg, 0.18 mmol) in DMF (10 mL) were added TEA (55 mg, 0.54 mmol), 2-methoxy-2-methylpropanoic acid (260 mg, 2.2 mmol) and HATU (840 mg, 2.2 mmol). After 2 h, the residue was diluted with EtOAc and washed with NaHCO₃ (aq.). The aqueous layer was extracted twice with EtOAc. The combined organic layers were washed with brine, dried over Na₂SO₄, filtered and concentrated. The residue was purified by flash column chromatography (silica gel, 0˜50% ethyl acetate in petroleum ether) to give crude product which was further purified by prep-HPLC to afford 2-(5-(azetidin-1-yl)-4-((2S,5R)-4-(2-methoxy-2-methylpropanoyl)-2,5-dimethylpiperazin-1-yl)-7H-pyrrolo[2,3-d]pyrimidin-7-yl)isonicotinonitrile (33 mg, 37%) as a yellow solid. LC/MS ESI (m/z): 489 (M+H)⁺. ¹H NMR (400 MHZ, CDCl₃) δ 9.35 (s, 1H), 8.58 (d, J=5.0 Hz, 1H), 8.44 (d, J=4.2 Hz, 1H), 7.47 (d, J=8.7 Hz, 1H), 7.30 (d, J=5.0 Hz, 1H), 5.33-5.13 (m, ˜1.5H), 4.77 (br. s, ˜0.5H), 4.48 (m, 1H), 4.18 (d, J=14.2 Hz, ˜0.5H), 3.99 (m, 1H), 3.92-3.78 (m, ˜2.5H), 3.68-3.49 (m, ˜3H), 3.35 (s, ˜1.5H), 3.25 (s, ˜1.5H), 2.31 (m, 2H), 1.55-1.40 (m, 6H), 1.26-1.20 (m, 3H), 1.07-0.95 (m, 3H); several peaks and their integrations are fractions of protons on account of presence of rotomers.

The following compounds were prepared by procedures analogous to the synthesis of compound 268 from the corresponding acids or acid chlorides and chiral amines. 4-Chloro-5-iodo-7H-pyrrolo[2,3-d]pyrimidine was used for the synthesis of compound 275 and compound 276.

| Cmpd | | |
|---|---|---|
| No. | Chemical Name | LCMS and ¹H NMR |
| 269 | (R)-2-(5-(azetidin-1-yl)-4-(4-(2- | LC/MS ESI (m/z): 475 (M + H)⁺. ¹H NMR (400 MHz, |
| | methoxy-2-methylpropanoyl)-3- | CDCl₃) δ 9.28 (s, 1H), 8.51 (dd, J = 5.0, 0.7 Hz, 1H), 8.36 |
| | methylpiperazin-1-yl)-7H- | (s, 1H), 7.41 (s, 1H), 7.23 (dd, J = 5.0, 1.3 Hz, 1H), 4.86- |
| | pyrrolo[2,3-d]pyrimidin-7- | 4.69 (m, ~2.5H), 4.64-4.26 (m, ~1.5H), 3.80-3.73 (m, |
| | yl)isonicotinonitrile | 2H), 3.66-3.49 (m, 3H), 3.33 (m, 1H), 3.25-3.18 (m, |
| | | 3H), 3.03-2.85 (m, 1H), 2.23 (m, 2H), 1.44-1.37 (m, |
| | | 6H), 1.15 (m, 1H), 0.98 (m, 2H); several peaks and their |
| | | integrations are fractions of protons on account of presence |
| | | of rotomers. |
| 270 | (R)-2-(5-(azetidin-1-yl)-4-(4-(1- | LC/MS ESI (m/z): 461 (M + H)⁺. ¹H NMR(400 MHz, |
| | fluorocyclopropane-1-carbonyl)- | CDCl₃) δ 9.28 (s, 1H), 8.51 (dd, J = 5.0, 0.7 Hz, 1H), 8.36 |
| | 3-methylpiperazin-1-yl)-7H- | (s, 1H), 7.41 (s, 1H), 7.23 (dd, J = 5.0, 1.3 Hz, 1H), 4.79 (d, |
| | pyrrolo[2,3-d]pyrimidin-7- | J = 12 Hz, 1H), 4.64 (br. s, 1H), 4.50 (d, J = 16 Hz, 1H), |
| | yl)isonicotinonitrile | 4.25 (br. s, 1H), 3.76 (m, 2H), 3.62-3.28 (m, 4H), 3.03 (m, |
| | | 1H), 2.25 (m, 2H), 1.28-1.14 (m, 4H), 1.12-0.99 (m, |
| | | 3H). |
| 271 | (R)-2-(5-(azetidin-1-yl)-4-(3- | LC/MS ESI (m/z): 459 (M + H)⁺. ¹H NMR (400 MHz, |
| | methyl-4-pivaloylpiperazin-1- | CDCl₃) δ 9.28 (s, 1H), 8.51 (dd, J = 5.0, 0.8 Hz, 1H), 8.35 |
| | yl)-7H-pyrrolo[2,3-d]pyrimidin- | (s, 1H), 7.40 (s, 1H), 7.22 (dd, J = 5.0, 1.3 Hz, 1H), 4.78- |
| | 7-yl)isonicotinonitrile | 4.63 (m, 2H), 4.42 (d, J = 12 Hz, 1H), 4.40-4.17 (br. s, |
| | | 1H), 3.75 (m, 2H), 3.59 (m, 2H), 3.50-3.27 (m, 2H), 2.93 |
| | | (m, 1H), 2.23 (m, 2H), 1.25 (s, 9H), 1.02 (m, 3H). |
| 272 | (R)-2-(5-(azetidin-1-yl)-4-(3- | LC/MS ESI (m/z): 459 (M + H)⁺ ¹H NMR (400 MHz, |
| | methyl-4-(3- | CDCl₃) δ 9.27 (s, 1H), 8.51 (d, J = 5.0 Hz, 1H), 8.35 (s, |
| | methylbutanoyl)piperazin-1-yl)- | 1H), 7.41 (s, 1H), 7.23 (dd, J = 5.0, 1.1 Hz, 1H), 4.87- |
| | 7H-pyrrolo[2,3-d]pyrimidin-7- | 4.73 (m, ~1.5H), 4.55-4.42 (m, ~1.5H), 4.14 (br. |
| | yl)isonicotinonitrile | s, ~0.5H), 3.79-3.73 (m, 2H), 3.66-3.54 (m, 3H), 3.34- |
| | | 3.16 (m, ~1.5H), 2.92 (m, 1H), 2.27-2.09 (m, 5H), 0.92 |
| | | (m, 9H); several peaks and their integrations are fractions of |
| | | protons on account of presence of rotomers. |
| 273 | (R)-2-(5-(azetidin-1-yl)-4-(4- | LC/MS ESI (m/z): 445 (M + H)⁺. ¹H NMR (400 MHz, |
| | isobutyryl-3-methylpiperazin-1- | CDCl₃) δ 9.28 (s, 1H), 8.51 (d, J = 5.0 Hz, 1H), 8.35 (s, |
| | yl)-7H-pyrrolo[2,3-d]pyrimidin- | 1H), 7.41 (s, 1H), 7.24-7.22 (m, 1H), 4.85-4.72 (m, ~1.5H), |
| | 7-yl)isonicotinonitrile | 4.55-4.39 (m, ~1.5H), 4.28-4.06 (br. s, ~0.5H), |
| | | 3.77-3.46 (m, 5H), 3.31 (br. s, 1H), 3.18 (br. s, ~0.5H), |
| | | 2.96 (br. s, 1H), 2.75 (m, 1H), 2.24 (m, 2H), 1.13-0.90 (m, |
| | | 9H); several peaks and their integrations are fractions of |
| | | protons on account of presence of rotomers. |
| 274 | (R)-2-(5-(azetidin-1-yl)-4-(4- | LC/MS ESI (m/z): 445 (M + H)⁺. ¹H NMR (400 MHz, |
| | butyryl-3-methylpiperazin-1-yl)- | CDCl₃) δ 9.27 (s, 1H), 8.51 (dd, J = 5.0, 0.7 Hz, 1H), 8.35 |
| | 7H-pyrrolo[2,3-d]pyrimidin-7- | (s, 1H), 7.41 (s, 1H), 7.23 (dd, J = 5.0, 1.3 Hz, 1H), 4.85- |
| | yl)isonicotinonitrile | 4.71 (m, ~1.5H), 4.53-4.40 (m, ~1.5H), 4.11 (br. |
| | | s, ~0.5H), 3.79-3.73 (m, 2H), 3.67-3.54 (m, 3H), 3.34- |
| | | 3.27 (m, ~1.5H), 3.06 (br. s, 1H), 2.32-2.17 (m, 4H), 1.62 |
| | | (m, 2H), 1.09-0.90 (m, 6H); several peaks and their |
| | | integrations are fractions of protons on account of presence |
| | | of rotomers. |
| 275 | 2-(5-(azetidin-1-yl)-4-((2S,5R)- | LC/MS ESI (m/z): 459 (M + H)⁺. ¹H NMR (400 MHz, |
| | 4-isobutyryl-2,5- | CDCl₃) δ 9.35 (s, 1H), 8.58 (d, J = 5.0 Hz, 1H), 8.44 (d, J = |
| | dimethylpiperazin-1-yl)-7H- | 6.5 Hz, 1H), 7.47 (d, J = 8.5 Hz, 1H), 7.30 (d, J = 4.9 Hz, |
| | pyrrolo[2,3-d]pyrimidin-7- | 1H), 5.20 (br. s, 1H), 4.86 (br. s, ~0.5H), 4.39 (d, J = 12.8 |
| | yl)isonicotinonitrile | Hz, ~0.5H), 4.22 (br. s, ~0.5H), 4.17-4.05 (m, 1H), 3.94- |
| | | 3.81 (m, ~2.5H), 3.74 (m, 1H), 3.66-3.60 (m, ~2.5H), 3.49 |
| | | (m, ~0.5H), 2.82 (m, 1H), 2.30 (m, 2H), 1.24-0.93 (m, |
| | | 12H); several peaks and their integrations are fractions of |
| | | protons on account of presence of rotomers. |
| 276 | 2-(5-(azetidin-1-yl)-4-((2S,5R)- | LC/MS ESI (m/z): 473 (M + H)⁺. ¹H NMR (400 MHz, |
| | 2,5-dimethyl-4- | CDCl₃) δ 9.38-9.33 (s, 1H), 8.58 (dd, J = 5.0, 0.7 Hz, 1H), |
| | pivaloylpiperazin-1-yl)-7H- | 8.44 (s, 1H), 7.47 (s, 1H), 7.30 (dd, J = 5.0, 1.3 Hz, 1H), |
| | pyrrolo[2,3-d]pyrimidin-7- | 5.19 (br. s, 1H), 4.67 (br. s, 1H), 4.36-3.97 (m, 2H), 3.87 |
| | yl)isonicotinonitrile | (app. q, J = 7.0 Hz, 2H), 3.78-3.59 (m, 4H), 2.31 (m, 2H), |
| | | 1.34 (s, 9H), 1.18-0.99 (m, 6H). |

### Example 50. Synthesis of 1-((2R,5S)-4-(5-(azetidin-1-yl)-7-(3-fluorophenyl)-7H-pyrrolo[2,3-d]pyrimidin-4-yl)-2,5-dimethylpiperazin-1-yl)-2-methoxy-2-methylpropan-1-one (Compound 277)

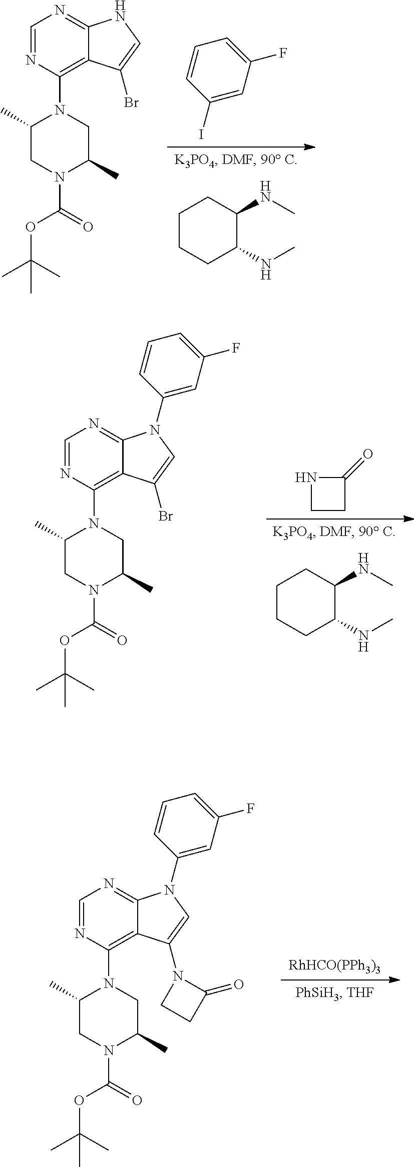

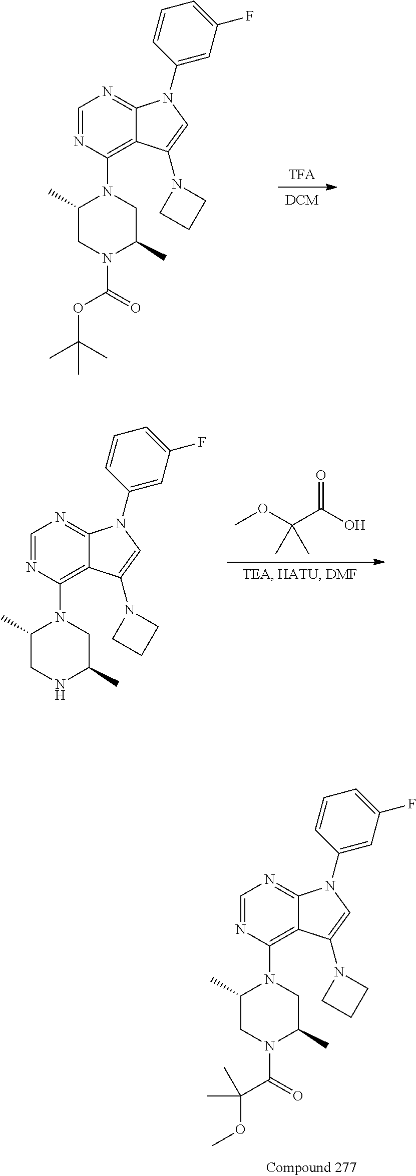

### Step 1. tert-Butyl (2R,5S)-4-(5-bromo-7-(3-fluorophenyl)-7H-pyrrolo[2,3-d]pyrimidin-4-yl)-2,5-dimethylpiperazine-1-carboxylate

To a solution of tert-butyl (2R,5S)-4-(5-bromo-7H-pyrrolo[2,3-d]pyrimidin-4-yl)-2,5-dimethylpiperazine-1-carboxylate (1.7 g, 4.2 mmol) in DMF (20 mL) were added 1-fluoro-3-iodobenzene (1.9 g, 8.3 mmol), trans-N,N′-dimethylcyclohexane-1,2-diamine (480 mg, 4.2 mmol), CuI (790 mg, 4.2 mmol) and K₃PO₄ (2.7 g, 12 mmol). The resulting mixture was heated at 90° C. overnight. After cooling to room temperature, the reaction was diluted with water and extracted twice with EtOAc. The combined organic layers were washed with brine, dried over Na₂SO₄, filtered and concentrated. The residue was purified by flash column chromatography (silica gel, 0˜50% ethyl acetate in petroleum ether) to afford tert-butyl (2R,5S)-4-(5-bromo-7-(3-fluorophenyl)-7H-pyrrolo[2,3-d]pyrimidin-4-yl)-2,5-dimethylpiperazine-1-carboxylate (1.7 g, 46%) as a white solid. LC/MS ESI (m/z): 504, 506 (M+H)⁺.

### Step 2. tert-Butyl (2R,5S)-4-(7-(3-fluorophenyl)-5-(2-oxoazetidin-1-yl)-7H-pyrrolo[2,3-d]pyrimidin-4-yl)-2,5-dimethylpiperazine-1-carboxylate

To a solution of tert-butyl (2R,5S)-4-(5-bromo-7-(4-chloropyridin-2-yl)-7H-pyrrolo[2,3-d]pyrimidin-4-yl)-2,5-dimethylpiperazine-1-carboxylate (1.7 g, 3.4 mmol) in DMF (20 mL) were added azetidin-2-one (480 mg, 6.8 mmol), trans-N,N′-dimethylcyclohexane-1,2-diamine (390 mg, 3.4 mmol), CuI (650 mg, 3.4 mmol) and K₃PO₄ (2.2 g, 10 mmol). The resulting mixture was heated at 90° C. overnight. After cooling to room temperature, the reaction was diluted with water and extracted twice with EtOAc. The combined organic layers were washed with brine, dried over Na₂SO₄, filtered and concentrated. The residue was purified by flash column chromatography (silica gel, 0˜50% ethyl acetate in petroleum ether) to afford tert-butyl (2R,5S)-4-(7-(3-fluorophenyl)-5-(2-oxoazetidin-1-yl)-7H-pyrrolo[2,3-d]pyrimidin-4-yl)-2,5-dimethylpiperazine-1-carboxylate (1.4 g, 84%) as a yellow solid. LC/MS ESI (m/z): 495 (M+H)⁺.

### Step 3. tert-Butyl (2R,5S)-4-(5-(azetidin-1-yl)-7-(3-fluorophenyl)-7H-pyrrolo[2,3-d]pyrimidin-4-yl)-2,5-dimethylpiperazine-1-carboxylate

To a solution of tert-butyl (2R,5S)-4-(7-(3-fluorophenyl)-5-(2-oxoazetidin-1-yl)-7H-pyrrolo[2,3-d]pyrimidin-4-yl)-2,5-dimethylpiperazine-1-carboxylate (1.3 g, 3.4 mmol) in THF (20 mL) at 0° C. were added RhHCO(PPh₃)₃ (370 mg, 0.40 mmol) and PhSiH₃ (2.2 g, 20 mmol) dropwise. After stirring at room temperature overnight under N₂, the reaction was partitioned between EtOAc and water. The aqueous layer was extracted twice. The combined organic layers were washed with brine, dried over Na₂SO₄, filtered and concentrated. The residue was purified by flash column chromatography (silica gel, 0˜35% ethyl acetate in petroleum ether) to afford tert-butyl (2R,5S)-4-(5-(azetidin-1-yl)-7-(3-fluorophenyl)-7H-pyrrolo[2,3-d]pyrimidin-4-yl)-2,5-dimethylpiperazine-1-carboxylate (850 mg, 52%) as a yellow solid. LC/MS ESI (m/z): 481 (M+H)⁺.

### Step 5. 5-(Azetidin-1-yl)-4-((2S,5R)-2,5-dimethylpiperazin-1-yl)-7-(3-fluorophenyl)-7H-pyrrolo[2,3-d]pyrimidine

To a solution of tert-butyl (2R,5S)-4-(5-(azetidin-1-yl)-7-(4-cyanopyridin-2-yl)-7H-pyrrolo[2,3-d]pyrimidin-4-yl)-2,5-dimethylpiperazine-1-carboxylate (850 mg, 1.8 mmol) in DCM (10 mL) at 0° C. was added TFA (3.0 mL). The resulting mixture was stirred at 0° C. for 2 h. After removal of solvent, the residue was diluted with DCM and washed with NaHCO₃ (aq.). The aqueous layer was extracted twice with DCM. The combined organic layers were washed with brine, dried over Na₂SO₄, filtered and concentrated. The residue was used in the next step directly. LC/MS ESI (m/z): 381 (M+H)⁺.

### Step 6. 2-(5-(Azetidin-1-yl)-4-((2S,5R)-4-(2-methoxy-2-methylpropanoyl)-2,5-dimethylpiperazin-1-yl)-7H-pyrrolo[2,3-d]pyrimidin-7-yl)isonicotinonitrile

To a solution of 2-(5-(azetidin-1-yl)-4-((2S,5R)-2,5-dimethylpiperazin-1-yl)-7H-pyrrolo[2,3-d]pyrimidin-7-yl)isonicotinonitrile (70 mg, 0.18 mmol) in DMF (10 mL) were added TEA (55 mg, 0.54 mmol), 2-methoxy-2-methylpropanoic acid (260 mg, 2.2 mmol) and HATU (840 mg, 2.2 mmol). After 2 h, the residue was diluted with EtOAc and washed with NaHCO₃ (aq.). The aqueous layer was extracted twice with EtOAc. The combined organic layers were washed with brine, dried over Na₂SO₄, filtered and concentrated. The residue was purified by flash column chromatography (silica gel, 0˜50% ethyl acetate in petroleum ether) to give crude product which was further purified by prep-HPLC to 1-((2R,5S)-4-(5-(azetidin-1-yl)-7-(3-fluorophenyl)-7H-pyrrolo[2,3-d]pyrimidin-4-yl)-2,5-dimethylpiperazin-1-yl)-2-methoxy-2-methylpropan-1-one (24 mg, 27%) as a white solid. LC/MS ESI (m/z): 481 (M+H)⁺. ¹H NMR (400 MHZ, CDCl₃) δ 8.38 (d, J=3.3 Hz, 1H), 7.57-7.53 (m, 2H), 7.45 (m, 1H), 7.00 (t, J=8.2 Hz, 1H), 6.64 (d, J=8.7 Hz, 1H), 5.28 (m, 1H), 5.16 (br. s, ˜0.5H), 4.78 (br. s, ˜0.5H), 4.49 (m, 1H), 4.17 (d, J=14.0 Hz, ˜0.5H), 3.99 (m, 1H), 3.88-3.76 (m, ˜2.5H), 3.67-3.52 (m, 3H), 3.35 (s, ˜1.5H), 3.25 (s, ˜1.5H), 2.31 (m, 2H), 1.55-1.45 (m, 6H), 1.25 (m, 3H), 1.02 (m, 3H); several peaks and their integrations are fractions of protons on account of presence of rotomers.

The following compounds were prepared by procedures analogous to the synthesis of compound 277 from the corresponding acids, aryl halides and lactam.

| Cmpd | | |
|---|---|---|
| No. | Chemical Name | LCMS and ¹H NMR |
| 278 | (S)-(4-(7-(3-chlorophenyl)-5- | LC/MS ESI (m/z): 483 (M + H)⁺. ¹H NMR (400 MHz, |
| | (pyrrolidin-1-yl)-7H- | CDCl₃) δ 8.39 (s, 1H), 7.74 (t, J = 2.0 Hz, 1H), 7.67 (m, |
| | pyrrolo[2,3-d]pyrimidin-4-yl)-3- | 1H), 7.42 (t, J = 8.1 Hz, 1H), 7.28 (m, 1H), 6.74 (s, 1H), |
| | methylpiperazin-1-yl)(1- | 5.11 (br. s, 1H), 4.56-4.15 (m, 3H), 3.70-3.40 (m, 2H), |
| | fluorocyclopropyl)methanone | 3.26-2.86 (m, 5H), 1.98 (m, 4H), 1.43-0.99 (m, 7H). |
| 279 | (R)-(4-(7-(3-chlorophenyl)-5- | LC/MS ESI (m/z): 483 (M + H)⁺. ¹H NMR (400 MHz, |
| | (pyrrolidin-1-yl)-7H- | CDCl₃) δ 8.38 (s, 1H), 7.74 (t, J = 2.0 Hz, 1H), 7.67 (dd, |
| | pyrrolo[2,3-d]pyrimidin-4-yl)-2- | J = 8.1, 1.1 Hz, 1H), 7.42 (t, J = 8.1 Hz, 1H), 7.28 (m, 1H), |
| | methylpiperazin-1-yl)(1- | 6.74 (s, 1H), 4.88 (d, J = 12.8 Hz, 1H), 4.74 (br. s, 1H), |
| | fluorocyclopropyl)methanone | 4.49 (d, J = 13.3 Hz, 1H), 4.31 (br. s, 1H), 3.60-3.19 (m, |
| | | 4H), 3.03 (m, 1H), 2.86 (m, 2H), 1.99 (m, 4H), 1.35-1.18 |
| | | (m, 7H). |
| 280 | ((2R,5S)-4-(7-(3-chlorophenyl)- | LC/MS ESI (m/z): 497 (M + H)⁺. ¹H NMR (400 MHz, |
| | 5-(pyrrolidin-1-yl)-7H- | CDCl₃) δ 8.39 (s, 1H), 7.75 (t, J = 2.0 Hz, 1H), 7.71-7.65 |
| | pyrrolo[2,3-d]pyrimidin-4-yl)- | (m, 1H), 7.42 (t, J = 8.1 Hz, 1H), 7.29 (m, 1H), 6.74 (s, |
| | 2,5-dimethylpiperazin-1-yl)(1- | 1H), 5.24 (br. s, 1H), 4.80-4.70 (m, 1H), 4.29-3.80 (m, |
| | fluorocyclopropyl)methanone | s, ~2.5H), 3.70-3.48 (m, ~1.5H), 3.30 (br. s, 2H), 2.83 (br. |
| | | 2H), 1.99 (s, 4H), 1.36-1.01 (m, 10H); several peaks and |
| | | their integrations are fractions of protons on account of |
| | | presence of rotomers. |

### Example 51. Synthesis of 2-(5-(2-fluorophenyl)-4-((2S,5R)-4-isobutyryl-2-methyl-5-(methyl-d₃)piperazin-1-yl)-7H-pyrrolo[2,3-d]pyrimidin-7-yl)isonicotinonitrile (Compound 281)

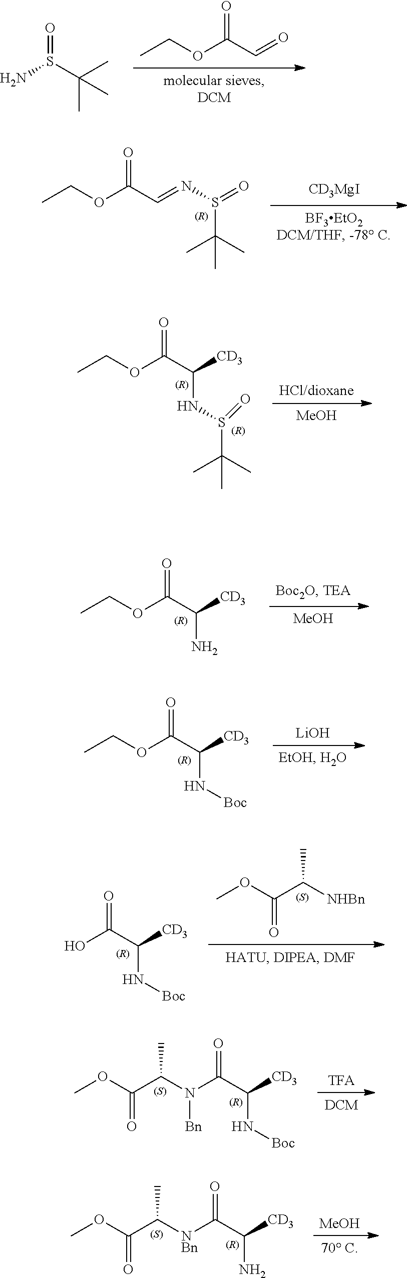

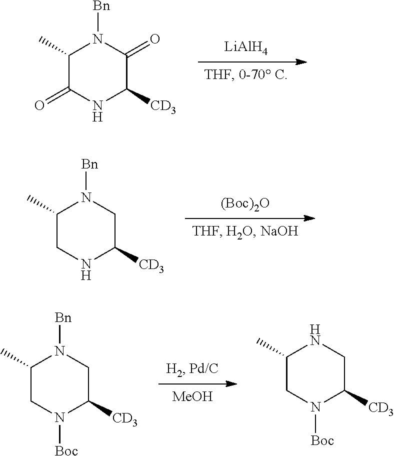

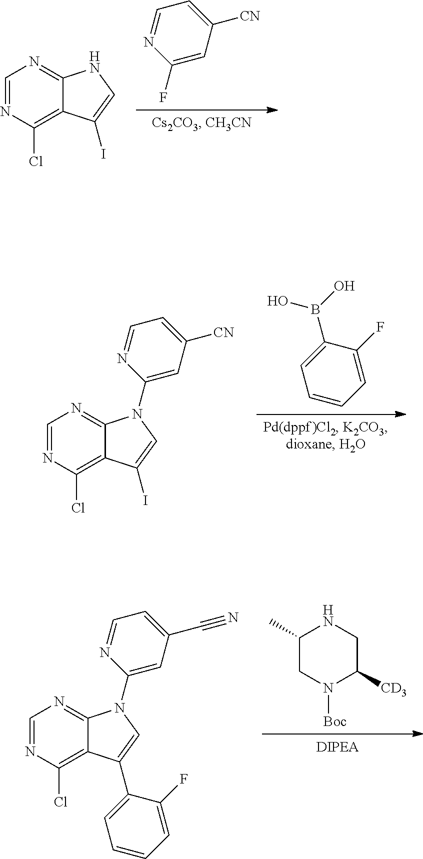

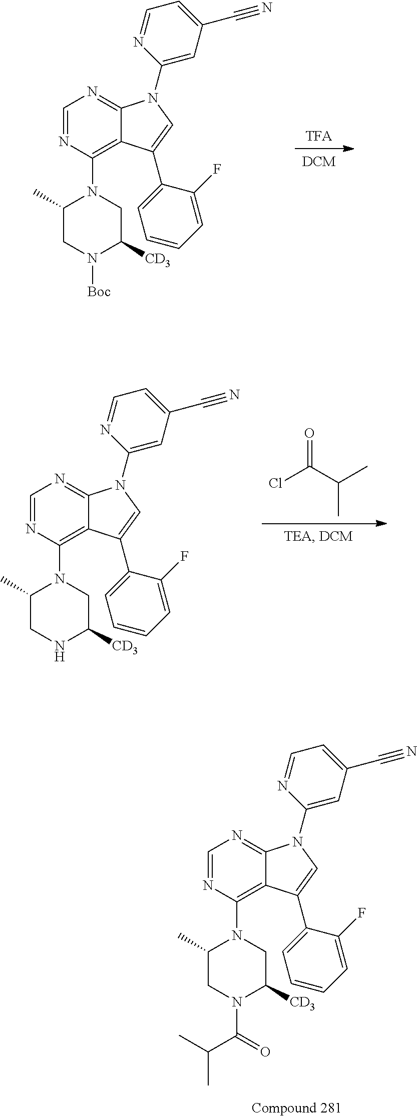

### Step 1. Ethyl (R,E)-2-((tert-butylsulfinyl)imino)acetate

To a solution of ethyl 2-oxoacetate (6.4 g, 63 mmol) in CH₂Cl₂ (300 mL) were added (R)-2-methylpropane-2-sulfinamide (7.6 g, 63 mmol) and 4 Å molecular sieves (20 g). After 72 h, the mixture was filtered through a bed of Celite and washed with EtOAc. The filtrate was dried over Na₂SO₄, filtered and concentrated. The residue was purified with flash column chromatography (silica gel, 0˜20% ethyl acetate in petroleum ether) to afford ethyl (R,E)-2-((ter-butylsulfinyl)imino)acetate (7.2 g. 56%) as a colorless oil.

### Step 2. Ethyl ((R)-tert-butylsulfinyl)-D-alaninate-3,3,3-d₃

To a solution of ethyl(R,E)-2-((tert-butylsulfinyl)imino)acetate (6.2 g, 30 mmol) in DCM (400 mL) and THF (120 mL) at −78° C. was added BF₃·Et₂O (7.4 mL, 60 mmol) dropwise under N₂ atmosphere. The resulting mixture was stirred at −78° C. for 5 min and treated with CD₃MgI (1.0 M in Et₂O, 60 mL, 60 mmol) dropwise. After stirring at −78° C. for 10 min, the reaction was quenched with NaHCO₃ (aq.) and partitioned between EtOAc and brine. The aqueous layer was extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to afford ethyl ((R)-tert-butylsulfinyl)-D-alaninate-3,3,3-d₃ (1.4 g, 21% yield, dr=97/3) as a white solid. LC/MS ESI (m/z): 225 (M+H)⁺. The configuration was verified by ¹H NMR (J. Org. Chem. 1999, 64, 3396-3397).

### Step 3. Ethyl D-alaninate-3,3,3-d₃

To a solution of ethyl ((R)-tert-butylsulfinyl)-D-alaninate-3,3,3-d₃ (1.4 g, 6.2 mmol) in MeOH (30 mL) was added HCl (5.0 mL, 4.0 M in dioxane). The resulting mixture was stirred at room temperature under N₂ for 2 h. The solvents were removed under vacuum to afford ethyl D-alaninate-3,3,3-d₃, as a white solid (1.0 g crude HCl salt). LC/MS ESI (m/z): 121 (M+H)⁺

### Step 4. Ethyl (tert-butoxycarbonyl)-D-alaninate-3,3,3-d₃

To a solution of ethyl D-alaninate-3,3,3-d₃ (1.0 g crude HCl salt) in MeOH (30 mL) were added TEA (1.7 mL, 12 mmol) and Boc₂O (1.5 g, 6.9 mmol). The resulting mixture was stirred at room temperature under N₂ for 2 h. The reaction was partitioned between EtOAc and NH₄Cl (aq.). The aqueous layer was extracted twice with EtOAc. The combined organic layers were washed with brine, dried over Na₂SO₄, filtered, and concentrated to afford crude product ethyl(tert-butoxycarbonyl)-D-alaninate-3,3,3-d₃ (1.4 g crude) as a white solid. LC/MS ESI (m/z): 221 (M+H)⁺.

### Step 5. (tert-Butoxycarbonyl)-D-alanine-3,3,3-d₃

To a solution of ethyl(tert-butoxycarbonyl)-D-alaninate-3,3,3-d₃ (1.4 g crude) in EtOH (20 mL) and H₂O (10 mL) in an ice bath was added LiOH (310 mg, 13 mmol). The resulting mixture was stirred at room temperature under N₂ for 1 h. The reaction was partitioned between EtOAc and brine. The aqueous layer was acidified by HCl (1.0 M) to pH 3 and extracted twice with EtOAc. The organic layer was washed with brine, dried over Na₂SO₄, filtered, and concentrated to afford (tert-butoxycarbonyl)-D-alanine-3,3,3-d₃ (900 mg, 75% yield over three steps) LC/MS ESI (m/z): 193 (M+H)⁺.

### Step 6. Methyl N-benzyl-N-((tert-butoxycarbonyl)-D-alanyl-3,3,3-d₃)-L-alaninate

To a solution of (tert-butoxycarbonyl)-D-alanine-3,3,3-d₃ (900 mg, 4.7 mmol) and methyl benzyl-L-alaninate hydrochloride (1.2 g, 5.2 mmol) in DMF (20 mL) were added DIPEA (2.4 g, 19 mmol) and HATU (2.7 g, 7.0 mmol). The resulting mixture was stirred at room temperature under N₂ overnight. The reaction was partitioned between EtOAc and aqueous NaHCO₃. The aqueous layer was extracted twice with EtOAc. The combined organic layers were washed with brine, dried over Na₂SO₄, filtered and concentrated. The residue was purified by flash column chromatography (silica gel, 0˜60%, ethyl acetate in petroleum ether) to afford methyl N-benzyl-N-((tert-butoxycarbonyl)-D-alanyl-3,3,3-d₃)-L-alaninate (1.4 g, 81% yield) as a yellow solid. LC/MS ESI (m/z): 368 (M+H)⁺.

### Step 7. Methyl N-(D)-alanyl-3,3,3-d₃)—N-benzyl-L-alaninate

To a solution of methyl N-benzyl-N-((tert-butoxycarbonyl)-D-alanyl-3,3,3-d₃)-L-alaninate (1.4 g, 3.8 mmol) in DCM (20 mL) at 0° C. was added TFA (10 mL). The resulting mixture was stirred at room temperature under N₂ for 3 h. The solvent of the reaction was removed under vacuum to afford crude product methyl N—(I)-alanyl-3,3,3-d₃)—N-benzyl-L-alaninate (1.5 g crude TFA salt) as a yellow solid. LC/MS ESI (m/z): 268 (M+H)⁺.

### Step 8. (3R,6S)-1-Benzyl-6-methyl-3-(methyl-d₃)piperazine-2,5-dione

A solution of methyl N—(I)-alanyl-3,3,3-d₃)—N-benzyl-L-alaninate (1.5 g crude TFA salt) in MeOH (10 mL) was heated at 70° C. under N₂ for 4 h. The reaction mixture was then partitioned between EtOAc and brine aqueous. The aqueous layer was extracted twice with EtOAc. The combined organic layers were washed with brine, dried over Na₂SO₄, filtered, and concentrated to afford (3R,6S)-1-benzyl-6-methyl-3-(methyl-d₃)piperazine-2,5-dione (1.0 g crude) as a yellow solid. LC/MS ESI (m/z): 236 (M+H)⁺.

### Step 9. tert-Butyl (2R,5S)-4-benzyl-5-methyl-2-(methyl-d₃)piperazine-1-carboxylate

A solution of (3R,6S)-1-benzyl-6-methyl-3-(methyl-d₃)piperazine-2,5-dione (1.0 g crude) in THF (10 mL) in an ice bath under N₂ was treated with LiAlH₄ (1.0 M in THF, 20 mL) dropwise. The reaction mixture was stirred at 70° C. for 3 h. The reaction mixture was carefully quenched with 20% NaOH (20 mL) and the resulting mixture was used in the next step directly. LC/MS ESI (m/z): 208 (M+H)⁺.

The solution was then treated with Boc₂O (1.7 g, 7.6 mmol). After stirring at room temperature overnight, the reaction mixture was filtered. The residue was purified by flash column chromatography (silica gel, 0˜10%, ethyl acetate in petroleum ether) to afford tert-butyl (2R,5S)-4-benzyl-5-methyl-2-(methyl-d₃)piperazine-1-carboxylate (990 mg, 85% yield dr=90/10) as a colorless oil. LC/MS ESI (m/z): 308 (M+H)⁺. ¹H NMR (400 MHZ, CDCl₃) δ 7.37-7.28 (m, 4H), 7.26-7.21 (m, 1H), 4.17 (d, J=3.9 Hz, 1H), 3.69-3.57 (m, 2H), 3.46 (d, J=13.6 Hz, 1H), 3.30 (dd, J=13.0, 3.7 Hz, 1H), 2.92 (dd, J=10.9, 5.9 Hz, 1H), 2.69 (dd, J=11.7, 4.4 Hz, 1H), 2.19 (dd, J=11.7, 1.9 Hz, 1H), 1.46 (s, 9H), 0.98 (d, J=6.5 Hz, 3H).

### Step 10. tert-Butyl (2R,5S)-5-methyl-2-(methyl-d₃)piperazine-1-carboxylate

To a solution of tert-butyl (2R,5S)-4-benzyl-5-methyl-2-(methyl-d₃)piperazine-1-carboxylate (550 mg, 1.7 mmol) in MeOH (15 mL) was added Pd(200 mg, 10% on carbon (wet)). After stirring at room temperature for 2 h under H₂ atmosphere (˜0.1 MPa), the reaction was filtered and the filtrate was concentrated to afford tert-butyl (2R,5S)-5-methyl-2-(methyl-d₃)piperazine-1-carboxylate (320 mg, 82%) as a yellow oil. LC/MS ESI (m/z): 218 (M+H)⁺.

### Step 11. 2-(4-Chloro-5-iodo-7H-pyrrolo[2,3-d]pyrimidin-7-yl)isonicotinonitrile

To a solution of 4-chloro-5-iodo-7H-pyrrolo[2,3-d]pyrimidine (7.0 g, 25 mmol) and 2-fluoroisonicotinonitrile (18 g, 150 mmol) in CH₃CN (70 mL) was added Cs₂CO₃ (12 g, 38 mmol). The resulting mixture was stirred at 25° C. under N₂ for 72 h. Crude product was collected by filtration, washed with water and CH₃CN to afford 2-(4-chloro-5-iodo-7H-pyrrolo[2,3-d]pyrimidin-7-yl)isonicotinonitrile (9.0 g, 94%) as a light brown solid. LC/MS ESI (m/z): 382 (M+H)⁺.

### Step 12. 2-(4-Chloro-5-(2-fluorophenyl)-7H-pyrrolo[2,3-d]pyrimidin-7-yl)isonicotinonitrile

To a solution of 2-(4-chloro-5-iodo-7H-pyrrolo[2,3-d]pyrimidin-7-yl)isonicotinonitrile (600 mg, 1.6 mmol) in dioxane (25 mL) and water (2 drop) were added (2-fluorophenyl) boronic acid (290 mg, 2.0 mmol), K₂CO₃ (1.1 g, 7.9 mmol) and Pd(dppf)Cl₂ (130 mg, 0.16 mmol). The resulting mixture was stirred at 80° C. overnight. After cooling to room temperature, solvent was removed and the residue was purified by flash column chromatography (silica gel, 0˜30% ethyl acetate in petroleum) to afford 2-(4-chloro-5-(2-fluorophenyl)-7H-pyrrolo[2,3-d]pyrimidin-7-yl)isonicotinonitrile (150 mg, 27%) as a white solid. LC/MS ESI (m/z): 350 (M+H)⁺.

### Step 13. tert-Butyl (2R,5S)-4-(7-(4-cyanopyridin-2-yl)-5-(2-fluorophenyl)-7H-pyrrolo[2,3-d]pyrimidin-4-yl)-5-methyl-2-(methyl-d₃)piperazine-1-carboxylate

To a solution of tert-butyl (2R,5S)-5-methyl-2-(methyl-d₃)piperazine-1-carboxylate (30 mg, 0.13 mmol) in DIPEA (3 mL) was added 2-(4-chloro-5-(2-fluorophenyl)-7H-pyrrolo[2,3-d]pyrimidin-7-yl)isonicotinonitrile (72 mg, 0.20 mmol). The resulting mixture was stirred at 150° C. for 2 h. After cooling to room temperature, the reaction was concentrated and the residue was purified by flash column chromatography (silica gel, 0˜30%, ethyl acetate in petroleum ether) to afford tert-butyl (2R,5S)-4-(7-(4-cyanopyridin-2-yl)-5-(2-fluorophenyl)-7H-pyrrolo[2,3-d]pyrimidin-4-yl)-5-methyl-2-(methyl-d₃)piperazine-1-carboxylate (30 mg, 41%) as a yellow solid. LC/MS ESI (m/z): 531 (M+H)⁺.

### Step 14. 2-(5-(2-Fluorophenyl)-4-((2S,5R)-2-methyl-5-(methyl-d₃)piperazin-1-yl)-7H-pyrrolo[2,3-d]pyrimidin-7-yl)isonicotinonitrile

To a solution of tert-butyl (2R,5S)-4-(7-(4-cyanopyridin-2-yl)-5-(2-fluorophenyl)-7H-pyrrolo[2,3-d]pyrimidin-4-yl)-5-methyl-2-(methyl-d₃)piperazine-1-carboxylate (15 mg, 0.028 mmol) in DCM (2 mL) at 0° C. was added TFA (0.50 mL, 6.7 mmol). After stirring at 0° C. for 1 h, the reaction was basified with NaHCO₃ (aq.) to pH 8 and extracted twice with DCM. The combined organic layers were dried over Na₂SO₄, filtered and concentrated. The residue was used directly in the next step. LC/MS ESI (m/z): 431 (M+H)⁺.

### Step 15. 2-(5-(2-Fluorophenyl)-4-((2S,5R)-4-isobutyryl-2-methyl-5-(methyl-d₃)piperazin-1-yl)-7H-pyrrolo[2,3-d]pyrimidin-7-yl)isonicotinonitrile

To a solution of 2-(5-(2-fluorophenyl)-4-((2S,5R)-2-methyl-5-(methyl-d₃)piperazin-1-yl)-7H-pyrrolo[2,3-d]pyrimidin-7-yl)isonicotinonitrile (10 mg, 0.023 mmol) and TEA (0.02 mL, 0.11 mmol) in DCM (2 mL) at 0° C. was added isobutyryl chloride (5.0 mg, 0.046 mmol). After stirring at room temperature for 1 h, the reaction was partitioned between DCM and water. The aqueous layer was extracted twice with DCM. The combined organic layers were washed with NaHCO₃ (aq.) and brine, dried over Na₂SO₄, filtered and concentrated. The residue was purified by prep-HPLC to afford 2-(5-(2-fluorophenyl)-4-((2S,5R)-4-isobutyryl-2-methyl-5-(methyl-d₃)piperazin-1-yl)-7H-pyrrolo[2,3-d]pyrimidin-7-yl)isonicotinonitrile (2.3 mg, 19%) as a white solid. LC/MS ESI (m/z): 501 (M+H)⁺. ¹H NMR (400 MHZ, CDCl₃) δ 9.34 (s, 1H), 8.56 (d, J=5.0 Hz, 1H), 8.51 (d, J=6.1 Hz, 1H), 8.23 (d, J=4.7 Hz, 1H), 7.43 (m, 1H), 7.36-7.29 (m, 2H), 7.19-7.11 (m, 2H), 4.64-4.30 (m, 1H), 4.05-3.70 (m, ˜1.5H), 3.55-3.05 (m, 3H), 2.75-2.45 (m, ˜1.5H), 1.09-1.01 (m, 6H), 1.00-0.95 (m, 3H); several peaks and their integrations are fractions of protons on account of presence of rotomers.

### Example 52. Synthesis of 2-(4-((2S,5R)-4-(2-methoxy-2-methylpropanoyl)-2,5-dimethylpiperazin-1-yl)-5-phenyl-7H-pyrrolo[2,3-d]pyrimidin-7-yl)isonicotinonitrile (Compound 282)

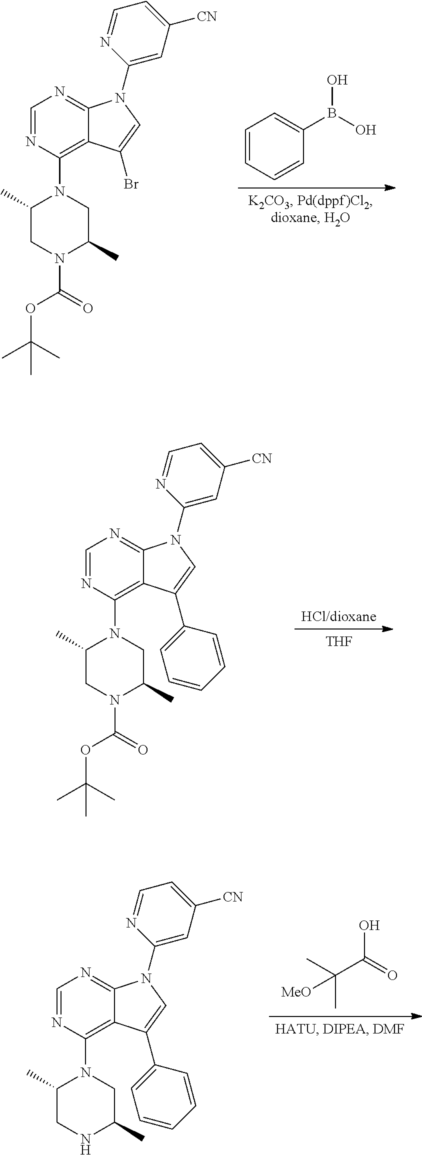

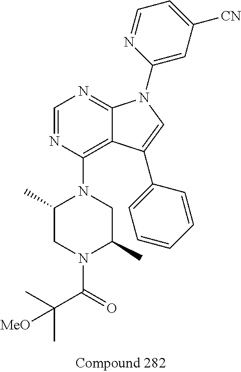 [SMILES: COC(C)(C)C(=O)N1C[C@H](C)N(c2ncnc3c2c(-c2ccccc2)cn3-c2cc(C#N)ccn2)C[C@H]1C]

### Step 1. tert-Butyl (2R,5S)-4-(7-(4-cyanopyridin-2-yl)-5-phenyl-7H-pyrrolo[2,3-d]pyrimidin-4-yl)-2,5-dimethylpiperazine-1-carboxylate

To a solution of tert-butyl (2R,5S)-4-(5-bromo-7-(4-cyanopyridin-2-yl)-7H-pyrrolo[2,3-d]pyrimidin-4-yl)-2,5-dimethylpiperazine-1-carboxylate (1.5 g, 2.9 mmol, prepared following a similar procedure outlined for compound 268 using 2-fluoroisonicotinonitrile) in dioxane (40 mL) and H₂O (8 mL) were added phenylboronic acid (0.71 g, 5.8 mmol), K₂CO₃ (1.2 g, 8.7 mmol) and Pd(dppf)Cl₂ (0.21 g, 0.29 mmol). The resulting mixture was stirred at 80° C. overnight. After cooling to room temperature, the reaction was concentrated and the residue was purified by flash column chromatography (silica gel, 0˜20%, ethyl acetate in petroleum ether) to afford tert-butyl (2R,5S)-4-(7-(4-cyanopyridin-2-yl)-5-phenyl-7H-pyrrolo[2,3-d]pyrimidin-4-yl)-2,5-dimethylpiperazine-1-carboxylate (1.00 g, 69%) as a yellow solid. LC/MS ESI (m/z): 510 (M+H)⁺.

### Step 2. 2-(4-((2S,5R)-2,5-Dimethylpiperazin-1-yl)-5-phenyl-7H-pyrrolo[2,3-d]pyrimidin-7-yl)isonicotinonitrile

To a solution of tert-butyl (2R,5S)-4-(7-(4-cyanopyridin-2-yl)-5-phenyl-7H-pyrrolo[2,3-d]pyrimidin-4-yl)-2,5-dimethylpiperazine-1-carboxylate (1.0 g, 2.0 mmol) in THF (10 mL) was added HCl in dioxane (10 mL, 4.0 M). After stirring at room temperature overnight, the reaction was concentrated, and the residue was used directly in the next step. LC/MS ESI (m/z): 410 (M+H)⁺.

### Step 3. 2-(4-((2S,5R)-4-(2-Methoxy-2-methylpropanoyl)-2,5-dimethylpiperazin-1-yl)-5-phenyl-7H-pyrrolo[2,3-d]pyrimidin-7-yl)isonicotinonitrile

To a solution of 2-(4-((2S,5R)-2,5-dimethylpiperazin-1-yl)-5-phenyl-7H-pyrrolo[2,3-d]pyrimidin-7-yl)isonicotinonitrile (100 mg, 0.24 mmol) in DMF (3 mL) were added 2-methoxy-2-methylpropanoic acid (0.040 mL, 0.29 mmol), HATU (190 mg, 0.48 mmol) and DIPEA (0.20 mL, 1.2 mmol). After 4 h, the reaction was partitioned between EtOAc and water. The aqueous layer was extracted twice with EtOAc. The combined organic layers were washed with brine, dried over Na₂SO₄, filtered and concentrated. The residue was purified by prep-HPLC to afford 2-(4-((2S,5R)-4-(2-methoxy-2-methylpropanoyl)-2,5-dimethylpiperazin-1-yl)-5-phenyl-7H-pyrrolo[2,3-d]pyrimidin-7-yl)isonicotinonitrile (50 mg, 40%) as a white solid. LC/MS ESI (m/z): 510 (M+H)⁺. ¹H NMR (400 MHZ, CDCl₃) δ 9.35 (s, 1H), 8.55 (d, J=5.0 Hz, 1H), 8.52 (s, 1H), 8.16 (d, J=5.2 Hz, 1H), 7.50 (t, J=7.0 Hz, 2H), 7.40 (m, 2H), 7.32 (m, 2H), 5.09 (br. s, ˜0.5H), 4.57 (br. s, ˜0.5H), 4.25-3.69 (m, 2H), 3.47-3.05 (m, ˜5.5H), 2.60 (m, ˜0.5H), 1.45-1.25 (m, ˜7.5H), 1.02 (d, J=6.7 Hz, ˜1.5H), 0.96 (d, J=6.7 Hz, ˜1.5H), 0.74 (d, J=6.7 Hz, ˜1.5H); several peaks and their integrations are fractions of protons on account of presence of rotomers.

The following compound was prepared by the procedure analogous to the synthesis of compound 282 from the corresponding carboxylic acid.

| Cmpd | | |
|---|---|---|
| No. | Chemical Name | LCMS and ¹H NMR |
| 283 | 2-(4-((2S,5R)-4-(1- | LC/MS ESI (m/z): 508 (M + H)⁺. ¹H NMR (400 MHz, |
| | methoxycyclopropane-1- | CDCl₃) δ 9.36 (s, 1H), 8.56 (d, J = 5.0 Hz, 1H), 8.52 (d, J = |
| | carbonyl)-2,5- | 4.8 Hz, 1H), 8.16 (d, J = 6.1 Hz, 1H), 7.51 (t, J = 8.5 Hz, |
| | dimethylpiperazin-1-yl)-5- | 2H), 7.40 (m, 2H), 7.32 (m, 2H), 4.80 (br. s, ~0.5H), 4.52 |
| | phenyl-7H-pyrrolo[2,3- | (br. s, ~0.5H), 4.26, (br. s, ~0.5H), 3.95 (br. s, ~0.5H), 3.80- |
| | d]pyrimidin-7- | 3.45 (m, ~1.5H), 3.40-3.15 (m, 5H), 2.63 (m, ~0.5H), |
| | yl)isonicotinonitrile | 1.27 (m, 2H), 1.04-0.92 (m, 4H), 0.88-0.69 (m, 4H); |
| | | several peaks and their integrations are fractions of protons |
| | | on account of presence of rotomers. |

### Example 53. Fluorescent TRPML Assays

### TRPML1 Assay

### Cell Culture

HEK-293 Trex cells were stably transfected with a construct consisting of the human coding sequence for TRPML1 cloned into the tet-inducible plasmid pCDNA5 T/O. Mutations were introduced into the TRPML1 sequence to facilitate expression on the cell surface (Silvia Vergarajauregui, Rosa Puertollano Traffic. 2006 March; 7 (3): 337-353). Briefly, the cells are cultured in 150 mm round tissue culture dishes containing 20 mL of media. The day before the assay the cells are rinsed with DPBS —Ca—Mg and then treated briefly with Trypsin-EDTA. The Trypsin-EDTA is diluted with growth media, and cells are counted. 38×10\hat{ }6 cells are re-plated into 150 mm round tissue culture dishes in media containing 0.5 ug/mL doxycycline to induce expression of hTRPML1.

### Dye Loading

The day of the experiment cells are lifted from the plates as above and collected by centrifugation. The cells are then suspended in dye loading buffer consisting of Ringer's solution supplemented with 0.1% Pluronic Acid and 1 micromolar Fluo4-AM dye. Cells are loaded for ˜60 minutes in the dark with occasional mixing. The cells are collected by centrifugation, the loading media aspirated, and the cells resuspended in 25 mL Ringer's solution and incubated ˜60 minutes in the dark. The cells are again collected by centrifugation, rinsed in Ringer's Solution and resuspended to 0.2×10\hat{ }6 cells/mL in modified Ringer's solution containing 10 mM calcium.

### Compound Assay Plates

Compounds are dissolved to a concentration of 10 millimolar with DMSO. Compound plates are created by dispensing compounds into 384 well black wall clear bottom plates (Greiner 781091). Positive and negative controls are included on each plate. Typically, different amounts of each compound are tested ranging from 100 nanoMoles (20 micromolar final concentration) decreasing in half-log steps to 31 picoMoles (6 nanomolar final concentration). Each concentration is typically tested in triplicate.

### Assay

50 microliters of dye-loaded cells are dispensed into each well of the compound assay plate created above. The fluorescence in each well is then determined with an excitation wavelength of 480 nM and an emission wavelength of 540 nM using either a Molecular Devices SpectraMax multimode plate reader or a Hamamatsu FDSS/uCell plate imager.

### Analysis and Statistics

The resulting fluorescence for each well is exported as an ascii file and loaded into our LIMS for analysis. The percent activity of each compound at each concentration is determined by comparison to the positive and negative control wells included in each plate.

### TRPML2 and TRPML3 Assays

Assays for TRPML2 and TRPML3 were performed as above for TRPML1, by substituting the appropriate TRPML2 or TRPML3 subtype for the TRPML1.

EC₅₀ values were calculated using a non-linear regression of Prism. The EC₅₀ determined for each compound using the assay is summarized in Table 3 below. The compound numbers correspond to those shown in Table 1. In the table, “A” indicates an EC₅₀ of less than 100 nM, “B” indicates an EC₅₀ range from 100 nM to 500 nM; “C” indicates an EC₅₀ range from 500 nM to 2 μM; and “D” indicates an EC₅₀ greater than 2 μM.

TABLE 3
Efficacy of exemplary compounds of the invention

| | Human | Human | Human |
|---|---|---|---|
| Cmpd | TRPML1 | TRPML2 | TRPML3 |
| No. | EC₅₀ | EC₅₀ | EC₅₀ |
| 101 | D | — | C |
| 102 | A | B | A |
| 103 | A | A | A |
| 104 | A | C | A |
| 105 | A | — | A |
| 106 | B | — | A |
| 107 | A | B | A |
| 108 | A | A | A |
| 109 | A | A | A |
| 110 | B | — | B |
| 111 | A | — | — |
| 112 | A | — | A |
| 113 | A | — | B |
| 114 | A | — | A |
| 115 | A | A | A |
| 116 | C | — | C |
| 117 | A | C | B |
| 118 | A | C | A |
| 119 | A | — | A |
| 120 | A | B | A |
| 121 | A | B | A |
| 122 | A | B | B |
| 123 | B | — | B |
| 124 | B | — | B |
| 125 | A | — | A |
| 126 | A | B | A |
| 127 | C | — | D |
| 128 | A | A | A |
| 129 | A | — | B |
| 130 | A | B | A |
| 131 | A | B | A |
| 132 | C | — | C |
| 133 | A | B | A |
| 134 | A | — | B |
| 135 | A | — | B |
| 136 | B | — | B |
| 137 | A | — | D |
| 138 | B | — | A |
| 139 | A | A | A |
| 140 | A | A | A |
| 141 | A | B | A |
| 142 | B | — | B |
| 143 | B | B | B |
| 144 | A | B | B |
| 145 | B | — | A |
| 146 | B | C | B |
| 147 | C | — | C |
| 148 | C | — | B |
| 149 | A | A | A |
| 150 | C | — | C |
| 151 | B | — | B |
| 152 | B | — | A |
| 153 | A | C | A |
| 154 | B | D | A |
| 155 | A | B | A |
| 156 | C | — | C |
| 157 | B | — | C |
| 158 | A | B | A |
| 159 | A | — | B |
| 160 | B | — | D |
| 161 | A | — | B |
| 162 | B | — | C |
| 163 | A | — | B |
| 164 | A | — | A |
| 165 | A | B | A |
| 166 | A | B | A |
| 167 | A | — | A |
| 168 | A | — | A |
| 169 | A | — | B |
| 170 | A | B | A |
| 171 | B | — | B |
| 172 | C | — | D |
| 173 | B | D | D |
| 174 | A | — | A |
| 175 | C | — | D |
| 176 | C | — | D |
| 177 | C | — | D |
| | Human | Human | Human |
| Cmpd | TRPML1 | TRPML2 | TRPML3 |
| No. | EC₅₀ (nM) | EC₅₀ (nM) | EC₅₀ (nM) |
| 201 | C | B | B |
| 202 | A | A | A |
| 203 | A | A | A |
| 204 | A | B | A |
| 205 | A | — | B |
| 206 | A | B | A |
| 207 | A | B | A |
| 208 | A | C | A |
| 209 | A | A | A |
| 210 | B | — | C |
| 211 | D | — | D |
| 212 | B | — | B |
| 213 | A | — | A |
| 214 | A | D | C |
| 215 | B | B | C |
| 216 | A | — | D |
| 217 | A | — | B |
| 218 | B | — | C |
| 219 | A | — | A |
| 220 | A | B | A |
| 221 | A | — | C |
| 222 | A | — | B |
| 223 | A | D | B |
| 224 | A | — | C |
| 225 | A | — | B |
| 226 | A | — | A |
| 227 | A | — | A |
| 228 | A | — | A |
| 229 | A | D | A |
| 230 | A | A | A |
| 231 | A | A | A |
| 232 | A | A | A |
| 233 | A | A | A |
| 234 | A | B | A |
| 235 | A | B | C |
| 236 | A | — | A |
| 237 | B | D | C |
| 238 | B | — | — |
| 239 | A | C | A |
| 240 | B | B | A |
| 241 | A | — | A |
| 242 | A | B | B |
| 243 | A | — | B |
| 244 | A | — | C |
| 245 | A | — | B |
| 246 | B | C | B |
| 247 | A | C | C |
| 248 | D | — | D |
| 249 | B | — | C |
| 250 | B | D | C |
| 251 | B | C | B |
| 252 | B | C | B |
| 253 | A | C | B |
| 254 | C | — | C |
| 255 | C | D | C |
| 256 | C | D | C |
| 257 | A | B | C |
| 258 | B | C | B |
| 259 | B | C | B |
| 260 | B | D | C |
| 261 | A | D | C |
| 262 | B | C | C |
| 263 | B | C | A |
| 264 | A | C | A |
| 265 | A | B | A |
| 266 | A | B | A |
| 267 | A | B | A |
| 268 | A | C | A |
| 269 | A | A | A |
| 270 | A | B | A |
| 271 | A | A | A |
| 272 | A | B | A |
| 273 | A | — | A |
| 274 | A | — | A |
| 275 | A | — | B |
| 276 | A | B | A |
| 277 | A | C | B |
| 278 | A | B | B |
| 279 | B | C | C |
| 280 | B | C | C |
| 281 | B | C | B |
| 282 | A | A | A |
| 283 | A | B | A |

### EQUIVALENTS

It will be recognized that one or more features of any embodiments disclosed herein may be combined and/or rearranged within the scope of the invention to produce further embodiments that are also within the scope of the invention.

Those skilled in the art will recognize, or be able to ascertain using no more than routine experimentation, many equivalents to the specific embodiments of the invention described herein. Such equivalents are intended to be within the scope of the present invention.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and/or rearranged in various ways within the scope and spirit of the invention to produce further embodiments that are also within the scope of the invention. Those skilled in the art will recognize, or be able to ascertain, using no more than routine experimentation, numerous equivalents to the specific embodiments described specifically in this disclosure. Such equivalents are intended to be encompassed in the scope of the following claims.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

1. A compound of formula (Ia) 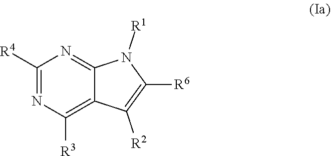
or a pharmaceutically acceptable salt thereof, wherein
R¹ is heteroaryl optionally substituted by 1-5 independently selected R⁷;
R² is aryl, heteroaryl, cycloalkyl, heterocycloalkyl, C₁₋₆ alkyl, or NR^{a}R^{b}, each R² optionally substituted by 1-5 independently selected R⁸;
R³ is 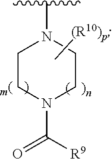
each of R⁴ and R⁶ is independently selected from the group consisting of H, hydroxy, halogen, cyano, C₁₋₆ alkyl, C₁₋₆ haloalkyl, C₁₋₆ alkoxy, C₁₋₆ haloalkoxy, C₁₋₆ alkylthio, and NR^{a}R^{b};
each of R⁷ and R⁸ are independently selected at each occurrence from the group consisting of hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇cycloalkyl is optionally substituted with substituents independently selected from the group consisting of halogen, hydroxyl, C₁₋₆ alkoxy and C₁₋₆ alkyl; or
when R¹ or R² is cycloalkyl or heterocycloalkyl, two R⁷ or two R⁸ can be taken together to form oxo, or any two R⁷ or two R⁸ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
R⁹ is C₁₋₆ alkyl or C₃₋₇ cycloalkyl, optionally substituted with 1-9 substituents independently selected from the group consisting of deuterium, halogen, C₁₋₃ alkyl, hydroxyl, and C₁₋₆ alkoxy, wherein C₁₋₃ alkyl and C₁₋₆ alkoxy are optionally substituted with 1-3 substituents independently selected from the group consisting of halogen and hydroxy;
each R¹⁰ is selected independently from the group consisting of C₁₋₆ alkyl, and C₁₋₆ haloalkyl, optionally substituted with 1-5 substituents independently selected from the group consisting of deuterium, hydroxyl, C₁₋₃ haloalkyl, C₁₋₃ alkoxy and C₁₋₃ alkyl; or
two R¹⁰ on the same carbon can be taken together to form oxo; or
any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of deuterium, halogen, hydroxyl, C₁₋₆ haloalkyl, and C₁₋₆ alkyl;
each R^{a} and R^{b} is independently selected from H, C₁₋₆ alkyl, —(CH₂)₀₋₂—C₃₋₇ cycloalkyl, and 3-7 membered heterocycloalkyl;
m is 1 or 2;
n is 1, 2, or 3;
p is 0, 1, 2, 3, 4, 5, 6, 7, or 8; and
wherein m+n is 2, 3, or 4.

2. The compound of claim 1 of formula (Ia) 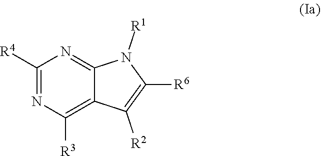
or a pharmaceutically acceptable salt thereof, wherein
R¹ is pyridyl optionally substituted by 1-5 independently selected R⁷;
R² is aryl, heteroaryl, cycloalkyl, heterocycloalkyl, C₁₋₆ alkyl, or NR^{a}R^{b}, each R² optionally substituted by 1-5 independently selected R⁸;
R³ is 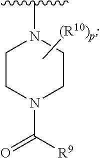
each of R⁴ and R⁶ is H;
each of R⁷ and R⁸ are independently selected at each occurrence from the group consisting of hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇cycloalkyl is optionally substituted with substituents independently selected from the group consisting of halogen, hydroxyl, C₁₋₆ alkoxy and C₁₋₆ alkyl;
R⁹ is C₁₋₆ alkyl or C₃₋₇ cycloalkyl, optionally substituted with 1-9 substituents independently selected from the group consisting of deuterium, halogen, C₁₋₃ alkyl, hydroxyl, and C₁₋₆ alkoxy, wherein C₁₋₃ alkyl and C₁₋₆ alkoxy are optionally substituted with 1-3 substituents independently selected from the group consisting of halogen and hydroxy;
each R¹⁰ is selected independently from the group consisting of C₁₋₆ alkyl, and C₁₋₆ haloalkyl, optionally substituted with 1-5 substituents independently selected from the group consisting of deuterium, hydroxyl, C₁₋₃ haloalkyl, C₁₋₃ alkoxy and C₁₋₃ alkyl; or
two R¹⁰ on the same carbon can be taken together to form oxo; or
any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of deuterium, halogen, hydroxyl, C₁₋₆ haloalkyl, and C₁₋₆ alkyl;
each R^{a} and R^{b} is independently selected from H, C₁₋₆ alkyl, —(CH₂)₀₋₂—C₃₋₇ cycloalkyl, and 3-7 membered heterocycloalkyl; and
p is 0, 1, 2, 3, 4, 5, 6, 7, or 8.

3. The compound of claim 1 of formula (Ia) 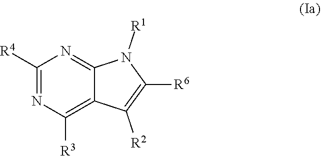
or a pharmaceutically acceptable salt thereof, wherein
R¹ is pyridyl optionally substituted by 1-5 independently selected R⁷;
R² is phenyl optionally substituted by 1-5 independently selected R⁸;
R³ is 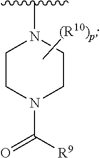
each of R⁴ and R⁶ is H;
each of R⁷ and R⁸ are independently selected at each occurrence from the group consisting of hydroxy, halogen, cyano, C₁₋₆ alkyl, and C₁₋₆ alkoxy;
R⁹ is C₁₋₆ alkyl or C₃₋₇ cycloalkyl, optionally substituted with 1-9 substituents independently selected from the group consisting of deuterium, halogen, C₁₋₃ alkyl, hydroxyl, and C₁₋₆ alkoxy, wherein C₁₋₃ alkyl and C₁₋₆ alkoxy are optionally substituted with 1-3 substituents independently selected from the group consisting of halogen and hydroxy;
each R¹⁰ is selected independently from the group consisting of C₁₋₆ alkyl, and C₁₋₆ haloalkyl, each optionally substituted with 1-5 deuterium; and
p is 0, 1, 2, 3, 4, 5, 6, 7, or 8.

4-6. (canceled)

7. The compound of claim 1, or a pharmaceutically acceptable salt thereof, wherein R¹ is pyridine, pyrimidine, pyrazine, pyridazine, thiazole, oxazole, pyrrole, imidazole, or pyrazole, optionally substituted by 1-4 independently selected R⁷.

8-10. (canceled)

11. The compound of claim 1, or a pharmaceutically acceptable salt thereof, wherein R¹ is

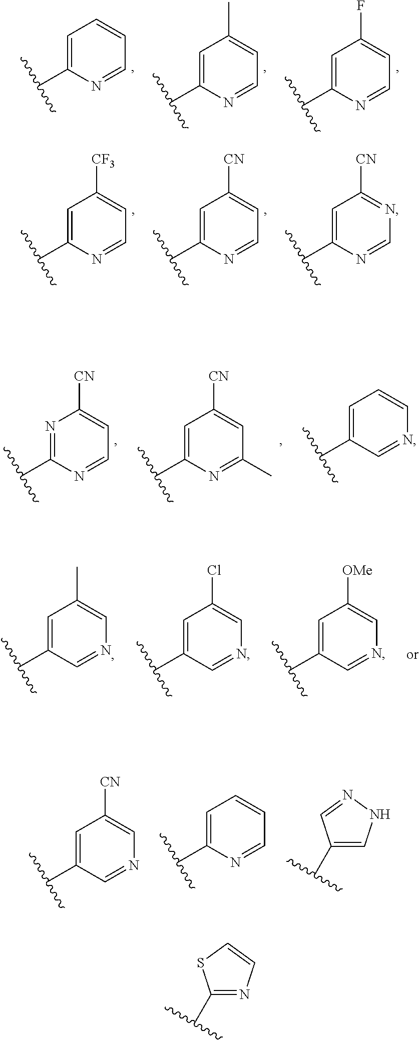

12-13. (canceled)

14. The compound of claim 1, or a pharmaceutically acceptable salt thereof, wherein R² is aryl, heteroaryl, cycloalkyl, heterocycloalkyl, C₁₋₆ alkyl, or NR^{a}R^{b}, each R² optionally substituted by 1-5 independently selected R⁸, and wherein R^{a} and R^{b} of the R² group are not both H.

15-24. (canceled)

25. The compound of claim 1, or a pharmaceutically acceptable salt thereof, wherein R² is pyridine, pyrimidine, pyrazine, pyridazine, thiazole, oxazole, pyrrole, imidazole, or pyrazole, optionally substituted by 1-4 independently selected R⁸, or cyclopropyl, cyclobutyl, cyclopentyl, or cyclohexyl each optionally substituted with 1-3 independently selected R⁸.

26-27. (canceled)

28. The compound of claim 1, or a pharmaceutically acceptable salt thereof, wherein R² is 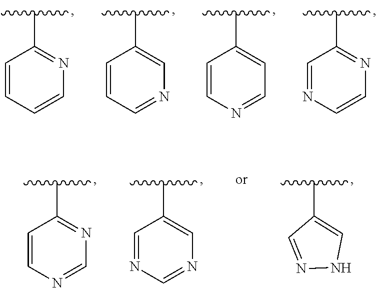
optionally substituted by 1-4 independently selected R⁸, or 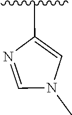 [SMILES: *c1cn(C)cn1]
and R² is not further substituted. 29. The compound of claim 28, wherein R² is 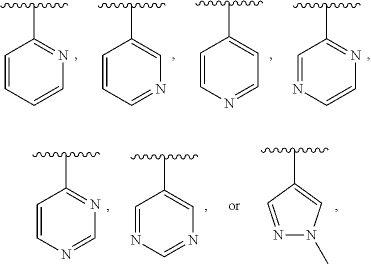
and wherein R² is not further substituted.

30. The compound of claim 28, wherein R² is

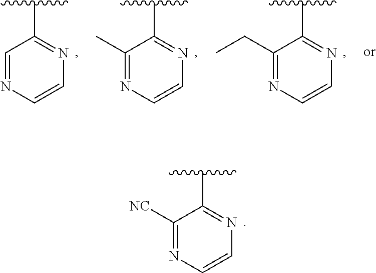

31. The compound of claim 20, wherein R² is C₃₋₈ cycloalkyl optionally substituted with 1-5 independently selected R⁸.

32. The compound of claim 20, wherein R² is C₃₋₈ cycloalkyl optionally substituted with 1-5 independently selected R⁸.

33. The compound of claim 32, wherein R² is cyclopropyl, cyclobutyl, cyclopentyl, or cyclohexyl, each optionally substituted with 1-3 independently selected R⁸.

34. The compound of any one of claims 31-33, wherein R² is not substituted.

35. The compound of claim 33, wherein R² is cyclopropyl.

36. The compound of claim 20, wherein R² is heterocycloalkyl optionally substituted by 1-5 independently selected R⁸.

37. The compound of claim 36, wherein R² is monocyclic heterocycloalkyl optionally substituted by 1-5 independently selected R⁸.

38. The compound of claim 37, wherein R² is monocyclic heterocycloalkyl of 4-6 ring atoms with 1, 2 or 3 ring atoms selected independently from N, O, and S, wherein R² is optionally substituted by 1-4 independently selected R⁸.

39. The compound of claim 1, or a pharmaceutically acceptable salt thereof, wherein R² is azetidine, oxetane, pyrrolidine, tetrahydrofuran, piperidine, piperazine, tetrahydropyran, or morpholine, optionally substituted by 1-4 independently selected R⁸.

40-49. (canceled)

50. The compound of claim 1, or a pharmaceutically acceptable salt thereof, wherein m is 1 and n is 1.

51. (canceled)

52. The compound of claim 1, or a pharmaceutically acceptable salt thereof, wherein each R¹⁰ is independently selected from the group consisting of C₁₋₆ alkyl and C₁₋₆ haloalkyl, each optionally substituted with 1-5 deuteriums.

53-54. (canceled)

55. The compound of claim 1, or a pharmaceutically acceptable salt thereof, wherein R³ is substituted with an edge fused or spiro fused cyclopropane; or R³ includes a one or two carbon bridge; and R³ is optionally additionally substituted by 1-4 R¹⁰.

56. The compound of claim 1, or a pharmaceutically acceptable salt thereof, wherein R³ is 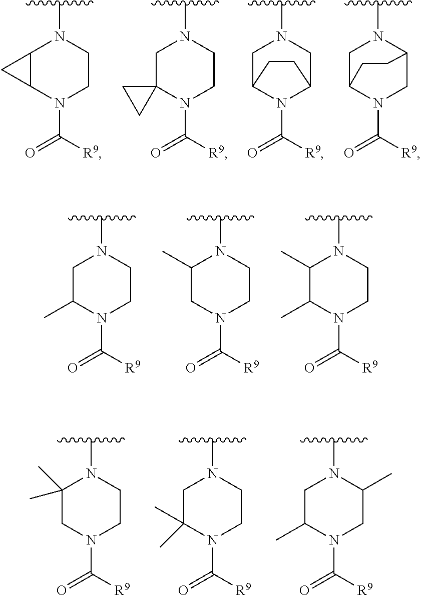
and R³ is optionally additionally substituted by 1-4 R¹⁰.

57-61. (canceled)

62. The compound of claim 1, or a pharmaceutically acceptable salt thereof, wherein R⁴ and R⁶ are H.

63-65. (canceled)

66. The compound of claim 1, or a pharmaceutically acceptable salt thereof, wherein R⁹ is t-butyl, ethyl, or isopropyl, optionally substituted with 1-9 substituents selected from deuterium, hydroxy, and halogen or with 1-3 hydroxyl.

67-73. (canceled)

74. A compound of claim 1, or a pharmaceutically acceptable salt thereof, wherein the compound achieves at least 50% of the maximal current obtained with 30 μM ML-SA1 in a patch clamp assay for TRPML1, and has an EC₅₀ less than 1 μM, or
wherein the compound achieves a maximal current obtained with 30 μM ML-SA1 in a patch clamp assay for TRPML1 which is at least 10 fold the maximal current achieved for any other TRPML.

75. (canceled)

76. A pharmaceutical composition comprising a pharmaceutically acceptable excipient and a compound of claim 1, or a pharmaceutically acceptable salt thereof.

77. A method of modulating TRPMLs, the method comprising administering to a patient in need thereof a therapeutically effective amount of a compound of claim 1, or a pharmaceutically acceptable salt thereof.

78. A method of treating a disorder which can be treated by modulation of TRPMLs or by modulation of lysosomes, the method comprising administering to a patient in need thereof a therapeutically effective amount of a compound of claim 1, or a pharmaceutically acceptable salt thereof.

79. (canceled)

80. A method of treating a disorder selected from the group consisting of a ciliopathy, neurodegenerative disorder, lysosomal storage disorder, lysosomal transport disorder, glycogen storage disorder, cholesteryl ester storage disease, a muscular disease, a disease related to aging, macular degeneration and cancer, the method comprising administering to a patient in need thereof a therapeutically effective amount of a compound of claim 1, or a pharmaceutically acceptable salt thereof.

81-93. (canceled)
